(19) United States
(12) Patent Application Publication
(10) Pub. No.: US 2004/0058903 A1
(43) Pub. Date: Mar. 25, 2004

(54) Benzamide compounds as apo b secretion inhibitors

(76) Inventors: Hisashi Takasugi, Osaka (JP); Takeshi Terasawa, Osaka (JP); Yoshikazu Inoue, Osaka (JP); Hideko Nakamura, Osaka (JP); Akira Nagayoshi, Osaka (JP); Hiroaki Ohtake, Osaka (JP); Yoshiro Furukawa, Osaka (JP); Masafumi Mikami, Osaka (JP); Kazumasa Hinoue, Osaka (JP); Makoto Ohtsubo, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/381,737

(22) PCT Filed: Sep. 28, 2001
(86) PCT No.: PCT/JP01/08581

(30) Foreign Application Priority Data
Oct. 5, 2000 (AU) PR 0583
Jul. 27, 2001 (AU) PR6666

(51) Int. Cl. A61K 31/55; A61K 31/4545; A61K 31/4439; C 07D 4 3/02

(52) U.S. Cl. 514/210.2; 514/217.04; 514/318; 514/340; 540/597; 546/194; 546/268.1; 546/276.4

(57) ABSTRACT

The present invention relates to compounds of the formula (I) wherein R₁ and R₂ are each independently lower alkyl lower alkenyl, acyl, amino, lower alkoxy, lower cycloalkyloxy, aryl, aryloxy, sulfooxy, mercapto, sulfo, hydrogen, halogen, nitro, cyano or hydroxy, or may form a ring structure; Q¹ is N or CH; L is optionally substituted unsaturated 3 to 10-membered heterocyclic group; X is optionally substituted monocyclic arylene or monocyclic heteroarylene; Y is -(A¹)ₘ-(A²)ₙ-(A⁴)ₖ-; Z is directbond, —CH2-, —NH— or —O—; and R is hydrogen or lower alkyl, or a salt thereof The compounds of the present invention inhibit apolipoprotein B (Apo B) secretion and are useful as a medicament for prophylactic and treatment of diseases or conditions resulting from elevated circulating levels of Apo B.

### TECHNICAL FIELD

This invention relates to new benzamide compounds and salts thereof which inhibit apolipoprotein B (Apo B) secretion and are useful as medicament.

### BACKGROUND ART

Apo B is the main component of lipoprotein such as VLDL (very low density lipoprotein), IDL (intermediate density lipoprotein) and LDL (low density lipoprotein). Compounds that inhibit Apo B secretion are useful for the treatment of diseases or conditions resulting from elevated circulating levels of Apo B, such as hyperlipemia, hyperlipidemia, hyperlipoproteinemia, hypercholesterolemia, hypertriglyceridemia, atherosclerosis, pancreatitis, non-insulin dependent diabetes mellitus (NIDDM), obesity and coronary heart diseases. Compounds that inhibit Apo B secretion have been described in WO96/40640, WO98/23593, WO98/56790 and WO00/32582. Compounds that inhibit Apo B secretion are also useful in reducing intestinal fat absorption, reducing food intake and treating obesity in combination with a known anti-obesity agent (EP 1 099 438, EP 1 099 439 and EP 1 099 441).

### DISCLOSURE OF INVENTION

This invention relates to new benzamide compounds.

One object of this invention is to provide the new and useful benzamide compounds and salts thereof that inhibit Apo B secretion.

A further object of this invention is to provide a pharmaceutical composition comprising said benzamide compound or a pharmaceutically acceptable, salt thereof.

Still further object of this invention is to provide a use of said benzamide compounds or pharmaceutically acceptable salts thereof as a medicament for prophylactic and therapeutic treatment of diseases or conditions resulting from elevated circulating levels of Apo B, such as hyperlipemia, hyperlipidemia, hyperlipoproteinemia, hypercholesterolemia, hypertriglyceridemia, atherosclerosis, pancreatitis, non-insulin dependent diabetes mellitus (NIDDM), obesity and coronary heart diseases.

The object benzamide compounds of the present invention are novel and can be represented by the following general formula (I)

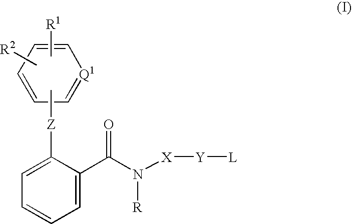

wherein

Q¹ is N or CH;

R¹ and R² are each independently lower alkyl, lower alkenyl, acyl, amino, lower alkoxy, lower cycloalkyloxy, aryl, aryloxy, sulfooxy (—O—SO₃H), mercapto or sulfo, each of which is optionally substituted by suitable substituent(s), hydrogen, halogen, nitro, cyano or hydroxy, or R¹ and R² together may form a ring structure,

L is unsaturated 3 to 10-membered heterocyclic group, which is optionally substituted by suitable substituent(s);

X is monocyclic arylene or monocyclic heteroarylene, each of which is optionally substituted by suitable substituent(s);

Y is -(A¹)ₘ-(A²)ₙ-(A⁴)ₖ-

in which

A¹ is lower alkylene or lower alkenylene, each of which is optionally substituted by suitable substituent(s),

A² is —N(R³)—, —CO—N(R³)—, —NH—CO—NH—, —CO—O—, —O—, —O—(CH₂)₂—N(R³)—, —S—, —SO— or —SO₂—, wherein R³ is hydrogen or suitable substituent(s),

A⁴ is lower alkylene, lower alkenylene or lower alkynylene, and

k, m and n are each independently 0 or 1;

Z is direct bond, —CH₂—, —NH— or —O—; and

R is hydrogen or lower alkyl,

or a salt thereof.

The preferred embodiments of the benzamide compound of the present invention represented by the general formula (I) are as follows.

(1) The benzamide compound of the general formula (I) wherein

R¹ and R² are each independently hydrogen, lower alkyl, lower alkenyl, hydroxy (lower) alkyl, lower alkanoyl, carboxy(lower)alkyl, optionally protected carboxy, lower alkylthio, lower alkylsulfonyl, halogen, trihalo(lower)alkyl, cyano, nitro, aryl, —N(R¹²) (R¹³) (wherein R¹² and R¹³ are each independently hydrogen, lower alkyl or amino protective group), hydroxy, aryloxy, lower alkylsulfonyloxy, arylsulfonyloxy, lower cycloalkyloxy, or lower alkoxy which is optionally substituted by suitable substituent(s), or R¹ and R² together may form 1,3-dioxole,

L is pyridinyl (also referred to as pyridyl), N-oxidopyridinyl, pyrimidinyl, pyrazinyl, thiazolyl, guinolinyl, isoquinolinyl, pyrazolyl, imidazolyl or benzimidazolyl, each of which is optionally substituted by suitable substituents) selected from the group consisting of lower alkyl, aryl(lower)alkyl and —(CH₂)ₛ—N(R¹⁴)(R¹⁵) (wherein R¹⁴ and R¹⁵ are each independently hydrogen, lower alkyl or amino protective group and s is 0 or 1);

X is

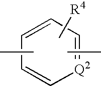

in which

Q² is N or CH, and

R⁴ is hydrogen, lower alkyl, lower alkoxy, lower alkanoyl, nitro, optionally protected amino or halogen; and

Y is -(A¹)ₘ-(A²)ₙ-(A⁴)ₖ-

in which

A¹ is lower alkylene or lower alkenylene, each of which is optionally substituted by oxo, hydroxy, hydroxy(lower)alkyl, optionally protected carboxy or optionally protected amino,

A² is —N(R³)—, —CO—N(R³)—, —NH—CO—NH—, —CO—O—, —O—, —O—(CH₂)₂—N(R³)—, —S—, —SO— or —SO₂—, wherein R³ is hydrogen, lower alkyl, pyridinyl(lower)alkyl or amino protective group,

A⁴ is lower alkylene, lower alkenylene or lower alkynylene, and

k, m and n are each independently 0 or 1,

or a salt thereof.

(2) The benzamide compound of (1) above wherein

R¹ and R² are each Independently hydrogen, lower alkyl, lower alkenyl, hydroxy(lower)alkyl, lower alkanoyl, carboxy(lower)alkyl, carboxy, lower alkoxycarbonyl, lower alkylthio, lower alkylsulfonyl, halogen, trihalo(lower)alkyl, cyano, nitro, phenyl, amino, di(lower)alkylamino, lower alkanoylamino, lower alkylsulfonylamino, aryl(lower)alkylsulfonylamino, (lower)alkoxycarbonylamino, bis[(lower)alkylsulfonyl]amino, bis[aryl(lower)alkylsulfonyl]amino, hydroxy, phenyloxy, lower alkylsulfonyloxy, tolylsulfonyloxy, lower cycloalkyloxy or lower alkoxy which is optionally substituted by suitable substituent(s) selected from the group consisting of lower alkoxy, lower alkoxycarbonyl, carboxy, halogen, hydroxy, phenyl, di(lower)alkylamino and optionally substituted carbamoyl, or R¹ and R² together may form 1,3-dioxole,

or a salt thereof.

(3) The benzamide compound of (2) above wherein

R¹ and R² are each independently hydrogen, methyl, ethyl, isopropyl, tert-butyl, vinyl, hydroxymethyl, hydroxyethyl, hydroxypropyl, formyl, acetyl, carboxymethyl, carboxyethyl, carboxy, methoxycarbonyl, methylthio, ethylthio, isopropylthio, methylsulfonyl, isopropylsulfonyl, fluoro, chloro, iodo, bromo, trifluoromethyl, cyano, nitro, phenyl, amino, dimethylamino, acetylamino, methylsulfonylamino, benzylsulfonylamino, methoxycarbonylamino, bis (methylsulfonyl) amino, bis (benzylsulfonyl) amino, hydroxy, methylsulfonyloxy, tolylsulfonyloxy, cyclohexyloxy, methoxy, ethoxy, isopropoxy, methoxyethoxy, ethoxycarbonylmethoxy, carboxymethoxy, trifluoromethoxy, trifluoroethoxy, tetrafluoropropoxy, hydroxyethoxy, phenyloxy, benzyloxy, dimethylaminoethoxy, dimethylaminopropoxy, carbamoylmethoxy, methylcarbamoylmethoxy, phenylcarbamoylmethoxy, methylsulfonylcarbamoylmethoxy or phenylsulfonylcarbamoylmethoxy, or R¹ and R² together may form 1,3-dioxole;

L is pyridinyl, N-oxidopyridinyl, pyrimidinyl, pyrazinyl, thiazolyl, quinolinyl, isoquinolinyl, pyrazolyl, imidazolyl or benzimidazolyl, each of which is optionally substituted by methyl, ethyl, amino, methylamino, formylamino, acetylamino, tert-butoxycarbonylamino, N-(tert-butoxycarbonyl)-N-methylamino, trityl, dimethylpyrrolyl or acetylaminomethyl;

X is

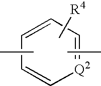

in which

Q² is N or CH, and

R⁴ is hydrogen, methyl, methoxy, nitro, amino, acetyl, acetylamino, fluoro, chloro or bromo; and

Y is direct bond or bivalent residue selected from the group consisting of

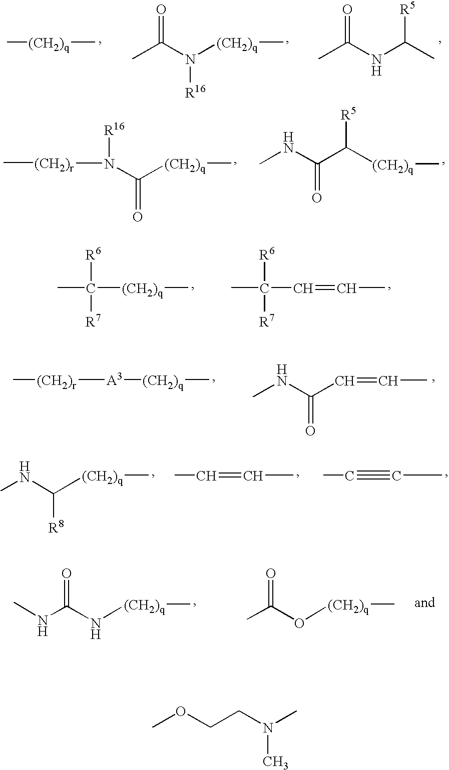

in which

A³ is —NH—, —N(CH₃)—, —N(CHO)—, —N(CH₃CO)—, —N(Boc)-,

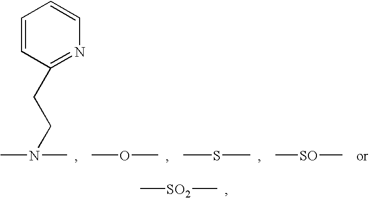

wherein Boc means tert-butoxycarbonyl,

R⁵ is methyl, amino, acetylamino or tert-butoxycarbonylamino,

R⁶ is hydroxy,

R⁷ is hydrogen, or

R⁶ and R⁷, together with the carbon atom to which they are bonded, form carbonyl,

R⁸ is hydroxymethyl or ethoxycarbonyl,

R¹⁶ is hydrogen or methyl, and

q and r are independently an integer of 0 to 3,

or a salt thereof.

In the present invention, Y represented by -(A¹)ₘ-(A²)ₙ-(A⁴)ₖ-includes a case where (A¹)ₘ is bonded to X and (A⁴)ₖ is bonded to L and a case where (A¹)ₘ is bonded to L and (A⁴)ₖ is bonded to X. That is, —X—Y-L may be —X— (A¹)ₘ-(A²)ₙ-(A⁴)ₖ-L or —X— (A⁴)ₖ-(A²)ₙ-(A¹)ₘ-L.

When A² is —CO—N(R³)—, the direction of bonding may be —CO—N(R³)— or —N(R³)—CO—. That is, —X—Y-L may be any of —X-(A¹)ₘ-CO—N(R³)-(A⁴)ₖ-L, —X-(A¹)ₘ-N(R³)—CO-(A⁴)ₖ-L —X-(A⁴)ₖ-CO—N(R³)-(A¹)ₘ-L and —X-(A⁴)ₖ-N(R³)—CO-(A¹)ₘ-R².

When A² is —CO—O—, the direction of bonding may be —CO—O—or —O—CO—. That is, —X—Y-L may be any of —X-(A¹)ₘ-CO—O-(A⁴)ₖ-L, —X-(A¹)ₘ-O—CO-(A⁴)ₖ-L, —X-(A⁴)ₖ-CO—O-(A¹)ₘ-L and —X-(A⁴)ₖ-O—CO-(A¹)ₘ-R².

Examples of a preferable group represented by Y include the following.

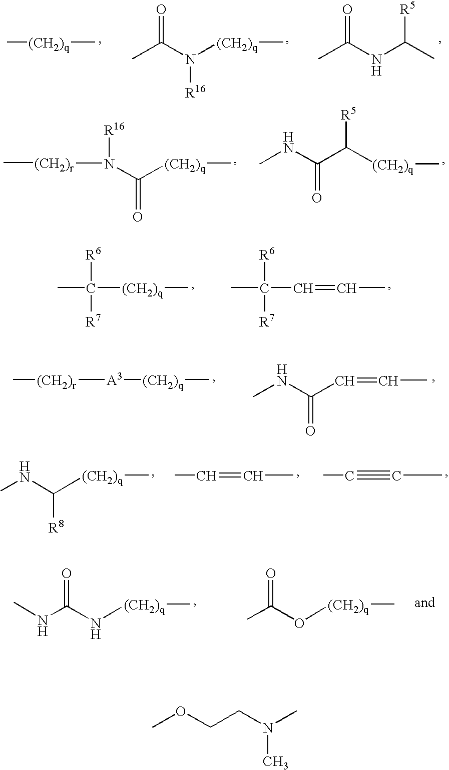

in which

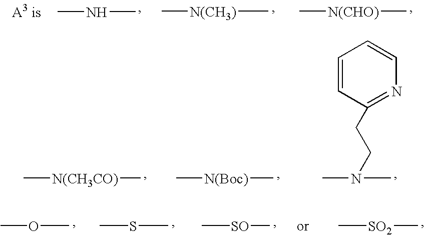

wherein Boc means tert-butoxycarbonyl,

R⁵ is methyl, amino, acetylamino or tert-butoxycarbonylamino,

R⁶ is hydroxy,

R⁷ is hydrogen, or

R⁶ and R⁷, together with the carbon atom to which they are bonded, form carbonyl,

R⁸ is hydroxymethyl or ethoxycarbonyl,

R¹⁶ is hydrogen or methyl, and

q and r are independently an integer of 0 to 3.

Examples of a preferable group represented by —X—Y-L include the following.

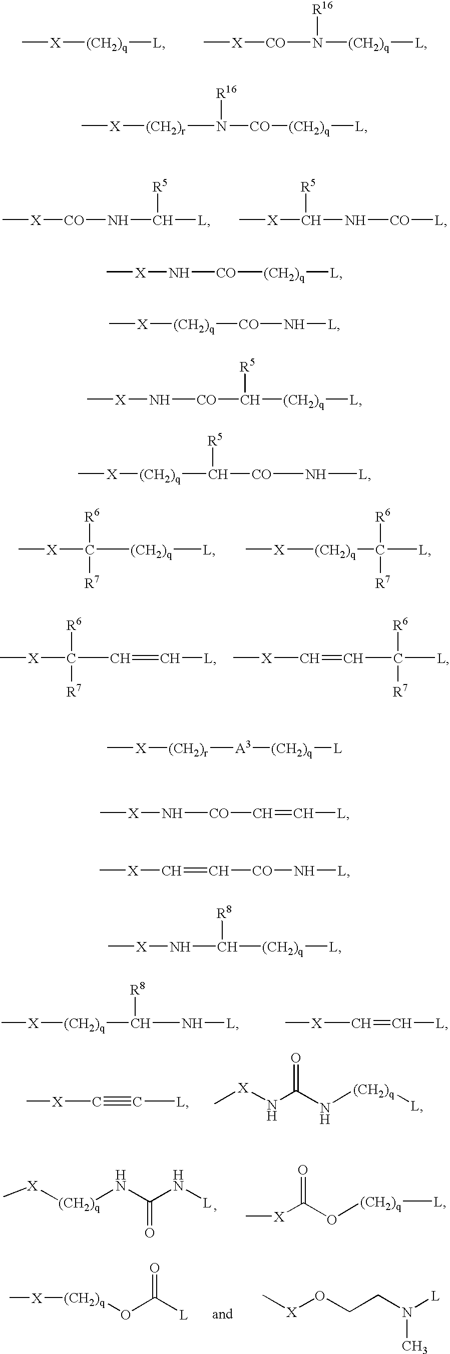

wherein X, R⁵, R⁶, R⁷, R⁸, R¹⁶, A³, L, r and q are as defined above.

More preferred embodiment of the benzamide compound of the present invention is as follows.

(A) A compound of the formula (I′)

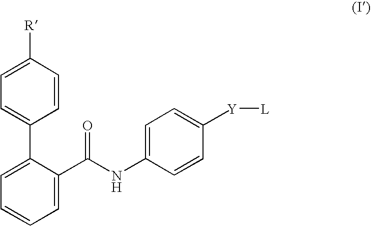

wherein

R′ is methyl or trifluoromethyl;

Y is —CH₂—, —(CH₂)₂—, —(CH₂)₃—, —NH—(CH₂)₂—, —O—(CH₂)₂—, —NH—CO—CH₂—, —CO—NH—CH₂— or —CO—NH—(CH₂)₂—; and

L is pyridinyl or thiazolyl, each of which is optionally substituted by methyl or amino,

or a salt thereof.

(B) The compound of (A) above, wherein

Y is —(CH₂)₃—, —NH—(CH₂)₂—, —O—(CH₂)₂—, —NH—CO—CH₂— or —CO—NH—CH₂—; and

L is pyridinyl aminopyridinyl, thiazolyl or aminothiazolyl,

or a salt thereof.

(C) The compound of (B) above, which is selected from the group consisting of

N-{4-[3-(2-pyridinyl)propyl]phenyl}-4′-(trifluoromethyl)-1,1′-biphenyl-2-carboxamide (Example 25),

N-{4-[3-(6-amino-2-pyridinyl)propyl)phenyl}-4′-(trifluoromethyl)-1,1′-biphenyl-2-carboxamide (Example 44),

N-[4-({[4′-(trifluoromethyl)-1,1′-biphenyl-2-yl]carbonyl}amino)benzyl]-2-pyridinecarboxamide (Example 53),

N-(4-{[(4′-methyl-1,1′-biphenyl-2-yl)carbonyl]amino}benzyl)-2-pyridinecarboxamide (Example 56),

N-(4-{[2-(2-pyridinyl)ethyl]amino}phenyl)-4′-(trifluoromethyl)1,1′-biphenyl-2-carboxamide (Example 59),

N-(4-[(2-pyridinylacetyl)amino]phenyl]-4′-(trifluoromethyl)-1,1′-biphenyl-2-carboxamide (Example 65),

4′-methyl-N-(4-{[2-(2-pyridinyl)ethyl]amino}phenyl)-1,1′-biphenyl-2-carboxamide (Example 68),

N-{4-[2-(2-pyridinyl)ethoxy]phenyl}-4′-(trifluoromethyl)-1,1′-biphenyl-2-carboxamide (Example 73),

N-(4-{[2-(2-amino-1,3-thiazol-4-yl)ethyl]amino}phenyl)-4′-(trifluoromethyl)-1,1′-biphenyl-2-carboxamide (Example 75),

N-(4-{[2-(6-amino-2-pyridinyl)ethyl]amino}phenyl)-4′-(trifluoromethyl)-1,1′-biphenyl-2-carboxamide (Example 77),

N-{4-[2-(2-amino-1,3-thiazol-4-yl)ethoxy]phenyl}-4′-(trifluoromethyl)-1,1′-biphenyl-2-carboxamide (Example 79),

N-{4-[2-(6-amino-2-pyridinyl)ethoxy]phenyl}-4,′-(trifluoromethyl)-1,1′-biphenyl-2-carboxamide (Example 81),

N-(4-{[2-(1,3-thiazol-4-yl)ethyl]amino}phenyl)-4′-(trifluoromethyl)-1,1′-biphenyl-2-carboxamide (Example 83),

N-(4-{[(6-amino-2-pyridinyl)acetyl]amino}phenyl)-4′-(trifluoromethyl)-1,1′-biphenyl-2-carboxamide (Example 175),

N-(4-{[2-(6-amino-2-pyridinyl)ethyl]amino}phenyl)-4′-methyl-1,1′-biphenyl-2-carboxamide (Example 189),

N-(4-{[(2-amino-1,3-thiazol-4-yl)acetyl]amino}phenyl)-4′-(trifluoromethyl)-1,1′-biphenyl-2-carboxamide (Example 195),

N-{4-[(1,3-thiazol-4-ylacetyl)amino]phenyl}-4′-(trifluoromethyl)-1,1′-biphenyl-2-carboxamide (Example 200),

N-(4-{[2-(2-amino-1,3-thiazol-4-yl)ethyl]amino}phenyl)-4′-methyl-1,1′-biphenyl-2-carboxamide (Example 211), and

N-{4-[2-(1,3-thiazol-4-yl)ethoxy]phenyl}-4′-(trifluoromethyl)-1,1′-biphenyl-2-carboxamide (Example 221), or a salt thereof.

Suitable salts of the object compound (I) may be pharmaceutically acceptable salts such as conventional non-toxic salts and include, for example, a salt with a base or an acid addition salt such as a salt with an inorganic base, for example, an alkali metal salt (e.g., sodium salt, potassium salt, etc.), an alkaline earth metal salt (e.g., calcium salt, magnesium salt, etc.), an ammonium salt; a salt with an organic base, for example, an organic amine salt (e.g., triethylamine salt, pyridine salt, picoline salt, ethanolamine salt, triethanolamine salt, dicyclohexylamine salt, N,N′-dibenzylethylenediamine salt, etc.); an inorganic acid addition salt (e.g., hydrochloride, hydrobromide, sulfate,, phosphate, etc.); an organic carboxylic or sulfonic acid addition salt (e.g., formate, acetate, trifluoroacetate, maleate, tartrate, citrate., fumarate, methanesulfonate, benzenesulfonate, toluenesulfonate, etc.); and a salt with a basic or acidic amino acid (e.g., arginine, aspartic acid, glutamic acid, etc.).

In the above and subsequent descriptions of the present specification, suitable examples and illustration of the various definitions which the present invention intends to include within the scope; thereof are explained in detail as follows.

The term “lower” is used to intend a group having 1 to 6, preferably 1 to 4, carbon atom(s), unless otherwise provided.

Suitable “lower alkyl” and “lower alkyl” moiety in the terms “hydroxy(lower)alkyl”, “carboxy(lower)alkyl”, “lower alkylthio”, “lower alkylsulfonyl”, “trihalo(lower)alkyl”, “lower alkylamino”, “di(lower)alkylamino”, “lower alkylsulfonylamino”, “aryl (lower)alkylsulfonylaminoo”, “bis[(lower)alkylsulfonyl]amino”, “bis[aryl (lower) alkylsulfonyl) amino”, “lower alkylsufonyloxy”, “N-(lower) alkanoyl-N-(lower) alkylamino”, “N-(lower) alkylsulfonyl-N-(lower)alkylamino”, “N-aryl(lower)alkylsulfonyl-N-(lower)alkylamino”, “N-(lower)alkoxycarbonyl-N-(lower)alkylamino”, “lower alkylcarbamoyl”, “lower alkylsulfonylcarbamoyl” and “aryl(lower)alkyl” include straight or branched one having 1 to 6 carbon atom(s), such as methyl, ethyl, propyl, isopropyl, butyl, isobutyl, sec-butyl, tert-butyl, pentyl, tert-pentyl and hexyl, in which more preferred one is C₁-C₄ alkyl.

Suitable “lower alkenyl” includes straight or branched alkenyl having 2 to 6 carbon atom(s), such as vinyl, 1-propenyl, 2-propenyl, isopropenyl, 1-butenyl, 2-butenyl, 3-butenyl, 2-methyl-2-propenyl, 1-pentenyl, 2-pentenyl, 3-pentenyl, 4-pentenyl, 1-hexenyl, 2-hexenyl, 3-hexenyl, 4-hexenyl and 5-hexenyl, in which more preferred one is C₂-C₄ alkenyl, and most preferred one is vinyl.

Suitable “acyl” includes lower alkanoyl and optionally protected carboxy.

Suitable “lower alkanoyl” and “lower alkanoyl” moiety in the terms “lower alkanoylamino” and “N-(lower)alkanoyl-N-(lower)alkylamino” include alkanoyl having 1 to 6 carbon atom(s) such as formyl, acetyl, propionyl, butyryl, isobutyryl, valeryl, isovaleryl, pivaloyl and hexanoyl, in which more preferred one is C₁-C₄ alkanoyl.

Suitable, “lower cycloalkoxy” includes cycloalkoxy having 3 to 7 carbon atoms, such as cyclopropoxy, cyclobutoxy, cyclopentyloxy, cyclohexyloxy and cycloheptyloxy, in which more preferred one is cyclohexyloxy.

Suitable “lower alkoxy” and “lower alkoxy” moiety in the terms “lower alkoxycarbonyl”, “(lower)alkoxycarbonylamino” and “N-(lower)alkoxycarbonyl-N-(lower)alkylamino” include straight or branched alkoxy having 1 to 6 carbon atom(s), such as methoxy, ethoxy, propoxy, isopropoxy, butoxy, isobutoxy, sec-butoxy, tert-butoxy, pentyloxy, tert-pentyloxy and hexyloxy, in which more preferred one is C₁-C₄ alkoxy.

Suitable “aryl” and “aryl” moiety in the terms “arylsulfonyloxy”, “aryl(lower)alkylsulfonylamino”, “bis[aryl(lower)alkylsulfonyl]amino”, “N-aryl(lower)alkyl-sulfonyl-N-(lower) alkylamino”, “arylcarbamoyl”, “arylsulfonylcarbamoyl”, “aryloxy” and “aryl(lower)alkyl” include aryl having 6 to 10 carbon atoms which is optionally substituted by suitable subtituent such as lower alkyl. Suitable examples of aryl moiety include phenyl, tolyl and naphthyl, in which more preferred ones are phenyl and tolyl.

Suitable “aryloxy” includes phenyloxy, tolyloxy and naphthyloxy, in which more preferred one is phenyloxy.

“Lower alkyl, lower alkenyl, acyl, amino, lower alkoxy, lower cycloalkyloxy, aryl, aryloxy, sulfooxy, mercapto or sulfo” at R¹ is optionally substituted by suitable substituent(s). Suitable examples of such substituent include halogen, hydroxy, carboxy, lower alkoxy, lower alkyl, amino protective group, lower alkoxycarbonyl, phenyl, optionally protected amino, optionally substituted carbamoyl and aryl.

Suitable “lower alkyl which is optionally substituted by suitable substituent(s)” includes lower alkyl optionally substituted by suitable substituent(s), preferably 1 to 3 substituents, selected from the group consisting of hydroxy, carboxy and halogen.

Suitable “hydroxy(lower)alkyl” includes hydroxymethyl, 2-hydroxyethyl, 1-hydroxyethyl, 3-hydroxypropyl, 2-hydroxypropyl, 1-hydroxypropyl and 4-hydroxybutyl.

Suitable “carboxy (lower) alkyl” includes carboxymethyl, 2-carboxyethyl, 1-carboxyethyl, 3-carboxypropyl, 2-carboxypropyl, 1-carboxypropyl and 4-carboxybutyl.

Suitable “acyl which is optionally substituted by suitable substituent(s)” includes lower alkanoyl (as defined above) and optionally protected carboxy such as carboxy and lower alkoxycarbonyl.

Suitable “lower alkoxycarbonyl” includes methoxycarbonyl, ethoxycarbonyl, propoxycarbonyl, isopropoxycarbonyl, butoxycarbonyl, isobutoxycarbonyl and tert-butoxycarbonyl.

Suitable “amino which is optionally substituted by suitable substituent(s)” includes —N(R¹²) (R¹³) wherein R¹² and R¹³ are each independently hydrogen, lower alkyl or amino protective group.

“Lower-alkoxy which is optionally substituted by suitable substituent(s)” includes lower alkoxy optionally substituted by suitable substituent(s), preferably 1 to 5 substituents, more preferably 1 to 3 substituents, selected from the group consisting of lower alkoxy, lower alkoxycarbonyl, carboxy, halogen, hydroxy, phenyl, optionally protected amino and optionally substituted carbamoyl.

Suitable examples of “optionally substituted carbamoyl” include carbamoyl, lower alkylcarbamoyl (e.g., methylcarbamoyl), arylcarbamoyl (e.g., phenylcarbamoyl), lower alkylsufonylcarbamoyl (e.g., methylsulfonylcarbamoyl) and arylsulfonylcarbamoyl (e.g., phenylsulfonylcarbamoyl).

Suitable examples of “lower alkoxy which is optionally substituted by suitable substituent(s)” includes lower alkoxy (e.g., methoxy, ethoxy, isopropoxy), (lower)alkoxy(lower)alkoxy (e.g., methoxyethoxy), lower alkoxycarbonyl(lower)alkoxy (e.g., ethoxycarbonylmethoxy), trihalo(lower)alkoxy (e.g., trifluoromethoxy, trifluoroethoxy), tetrahalo(lower)alkoxy (e.g., tetrafluoropropoxy), hydroxy(lower)alkoxy (e.g., hydroxyethoxy), phenyl(lower)alkoxy (e.g., benzyloxy), optionally protected amino(lower)alkoxy (e.g., dimethylaminoethoxy, dimethylaminopropoxy), optionally substituted carbamoyl(lower)alkoxy (e.g., carbamoylmethoxy, methylcarbamoylmethoxy, phenylcarbanoylmethoxy, methylsulfonylcarbamoylmethoxy, phenylsulfonylcarbamoylmethoxy), and carboxy(lower)alkoxy (e.g., carboxymethoxy).

Suitable “sulfooxy which is optionally substituted by suitable substituent(s)” includes sulfooxy and lower alkylsulfonyloxy and arylsulfonyloxy.

Suitable “lower alkylsulfonyloxy” includes methylsulfonyloxy, ethylsulfonyloxy, propylsulfonyloxy, isopropylsulfonyloxy, butylsulfonyloxy, isobutylsulfonyloxy, sec-butylsulfonyloxy, tert-butylsulfonyloxy, pentylsulfonyloxy and hexylsulfonyloxy, in which more preferred one is methylsulfonyloxy.

Suitable “arylsulfonyloxy” includes phenylsulfonyloxy and tolylsulfonyloxy (e.g., o-tolylsulfonyloxy, m-tolylsulfonyloxy, p-tolylsulfonyloxy), in which more preferred one is tolylsulfonyloxy.

Suitable “mercapto which is optionally substituted by suitable substituent(s)” includes-mercapto and lower alkylthio.

Suitable “lower alkylthio” includes methylthio, ethylthio, propylthio, isopropylthio, butylthio, isobutylthio, sec-butylthio, tert-butylthio, pentylthio and hexylthio.

Suitable “sulfo which is optionally substituted by suitable substituent(s)” includes sulfo and lower alkylsulfonyl.

Suitable “lower alkylsulfonyl” includes methylsulfonyl, ethylsulfonyl, propylsulfonyl, isopropylsulfonyl, butylsulfonyl, isobutylsulfonyl, sec-butylsulfonyl, tert-butylsulfonyl, pentylsulfonyl and hexylsulfonyl.

Suitable “halogen” and “halogen” moiety in the terms “trihalo(lower)alkyl”, “trihalo(lower)alkoxy” and “tetrahalo(lower)alkoxy” include, for example, fluorine, bromine, chlorine and iodine.

Suitable “trihalo (lower) alkyl” includes trifluoromethyl, trichloromethyl and tribromomethyl, in which more preferred one is trifluoromethyl.

Suitable examples of a ring structure formed by R¹ and R² include 1,3-dioxole.

Suitable “unsaturated 3 to 10-membered heterocyclic group” includes unsaturated 3 to 10-membered heteromonocyclic or fused heterocyclic group, and preferably include

5 or 6-membered aromatic heteromonocyclic group containing I to 4 heteroatom(s) selected from sulfur, oxygen and nitrogen such as pyridinyl (also referred to as pyridyl), N-oxidopyridinyl, pyrimidinyl, pyrazinyl, pyridazinyl, thiazolyl, isothiazolyl, thiadiazolyl, oxazolyl, isoxazolyl, imidazolyl, pyrazolyl, furanyl, thienyl and pyrrolyl; and

8 to 10-membered aromatic fused heterocyclic group containing 1 to 4 heteroatom(s) selected from sulfur, oxygen and nitrogen such as quinolinyl, isoquinolinyl, purinyl and benzimidazolyl.

Suitable examples of “unsaturated 3 to 10-membered heterocyclic group” include pyridinyl, N-oxidopyridinyl, pyrimidinyl, pyrazinyl, thiazolyl, imidazolyl, pyrazolyl, pyrrolyl, quinolinyl, isoquinolinyl, purinyl and benzimidazolyl, and more preferred one is pyridinyl.

“Unsaturated 3 to 10-membered heterocyclic group” at L is optionally substituted by suitable substituent(s). Suitable examples of such substituent include lower alkyl, aryl(lower)alkyl and —(CH₂)ₛ—N(R¹⁴) (R¹⁵) (wherein R¹⁴ and R¹⁵ are each independently hydrogen, lower alkyl or amino protective group and s is 0 or 1).

Suitable “aryl(lower)alkyl” includes mono(or di or tri)phenyl(lower)alkyl (e.g., benzyl, phenethyl, benzhydryl, trityl, etc.), in which more preferred one is mono(or di or tri)phenyl (C₁-C₄) alkyl.

Suitable “monocyclic arylene” includes phenylene (e.g., 1,4-phenylene, 1,3-phenylene, 1,2-phenylene).

“Monocyclic heteroarylene” means bivalent aromatic heteromonocyclic group, in which more preferred one is bivalent 5 or 6-membered aromatic heteromonocyclic group containing 1 to 3 heteroatom(s) selected from sulfur, oxygen and nitrogen. Suitable examples of monocyclic heteroarylene include pyridinediyl (e.g., pyridine-2,5-diyl), pyrimidinediyl, pyrazinediyl, pyridazinediyl, thiazolediyl, isothiazolediyl, oxazolediyl, isoxazolediyl, imidazolediyl, pyrazolediyl, furandiyl, thiophenediyl and pyrrolediyl, in which more preferred one is pyridinediyl.

“Monocyclic arylene” and “monocyclic heteroarylene” are optionally substituted by suitable substituent(s), preferably by 1 to 3 substituents. Suitable examples of such substituent include lower alkyl, lower alkoxy, lower alkanoyl, nitro, optionally protected amino and halogen.

Suitable “lower alkylene” includes straight or branched alkylene having 1 to 6 carbon atoms, such as methylene, ethylene, trimethylene, tetramethylene, propylene, ethylidene and propylidene, in which more preferred one is C₁-C₃ alkylene.

Suitable “lower alkenylene” includes straight or branched alkenylene having 2 to 6 carbon atoms, such as —CH═CH—, —CH═CH—CH₂—, —CH₂—CH═CH—, —CH═CH—CH₂—CH₂—, —CH₂—CH═CH—CH₂—, —CH₂—CH₂—CH═CH—, —CH═CH—CH(CH₃)— and —CH(CH₃)—CH═CH—, in which more preferred one is C₂-C₄ alkenylene.

Suitable “lower alkynylene” includes straight or branched alkynylene having 2 to 6 carbon atoms, such as —C≡C—, —C≡C—CH₂—, —CH₂—C≡C—, —C≡C—CH₂—CH₂—, —CH₂—C≡CH₂—, —CH₂—CH₂—C≡C—, —C≡C—CH(CH₃)— and —CH(CH₃)—C≡C—, in which more preferred one is C₂-C₄ alkynylene, and most preferred one is —C≡C—.

“Lower alkylene or lower alkenylene” at A¹ is optionally substituted by suitable substituent(s). Suitable examples of such substituent include oxo, hydroxy, hydroxy(lower)alkyl, optionally protected carboxy or optionally protected amino.

Suitable examples of “amino protective group” include acyl such as lower alkanoyl (e.g., formyl, acetyl, etc.), lower alkoxycarbonyl (e.g., tert-butoxycarbonyl, etc.), mono(or di or tri)phenyl(lower)alkoxy carbonyl (e.g., benzyloxycarbonyl, etc.), and a conventional protective group such as mono(or di or tri)aryl(lower)alkyl, for example, mono(or di or tri)phenyl(lower)alkyl (e.g., benzyl, trityl, etc.), lower alkylsulfonyl (e.g., methylsulfonylanino, etc.), aryl(lower)alkylsulfonyl (e.g., benzylsulfonyl, etc.) and

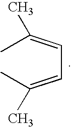

“Optionally protected amino” include amino and protected amino. Suitable examples of protected amino include lower alkanoylamino, lower alkylsulfonylamino, aryl (lower) alkylsulfonylamino, (lower) alkoxycarbonylamino, bis[(lower)alkylsulfonyl]amino, bis[aryl(lower)alkylsulfonyl]amino and

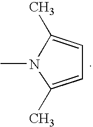

Suitable examples of —N(R¹²) (R¹³) and —N(R¹⁴) (R¹⁵) include amino, lower alkylamino, di(lower)alkylamino, lower alkanoylamino, lower alkylsulfonylamino, aryl(lower)alkylsulfonylamino, (lower)alkoxycarbonylamino, bis[(lower)alkylsulfonyl]amino, bis[aryl(lower)alkylsulfonyl]amino, N-(lower)alkanoyl-N-(lower)alkylamino, N-(lower)alkylsulfonyl-N-(lower)alkylamino, N-aryl(lower)alkylsulfonyl-N-(lower)alkylamino and N-(lower)alkoxycarbonyl-N-(lower)alkylamino.

Suitable “lower alkylamino” includes methylamino, ethylamino, propylamino, isopropylamino, butylamino, isobutylamino, sec-butylamino, tert-butylamino, pentylamino and hexylamino, in which more preferred one is methylamino.

Suitable “di (lower) alkylamino” includes dimethylamino, diethylamino, dipropylamino, diisopropylamino, dibutylamino, dipentylaamino, dihexylamino, ethylmethlylamino, methylpropylamino, and ethylpropylamino, in which more preferred one is dimethylamino.

Suitable “lower alkanoylamino” includes formylamino, acetylamino, propionylamino, butyrylamino, isobutyrylamino, valerylamino, isovalerylamino, pivaloylamino and hexanoylamino, in which more preferred ones are formylamino and acetylamino.

Suitable “lower alkylsulfonylamino” includes methylsulfonylamino, ethylsulfonylamino, propylsulfonylamino, isopropylsulfonylamino, butylsulfonylamino, isobutylsulfonylamino, sec-butylsulfonylamino, tert-butylsulfonylamino, pentylsulfonylamino and hexylsulfonylamino, in which more preferred one is methylsulfonylamino.

Suitable “aryl(lower)alkylsulfonylamino” includes benzylsulfonylamino, phenylethylsulfonylamino and phenylpropylsulfonylamino, in which more preferred one is benzylsulfonylamino.

Suitable “(lower)alkoxycarbonylamino” includes methoxycarbonylamino, ethoxycarbonylamino, propoxycarbonylamino, isopropoxycarbonylamino, butoxycarbonylamino, isobutoxycarbonylamino, sec-butoxycarbonylamino, tert-butoxycarbonylamino, pentyloxycarbonylamino, tert-pentyloxycarbonylamino and hexyloxycarbonylamino, in which more preferred ones are methoxycarbonylamino and tert-butoxycarbonylamino.

Suitable “bis[(lower)alkylsulfonyl]amino” includes bis(methylsulfonyl)amino, bis(ethylsulfonyl)amino, bis (propylsulfonyl) amino, bis (isopropylsulfonyl) amino, bis (butylsulfonyl)-amino, bis (isobutylsulfonyl) amino, bis (sec-butylsulfonyl)amino, bis(tert-butylsulfonyl)amino, bis(pentylsulfonyl)amino and bis(hexylsulfonyl)amino, in which more preferred one is bis(methylsulfonyl)amino.

Suitable “bis[aryl(lower)ialkylsulfonyl]amino” includes bis(benzylsulfonyl)amino, bis(phenylethylsulfonyl)amino and bis(phenylpropylsulfonyl)amino, in which more preferred one is bis (benzylsulfonyl) amino.

Suitable “N-(lower)alkanoyl-N-(lower)alkylamino” includes N-formyl-N-methylamino, N-acetyl-N-methylamino, N-methyl-N-propionylamino, N-butyryl-N-methylamino, N-isobutyryl-N-methylamino, N-methyl-N-valerylamino, N-isovaleryl-N-methylamino, N-methyl-N-pivaloylamino and N-hexanoyl-N-methylamino, in which more preferred ones are N-formyl-N-methylamino and N-acetyl-N-methylamino.

Suitable “N-(lower)alkylsulfonyl-N-(lower)alkylamino” includes N-methylsulfonyl-N-methylamino, N-ethylsulfonyl-N-methylamino, N-methyl-N-propylsulfonylamino, N-isopropylsulfonyl-N-methylamino, N-butylsulfonyl-N-methylamino, N-isobutylsulfonyl-N-methylamino, N-(sec-butylsulfonyl)-N-methylamino, N-(tertbutylsulfonyl)-N-methylamino, N-methyl-N-pentylsulfonylamino and N-hexylsulfonyl-N-methylamino, in which more preferred one is N-methylsulfonyl-N-methylamino.

Suitable “N-aryl(lower)alkylsulfonyl-N-(lower)alkylamino” includes N-benzylsulfonyl-N-methylamino, N-methyl-N-phenylethylsulfonylamino and N-methyl-N-phenylpropylsulfonylamino, in which more preferred one is N-benzylsulfonyl-N-methylamino.

Suitable “N-(lower)alkoxycarbonyl-N-(lower)alkylamino” includes N-methoxycarbonyl-N-methylamino, N-ethoxycarbonyl-N-methylamino, N-methyl-N-propoxycarbonylamino, N-isopropoxycarbonyl-N-methylamino, N-butoxycarbonyl-N-methylamino, N-isobutoxycarbonyl-N-methylamino, N-(sec-butoxycarbonyl)-N-methylamino, N-(tert-butoxycarbonyl)-N-methylamino, N-methyl-N-pentyloxycarbonylamino, N-methyl-N-(tert-pentyloxycarbonyl)amino and N-hexyloxycarbonyl-N-methylamino, in which more preferred ones are N-methoxycarbonyl-N-methylamino and N-(tert-butoxycarbonyl)-N-methylamino.

Suitable examples of “carboxy protective group” include lower alkyl (e.g., methyl, ethyl, tert-butyl, etc.) and mono(or di or tri)phenyl(lower)alkyl optionally substituted by nitro (e.g., benzyl, 4-nitrobenzyl, benzhydryl, trityl, etc.).

“Optionally protected carboxy” include carboxy and protected carboxy. Suitable examples of protected carboxy include lower alkoxycarbonyl (e.g., methoxycarbonyl, ethoxycarbonyl, propoxycarbonyl, isopropoxycarbonyl, butoxycarbonyl, isobutoxycarbonyl, tert-butoxycarbonyl, etc.) and mono(or di or tri)phenyl(lower)alkoxycarbonyl optionally substituted by nitro (e.g., benzyloxycarbonyl, 4-nitrobenzyloxycarbonyl, benzhydryloxycarbonyl, trityloxycarbonyl, etc.).

The object compound (I) of the present invention can be prepared by the following processes.

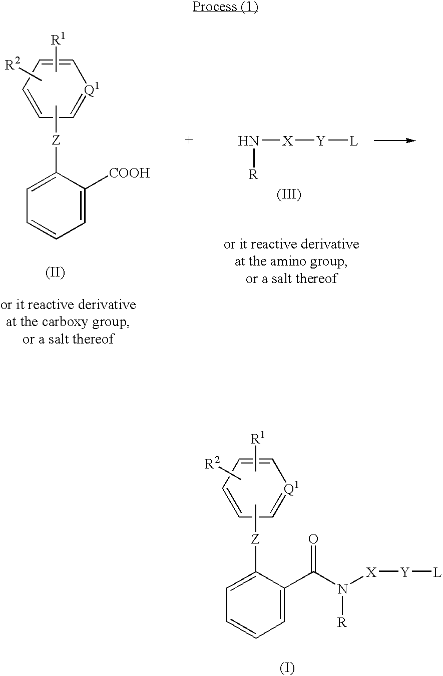

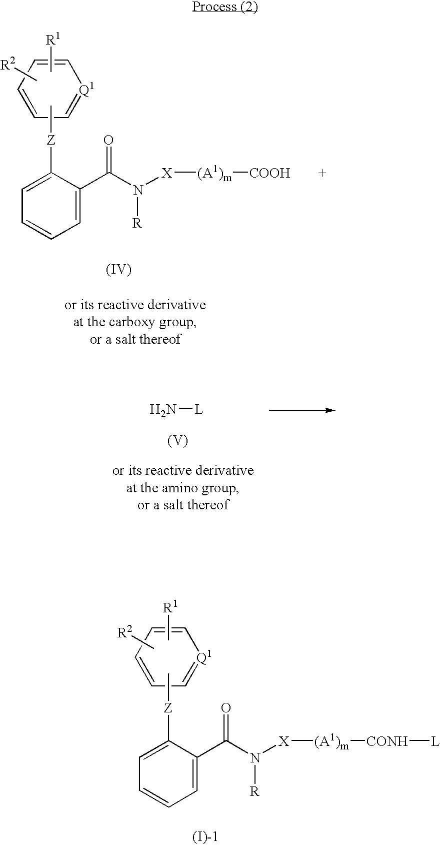

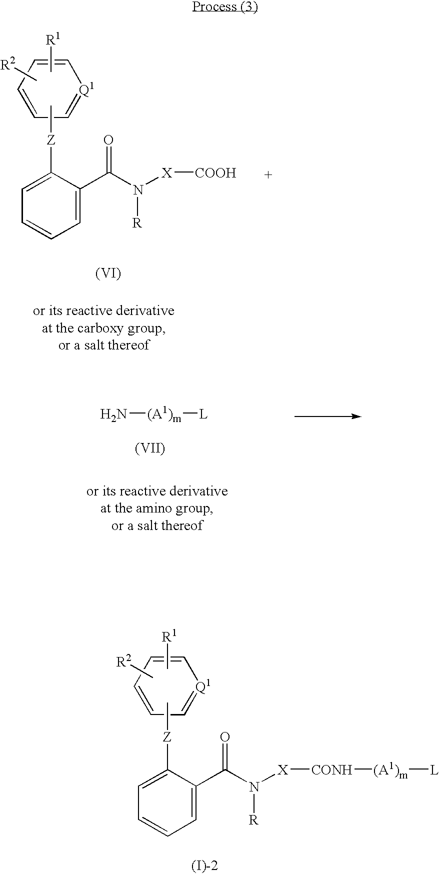

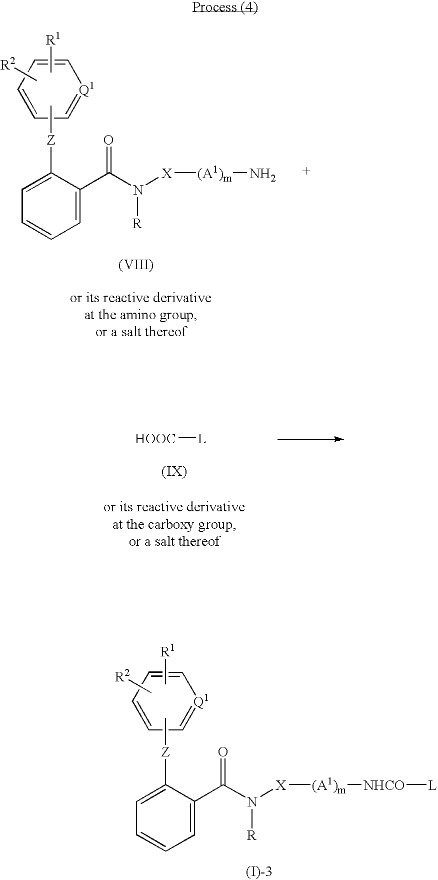

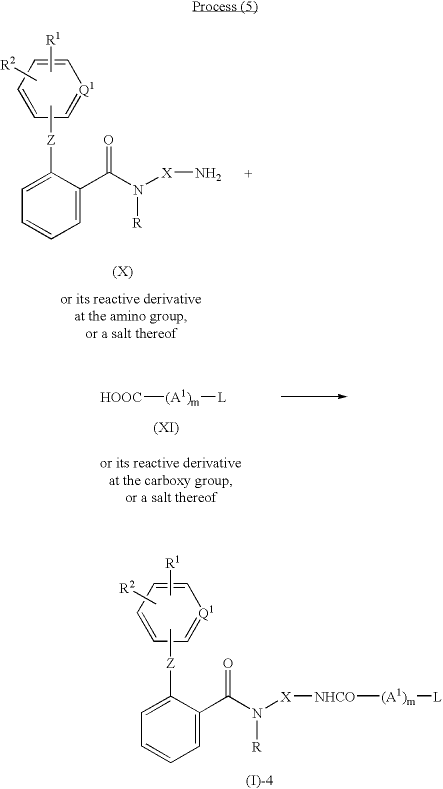

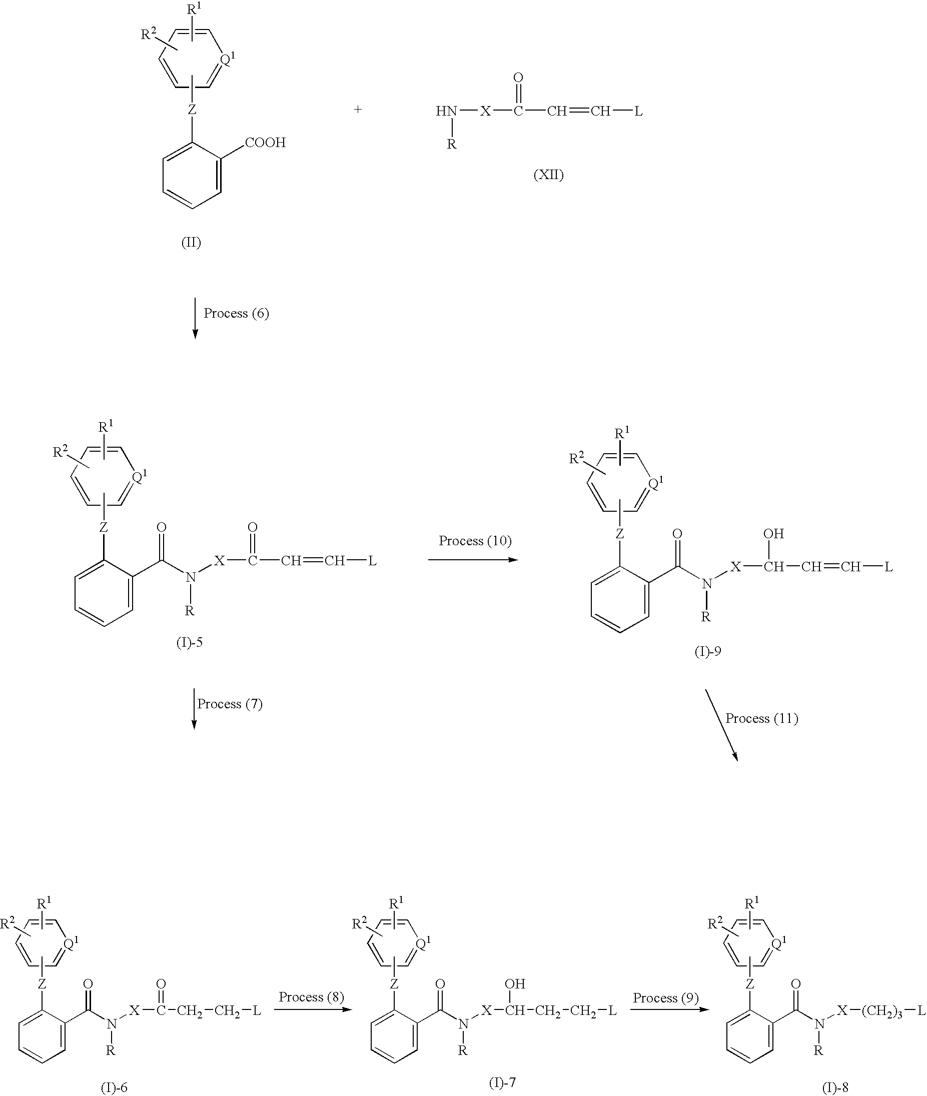

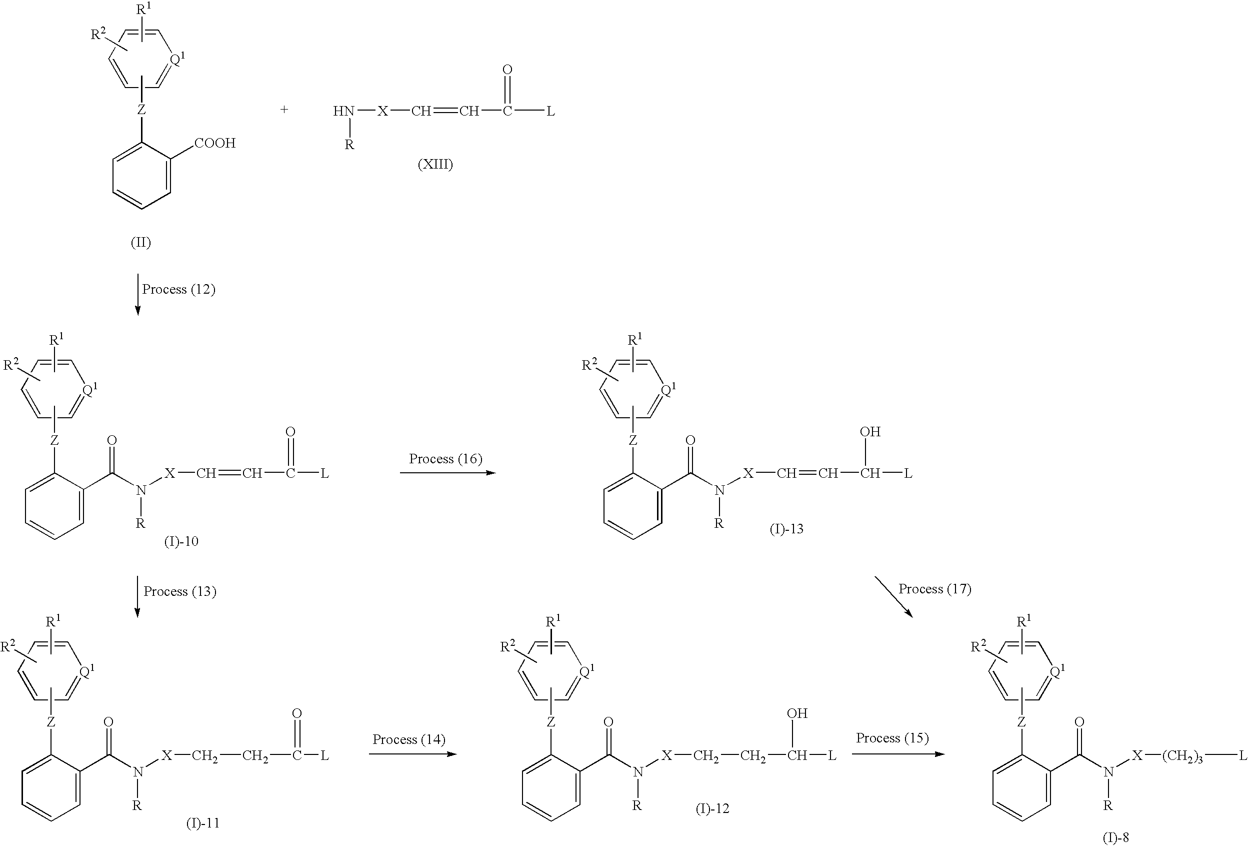

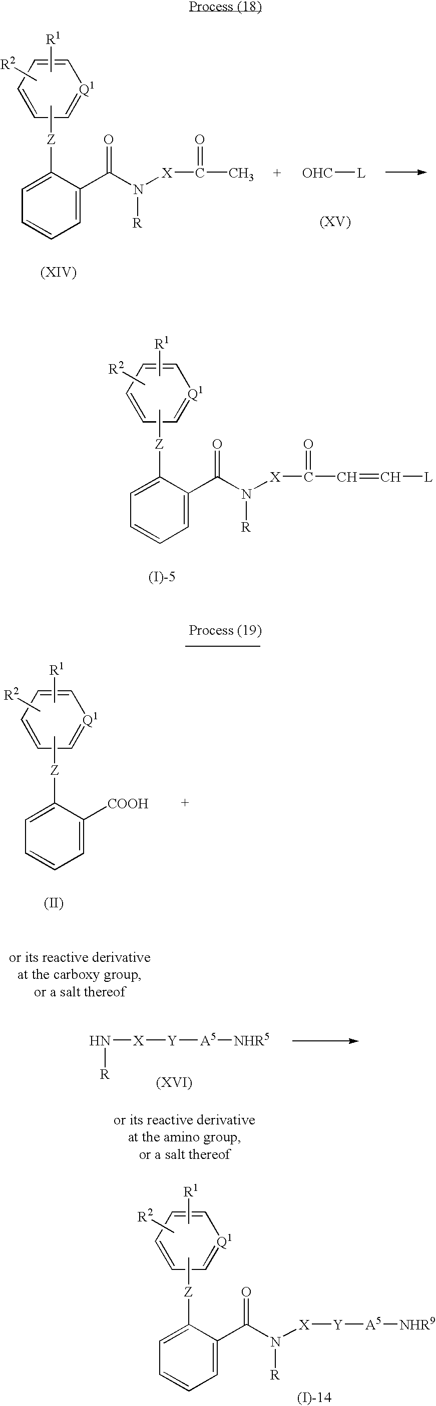

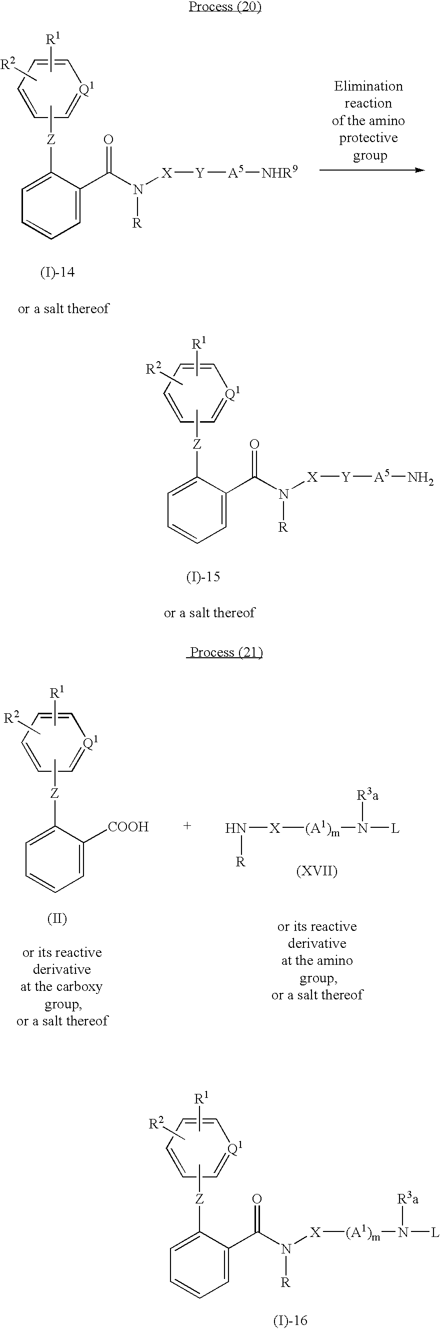

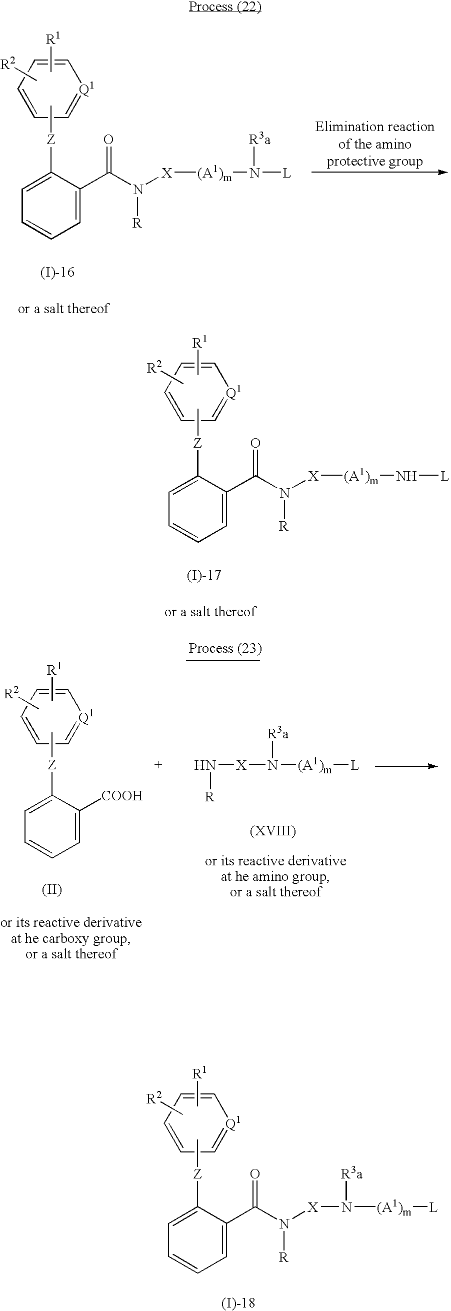

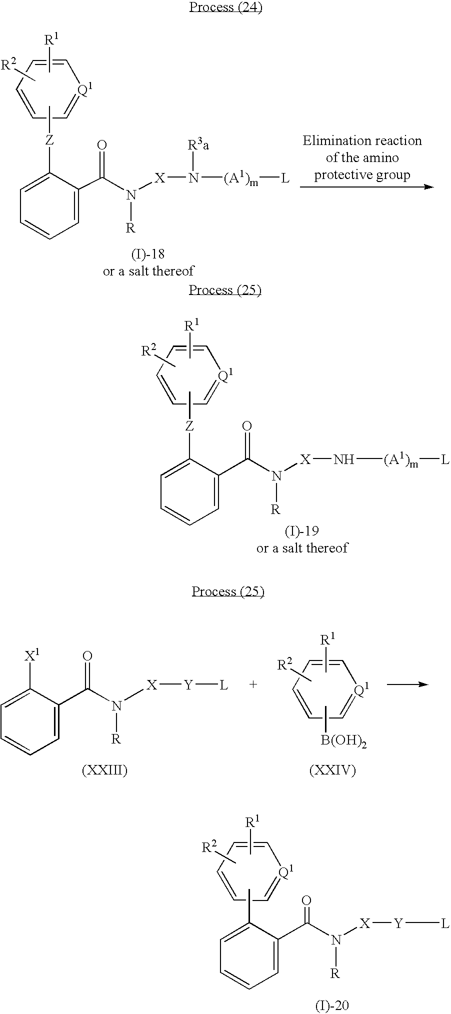

wherein Q¹, R¹, R², L, X, Y, Z, R, A¹ and m are as defined above, R³a and R⁹ are each amino protective group, A⁵ is unsaturated 3 to 10-membered heterocyclic group, and X¹ is halogen atom.

The starting compounds can be prepared by the following processes or by the method of Preparation mentioned below or by a process known in the art for preparing their structurally analogous compounds.

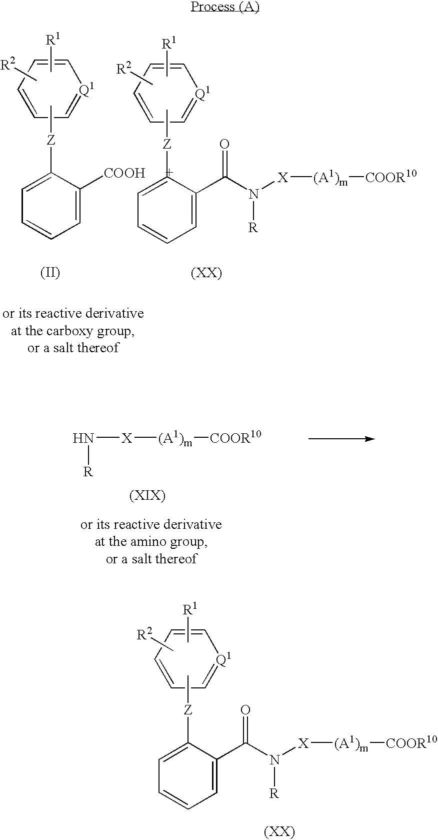

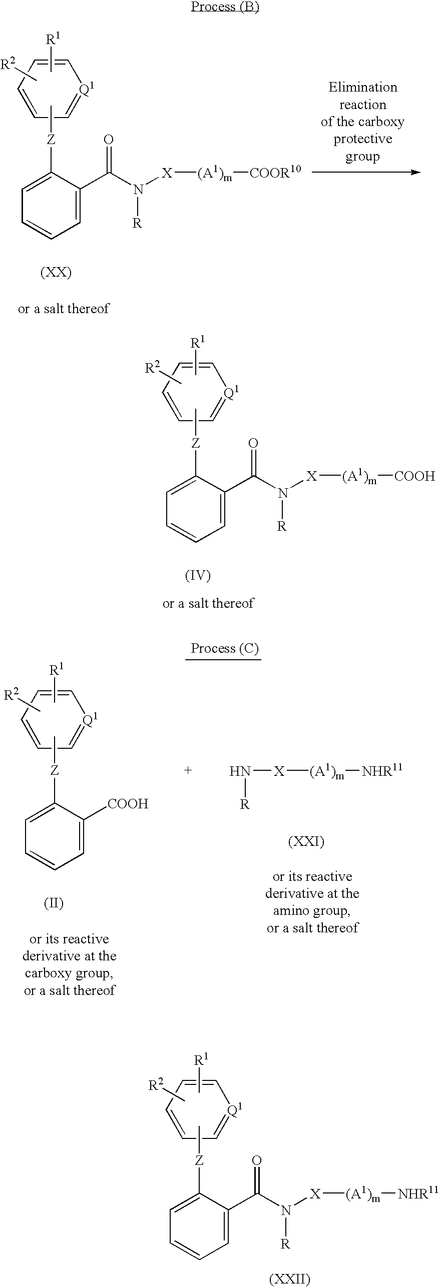

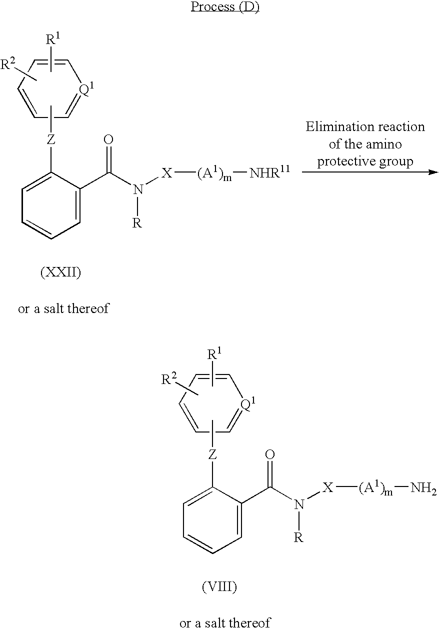

or a salt thereof

wherein Q¹, R¹, R², X, Z, R, A¹ and m are as defined above,

R¹⁰ is carboxy protective group, and

R¹¹ is amino protective group

The processes for preparing the object and starting compounds are explained in detail in the following.

Process (1)

The compound (I) or a salt thereof can be prepared by reacting the compound (II) or its reactive derivative at the carboxy group, or a salt thereof with the compound (III) or its reactive derivative at the amino group, or a salt thereof.

Suitable reactive derivative of the compound (III) includes Schiff's base type imino or its tautomeric enamine type isomer formed by the reaction of the compound (III) with a carbonyl compound such as aldehyde, ketone or the like; a silyl derivative formed by the reaction of the compound (III) with a silyl compound such as N,O-bis(trimethylsilyl)acetamide, N-trimethylsilylacetamide or the like; a derivative formed by the reaction of the compound (III) with phosphorus trichloride or phosgene.

Suitable reactive derivative of the compound (II) includes an acid halide, an acid anhydride and an activated ester. The suitable example may be an acid chloride; an acid azide; a mixed acid anhydride with an acid such as substituted phosphoric acid (e.g., dialkylphosphoric acid, phenylphosphoric acid, diphenylphosphoric acid, dibenzylphosphoric acid, halogenated phosphoric acid, etc.), dialkylphosphorous acid, sulfurous acid, thiosulfuric acid, alkanesulfonic acid (e.g., inethanesulfonic acid, ethanesulfonic acid, etc.), sulfuric acid, alkylcarbonic acid, aliphatic carboxylic acid (e.g., pivalic acid, pentanoic acid, isopentanoic acid, 2-ethylbutyric acid, trichloroacetic acid, etc.); aromatic carboxylic acid (e.g., benzoic acid, etc.); a symmetrical acid anhydride; an activated amide with imidazole, 4-substituted imidazole, dimethylpyrazole, triazole or tetrazole; an activated ester (e.g., cyanomethyl ester, methoxymethyl ester, dimethyliminomethyl [(CH₃)₂N⁺═CH—] ester, vinyl ester, propargyl ester, p-nitrophenyl ester, 2,4-dinitrophenyl ester, trichlorophenyl ester, pentachlorophenyl ester, mesylphenyl ester, phenylazophenyl ester, phenyl thioester, p-nitrophenyl thioester, p-cresyl thioester, carboxymethyl thioester, pyranyl ester, pyridinyl ester, piperidyl ester, 8-quinolyl thioester, etc.); or an ester with an N-hydroxy compound (e.g., N,N-dimethylhydroxylatine, 1-hydroxy-2-(1H)-pyridone, N-hydroxysuccinimide, N-hydroxybenzotriazole, N-hydroxyphthalimide, 1-hydroxy-6-chloro-1H-benzotriazole, etc.). These reactive derivatives can optionally be selected from them according to the kind of the compound (II) to be used.

The reaction is usually carried out in a conventional solvent such as water, acetone, dioxane, acetonitrile, chloroform, methylene chloride, ethylene dichloride, tetrahydrofuran, ethyl acetate, N,N-dimethylformamide, pyridine or any other organic solvents which do not adversely affect the reaction, or a mixture thereof.

When the compound (II) is used in free acid form or its salt form in the reaction, the reaction is preferably carried out in the presence of a conventional condensing agent such as N,N′-dicyclohexylcarbodiimide; N-cyclohexyl-N′-morpholinoethylcarbodiimide; N-cyclohexyl-N′-(4-diethylaminocyclohexyl)-carbodiimide; N,N′-diisopropylcarbodiimide; N-ethyl-N′-(3-dimethylaminopropyl)carbodiimide; N,N-carbonyl-bis-(2-methylimidazole); pentamethyleneketene-N-cyclohexylimine; diphenylketene-N-cyclohexylimine; ethoxyacetylene; 1-alkoxy-1-chloroethylene; trialkyl phosphite; isopropyl polyphosphate; phosphorus oxychloride (phosphoryl chloride); phosphorus trichloride; thionyl chloride; oxalyl chloride; triphenylphosphine; 2-ethyl-7-hydroxybenzisoxazolium salt; 2-ethyl-5-(m-sulfophenyl)isoxazolium hydroxide intramolecular salt; 1-(p-chlorobenzenesulfonyloxy)-6-chloro-1H-benzotriazole; so-called Vilsmeier reagent prepared by the reaction of N,N-dimethylformamide with thionyl chloride, phosgene, phosphorus oxychloride, etc.; or the like.

The reaction may also be carried out in the presence of an organic or inorganic base such as an alkali metal bicarbonate, tri(lower)alkylamine, pyridine, N-(lower)alkylmorpholine, N,N-di(lower)alkylbenzylamine, or the like.

The reaction temperature is not critical, and the reaction is usually carried out under cooling to heating.

### Process (2)

The compound (I)-1 or a salt thereof can be prepared by reacting the compound (IV) or its reactive derivative at the carboxy group, or a salt thereof with the compound (V) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (3)

The compound (I)-2 or a salt thereof can be prepared by reacting the compound (VI) or its reactive derivative at the carboxy group, or a salt thereof with the compound (VII) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (4)

The compound (I)-3 or a salt thereof can be prepared by reacting the compound (VIII) or its reactive derivative at the amino group, or a salt thereof with the compound (IX) or its reactive derivative at the carboxy group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (5)

The compound (I)-4 or a salt-thereof can be prepared by reacting the compound (X) or its reactive derivative at the amino group, or a salt thereof with the compound (XI) or its reactive derivative at the carboxy group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (6)

The compound (I)-5 or a salt thereof can be prepared by reacting the compound (II) or its reactive derivative at the carboxy group, or a salt thereof with the compound (XII) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (7)

The compound (I)-6 can be prepared by subjecting the compound (I)-5 to catalytic hydrogenation.

Suitable catalysts to be used in the catalytic hydrogenation are conventional ones such as platinum catalysts (e.g., platinum plate, spongy platinum, platinum black, colloidal platinum, platinum oxide, platinum wire, etc.), palladium catalysts (e.g., spongy palladium, palladium black, palladium oxide, palladium on carbon, palladium hydroxide on carbon, colloidal palladium, palladium on barium sulfate, palladium on barium carbonate, etc.), and the like.

The hydrogenation is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical, and the reaction is usually carried out under cooling to warming.

Process (8)

The compound (I)-7 can be prepared by subjecting the compound (I)-6 to reduction using a suitable reducing agent.

Suitable reducing agents to be used in the reduction are hydrides (e.g., sodium borohydride, sodium cyanoborohydride, lithium aluminum hydride, etc.).

The reduction is usually-carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformafide, N,N-dimethylacetamide or any other organic solvents which do not adversely affect the reaction, or a mixture thereof.

The reaction temperature is not critical, and the reaction is usually carried out under cooling to warming.

Process (9)

The compound (I)-8 can be prepared by subjecting the compound (I)-7 to catalytic hydrogenation in the presence of an acid.

Suitable catalysts to be used in the catalytic hydrogenation are conventional ones such as platinum catalysts (e.g., platinum plate, spongy platinum, platinum black, colloidal platinum, platinum oxide, platinum wire, etc.), palladium catalysts (e.g., spongy palladium, palladium black, palladium oxide, palladium on carbon, palladium hydroxide on carbon, colloidal palladium, palladium on barium sulfate, palladium on barium carbonate, etc.), and the like.

Suitable acid to be used in the catalytic hydrogenation includes hydrochloric acid, hydrogen chloride, and the like.

The hydrogenation is usually carried out in a conventional solvent such as water, alcohol (e.g., methanol, ethanol, isopropyl alcohol, etc.), tetrahydrofuran, dioxane, toluene, methylene chloride,-ethylene dichloride, chloroform, N,N-dimethylformamide, N,N-dimethylacetamide or any other organic solvents which do not adversely affect the reaction, or a mixture thereof.

The reaction temperature is not critical, and the reaction is usually carried out under cooling to warming.

Process (10)

The compound (I)-9 can be prepared by subjecting the compound (I)-5 to reduction using a suitable reducing agent.

This reaction can be carried out in the same manner as in the aforementioned Process (8), and therefore the reagents to be used and the reaction conditions (e.g., solvent, reaction temperature, etc.) can be referred-to those of Process (8).

Process (11)

The compound (I)-8 can be prepared by subjecting the compound (I)-9 to catalytic hydrogenation in the presence of an acid.

This reaction can-be carried out in the same manner as in the aforementioned Process (9), and therefore the reagents to be used and the reaction conditions (e.g., solvent, reaction temperature, etc.) can be referred to those of Process (9).

Process (12)

The compound (I)-10 or a salt thereof can be prepared by reacting the compound (II) or its reactive derivative at the carboxy group, or a salt thereof with the compound (XIII) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (13)

The compound (I)-11 can be prepared by subjecting the compound (I)-10 to catalytic hydrogenation.

This reaction can be carried out in the same manner as in the aforementioned Process (7), and therefore the reagents to be used and the reaction conditions (e.g., solvent, reaction temperature, etc.) can be referred to those of Process (7).

Process (14)

The compound (I)-12 can be prepared by subjecting the compound (I)-11 to reduction using a suitable reducing agent.

This reaction can be carried out in the same manner as in the aforementioned Process (8), and therefore the reagents to be used and the reaction conditions (e.g., solvent, reaction temperature, etc.) can be referred to those of Process (8).

Process (15)

The compound (I)-8 can be prepared by subjecting the compound (I)-12 to catalytic hydrogenation in the presence of an acid.

This reaction can be carried out in the same manner as in the aforementioned Process (9), and therefore the reagents to be used and the reaction conditions (e.g.,-solvent, reaction temperature, etc.) can be referred to those of Process (9).

Process (16)

The compound (I)-13 can be prepared by subjecting the compound (I)-10 to reduction using a suitable reducing agent.

This reaction can be carried out in the same manner as in the aforementioned Process (8), and therefore the reagents to be used and the reaction conditions (e.g., solvent, reaction temperature, etc.) can be referred to those of Process (8).

Process (17)

The compound (I)-8 can be prepared by subjecting the compound (I)-13 to catalytic hydrogenation in the presence of an acid.

This reaction can be carried out in the same manner as in the aforementioned Process (9), and therefore the reagents to be used and the reaction conditions (e.g., solvent, reaction temperature, etc.) can be referred to those of Process (9).

Process (18)

The compound (I)-5 can be prepared by reacting the compound (XIV) with the compound (XV) in the presence of a base or an acid.

Suitable base to be used in the reaction includes an inorganic base-and an organic base such as alkali metal hydroxide (e.g., sodium hydroxide, potassium hydroxide, etc.), alkaline earth metal hydroxide (e.g., magnesium hydroxide, calcium hydroxide, barium hydroxide, etc.), alkali metal carbonate (e.g., sodium carbonate, potassium carbonate, etc.), alkaline earth metal carbonate (e.g., magnesium carbonate, calcium carbonate, barium carbonate, etc.), alkoxide (e.g., sodium methoxide, sodium ethoxide, etc.), trialkylamine (e.g., trimethylamine, triethylamine,. etc.), and the like.

Suitable acid to be used in the reaction includes hydrochloric acid, hydrobromic acid, hydrogen chloride, hydrogen bromide, and the like.

This reaction is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, or any other organic solvents which do not adversely affect the reaction, or a mixture thereof.

The reaction temperature is not critical, and the reaction is usually carried out under cooling to heating.

Process (19)

The compound (I)-14 or a salt thereof can be prepared by reacting the compound (II) or its reactive derivative at the carboxy group, or a salt thereof with the compound (XVI) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (20)

The compound (I)-15 or a salt thereof can be prepared by subjecting the compound (I)-14 or a salt thereof to elimination reaction of the amino protective group.

Suitable method of this elimination reaction includes conventional one such as hydrolysis, reduction and the like.

(i) For Hydrolysis:

The hydrolysis is preferably carried out in the presence of a base or an acid including Lewis acid.

Suitable base includes an inorganic base and an organic base such as an alkali metal [e.g., sodium, potassium, etc.], an alkaline earth metal [e.g., magnesium, calcium, etc.], the hydroxide or carbonate or hydrogencarbonate thereof, trialkylamine [e.g., trimethylamine, triethylamine, etc.], picoline, 1,5-diazabicyclo[4.3.0]non-5-one, or the like.

Suitable acid includes an organic acid [e.g., formic acid, acetic acid, propionic acid, trichloroacetic acid, trifluoroacetic acid, etc.], and an inorganic acid [e.g., hydrochloric acid, hydrobromic acid, sulfuric acid, hydrogen chloride, hydrogen bromide, etc.].

The elimination using Lewis acid such as trihaloacetic acid [e.g., trichloroacetic acid, trifluoroacetic acid, etc.], or the like is preferably carried out in the presence of cation trapping agents [e.g., anisole, phenol, etc.]. This reaction is usually carried out without solvent.

The reaction may be carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical and the reaction is usually carried out under cooling to warming.

(ii) For Reduction:

Reduction is carried out in a conventional manner, including chemical reduction and catalytic reduction.

Suitable reducing reagent to be used in chemical reduction are hydrides (e.g., hydrogen iodide, hydrogen sulfide, lithium aluminum hydride, sodium borohydride, sodium cyanoborohydride, etc.), or a-combination of a metal (e.g., tin, zinc, iron, etc.) or metallic compound (e.g., chromium chloride, chromium acetate, etc.) and an organic acid or inorganic acid (e.g., formic acid, acetic acid, propionic acid, trifluoroacetic acid, p-toluenesulfonic acid, hydrochloric acid, hydrobromic acid, etc.).

Suitable catalysts to be used in catalytic reduction are conventional ones such as platinum catalysts (e.g., platinum plate, spongy platinum, platinum black, colloidal platinum, platinum oxide, platinum wire, etc.), palladium catalysts (e.g., spongy palladium, palladium black, palladium oxide, palladium on carbon, palladium hydroxide on carbon, colloidal palladium, palladium on barium sulfate, palladium on barium carbonate, etc.), nickel catalysts (e.g., reduced nickel, nickel oxide, Raney nickel, etc.), cobalt catalysts (e.g., reduced cobalt, Raney cobalt, etc.), iron catalysts (e.g., reduced iron, Raney iron, Ullman iron, etc.), and the like.

The reduction is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

Additionally, in case that the above-mentioned acids to be used in chemical reduction are in a liquid state, they can also be used as a solvent.

The reaction temperature of this reduction is not critical and the reaction is usually carried out under cooling to warming.

Process (21)

The compound (I)-16 or a salt thereof can be prepared by reacting the compound (II) or its reactive derivative at the carboxy group, or a salt thereof with the compound (XVII) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (22)

The compound (I)-17 or a salt thereof can be prepared by subjecting the compound (I)-16 or a salt thereof to elimination reaction of the, amino protective group.

This reaction can be carried out in the same manner as in the aforementioned Process (20), and therefore the reagents to be used and the reaction conditions (e.g., solvent, reaction temperature, etc.) can be referred to those of Process (20).

Process (23)

The compound (I)-18 or a salt thereof can be prepared by reacting the compound (II) or its reactive derivative at the carboxy group, or a salt thereof with the compound (XVIII) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (24)

The compound (I)-19 or a salt thereof can be prepared by subjecting the compound (I)-18 or a salt thereof to elimination reaction of the amino protective group.

This reaction can be carried out in the same manner as in the aforementioned Process (20), and therefore the reagents to be used and the reaction conditions (e.g., solvent, reaction temperature, etc.) can be referred to those of Process (20).

Process (25)

The compound (I)-20 or a salt thereof can be prepared by reacting the compound (XXIII) and the compound (XXIV) in the presence of tetrakis(triphenylphosphine)palladium and a base such as triethylamine.

This reaction can be carried out in a solvent such as N,N dimethylformamide which does not adversely affect the reaction. The reaction temperature is not critical and the reaction is usually carried out under cooling to heating.

This reaction can be carried out in a similar manner as in Example 90 mentioned below.

Process (A)

The compound (XX) or a salt thereof can be prepared by reacting the compound (II) or its reactive derivative at the carboxy group, or a salt thereof with the compound (XIX) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (B)

The compound (IV) or a salt thereof can be prepared by subjecting the compound (XX) or a salt thereof to elimination reaction of the carboxy protective group.

Suitable method of this elimination reaction includes conventional one such as hydrolysis.

The hydrolysis can be carried out in the same manner as in the aforementioned Process (20), and therefore the reagents to be used and the reaction conditions (e.g., solvent, reaction temperature, etc.) can be referred to those of Process (20).

Process (C)

The compound (XXII) or a salt thereof can be prepared by reacting the compound (II) or its reactive derivative at the carboxy group, or a salt thereof with the compound (XXI) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (D)

The compound (VIII) or a salt thereof can be prepared by subjecting the compound (XXII) or a salt thereof to elimination reaction of the amino protective group.

This reaction can be carried out in the same manner as in the aforementioned Process (20), and therefore the reagents to be used and the reaction conditions (e.g., solvent, reaction temperature, etc.) can be referred to those of Process (20).

Suitable salts of the starting compounds and their reactive derivatives in Processes (1) to (25) and (A) to (D) can be referred to the ones as exemplified for the compound (I).

The compounds obtained by the above processes can be isolated and purified by a conventional method such as pulverization, recrystallization, column chromatography, reprecipitation, or the like.

It is to be noted that the compound (I) and the other compounds may include one or more stereoisomer(s) such as optical isomer(s) and geometrical isomer(s) due to asymmetric carbon atom(s) and double bond(s), and all of such isomers and mixtures thereof are included within the scope of this invention.

The object compounds (I) and pharmaceutically acceptable salts thereof include solvates [e.g., enclosure compounds (e.g., hydrate, etc.)].

The object compounds (I) and pharmaceutically acceptable salts thereof possess a strong inhibitory activity on the secretion of Apo B.

Accordingly, the object compounds (I) and pharmaceutically acceptable salts thereof are useful as an Apo B secretion inhibitor.

The object compounds (I) and pharmaceutically acceptable salts thereof are useful as a medicament for the prophylaxis or treatment of diseases or conditions resulting from elevated circulating levels of Apo B such as hyperlipemia, hyperlipidemia, hyperlipoproteinemia, hypoalphalipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis and Syndrome X.

The present invention therefore provides a method for inhibiting or decreasing Apo B secretion in a mammal, in particular in human, which comprises administering an Apo B secretion inhibiting or decreasing amount of a compound of formula (I) or a pharmaceutically acceptable salt thereof to the mammal.

The present invention also provides a method for preventing or treating diseases or conditions resulting from elevated circulating levels of Apo B in a mammal, in particular in human, which comprises administering an effective amount of a compound of formula (I) or a pharmaceutically acceptable salt thereof to the mammal.

The object compounds (I) and pharmaceutical acceptable salts thereof are also useful in reducing intestinal fat absorption and reducing food intake for the prophylaxis or treatment of obesity. Furthermore, the object compounds (I) and pharmaceutical acceptable salts thereof possess an inhibitory activity on the lipid transfer of microsomal triglyceride transfer protein (MTP).

In order to illustrate the usefulness of the object compound (I), the pharmacological test result of the compound (I) is shown in the following.

Test Compounds:

N-{4-[3-(2-pyridinyl)propyl]phenyl}-4′-(trifluoromethyl)-1,1′-biphenyl-2-carboxamide (Example 25)

N-{4-[3-(6-amino-2-pyridinyl)propyl]phenyl}-4′-(trifluoromethyl)-1,1′-biphenyl-2-carboxamide (Example 44)

N-(4-{[2-(2-pyridinyl)ethyl]amino}phenyl)-4′-(trifluoromethyl)-1,1′-biphenyl-2-carboxamide (Example 59)

N-(4-{[2-(2-pyridinyl)ethyl]amino}phenyl)-2-[3-(trifluoromethyl)-anilino]benzamide (Example 64)

N-{4-[(2-pyridinylacetyl)amino]phenyl]-4′-(trifluoromethyl)-1,1′-biphenyl-2-carboxamide (Example 65)

Test 1: Measurement of Inhibition of Apo B Secretion

HepG2 cells were seeded in Eagles medium containing 10% fetal calf serum (FCS) at a density of 30000 cells/well in 96-well plates and allowed to grow for 3 days before treatment. At this time, the medium was replaced with fresh medium containing 0.1% dimethyl sulfoxide (DMSO) and the indicated concentrations of a test compound. After 15-hour incubation, the amount of Apo B and Apo AI accumulated in the media was determined by ELISA.

The assay was carried out at room temperature. A flat bottomed micro ELISA plate (manufactured by Nunc) was coated with an anti Apo B monoclonal antibody solution (5 mg/ml in 0.05% carbonate buffer, pH 9.6) by adding the antibody solution at a volume of 100 μl per well. After 1-hour incubation on a plate mixer, the unbound materials were removed by washing the well 3 times with a washing buffer (phosphate buffered saline, pH 7.2 containing 0.1% bovine serum albumin and 0.05% Tween-20). Then 20 μl of a solution of the test compound (dissolved in the culture medium) and 100 μl of a solution of peroxidase coupled anti Apo B antibody were added. After 1-hour incubation on a plate mixer, washing was performed 3 times to remove the unbound materials. A freshly prepared substrate solution (2.5 mg/ml ortho-phenylene diamine and 0.018% H₂O_{z} in 0.11 M Na₂HPO₄-0.044 M sodium citrate buffer, pH 5.4) at a volume of 200 μl was then added to each well. After 20-minute incubation, the enzyme reaction was terminated by adding 50 μl of 0.5 M sulfuric acid. Absorbance of each well was determined at 490 nm using a microplate reader. Apo B concentration was calculated from a standard curve generated from purified Apo B standard that was run in parallel in the same plate. Inhibition of Apo B secretion by the test compound is calculated taking 0-1% DMSO treated cells as controls.

Measurement of Apo AI was performed similar to that of Apo B, except for diluting the sample 11-fold with a dilution buffer (phosphate buffered saline, pH 7.2 containing 0.5% bovine serum albumin and 0.05% Tween-20).

Apo B secretion inhibitors are identified as compounds that decrease Apo B secretion without affecting the secretion of Apo AI.

Test Results:

TABLE 1

| | Inhibition of Apo B |
|---|---|
| Test compound | secretion at 10⁻⁶ M |
| (Example No.) | (%) |
| 44 | 100 |
| 64 | 100 |
| 65 | 92.2 |

Test 2: Lipids Lowering Effect on ddY-Mice

Male ddy-mice were housed in temperature- and humidity-controlled rooms and fed with laboratory chow. The animals were randomized according to their body weight and deprived of food just before the experiment. A blood sample (baseline blood sample) was collected from the retro orbital venous plexus before administration of the test drug, and then the animals were orally dosed with the test drug in a vehicle (aqueous solution of 0.5% methylcellulose). Blood samples were drawn at 2 hours after drug administration for the measurement of cholesterol and triglyceride.

Plasma total-cholesterol and plasma triglyceride were determined by conventional enzyme-methods using commercially available kits. The cholesterol CII-Test Wako (Wako Pure Chemical Industries, Ltd.) was used for the measurement of cholesterol, and the triglyceride E-test Wako (Wako Pure Chemical Industries, Ltd.) was used for the measurement of triglyceride.

Lipids lowering effects were shown in percent relative to the baseline level (level at 0 hr).

Test Results:

TABLE 2

| | | Cholesterol | Triglyceride |
|---|---|---|---|
| Test compound | Dose | (% of 0 hr) | (% of 0 hr) |
| (Example No.) | (mg/kg) | 2 hr | 2 hr |
| 25 | 32 | 78 | 23 |
| 65 | 32 | 83 | 36 |
| 59 | 32 | 77 | 15 |

For therapeutic administration, the object compound (I) of the present invention and pharmaceutically acceptable salts thereof are used in the form of a conventional pharmaceutical preparation in admixture with a conventional pharmaceutically acceptable carrier such as an organic or inorganic solid or liquid excipient which is suitable for oral, parenteral or external administration. The pharmaceutical preparation may be compounded in a solid form such as granule, capsule, tablet, dragee, suppository or ointment, or in a liquid form such as solution, suspension or emulsion for injection, intravenous drip, ingestion, eye drop, endermism, inhalation, etc. If needed, there may be included in the above preparation auxiliary substance such as stabilizing agent, wetting or emulsifying agent, buffer or any other commonly used additives.

The effective ingredient may usually be administered in a unit dose of 0.01 mg/kg to 100 mg/kg, preferably 0.1 mg/kg to 10 mg/kg, 1 to 4 times a day. However, the above dosage may be increased or decreased according to age, body weight and conditions of the patient or administering method.

Suitable mammal to which the object compounds (I) and pharmaceutical acceptable salts thereof or above preparations are applied, includes a human being, a companion animal such as a dog and a cat, livestock such as a cow and a pig, and the like.

The object compounds (I) and pharmaceutical acceptable salts thereof may, if desired, be administered with one or more therapeutic agents and formulated for administration by any convenient route in a conventional manner. Appropriate doses will be readily appreciated by those skilled in the art. For example, the object compounds (I) and pharmaceutical acceptable salts thereof may be administered in combination with an HMG CoA reductase inhibitor. The object compounds (I) and pharmaceutical acceptable salts thereof may be also administered in combination with a known anti-obesity agent, for example, β₃-adrenergic receptor agonist, a cholecystokinin-A agonist, a monoamine reuptake inhibitor, a sympathomimetic agent, a serotoninergic agent, a dopamine agonist, a melanocyte-stimulating hormone receptor agonist or mimetic, a melanocyte-stimulating hormone receptor analog, a cannabinoid receptor Antagonist, a melanin concentrating hormone antagonist, leptin, a leptin analog, a leptin receptor agonist, a galanin antagonist, a lipase inhibitor, a bombesin agonist, a Neuropeptide-Y antagonist, a thyromimetic agent, dehydroepiandrosterone or an analog thereof, a glucocorticoid receptor agonist or antagonist, an orexin receptor antagonist, a urocortin binding protein antagonist, a glucagonlike peptide-1 receptor agonist, a ciliary neurotrophic factor, a human agouti-related protein antagonist, and the like, for the prophylaxis or treatment of obesity.

The following Preparations and Examples are given for the purpose of-illustrating the present invention in detail.

Preparation 1

A solution of 4′-(trifluoromethyl)-1,1′-biphenyl-2-carboxylic acid (4.0 g) and 1-hydroxybenzotriazole hydrate (HOBT.H₂O) (2.03 g) and 1-[3-(dimethylamino)propyl]-3-ethylcarbodiimide hydrochloride (WSC.HCl) (3.2 g) in N,N-dimethylformamide (30 ml) was stirred at ambient temperature for an hour. Ethyl (4-aminophenyl)acetate (2.95 g) was added to the above mixture and the reaction mixture was stirred at ambient temperature for 20 hours. The resultant mixture was poured into a mixture of ethyl acetate and water. The organic layer was washed with brine and dried over magnesium sulfate. The solvent was evaporated in vacuo and the residue was recrystallized from a mixture of ethyl acetate and diisopropyl ether to give ethyl [4-({[4′-(trifluoromethyl)-1,1′-biphenyl-2-yl]carbonyl}amino)phenyl]acetate (5.6 g).

¹H-NMR (DMSO-d₆): δ1.70 (3H, t, J=7.10 Hz), 3.59 (2H, s), 4.06 (2H, q, J=7.10 Hz), 7.12 (2H, d, J=8.44 Hz), 7.48 (2H, d, J=8.44 Hz), 7.53-7.66 (6H, m), 7.76 (2H, d, J=8.32 Hz), 10.37 (1H, s).

Preparation 2

A solution of ethyl [4-({[4′-(trifluoromethyl)-1,1′-biphenyl-2-yl]carbonyl)amino)phenyl)acetate (5.5 g) and 1N sodium hydroxide solution (19.3 ml) in methanol (80 ml) and tetrahydrofuran (20 ml) was refluxed under stirring for 1.5 hours. The reaction mixture was evaporated in vacuo and the residue was dissolved in a mixture of ethyl acetate and water. The aqueous layer was adjusted to pH 1.5 with 6N hydrochloric acid solution and extracted with ethyl acetate. The organic layer was washed with brine and dried over magnesium sulfate. The solvent was evaporated in vacuo and the residue was washed with diisopropyl ether to give [4-{[4′-(trifluoromethyl)-1,1′-biphenyl-2-yl]carbonyl}amino)phenyl]acetic acid (4.50 g).

¹H-NMR (DMSO-₆): δ3.60 (2H, s), 7.17 (2H, d, J=8.46 Hz), 7.42 (2H, d, J=8.46 Hz), 7.52-7.62 (6H, m), 10.36 (1H, s), 12.29 (1H,s).

### EXAMPLE 1

A solution of [4-({[4′-(trifluoromethyl)-1,1′-biphenyl-2-yl]carbonyl}amino)phenyl]acetic acid (1.6 g) and HOBT.H₂O (0.6 g) and WSC.HCl (0.92 g) in N,N-dimethylformamide (20 ml) was stirred at ambient temperature for an hour. 2-Aminopyridine (0.49 g) was added to the above mixture and the reaction mixture was stirred at ambient temperature for 20 hours. The resultant mixture was poured into a mixture of ethyl acetate and water. The organic layer was washed with brine and dried over magnesium sulfate. The solvent was evaporated in vacuo and the residue was recrystallized from a mixture of ethyl acetate and diisopropyl ether to give N-{4-[2-oxo-2-(2-pyridinylamino)ethyl]phenyl}-4′-(trifluoromethyl)-1,1′-biphenyl-2-carboxamide (1.20 g).

¹H-NMR (DMSO-d₆): δ3.49 (2H, s), 7.05-7.08 (2H, m), 7.25 (2H, d, J=8.42 Hz), 7.48 (2H, d, J=8.48 Hz), 7.51-8.03 (5H, m), 7.76 (1H, d, J=8.36 Hz), 8.05 (2H, d, J=8.36 Hz), 8.31 (1H, d, J=3.82 Hz), 10.22 (1H, s), 10.65 (1H, s).

### EXAMPLE 2

A solution of [4-({[4′-(trifluoromethyl)-1,1′-biphenyl-2-yl]carbonyl}amino)phenyl]acetic acid (240 mg) and HOBT.H₂O (89 mg) and WSC.HCl (138 mg) in N,N-dimethylformamide (10 ml) was stirred at ambient temperature for an hour. 3-Methyl-2-aminopyridine (78 mg) was added to the above mixture and the reaction mixture was stirred at ambient temperature for 5 hours. The resultant mixture was poured into a mixture of ethyl acetate and water. The organic layer was washed with brine and dried over magnesium sulfate. The solvent was evaporated in vacuo and the residue was recrystallized from a mixture of ethyl acetate and diisopropyl ether to give N-(4-{2-[(3-methyl-2-pyridinyl)amino]-2-oxoethyl}phenyl)-4′-(trifluoromethyl)-1,1′-biphenyl-2-carboxamide (147 mg).

¹H-NMR (DMSO-d₆): δ2.05 (3H, s), 3.60 (2H, s), 7.16-7.19 (1H, m), 7.24 (2H, d, J=8.46 Hz), 7.42 (2H, d, J=8.46 Hz), 7.49-7.66 (7H, m), 7.76 (2H, d, J=8.24 Hz)) 6.23 (1H, d, J=3.22 Hz), 10.18 (1H, s), 10.34 (1H, s)

### EXAMPLE 3

A solution of [4-({[4′-(trifluoromethyl)-1,1′-biphenyl-2-yl]carbonyl}amino)phenyl]acetic acid (400 mg) and HOBT.H₂O (180 mg) and WSC.HCl (290 mg) in N,N-dimethylformamide (10 ml) was stirred at ambient temperature for an hour. 4-Methyl-2-aminopyridine (120 mg) was added to the above mixture and the reaction mixture was stirred at ambient temperature for 5 hours. The resultant mixture was poured into a mixture of ethyl acetate and water. The organic layer was washed with brine and dried over magnesium sulfate. The solvent was evaporated in vacuo and the residue was recrystallized from a mixture of ethyl acetate and diisopropyl ether to give N-(4-{2-[(4-methyl-2-pyridinyl)amino]-2-oxoethyl}phenyl)-4′-(trifluoromethyl)-1,1′-biphenyl-2-carboxamide (205.8 mg).

¹H-NMR (DMSO-d₆): δ2.28 (3H, s), 3.64 (2H, s), 6.92 (1H, d, J=4.70 Hz), 7.24 (2H, d, J=8.48 Hz), 7.45 (2H, d, J=8.48 Hz), 7.49-7.65 (6H, m), 7.76 (2H, d, J=8.42 Hz), 7.89 (1H, s), 8.15 (1H, d, J=5.02 Hz), 10.35 (1H, s), 10.55 (1H, s).

### EXAMPLE 4

A solution of [4-({[4′-(trifluoromethyl)-1,1′-biphenyl-2-yl]carbonyl)amino)phenyl]acetic acid (240 mg) and HOBT.H₂O (89 mg) and WSC.HCl (138 mg) in N,N-dimethylformamide (10 ml) was stirred at ambient temperature for an hour. 5-Methyl-2-aminopyridine (78 mg) was added to the above mixture and the reaction mixture was stirred at ambient temperature for 5 hours. The resultant mixture was poured into a mixture of ethyl acetate and water. The organic layer was washed with brine and dried over magnesium sulfate. The solvent was evaporated in vacuo and the residue was recrystallized from a mixture of ethyl acetate and diisopropyl ether to give N-(4-{2-[(5-methyl-2-pyridinyl)amino]-2-oxoethyl}phenyl)-4′-(trifluoromethyl)-1,1′-bipehenyl-2-carboxamide (202 mg).

¹H-NMR (DMSO-d₆): δ2.28 (3H, s), 3.63 (2H, s), 7.24 (2H, d, J=8.46 Hz), 7.47 (2H, d, J=8.46 Hz), 7.49-7.65 (7H, m), 7.76 (2H, d, J=8.30 Hz), 7.94 (1H, d, J=8.36 Hz), 8.14 (1H, s), 10.35 (1H, s), 10.55 (1H, s).

Preparation 3

A solution of 4′-methyl-1,1′-biphenyl-2-carboxylic acid (1.06 g) and HOBT.H₂O (0.74 g) and WSC.HCl (1.15 g) in N,N-dimethylformamide (30 ml) was stirred at ambient temperature for an hour. Ethyl (4-aminophenyl)acetate (2.95 g) was added to the above mixture and the reaction mixture was stirred at ambient temperature for 20 hours. The resultant mixture was poured into a mixture of ethyl acetate and water. The organic layer was washed with brine and dried over magnesium sulfate. The solvent was evaporated in vacuo and the residue was recrystallized from a mixture of ethyl acetate and diisopropyl ether to give ethyl (4-{([(4′-methyl-1,1′-biphenyl-2-yl)carbonyl]amino}phenyl)acetate (1.46 g).

¹H-NMR (DMSO-d₆): δ1.73 (3H, t, J=7.10 Hz), 2.28 (3H, s), 3.58 (2H, s), 4.06 (2H, q, J=7.10 Hz), 7.13-7.19 (4H, m), 7.34 (2H, d, J=8.04 Hz), 7.41-7.58 (6H, m), 10.37 (1H, s).

Preparation 4

A solution of ethyl (4-{[(4′-methyl-1,1′-biphenyl-2-yl)carbonyl]amino}phenyl)acetate (1.4 g) and 1N sodium hydroxide solution (19.3 ml) in methanol (80 ml) and tetrahydrofuran (20 ml) was refluxed under stirring for 1.5 hours. The reaction mixture was evaporated in vacuo and the residue was dissolved in a mixture of ethyl acetate and water. The aqueous layer was adjusted to pH 1.5 with 6N hydrochloric acid solution and extracted with ethyl acetate. The organic layer was washed with brine and dried over magnesium sulfate. The solvent was evaporated in vacuo and the residue was washed with diisopropyl ether to give [4-{[(4′-methyl-1,1-biphenyl-2-yl)carbonyl]amino}phenyl]acetic acid (1.19 g).

¹H-NMR (DMSO-d₆): δ2.28 (3H, s), 3.49 (2H, s), 7.13-7.20 (4H, m), 7.34 (2H, d J=8.04 Hz), 7.36-7.58 (8H, m), 10.22 (1H, s), 12.28 (1H, s).

### EXAMPLE 5

A solution of [4-{[(4′-methyl-1,1′-biphenyl-2-yl)carbonyl]amino}phenyl]acetic acid (330 mg) and HOBT.H₂O (149 mg) and WSC.HCl (230 mg) in N,N-dimethylformamide (10 ml) was stirred at ambient temperature for an hour. 2-Aminopyridine (113 mg) was added to the above mixture and the reaction mixture was stirred at ambient temperature for 20 hours. The resultant mixture was poured into a mixture of ethyl acetate and water. The organic layer was washed with brine and dried over magnesium sulfate. The solvent was evaporated in vacuo and the residue was recrystallized from a mixture of ethyl acetate and diisopropyl ether to give 4′-methyl-N-{4-[2-oxo-2-(2-pyridinylamino)ethyl]phenyl}-1,1′-biphenyl-2-carboxamide (150 mg).

¹H-NMR (DMSO-d₆): δ2.28 (3H, s), 3.66 (2H, s), 7.05-7.58 (13H, m), 7.71-7.78 (1H, m), 8.04 (1H, d, J=8.38 Hz), 8.31 (1H, d, J=3.46 Hz), 10.22 (1H, s), 10.65 (1H, s).

Preparation 5

A solution of 4′-(trifluoromethyl)-1,1′-biphenyl-2-carboxylic acid (4.0 g) and HOBT.H₂O (2.03 g) and WSC.HCl (3.2 g) in N,N-dimethylformamide (30 ml) was stirred at ambient temperature for an hour. Methyl 4-aminobenzoate (3.20 g) was added to the above mixture and the reaction mixture was stirred at ambient temperature for 20 hours. The resultant mixture was poured into a mixture of ethyl acetate and water. The organic layer was washed with brine and dried over magnesium sulfate. The solvent was evaporated in vacuo and the residue was recrystallized from a mixture of ethyl acetate and diisopropyl ether to give methyl 4-({[4′-(trifluoromethyl)-1,1′-biphenyl-2-yl]carbonyl}amino)benzoate (3.16 g)

¹H-NMR (DMSO-d₆): δ3.82 (3H, s), 7.52-7.74 (13H, m), 7.90 (2H, d, J=8.65 Hz), 10.73 (1H, s).

Preparation 6

A solution of methyl 4-({[4′-(trifluoromethyl)-1,1′-biphenyl-2-yl]carbonyl}amino)benzoate (1.4 g) and 1N sodium hydroxide solution (7 ml) in methanol (15 ml) and tetrahydrofuran (10 ml) was refluxed-under stirring for 6 hours. The reaction mixture was evaporated in vacuo and the residue was dissolved in a mixture of ethyl acetate and water. The aqueous layer was adjusted to pH 1.0 with 6N hydrochloric acid solution and extracted with ethyl acetate. The organic layer was washed with brine and dried over magnesium sulfate. The solvent was evaporated in vacuo and the residue was washed with diisopropyl ether to give 4-({[4′-(trifluoromethyl)-1,1′-biphenyl-2-yl]carbonyl}amino)benzoic acid (1.28 g).

¹H-NMR (DMSO-d₆): δ7.52-7.78 (13H, m), 7.87 (2H, d, J=8.62 Hz), 10.69 (1H, s), 12.73 (1H, brs).

### EXAMPLE 6

A solution of 4-({[4′-(trifluoromethyl)-1,1′-biphenyl-2-yl]carbonyl}amino)benzoic acid (372 mg) and HOBT.H₂O (149 mg) and WSC.HCl (230 mg) in N,N-dimethylformamide (16 ml) was stirred at ambient temperature for an hour. 2-Aminomethylpyridine (131 mg) was added to the above mixture and the reaction mixture was stirred at ambient temperature for 20 hours. The resultant mixture was poured into a mixture of ethyl acetate and water. The organic layer was washed with brine and dried over magnesium sulfate. The solvent was evaporated in vacuo and the residue was recrystallized from a mixture of ethyl acetate and diisopropyl ether to give N-(4-{[(2-pyridinylmethyl)amino]carbonyl}phenyl)-4′-(trifluoromethyl)-1,1′-biphenyl-2-carboxamide (430 mg).

¹H-NMR (DMSO-d₆): δ4.56 (2H, d, J=5.81 Hz), 7.23-7.33 (2H, m), 7.52-7.79 (11H, m), 7.87 (2H, d, J=8.64 Hz), 8.51 (1H, d, J=4.36 Hz), 8.98-9.04 (1H, m), 10.62 (1H, s).

### EXAMPLE 7

A solution of 4-({[4′-(trifluoromethyl)-1,1′-biphenyl-2-yl]carbonyl}amino)benzoic acid (372 mg) and HOBT¹H₂O (149 mg) and WSC.HCl (230 mg) in N,N-dimethylformamide (10 ml) was stirred at ambient temperature for an hour. 2-(2-Pyridinyl)ethylamine (134 mg) was added to the above mixture and the reaction mixture was stirred at ambient temperature for 20 hours. The resultant mixture was poured into a mixture of ethyl acetate and water. The organic layer was washed with brine and dried over magnesium sulfate. The solvent was evaporated in vacuo and the residue was recrystallized from a mixture of ethyl acetate and diisopropyl ether to give N-[4-({[2-(2-pyridinyl)ethyl]amino}carbonyl)phenyl]-4′-(trifluoromethyl)-1,1′-biphenyl-2-carboxamide (380 mg).

¹H-NMR (DMSO-d₆): δ2.95 (2H, t, J=6.95 Hz), 3.55-3.65 (2H, m), 7.19-7.28 (2H, m), 7.5.1-7.78 (10H, m), 7.76 (2H, d, J=8.61 Hz), 8.43-8.52 (2H, m), 10.57 (1H, s).

### EXAMPLE 8

A solution of 4-({[4′-(trifluoromethyl)-1,1′-biphenyl-2-yl]carbonyl}amino)benzoic acid (372 mg) and 1-hydroxy-benzotriazole hydrate (149 mg) and WSC.HCl (210 mg) in N,N-dimethylformamide (20 ml) was stirred at ambient temperature for an hour. 2-Aminomethyl-5-methylpyrazine (135 mg) was added to the above mixture and the reaction mixture was stirred at ambient temperature for 20 hours. The resultant mixture was poured into a mixture of ethyl acetate and water. The organic layer was washed with brine and dried over magnesium sulfate. The solvent was evaporated in vacuo and the residue was recrystallized from a mixture of ethyl acetate and diisopropyl ether to give 4′-(trifluoromethyl)-biphenyl-2-carboxylic acid N-(4-({[(5-methyl-2-pyrazinyl)methyl]amino}carbonyl)phenyl]-4′-(trifluoromethyl)-1,1′-biphenyl-2-carboxamide (430 mg).

¹H-NMR (DMSO-d₆): δ2.47 (3H, s), 4.55 (2H, d, J=5.64 Hz), 7.52-7.95 (12H, m), 8.48 (2H, s), 9.00-9.06 (1H, m), 10.61 (1H, s).

### EXAMPLE 9

A solution of 4-({[4′-(trifluoromethyl)-1,1′-biphenyl-2-yl]carbonyl}amino)benzoic acid (260 mg) and 1-hydroxy-benzotriazole hydrate (104 mg) and WSC.HCl (161 mg) in N,N-dimethylformamide (10 ml) was stirred at ambient temperature for an hour. 2-Aminopyridine (131 mg) was added to the above mixture and the reaction mixture was stirred at ambient temperature for 20 hours and at 70-80° C. for 2 hours. The resultant mixture was poured into a mixture of ethyl acetate and water. The organic layer was washed with brine and dried over magnesium sulfate. The solvent was evaporated in vacuo and the residue was recrystallized from a mixture of ethyl acetate and diisopropyl ether to give N-{4-[(2-pyridinylamino)carbonyl]phenyl}-4′-(trifluoromethyl)-1,1′-biphenyl-2-carboxamide (151 mg).

¹H-NMR (DMSO-d₆): δ7.12-7.18 (1H, m), 7.53-7.87 (11H, m), 8.01 (2H, d, J=8.50 Hz), 8.20 (2H, d, J=8.30 Hz), 8.38 (1H, d, J=4.22 Hz), 10.66 (1H, m), 10.68 (1H, s).

### EXAMPLE 10

A solution of 4′-(trifluoromethyl)-1,1′-biphenyl-2-carbonyl chloride (854 mg) in tetrahydrofuran (5 ml) was added to a mixture of 4-(2-pyridinyl)phenylamine (510 mg) and triethylamine (606 mg) in tetrahydrofuran (25 ml) at ambient temperature. The mixture was stirred at ambient temperature for 3 hours. The resultant mixture was poured into a mixture of ethyl acetate and water and organic layer was washed with 5% aqueous potassium carbonate solution and brine and dried over magnesium sulfate. The solvent was evaporated in vacuo and the residue was recrystallized from a mixture of ethyl acetate and diisopropyl ether to give N-[4-(2-pyridinyl)phenyl]-4′-(trifluoromethyl)-1,1′-biphenyl-2-carboxamide (1.20 g).

¹H-NMR (DMSO-d₆): δ7.30-7.34 (1H, m), 7.51-7.94 (12H, m), 8.04 (2H, d, J=8.68 Hz), 8.364 (1H, d, J=4.56 Hz), 10.55 (1H, s).

### EXAMPLE 11

N-[4-(2-Pyridinylmethyl)phenyl]-4′-(trifluoromethyl)-1,1′-biphenyl-2-carboxamide

The title compound was obtained from 4′-(trifluoromethyl)-1,1′-biphenyl-2-carbonyl chloride and 4-(2-pyridinylmethyl)-phenylamine in the same manner as in Example 10.

¹H-NMR (DMSO-d₆): δ4.02 (2H, m), 7.18 (2H, d, J=8.46 Hz), 7.16-7.47 (2H, m), 7.45 (2H, d, J=8.46 Hz), 7.25 -7.71 (7H, m), 7.75 (1H, d, J=8.36 Hz), 8.41 (1H, d, J=4.18 Hz), 10.33 (1H, s).

### EXAMPLE 12

A mixture of 1,1′-biphenyl-2-carboxylic acid (397 mg) and HOBT.H₂O (300 mg) and WSC.HCl (420 mg) in N,N-dimethylformamide (15 ml) was stirred at ambient temperature for an hour. 4-(2-Pyridinylmethyl)phenylamine (368 mg) was added to the above mixture and the reaction mixture was stirred at ambient temperature for 20 hours. The resultant mixture was poured into a mixture of ethyl acetate and water. The organic layer was washed with 5% aqueous potassium carbonate solution and brine and dried over magnesium sulfate. The solvent was evaporated in vacuo and the residue was recrystallized from a mixture of ethyl acetate and diisopropyl ether to give N-[4-(2-pyridinylmethyl)phenyl]-1,1′-biphenyl-2-carboxamide (557 mg).

¹H-NMR (DMSO-d₆): δ4.00 (2H, s), 7.14-7.95 (13H, m), 7.16 (2H, d, J=8.36 Hz), 8.46 (1H, d, J=4.66 Hz), 10.17 (1H, s).

### EXAMPLE 13

A mixture of 4′-chloro-1,1′-biphenyl-2-carboxylic acid (233 mg) and HOBT.H₂O (149 mg) and WSC.HCl (210 mg) in N,N-dimethylformamide (15 ml) was stirred at ambient temperature for an hour. 4-(2-Pyridinylmethyl)phenylamine (368 mg) was added to the above mixture and the reaction mixture was stirred at ambient temperature for 20 hours. The resultant mixture was poured into a mixture of ethyl acetate and water. The organic layer was washed with 5% aqueous potassium carbonate solution and brine and dried over magnesium sulfate. The solvent was evaporated in vacuo and the residue was recrystallized from a mixture of ethyl acetate and diisopropyl ether to give 4′-chloro-N-[4-(2-pyridinylmethyl)phenyl]-1,1′-biphenyl-2-carboxamide (237 mg).

¹H-NMR (DMSO-d₆): δ4.01 (2H, s), 7.16-7.33 (2H, m), 7.18 (2H, d, J=8.40 Hz), 7.43-7.73 (11H, m), 8.47 (1H, d, J=4.82 Hz), 10.25 (1H, s)

### EXAMPLE 14

A mixture of 4′-methyl-1,1′-biphenyl-2-carboxylic acid (425 mg) and HOBT.H₂O (300 mg) and WSC.HCl (420 mg) in N,N-dimethylformamide (15 ml) was stirred at ambient temperature for an hour. 4-(2-Pyridinylmethyl)phenylamine (368 mg) was added to the above mixture and the reaction mixture was stirred at ambient temperature for 20 hours. The resultant mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (1:1). The fraction containing the objective compound was evaporated and the residue was recrystallized from a mixture of ethyl acetate and diisopropyl ether to give 4′-methyl-N-[4-(2-pyridinylmethyl)phenyl]-1,1′-biphenyl-2-carboxamide (508 mg).

¹H-NMR (DMSO-d₆): δ2.73 (3H, m), 4.01 (2H, s), 6.51-7.72 (15H, m), 8.47 (1H, d, J=4.02 Hz), 10.20 (1H, s).

### EXAMPLE 15

A mixture of 4′-(trifluoromethyl)-1,1′-biphenyl-2-carboxylic acid (400 mg) and HOBT.H₂O (223 mg) and WSC.HCl (315 mg) in N,N-dimethylformamide (15 ml) was stirred at ambient temperature for an hour. 4-[2-(2-Pyridinyl)ethyl]phenylamine (298 mg) was added to the above mixture and the reaction mixture was stirred at ambient temperature for 20 hours. The resultant mixture was poured into a mixture of ethyl acetate and water. The organic layer was washed with 6N hydrochloric acid. The aqueous layer was adjusted to pH 9.0 with 20% aqueous potassium carbonate solution and extracted with ethyl acetate. The combined extracts were washed with brine and dried over magnesium sulfate. The solvent was evaporated in vacuo and the residue was recrystallized from a mixture of ethyl acetate and diisopropyl ether to give N-(4-[2-(2-pyridinyl)ethyl]phenyl}-4′-(trifluoromethyl)-1,1′-biphenyl-2-carboxamide (300 mg).

¹H-NMR (DMSO-d₆): δ2.89-3.05 (4H, m), 7.12 (2H, d, J=8.42 Hz), 7.14-7.24 (2H, m), 7.42 (2H, d, J=8.42 Hz), 7.33-7.74 (7H, m), 7.76 (1H, d, J=8.32 Hz), 8.49 (1H, d, J=4.04 Hz), 10.28 (1H, s).

### EXAMPLE 16

A solution of 4′-(trifluoromethyl)-1,1′-biphenyl-2-carbonyl chloride (1.64 g) in ethyl acetate (5 ml) was added to a mixture of 4-[2-(4-methyl-2-pyridinyl)ethyl]phenylamine (1.21 g) and triethylamine (1.162 g) in ethyl acetate (30 ml) at ambient temperature. The mixture was stirred at ambient temperature for 2 hours. The resultant mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (5:5). The fraction containing the objective compound was evaporated and the residue was recrystallized from a mixture of ethyl acetate and diisopropyl ether to give N-{4-[2-(4-methyl-2-pyridinyl)ethyl]phenyl}-4′-(trifluoromethyl)-1,1′-biphenyl-2-carboxamide (1.48 g).

¹H-NMR (DMSO-d₆): δ2.26 (3H, s), 2.93 (4H, s), 7.12 (2H, d, J=8.40 Hz), 7.00-7.14 (2H, m), 7.43 (2H, d, J=8.40 Hz), 7.49-7.66 (6H, m), 7.76 (2H, d, J=8.36 Hz), 8.34 (1H, d, J=4.98 Hz), 10.29 (1H, s).

### EXAMPLE 17

A solution of 4′-(trifluoromethyl)-1,1′-biphenyl-2-carbonyl chloride (598 mg) in tetrahydrofuran (5 ml) was added to a mixture of 4-[2-(4-pyrimidinyl)ethyl]phenylamine (598 mg) and triethylamine (606 mg) in tetrahydrofuran (15 ml) at ambient temperature. The mixture was stirred at ambient temperature for 3 hours. The resultant mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (6:4). The fraction contaning the objective compound was evaporated and the residue was recrystallized from a mixture of ethyl acetate and diisopropyl ether to give N-{4-[2-(4-pyrimidinyl)ethyl]phenyl}-4′-(trifluoromethyl)-1,1′-carboxamide (662 mg)

¹H-NMR (DMSO-d₆): δ2.93-3.04 (4H, s), 7.13 (2H, d, J=8.40 Hz), 7.37-7.66 (7H, m), 7.43 (2H, d, J=8.40 Hz), 7.76 (2H, d, J=8.34 Hz), 8.69 (1H, d, J=5.16 Hz), 9.08 (1H, s), 10.30 (1H, s).

### EXAMPLE 18

A solution of 4′-(trifluoromethyl)-1,1′-biphenyl-2-carbonyl chloride (374 mg) in tetrahydrofuran (5 ml) was added to a mixture of N-{4-[2-(4-aminophenyl)ethyl]-2-pyrimidinyl}acetamide (335 mg) and triethylamine (265 mg) in tetrahydrofuran (25 ml) and N,N-dimethylformamide (10 ml) at ambient temperature. The mixture was stirred at ambient temperature for 2 hours. The resultant mixture was poured into a mixture of ethyl acetate and water. The organic layer was washed with 5% aqueous potassium carbonate solution and brine and dried over magnesium sulfate. The solvent was evaporated in vacuo and the residue was recrystallized from a mixture of ethyl acetate and diisopropyl ether to give N-(4-{2-[2-(acetylamino)-4-pyrimidinyl]ethyl}-phenyl)-4′-(trifluoromethyl)-1,1′-biphenyl-2-carboxamide (324 mg).

¹H-NMR (DMSO-d₆): δ2.19 (3H, s), 2.93 (2H, s), 7.01 (1H, d, J=5.06 Hz), 7.13 (2H, d, J=8.42 Hz), 7.42 (2H, d, J=8.42 Hz), 7.40-7.65 (6H, m), 7.75 (2H, d, J=8.34 Hz), 8.47 (1H, d, J=5.06 Hz), 10.29 (1H, s), 10.43 (1H, s).

### EXAMPLE 19

A mixture of N-(4-{2-[2-(acetylamino)-4-pyrimidinyl]ethyl}-phenyl)-4′-(trifluoromethyl)-1,1′-biphenyl-2-carboxamide (888 mg) and 6N hydrochloric acid (10 ml) in ethanol (10 ml) was refluxed under stirring for 6 hours. The resultant mixture was evaporated in vacuo and the residue was dissolved in a mixture of ethyl acetate and water. The mixture was adjusted to pH 9.0 with 20% aqueous potassium carbonate solution. The organic layer was washed with brine and dried over magnesium sulfate. The solvent was evaporated in vacuo and the residue was chromatographed on silica gel eluting with chloroform and methanol (95:5). The fraction containing the objective compound was evaporated and the residue was washed with ethyl acetate to give N-{4-[2-(2-amino-4-pyrimidinyl)ethyl]phenyl}-4′-(trifluoromethyl)-1,1′-biphenyl-2-carboxamide (284 mg).

¹H-NMR (DMSO-d₆): δ2.69-2.91 (4H, m), 6.44 (1H, d, J=5.00 Hz), 6.50 (2H, s), 6.85 (2H, d, J=8.40 Hz), 7.12 (2H, d, J=8.40 Hz), 7.41-7.65 (6H, m), 7.76 (2H, d, J=8.34 Hz), 8.08 (1H, d, J=5.00 Hz), 10.29 (1H, s).

### EXAMPLE 20

A solution of 4′-(trifluoromethyl)-1,1′-biphenyl-2-carbonyl chloride (472 mg) in tetrahydrofuran (5 ml) was added to a mixture of N-{6-[2-(4-aminophenyl)ethyl]-2-pyridinyl}acetamide (423 mg) and triethylamine (335 mg) in tetrahydrofuran (25 ml) at ambient temperature. The mixture was stirred at ambient temperature for 3 hours. The resultant mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (6:4). The fraction containing the objective compound was evaporated and the residue was recrystallized from a mixture of ethyl acetate and diisopropyl ether to give N-(4-{2-[6-(acetylamino)-2-pyridinyl]ethyl}phenyl)-4′-(trifluoromethyl)-1,1′-biphenyl-2-carboxamide (133 mg).

¹H-NMR (DMSO-d₆): δ2.08 (3H, s), 2.91 (4H, s), 6.93 (1H, d, J=7.36 Hz), 7.11 (2H, d, J=8.38 Hz), 7.42 (2H, d, J=8.38 Hz), 7.49-7.67 (7H, m), 7.75 (2H, d, J=8.34 Hz), 7.89 (1H, d, J=8.18 Hz), 10.28 (1H, s), 10.41 (1H, s)

### EXAMPLE 21

A mixture of N-(4-{2-[6-(acetylamino)-2-pyridinyl]ethyl}-phenyl)-4′-(trifluoromethyl)-1,1′-biphenyl-2-carboxamide (472 mg) and 6N hydrochloric acid (10 ml) in methanol (10 ml) was refluxed under stirring for 6 hours. The resultant mixture was evaporated in vacuo and the residue was dissolved in a mixture of ethyl acetate and water. The mixture was adjusted to pH 9.0 with 20% aqueous potassium carbonate solution. The organic layer was washed with brine and dried over magnesium sulfate. The solvent was evaporated in vacuo and the residue was chromatographed on silica gel eluting with ethyl acetate and n-hexane (6:4-8:2). The fraction containing the objective compound was evaporated and the residue was recrystallized from a mixture of ethyl acetate and diisopropyl ether to give N-{4-[2-(6-amino-2-pyridinyl)ethyl]-phenyl}-4′-(trifluoromethyl)-1,1′-biphenyl-2-carboxamide (126 mg)

¹H-NMR (DMSO-d₆): δ2.51-2.85 (4H, m), 5.80 (2H, s), 6.24 (1H, d, J=8.10 Hz), 6.32 (1H, d, J=7.16 Hz), 7.11 (2H, d, J=8.38 Hz), 7.42 (2H, d, J=8.38 Hz), 7.49-7.65 (6H, m), 7.76 (2H, d, J=8.34 Hz), 10.28 (1H, s).

Preparation 7

A mixture of (2E)-3-(4-nitrophenyl)-1-(2-pyridinyl)-2-propen-1-one (2.0 g), iron (2.36 g) and ammonium chloride (0.27 g) in ethanol (60 ml) and water (5 ml) was refluxed under stirring for 3 hours. After removal of the catalyst, the solvent was evaporated in vacuo. The residue was dissolved in a mixture of water and ethyl acetate and adjusted to pH 8.0 with 5% aqueous potassium carbonate solution. The organic layer was washed with brine and dried over magnesium sulfate. The solvent was evaporated in vacuo and the residue was chromatographed on silica gel eluting with ethyl acetate and n-hexane (5:5). The fraction containing the objective compound was evaporated and the residue was crystallized from a mixture of ethyl acetate and diisopropyl ether to give (2E)-3-(4-aminophenyl)-1-(2-pyridinyl)-2-propen-1-one (100 mg).

¹H-NMR (DMSO-d₆): δ5.99 (2H, s), 6.62 (2H, d, J=8.52 Hz), 7.52 (2H, d, J=8.52 Hz), 7.56-8.10 (5H, m), 8.77 (1H, d, J=4.68 Hz).

### EXAMPLE 22

A solution of 4′-(trifluoromethyl)-1,1′-biphenyl-2-carbonyl chloride (127 mg) in tetrahydrofuran (5 ml) was added to a mixture of (2E)-3-(4-aminophenyl)-1-(2-pyridinyl)-2-propen-1-one (100 mg) and triethylamine (90 mg) in tetrahydrofuran (20 ml) at ambient temperature. The mixture was stirred at ambient temperature for 3 hours. The resultant mixture was poured into a mixture of ethyl acetate and water. The organic layer was washed with 5% aqueous potassium carbonate solution and brine and dried over magnesium sulfate. The solvent was evaporated in vacuo and the residue was recrystallized from a mixture of ethyl acetate and diisopropyl ether to give N-{4-[(1E)-3-oxo-3-(2-pyridinyl)-1-propenyl]phenyl}-4′-(trifluoromethyl)-1,1′-biphenyl-2-carboxamide (100 mg).

¹H-NMR (DMSO-d₆): δ7.52-7.85 (14H, m), 8.02-8.15 (2H, m), 8.18 (1H, d, J=16.1 Hz), 8.80 (1H, d, J=4.58 Hz), 10.34 (1H, s).

Preparation 8

A solution of (2E)-3-(4-nitrophenyl)-1-(2-pyridinyl)-2-propen-1-one (3.2 g) in methanol (150 ml) and tetrahydrofuran (50 ml) was hydrogenated over 10% palladium on carbon (1.5 g) under an atmospheric pressure of hydrogen at ambient temperature under stirring for 3 hours. After removal of the catalyst, the solvent was evaporated in vacuo to give a mixture of 3-(4-aminophenyl)-1-(2-pyridinyl)-1-propanone and 3-(4-aminophenyl)-1-(2-pyridinyl)-1-propanol (2.85 g).

¹H-NMR (DMSO-d₆): δ1.566-1.92 (2H, m), 2.36-2.49 (2H, m), 4.44 (1H, brs), 4.66 (2H, brs), 5.26 (1H, brs), 6.36 (2H, d, J=8.26 Hz), 6.72 (1H, d, J=8.26 Hz), 7.04-7.11 (1H, m), 7.37 (1H, d, J=7.84 Hz), 7.53-7.67 (1H, m), 8.33 (1H, d, J=4.15 Hz).

### EXAMPLE 23

A solution of 4′-(trifluoromethyl)-1,1′-biphenyl-2-carbonyl chloride (738 mg) in tetrahydrofuran (5 ml) was added to a mixture of 3-(4-aminophenyl)-1-(2-pyridinyl)-1-propanone and 3-(4-aminophenyl)-1-(2-pyridinyl)-1-propanol (3.52 g) and triethylamine (525 mg) in tetrahydrofuran (20 ml) at ambient temperature. The mixture was stirred at ambient temperature for 3 hours. The resultant mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (5:5-7:3). The first fraction was evaporated and the residue was recrystallized from a mixture of ethyl acetate and diisopropyl ether to give N-{4-[3-oxo-3-(2-pyridinyl)propyl]-phenyl}-4′-(trifluoromethyl)-1,1′-biphenyl-2-carboxamide (83 mg). The second fraction was evaporated to give N-{4-[3-hydroxy-3-(2-pyridinyl)propyl]phenyl}-4′-(trifluoromethyl-1,1′-biphenyl-2-carboxamide (320 mg).

N-{4-[3-Oxo-3-(2-pyridinyl)propyl]phenyl}-4′-(trifluoromethyl)-1,1′-biphenyl-2-carboxamide

¹H-NMR (DMSO-d₆): δ2.90 (2H, t, J=7.56 Hz), 3.48 (2H, t, J=7.56 Hz), 7.16 (2H, d, J=8.38 Hz), 7.43 (2H, d, J=8.38 Hz), 7.45-7.69 (7H, m), 7.76 (2H, d, J=8.36 Hz), 7.79-8.04 (2H, m), 8.11 (1H, d, J=4.58 Hz), 10.29 (1H, s).

N-{4-[3-Hydroxy-3-(2-pyridinyl)propyl]phenyl}-4′-(trifluoromethyl)-1,1′-biphenyl-2-carboxamide

¹H-NMR (DMSO-d₆): δ1.75-2.08 (2H, m), 2.59-2.66 (2H, m), 4.52-4.61 (1H, m), 5.06 (1H, d, J=5.06 Hz), 7.14-7.28 (4H, m), 7.11 (2H, d, J=8.38 Hz), 7.23 (1H, dd, J=5.40 Hz, 6.92 HZ), 7.44 (2H, d, J=8.38 Hz), 7.40-7.82 (8H, m), 7,76 (2H, d, J=8.08 Hz), 8.46 (1H, d, J=4.80 Hz), 10.30 (1H, s).

### EXAMPLE 24

A solution of 4′-(trifluoromethyl)-1,1′-biphenyl-2-carbonyl chloride (3.55 g) in ethyl acetate (10 ml) was added to a mixture of 3-(4-aminophenyl)-1-(2-pyridinyl)-1-propanone and 3-(4-aminophenyl)-1-(2-pyridinyl)-1-propanol (2.845 g) and N,O-bis(trimethylsilyl)acetamide (12 ml) in ethyl acetate (80 ml) at ambient temperature. The mixture was stirred at ambient temperature for 3 hours. The mixture was poured into a mixture of ethyl acetate and water. The organic layer was washed with 5% aqueous potassium carbonate solution and brine and dried over magnesium sulfate. The solvent was evaporated in vacuo and the residue was dissolved in methanol (50 ml). Sodium borohydride (474 mg) was added to the above solution and the mixture was stirred at ambient temperature for 2 hours. The reaction mixture was evaporated in vacuo. The residue was dissolved in a mixture of ethyl acetate and water. The organic layer was washed with brine and dried over magnesium sulfate. The solvent was evaporated in vacuo and the residue was chromatographed on silica gel eluting with ethyl acetate and n-hexane (6:4-7:3). The fraction containing the objective compound was evaporated and the residue was crystallized from a mixture of ethyl acetate and diisopropyl ether to give N-{4-[3-hydroxy-3-(2-pyridinyl)propyl]-phenyl}-4′-(trifluoromethyl)-1,1′-biphenyl-2-carboxamide (3.44 g).

¹H-NMR (DMSO-d₆): δ1.75-2.08 (2H, m), 2.59-2.66 (2H, m), 4.52-4.61 (1H, m), 5.06 (1H, d, J=5.06 Hz), 7.14-7.28 (4H, m), 7.11 (2H, d, J=8.38 Hz), 7.23 (1H, dd, J=5.40 Hz, 6.92 HZ), 7.44 (2H, d, J=8.38 Hz), 7.40-7.82 (8H, m), 7.76 (2H, d, J=8.08 Hz), 8.46 (1H, d, J=4.80 Hz), 10.30 (1H, s).

### EXAMPLE 25

A solution of N-{-[3-hydroxy-3-(2-pyridinyl)propyl]-phenyl}-4′-(trifluoromethyl)-1,1′-biphenyl-2-carboxamide (3.4 g) in methanol (50 ml) and 4N hydrogen chloride-dioxane solution (5 ml) was hydrogenated over 10% palladium on carbon (2 g) under an atmospheric pressure of hydrogen at ambient temperature under stirring for 20 hours. After removal of the catalyst, the solvent was evaporated in vacuo. The residue was dissolved in a mixture of water and ethyl acetate and adjusted to pH 8.0 with 5% aqueous potassium carbonate solution. The organic layer was washed with brine and dried over magnesium sulfate. The solvent was evaporated in vacuo and the residue was chromatographed on silica gel eluting with ethyl acetate and n-hexane (5:5). The fraction containing the objective compound was evaporated and the residue was crystallized from a mixture of ethyl acetate and diisopropyl ether to give N-{4-[3-(2-pyridinyl)propyl]phenyl)-4′-(trifluoromethyl)-1,1′-biphenyl-2-carboxamide (1.25 g).

¹H-NMR (DMSO-d₆): δ1.87-2.02 (2H, m), 2.56 (2H, t, J=7.404 Hz), 2.72 (2H, t, J=7.40 Hz), 7.14-7.21 (1H, m), 7.08 (1H, d, J=8.44 Hz), 7.523 (1H, d, J=7.72 Hz), 7.47 (2H, d, J=8.44 Hz), 7.49-7.74 (6H, m), 7.46 (2H, d,=8.40 Hz), 8.48 (1H, d, J=4.74 Hz), 10.32 (1H, s).

Preparation 9

An aqueous solution of 4N NaOH (12 ml) was added to a solution of 4′-aminoacetophenone (5.4 g) and 2-pyridinecarbaldehyde (4.5 g) in ethanol (50 ml) at ambient temperature under stirring and the resultant mixture was stirred at ambient temperature for 2 hours. The reaction mixture was adjusted to pH 8.0 with 6N hydrochloric acid and concentrated in vacuo to about ½ volume. Water (150 ml) was added to the above resultant mixture and the mixture was stirred at ambient temperature for 0.5 hour. The precipitate was collected by filtration, washed with water and dried to give (2E)-1-(4-aminophenyl)-3-(2-pyridinyl)-2-propen-1-one (7.0 g).

¹H-NMR (DMSO-d₆): δ6.22 (2H, s), 6.47 (2H, d, J=8.64 Hz), 7.32-7.48 (1H, m), 7.64 (1H, d, J=15.35 Hz), 7.79-8.00 (4H, m), 8.15 (1H, d, J=15.35 Hz), 8.68 (1H, d, J=4.67H).

Preparation 10

A solution of (2E)-1-(4-aminophenyl)-3-(2-pyridinyl)-2-propen-1-one (2.52 g) in methanol (100 ml) was hydrogenated over 10% palladium on carbon (1.25 g) under an. atmospheric pressure of hydrogen at ambient temperature under stirring for 2.5 hours. After removal of the catalyst, the solvent was evaporated in vacuo and the residue was triturated with a mixture of ethyl acetate and diisopropyl ether to give 1-(4-aminophenyl)-3-(2-pyridinyl)-1-propanone (3.52 g).

¹H-NMR (DMSO-d₆): δ3.05 (2H, t, J=7.01 Hz), 3.29 (2H, t, J=7.01 Hz), 6.03 (2H, s), 6.57 (2H, d, J=8.62 Hz), 7.14 (1H, m), 7.31 (1H, d, J=7.76 Hz), 7.71 (2H, d, J=8.62 Hz), 7.63-7.69 (1H, m), 8.45 (1H, d, J=4.51 Hz).

Preparation 11

Sodium borohydride (906 mg) was added to a solution of 1-(4-aminophenyl)-3-(2-pyridinyl)-1-propanone (3.6 g) in methanol (50 ml) at ambient temperature under stirring. The mixture was stirred at ambient temperature for 2 hours. The resultant solution was evaporated in vacuo and the residue was dissolved in a mixture of ethyl acetate and water. The organic layer was washed with 5% aqueous potassium carbonate solution and brine and dried over magnesium sulfate. The solvent was evaporated in vacuo to give 1-(4-aminophenyl)-3-(2-pyridinyl)-1-propanol (3.52 g).

¹H-NMR (DMSO-d₆): δ1.79-2.02 (2H, m), 2.56-2.83 (2H, m), 4.33-4.41 (1H, m), 4.89 (2H, s), 4.95 (1H, d, J=4.25H), 7.62-7.72 (2H, m), 8.45 (1H, d, J=4.73 Hz).

### EXAMPLE 26

A solution of 4′-(trifluoromethyl)-1,1-biphenyl-2-carbonyl chloride (427 mg) in tetrahydrofuran (5 ml) was added to a mixture of (2E)-1-(4-aminophenyl)-3-(2-pyridinyl)-2-propen-1-one (378 mg) and triethylamine (303 mg) in tetrahydrofuran (15 ml) at ambient temperature. The mixture was stirred at ambient temperature for 3 hours. The resultant mixture was poured into a mixture of ethyl acetate and water. The organic layer was washed with 5% aqueous potassium carbonate solution and brine and dried over magnesium sulfate. The solvent was evaporated in vacuo and the residue was recrystallized from mixture of ethyl acetate and diisopropyl ether to give N-{4-[(2E)-3-(2-pyridinyl)-2-propenoyl]phenyl}-4′-(trifluoromethyl)-1,1′-biphenyl-2-carboxamide (0.54 g).

¹H-NMR (DMSO-d₆): δ7.30-7.91 (12H, m), 8.10 (2H, d, J=8.68 Hz), 8.15 (1H, d, J=15.58 Hz), 8.70 (1H, d, J=4.64 Hz), 10.80 (1H, s).

### EXAMPLE 27

A solution of 4′-(trifluoromethyl)-1,1′-biphenyl-2-carbonyl chloride (2.0 g) in ethyl acetate (5 ml) was added to a mixture of 1-(4-aminophenyl)-3-(2-pyridinyl)-1-propanone (1.6 g) and triethylamine (1.41 g) in ethyl acetate (50 ml) at ambient temperature. The mixture was stirred at ambient temperature for 3 hours. The resultant mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (7:3). The fraction containing the objective compound was evaporated and the residue was recrystallized from a mixture of ethyl acetate and diisopropyl ether to give N-{4-[3-(2-pyridinyl)propanoyl]phenyl}-4′-(trifluoromethyl)-1,1′-biphenyl-2-carboxamide (2.58 g).

¹H-NMR (DMSO-d₆): δ3.10 (2H, t, J=6.84 Hz), 3.42 (2H, t, J=6.84 Hz), 7.17-7.31 (1H, m), 7.33 (1H, d, J=6.66 Hz), 7.56-7.78 (11H, m), 7.95 (2H, d, J=8.72 Hz), 8.45 (1H, d, J=3.94 Hz), 10.72 (1H, s).

### EXAMPLE 28

A solution of 4′-(trifluoromethyl)-1,1′-biphenyl-2-carbonyl chloride (4.39 g) in ethyl acetate (10 ml) was added to a mixture of 1-(4-aminophenyl)-3-(2-pyridinyl)-1-propanol (3.52 g) and N,O-bis(trimethylsilyl)acetamide (15 ml) in ethyl acetate (100 ml) at ambient temperature. The mixture was stirred at ambient temperature for 3 hours. The resultant mixture was poured into a mixture of ethyl acetate and water. The organic layer was washed with 5% aqueous potassium carbonate solution and brine and dried over magnesium, sulfate. The solvent was evaporated in vacuo and the residue was chromatographed on silica gel eluting with ethyl acetate and n-hexane (6:4-7:3). The fraction containing the objective compound was evaporated and the residue was recrystallized from a mixture of ethyl acetate and diisopropyl ether to give N-{4-[1-hydroxy-3-(2-pyridinyl)propyl]phenyl}-4′-(trifluoromethyl)-1,1′-biphenyl-2-carboxamide (4.49 g).

¹H-NMR (DMSO-d₆). δ1.92-2.03 (2H, m), 2.62-2.87 (2H, m), 4.49-4.57 (1H, m), 5.27 (1H, d, J=4.28 Hz), 7.14-7.28 (4H, m), 7.50 (2H, d, J=8.50 Hz), 7.52-7.74 (7H, m), 7.76 (2H, d, J=8.40 Hz), 8.46 (1H, d, J=4.10 Hz), 10.34 (1H, s).

### EXAMPLE 29

A solution of N-{4-[(2E)-3-(2-pyridinyl)-2-propenoyl]-phenyl}-4′-(trifluoromethyl)-1,1′-biphenyl-2-carboxamide (3.28 g) in methanol (100 ml) and tetrahydrofuran (50 ml) was hydrogenated over 10% palladium on carbon (1 g) under an atmospheric pressure of hydrogen at ambient temperature under stirring for 4 hours. After removal of the catalyst, the solvent was evaporated in vacuo and the residue was chromatographed on silica gel eluting with ethyl acetate and n-hexane (5:5-7:3). The fraction containing the objective compound was evaporated and the residue was crystallized from a mixture of ethyl acetate and diisopropyl ether to give N-≡4-[3-(2-pyridinyl)propanoyl]phenyl}-4′-(trifluoromethyl)-1,1′-biphenyl-2-carboxamide (2.54 g).

¹H-NMR (DMSO-d₆): δ3.10 (2H, t, J=6.84 Hz), 3.42 (2H, t, J=6.84 Hz), 7.17-7.31 (1H, m), 7.33 (1H, d, J=6.66 Hz), 7.56-7.78 (11H, m), 7.95 (2H, d, J=8.72 Hz), 8.45 (1H, d, J=3.94 Hz), 10.72 (1H, s).

### EXAMPLE 30

Sodium borohydride (211 mg) was added to a solution of N-(4-[3-(2-pyridinyl)propanoyl]phenyl}-4′-(trifluoromethyl)-1,1′-biphenyl-2-carboxamide (2.4 g) in methanol (50 ml) at ambient temperature under stirring. The mixture was stirred at ambient temperature for 4 hours. The resultant solution was evaporated in vacuo and the residue was dissolved in a mixture of ethyl acetate and water. The organic layer was washed with 5% aqueous potassium carbonate solution and brine and dried over magnesium sulfate. The solvent was evaporated in vacuo to give N-{4-[1-hydroxy-3-(2-pyridinyl)propyl]phenyl}-4′-(trifluoromethyl)-1,1′-biphenyl-2-carboxamide (2.4 g).

¹H-NMR (DMSO-d₆):.δ1.92-2.03 (2H, m), 2.62-2.87 (2H, m), 4.49-4.57 (1H, m), 5.27 (1H, d, J=4.28 Hz), 7.14-7.28 (4H, m), 7.50 (2H, d, J=8.50 Hz), 7.52-7.74 (7H, m), 7.76 (2H, d, J=8.40 Hz), 8.46 (1H, d, J=4.10 Hz), 10.34 (1H, s).

### EXAMPLE 31

A solution of N-{4-[1-hydroxy-3-(2-pyridinyl)propyl]-phenyl}-4′-(trifluoromethyl)-1,1′-biphenyl-2-carboxamide (2.4 g) in methanol (50 ml) and 4N hydrogen chloride-dioxane solution (5 ml) was hydrogenated over 10% palladium on carbon (1 g) under an atmospheric pressure of hydrogen at ambient temperature under stirring for 2 hours. After removal of the catalyst; the solvent was evaporated in vacuo. The residue was dissolved in a mixture of water and ethyl acetate and adjusted to pH 8.0 with 5% aqueous potassium carbonate solution. The organic layer was washed with brine and dried over magnesium sulfate. The solvent was evaporated in vacuo and the residue was chromatographed on silica gel eluting with ethyl acetate and n-hexane (5:5). The fraction containing the objective compound was evaporated and the residue was crystallized from a mixture of ethyl acetate and diisopropyl ether to give N-{4-[3-(2-pyridinyl)propyl]phenyl}-4′-(trifluoromethyl)-1,1′-biphenyl-2-carboxamide (1.42 g).

¹H-NMR (DMSO-d₆): δ1.87-2.02 (2H, m), 2.56 (2H, t, J=7.404 Hz), 2.72 (2H, t, J=7.40 Hz), 7.14-7.21 (1H, m), 7.08 (1H, d, J=8.44 Hz), 7.523 (1H, d, J=7.72 Hz), 7.47 (2H, d, J=8.44 Hz), 7.49-7.74 (6H, m), 7.46 (2H, d, J=8.40 Hz), 8.48 (1H, d, J=4.74 Hz), 10.32 (1H, s).

### EXAMPLE 32

Sodium borohydride (0.113 g) was added to a solution of N-(4-[(2E)-3-(2-pyridinyl)-2-propenoyl]phenyl}-4′-(trifluoromethyl)-1,1′-biphenyl-2-carboxamide (1.42 g) in methanol (30 ml) and tetrahydrofuran (20 ml) at ambient temperature under stirring. The mixture was stirred at ambient temperature for an hour. The resultant solution was evaporated in vacuo and the residue was dissolved in a mixture of ethyl acetate and water. The organic layer was washed with 5% aqueous potassium carbonate solution and brine and dried over magnesium sulfate. The solvent was evaporated in vacuo to give N-{4-[(2E)-1-hydroxy-3-(2-pyridinyl)-2-propenyl]phenyl}-4′-(trifluoromethyl)-1,1′-biphenyl-2-carboxamide (0.54 g).

¹H-NMR (DMSO-d₆): δ5.23-5.28 (1H, m), 5.64 (1H, d, J=4.32 Hz), 6.66 (1H, d, J=15.72 Hz), 6.77-6.88 (1H, m), 7.13-7.72 (13H, m), 7.76 (2H, d, J=8.308 Hz), 8.48 (1H, d, J=4.16 Hz), 10.36 (1H, s).

### EXAMPLE 33

A solution of N-{4-[(2E)-1-hydroxy-3-(2-pyridinyl)-2-propenyl]phenyl}-4′-(trifluoromethyl)-1,1′-biphenyl-2-carboxamide (1.4 g) in methanol (50 ml) and 4N hydrogen chloride-dioxane solution (2 ml) was hydrogenated over 10% palladium on carbon (0.7 g) under an atmospheric pressure of hydrogen at ambient temperature under stirring for 2 hours. After removal of the catalyst, the solvent was evaporated in vacuo. The residue was dissolved in a mixture of water and ethyl acetate and adjusted to pH 8.0 with 5% aqueous potassium carbonate solution. The organic layer was washed with brine and dried over magnesium sulfate. The solvent was evaporated in vacuo and the residue was chromatographed on silica gel eluting with ethyl acetate and n-hexane (5:5). The fraction containing the objective compound was evaporated and the residue was crystallized from a mixture of ethyl acetate and diisopropyl ether to give N-{4-[3-(2-pyridinyl)propyl]phenyl}-4′-(trifluoromethyl)-1,1′-biphenyl-2-carboxamide (320 mg).

¹H-NMR (DMSO-d₆): δ1.87-2.02 (2H, m), 2.56 (2H, t, J=7.404 Hz), 2.72 (2H, t, J=7.40 Hz), 7.14-7.21 (1H, m), 7.08 (1H, d, J=8.44 Hz), 7.523 (1H, d, J=7.72 Hz), 7.47 (2H, d, J=8.44 Hz), 7.49-7.74 (6H, m), 7.46 (2H, d, J=8.40 Hz), 8.48 (1H, d, J=4.74 Hz), 10.32 (1H, s).

### EXAMPLE 34

An aqueous solution of 4N NaOH (4.1 ml) was added to a solution of N-(4-acetylphenyl)-4′-(trifluoromethyl)-1,1′-biphenyl-2-carboxamide (5.4 g) and 2-pyridinecarbaldehyde (1.69 g) in ethanol (50 ml) at ambient temperature under stirring and the resultant mixture was stirred for 2 hours at ambient temperature. The reaction mixture was poured into a mixture of ethyl acetate and water and adjusted to pH 4. 0 with 6N hydrochloric acid. The organic layer was washed with 5% aqueous potassium carbonate solution and brine and dried over magnesium sulfate. The solvent was evaporated in vacuo to give N-{4-[(2E)-3-(2-pyridinyl)-2-propenoyl]phenyl}-4′-(trifluoromethyl)-1,1′-biphenyl-2-carboxamide (4.71 g).

¹H-NMR (DMSO-d₆); δ7.30-7.91 (12H, m), 8.10 (2H, d, J=8.68 Hz), 8.15 (1H, d, J=15.58 Hz),. 8.70 (1H, d, J=4.64 Hz), 10.80 (1H, s).

Preparation 12

A solution of 4′-(trifluoromethyl)-1,1′-biphenyl-2-carbonyl chloride (5.7 g) in tetrahydrofuran (10 ml) was added to a mixture of 4′-aminoacetophenone (2.7 g) and triethylamine (4.09 g) in tetrahydrofuran (50 ml) at ambient temperature. The mixture was stirred at ambient temperature for 3 hours. The resultant mixture was poured into a mixture of ethyl acetate and water. The organic layer was washed with 5% aqueous potassium carbonate solution and brine and dried over magnesium sulfate. The solvent was evaporated in vacuo and the residue was recrystallized from a mixture of ethyl acetate and diisopropyl ether to give N-(4-acetylphenyl)-4′-(trifluoromethyl)-1,1′-biphenyl-2-carboxamide (5.7 g).

¹H-NMR (DMSO-d₆): δ2.52 (3H, s), 7.53-7.78 (10H, m), 7.91 (2H, d, J=8.68 Hz), 10.72 (1H, s).

### EXAMPLE 35

N-{4-[(2E)-3-(6-Methyl-2-pyridinyl)-2-propenoyl]phenyl}-4′-(trifluoromethyl)-1,1′-biphenyl-2-carboxamide

The title compound was obtained from N-(4-acetylphenyl)-4′-(trifluoromethyl)-1,1′-biphenyl-2-carboxamide and 6-methyl-2-pyridinecarbaldehyde in the same manner as in Example 34.

¹H-NMR (DMSO-d₆): δ2.54 (3H, s), 7.30 (1H, d, J=7.26 Hz), 7.52-8.06 (12H, m), 8.06-8.14 (2H, m), 10.80 (1H, s)

### EXAMPLE 36

A solution of N-{4-[(2E)-3-(6-methyl-2-pyridinyl)-2-propenoyl]phenyl}-4′-(trifluoromethyl)-1,1′-biphenyl-2-carboxamide (0.98 g) in methanol (50 ml) was hydrogenated over 10% palladium on carbon (0.5 g) under an atmospheric pressure of hydrogen at ambient temperature under stirring for 4 hours. After removal of the catalyst, the solvent was evaporated in vacuo and the residue was dissolved in a methanol (20 ml). Sodium borohydride (77 mg) was added to the above solution and the mixture was stirred for 3 hours at ambient temperature. The reaction mixture was evaporated in vacuo and the residue was dissolved in a mixture of ethyl acetate and water. The organic layer was washed with 5% aqueous potassium carbonate solution and brine and dried over, magnesium sulfate. The solvent was evaporated in vacuo and the residue was chromatographed on silica gel eluting with ethyl acetate and n-hexane (5:5-7:3). The fraction containing the objective compound was evaporated to give N-{4-[1-hydroxy-3-(6-methyl-2-pyridinyl)propyl]phenyl}-4′-(trifluoromethyl)-1,1′-biphenyl-2-carboxamide (0.6 g).

¹H-NMR (DMSO-d₆): δ1.88-1.99 (2H, m), 2.55-2.81 (2H, m), 4.48 (1H, m), 4.27 (1H, d, J=4.36 Hz), 6.98-7.04 (2H, m), 7.25 (2H, d, J=8.42 Hz), 7.46-7.66 (10H, m), 7.76 (2H, d, J=8.34 Hz), 10.32 (1H, s).

### EXAMPLE 37

N-{4-[3-(6-Methyl-2-pyridinyl)propyl]phenyl}-4′-(trifluoromethyl)-1,1′-biphenyl-2-carboxamide

The title compound was obtained from N-{4-[1-hydroxy-3-(6-methyl-2-pyridinyl)propyl]phenyl}-4′-(trifluoromethyl)-1,1′-biphenyl-2-carboxamide in the same manner as in Example 31.

¹H-NMR (DMSO-d₆): δ1.83-1.99 (2H, m), 2.42-2.70 (4H, m), 2.42 (3H, s), 7.02 (1H, dd, J=2.96 Hz, 8.68 Hz), 7.12 (2H, d, J=8.36 Hz), 7.45 (2H, d, J=8.36 Hz), 7.49-7.66 (8H, m), 7.76 (2H, d, J=8.34 Hz), 10.31 (1H, s).

### EXAMPLE 38

An aqueous solution of 4N NaOH (0.83 ml) was added to a solution-of N-(4-acetylphenyl)-4′-(trifluoromethyl)-1,1′-biphenyl-2-carboxamide (1.15 g) and N-(6-formyl-2-pyridinyl)acetamide (0.5 g) in ethanol (20 ml) at ambient temperature under stirring and the resultant mixture was stirred for 1.5 hours at ambient temperature. The reaction mixture was poured into a mixture of water and ethyl acetate and extracted with ethyl acetate. The extract was washed with brine and dried over magnesium sulfate. The solvent was evaporated in vacuo and the residue was crystallized from a mixture of ethyl acetate and diisopropyl ether to give N-(4-{(2E)-3-[6-(acetylamino)-2-pyridinyl]-2-propenoyl}phenyl)-4′-(trifluoromethyl)-1,1′-biphenyl-2-carboxamide (0.89 g).

¹H-NMR (DMSO-d₆): δ2.14 (3H, s), 7.53-8.14 (17H, m), 10.55 (1H, s), 10.80 (1H, s).

### EXAMPLE 39

A solution of N-(4-{(2E)-3-(6-(acetylamino)-2-pyridinyl]-2-propenoyl}phenyl)-4′-(trifluoromethyl)-1,1′-biphenyl-2-carboxamide (3.04 g) in methanol (60 ml) and tetrahydrofuran (30 ml) was hydrogenated over 10% palladium on carbon (0.6 g) under an atmospheric pressure of hydrogen at ambient temperature under stirring for 4-hours. After removal of the catalyst, the solvent was evaporated in vacuo and the residue was chromatographed on silica gel eluting with ethyl acetate and n-hexane (5;5-6.4). The first fraction was evaporated to give N-(4-{3-[6-(acetylamino)-2-pyridinyl]propanoyl}phenyl)-4′-(trifluoromethyl)-1,1′-biphenyl-2-carboxamide (1.19 g). The second fraction was evaporated to give N-(4-{3-[6-(acetylamino)-2-pyridinyl]-1-hydroxypropyl}phenyl)-4′-(trifluoromethyl)-1,1′-biphenyl-2-carboxamide (1.50 g).

N-(4-{3-[6-(Acetylamino)-2-pyridinyl]propanoyl}phenyl)-4′-(trifluoromethyl)-1,1′-biphenyl-2-carboxamide

¹H-NMR (DMSO-d₆): δ2.12 (3H, s), 3.10 (2H, t, J=7.16 Hz), 3.38 (2H, t, J=7.16 Hz), 7.02 (1H, d, J=7.24 Hz), 7.52-7.96 (11H, m), 7.78-7.92 (1H, m), 7.94 (1H, d, J=8.76 Hz), 10.34 (1H, s).

N-(4-{3-[6-(Acetylamino)-2-pyridinyl]-1-hydroxypropyl}-phenyl)-4′-(trifluoromethyl)-1,1′-biphenyl-2-carboxamide

¹H-NMR (DMSO-d6): δ1.74-1.99 (2H, m), 2.50-2.74 (2H, m), 4.48-4.59 (1H, m), 5.21 (1H, d, J=4.28 Hz), 6.92 (1H, d, J=7.24 Hz), 7.25 (2H, d, J=8.46 Hz), 7.47 (2H, d, J=8.46 Hz), 7.49-7.67 (7H, m), 7.76 (2H, d, J=8.36 Hz), 7.82 (1H, d, J=8.36 Hz), 10.32 (1H, s), 10.35 (1H, s)

### EXAMPLE 40

N-(4-{3-[6-(Acetylamino)-2-pyridinyl]-1-hydroxypropyl}-phenyl)-4′-(trifluoromethyl)-1,1′-biphenyl-2-carboxamide

The title compound was obtained from N-(4-{3-[6-(acetylamino)-2-pyridinyl]propanoyl}phenyl)-4′-(trifluoromethyl)-1,1′-biphenyl-2-carboxamide in the same manner as in Example 30.

¹H-NMR (DMSO-d₆): δ1.74-1.99 (2H, m), 2.50-2.74 (2H, m), 4.48-4.59 (1H, m), 5.21 (1H, d, J=4.28 Hz), 6.92 (1H, d, J=7.24 Hz), 7.25 (2H, d, J=8.46 Hz), 7.47 (2H, d, J=8.46 Hz), 7.49-7.67 (7H, m), 7.76 (2H, d, J=8.36 Hz), 7.82 (1H, d, J=8.36 Hz), 10.32 (1H, s), 10.35 (1H, s).

### EXAMPLE 41

N-(4-{3-[6-(Acetylamino)-2-pyridinyl]propyl}phenyl)-4′-(trifluoromethyl)-1,1′-biphenyl-2-carboxamide

The title compound was obtained from N-(4-{3-[6-(acetylamino)-2-pyridinyl]-1-hydroxypropyl)phenyl)-4′-(trifluoromethyl)-1,1′-biphenyl-2-carboxamide in the same manner as in Example 31.

¹H-NMR (DMSO-d₄): δ1.87-1.99 (2H, m), 2.07 (3H, s), 2.50-2.67 (4H, m), 6.94 (1H, d, J=7.18 Hz), 7.11 (2H, d, J=8.36 Hz), 7.76 (2H, d, J=8.36 Hz), 7.41-7.69 (10H, m), 7.76 (2H, d=8.36 Hz), 7.78 (1H, d, J=8.17 Hz), 10.29 (1H, s), 10.36 (1H, s).

### EXAMPLE 42

N-{4-[(2E)-3-(6-Amino-2-pyridinyl)-2-propenoyl]phenyl}-4′-(trifluoromethyl)-1,1′-biphenyl-2-carboxamide

The title compound was obtained from N-(4-{(2E)-3-[6-(acetylamino)-2-pyridinyl]-2-propenoyl}phenyl)-4′-(trifluoromethyl)-1,1′-biphenyl-2-carboxamide in the same manner as in Example 21.

¹H-NMR (DMSO-d₆): δ6,.16 (2H, s), 6.54 (1H, d, J=8.25 Hz), 6.95 (1H, d, J=7.12 Hz), 7.43-7.97 (14H, m), 8.01 (2H, d, J=8.65 Hz), 10.79 (1H, s).

### EXAMPLE 43

N-{4-[3-(6-Amino-2-pyridinyl)propanoyl]phenyl}-4′-(trifluoromethyl)-1,1′-biphenyl-2-carboxamide

The title compound was obtained from N-(4-{3-[6-(acetylamino)-2-pyridinyl]propanoyl}phenyl)-4′-(trifluoromethyl)-1,1′-biphenyl-2-carboxamide in the same manner as in Example 21.

¹H-NMR (DMSO-d₆): δ2.82 (2H, t, J=7.29 Hz), 3.22 (2H, t, J=7.29 Hz), 5.76 (2H, s), 6.25 (1H, d, J=8.08 Hz), 6.39 (1H, d, J=7.13 Hz), 7.22-7.29 (1H, m), 7.52-7.78 (10H, m), 7.93 (2H, d, J=8.70 Hz), 10.71 (1H, s).

### EXAMPLE 44

N-{4-[3-(6-Amino-2-pyridinyl)propyl]phenyl}-4′-(trifluoromethyl)-1,1′-biphenyl-2-carboxamide

The title compound was obtained from N-(4-{3-[6-(acetylamino)-2-pyridinyl]propyl}phenyl)-4′-(trifluoromethyl)-1,1′-biphenyl-2-carboxamide in the same manner as in Example 21.

¹H-NMR (DMSO-d₆): δ1.81-1.91 (2H, m), 2.42-2.57 (4H, m), 5.76 (2H, s), 6.24 (1H, d, J=8.12 Hz), 6.32 (1H, d, J=7.12 Hz), 7.10 (2H, d, J=8.38 Hz), 7.25 (1H, d, J=7.58 Hz), 7.43 (2H, d, J=8.38 Hz), 7.49-7.66 (6H, m), 7.75 (2H, d, J=8.30 Hz), 10.29 (1H, s).

### EXAMPLE 45

N-{4-[(2E)-3-(3-Pyridinyl)-2-propenoyl]phenyl}-4′-(trifluoromethyl)-1,1′-biphenyl-2-carboxamide

The title compound was obtained from N-(4-acetylphenyl)-4′-(trifluoromethyl)-1,1′-biphenyl-2-carboxamide and 3-pyridinecarbaldehyde in the same manner as in Example 34.

¹H-NMR (DMSO-d6): δ7.47-7.79 (12H, m), 8.08 (1H, d, J=15.8 Hz), 8.16 (2H, d, J=8.69 Hz), 8.33-8.49 (1H, m), 8.61-8.64 (1H, m), 9.03 (1H, d, J=1.74 Hz), 10.78 (1H, s).

Preparation 13

(2E)-1-(4-Aminophenyl)-3-(3-pyridinyl)-2-propen-1-one

The title compound was obtained from 4′-aminoacetophenone and 3-pyridinecarbaldehyde in the same manner as in Preparation 9.

¹H-NMR (DMSO-d₆): δ6.21 (2H, s), 6.60-6.68 (2H, m), 7.41 (1H, dd, J=4.92 Hz,7.97 Hz), 7.63 (1H, d, J=8.65 Hz), 7.95-8.06 (3H, m), 8.29-8.35 (1H, m), 8.99 (1H, d, J=1.96 Hz).

Preparation 14

1-(4-Aminophenyl)-3-(3-pyridinyl)-1-propanone

The title compound was obtained from (2E)-1-(4-aminophenyl)-3-(3-pyridinyl)-2-propen-1-one in the same manner as in Preparation 10.

¹H-NMR (DMSO-d₆): δ2.90 (2H, t, J=7.33 Hz), 3.18 (2H, t, J=7.33 Hz), 6.04 (2H, s), 6.52 (2H, d, J=8.64 Hz), 7.25-7.31 (1H, m), 7.80 (2H, d, J=8.64 Hz), 7.72-7.82 (1H, m), 8.36-8.40 (1H, m), 8.48 (1H, s).

### EXAMPLE 46

N-{4-[3-(3-Pyridinyl)propanoyl]phenyl}-4′-(trifluoromethyl)-1,1′-biphenyl-2-carboxamide

The title compound was obtained from 1-(4-aminophenyl)-3-(3-pyridinyl)-1-propanone and 4′-(trifluoromethyl)-1,1′-biphenyl-2-carbonyl chloride in the same manner as in Example 27.

¹H-NMR-(DMSO-d₆): δ2.95 (2H, t, J=7.22 Hz), 3.37 (2H, t, J=7.22 Hz), 7.23-7.33 (1H, m), 7.44-7.78 (11H, m), 7.95 (2H, d, J=8.65 Hz), 8.39-8.42 (1H, m), 8.52 (1H, d, J=2.08 Hz), 10.31 (1H, s).

### EXAMPLE 47

N-{4-[1-Hydroxy-3-(3-pyridinyl)propyl]phenyl}-4′-(trifluoromethyl)-1,1′-biphenyl-2-carboxamide

The title compound was obtained from N-{4-[3-(3-pyridinyl)propanoyl]phenyl}-4′-(trifluoromethyl)-1,1′-biphenyl-2-carboxamide in the same manner as in Example 30.

¹H-NMR (DMSO-d₆): δ1.81-1.92 (2H, m), 2.51-2.75 (2H, m), 4.43-4.51 (1H, m), 5.24 (1H, d, J=4.46 Hz), 7.24 (2H, d, J=8.28 Hz), 7.25-7.32 (1H, m), 7.47 (2H, d, J=8.28 Hz), 7.49-7.66 (7H, m), 7.76 (2H, d, J=8.38 Hz), 8.37 (2H, m), 10.32 (1H, s).

### EXAMPLE 48

N-{4-[3-(3-Pyridinyl)propyl]phenyl}-4′-(trifluoromethyl)-1,1′-biphenyl-2-carboxamide

The title compound was obtained from N-{4-[1-hydroxy-3-(3-pyridinyl)propyl]phenyl}-4′-(trifluoromethyl)-1,1′-1,1′-biphenyl-2-carboxamide in the same manner as in Example 31.

¹H-NMR (DMSO-d₆): δ1.79-2.00 (2H, m), 2.51-2.63 (4H, m), 7.11 (2H, d, J=8.36 Hz), 7.27-7.46 (1H, m), 7.47-7.66 (9H, m), 7.75 (2H, d, J=8.34 Hz), 8.38-8.43 (1H, m), 10.30 (1H, s).

Preparation 15

To a suspension of 4-nitrobenzylamine hydrochloride (3.77 g), 2-pyridinecarboxylic acid (2.46 g) and HOBT.H₂O (2.70 g) in dichloromethane (100 ml) was added WSC.HCl (3.83 g), followed by addition of triethylamine (4.05 g) at 5° C. The resulting solution was stirred at room temperature for 24 hours and poured into water. The separated organic layer was washed with brine, dried over magnesium sulfate and evaporated in vacuo. The residue was purified by column chromatography on silica gel eluting with hexane:ethyl acetate (1:1) to give N-(4-nitrobenzyl)-2-pyridinecarboxamide (3.92 g) as a yellow solid.

¹H-NMR (DMSO-d₆): δ4.62 (2H, d, J=6.4 Hz), 7.58 (2H, d, J=8.7 Hz), 7.6-7.7 (1H, m), 8.0-8.1 (2H, m), 8.19 (2H, d, J=8.7 Hz), 8.68 (1H, d, J=4.6 Hz), 9.57 (1H, t, J=6.4 Hz).

APCI-MS (m/z): 258 (M⁺+1)

Preparation 16

To a suspension of N-(4-nitrobenzyl)-2-pyridinecarboxamide (3.90 g) in ethanol (100 ml) were added iron(III) chloride (anhydrous) (49 mg) and active-charcoal (4 g) and the mixture was heated to 80° C. To the mixture was added dropwise hydrazine hydrate (3.04 g) and the mixture was stirred at the same temperature for 3 hours. The active-charcoal was filtered off by celite and washed with ethanol. The filtrate was evaporated in vacuo and the residue was purified by column chromatography on silica gel eluting with ethyl acetate to give N-(4-aminobenzyl)-2-pyridinecarboxamide (2.80 g) as a light brown solid.

¹H-NMR (DMSO-d₆) δ4.31 (2H, d, J=6.3 Hz), 5.00 (2H, brs), 5.50 (2H, d, J=8.3 Hz), 7.00 (2H, d, J=8.3 Hz), 7.55-7.7 (1H, m), 7.95-8.1 (2H, m) 8.62 (2H, d, J=4.7 Hz), 9.0 (15H, t, J=6.3 Hz).

APCI-MS (m/z): 228 (M⁺+1)

### EXAMPLE 49

To a suspension of 4′-(trifluoromethyl)-1,1′-biphenyl-2-carboxylic acid (799 mg) in toluene (10 ml) were added thionyl chloride (713 mg) and N,N-dimethylformamide (5 drops) and the mixture was stirred at 100° C. for 3 hours. The mixture was evaporated in vacuo and the residue was dissolved in dichloromethane (10 ml). The acid chloride solution was added to a solution of N-(4-aminobenzyl)-2-pyridinecarboxamide (454 mg) and triethylamine (405 mg) in dichloromethane (40 ml) at 5° C. and the mixture was stirred at the same temperature for 16 hours. The mixture was poured into water and the separated organic layer was washed with brine, dried over magnesium sulfate and evaporated in vacuo. The residue was purified by column chromatography on silica gel eluting with ethyl acetate to give N-[4-({[4′-(trifluoromethyl)-1,1′-biphenyl-2-yl]carbonyl}amino)benzyl]-2-pyridinecarboxamide (745 mg) as white crystals.

¹H-NMR (DMSO-d₆): δ4.43 (2H, d, J=6.3 Hz), 7.23 (2H, d, J=8.4 Hz), 7.45 (2H, d, J=8.4 Hz), 7.5-7.9 (9H, m), 7.95-8.1 (2H, m), 8.65 (2H, d, J=4.7 Hz), 9.27 (1H, t, J=6.3 Hz), 10.33 (1H, s).

APCI-MS (m/z): 476 (Me⁺+1)

Preparation 17

N-(3-Nitrobenzyl)-2-pyridinecarboxamide

The title compound was obtained from 3-nitrobenzylamine hydrochloride and 2-pyridinecarboxylic acid in the same manner as in Preparation 15 as a yellow solid.

¹H-NMR (DMSO-₆); δ4.60 (2H, d, J=6.4 Hz), 7.55-7.7 (2H, m), 7.80 (1H, d, J=7.7 Hz), 7.95-8.15 (3H, m), 8.21 (1H, s), 8.68. (1H, d, J=4.7 Hz), 9.50 (1H, t, J=6.4 Hz).

APCI-MS (m/z): 258 (M⁺+1)

Preparation 18

N-(3-Aminobenzyl)-2-pyridinecarboxamide

The title compound was obtained from N-(3-nitrobenzyl)-2-pyridinecarboxamide in the same manner as in Preparation 16 as a light brown solid.

¹H-NMR (DMSO-d₆): δ4.37 (2H, d, J=6.4 Hz), 5.47 (2H, brs), 6.4-6.6 (2H, m), 6.9-7.1 (1H, m), 7.6-7.7 (1H, m), 7.95-8.1 (2H, m), 8.65 (1H, d, J=4.6 Hz), 9.14 (1H, t, J=6.4 Hz).

APCI-MS (m/z): 228 (M⁺+1)

### EXAMPLE 50

N-{3-[({4′-(Trifluoromethyl)-1,1′-biphenyl-2-yl}carbonyl)amino]benzyl}-2-pyridinecarboxamide

The title compound was obtained from N-(3-aminobenzyl)-2-pyridinecarboxamide and 4′-(trifluoromethyl)-1,1′-biphenyl-2carboxylic acid in the same manner as in Example 49 as white crystals.

¹H-NMR (DMSO-d₆): δ4.45 (2H, d, J=6.4 Hz), 7.02 (2H, d, J=7.8 Hz), 7.21 (2H, dd, J=7.7 and 7.7 Hz), 7.42 (1H, d, J=7.8 Hz) 7.5-7.8 (9H, m), 8.0-8.15 (2H, m), 8.65 (2H, d, J=4.7 Hz), 9.31 (1H, t, J=6.4 Hz), 10.39 (1H, s).

APCI-MS (m/z): 476 (M⁺+1)

### EXAMPLE 51

To a suspension of N-(4-aminobenzyl)-2-pyridinecarboxamide (514 mg), 1,1′-biphenyl-2-carboxylic acid (388 mg) and HOBT.H₂O (306 mg) in dichloromethane (30 ml) was added WSC.HCl (422 mg), followed by addition of triethylamine (223 mg) at room temperature. The resulting solution was stirred at room temperature for 20 hours and poured into water. The separated organic layer was washed with brine, dried over magnesium sulfate and evaporated in vacuo. The residue was purified by column chromatography on silica gel eluting with hexane:ethyl acetate (1:2) to give N-{4-[(1,1′-biphenyl-2-ylcarbonyl)amino]benzyl}-2-pyridinecarboxamide (517 mg) as white crystals.

¹H-NMR (DMSO-d₆): δ4.42 (2H, d, J=6.3 Hz), 7.2-7.7 (14H, m), 7.9-8.1 (2H, m), 8.64 (2H, d, J=4.7 Hz), 9.26 (1H, t, J=6.3 Hz), 10.18 (1H, s).

APCI-MS (m/z): 422 (M⁺+1)

### EXAMPLE 52

N-[4-({2-[3-(Trifluoromethyl)anilino]benzoyl}amino)benzyl]-2-pyridinecarboxamide

The title compound was obtained from N-(4-aminobenzyl)-2-pyridinecarboxamide and 2-[3-(trifluoromethyl)anilino]benzoic acid in the same manner as in Example 51 as a light brown solid.

¹H-NMR (DMSO-d₆): δ4.47 (2H, d, J=6.4 Hz), 7.0-7.8 (13H, m), 7.95 -8.15 (2H, m), 8.65-8.75 (1H, m), 9.09 (1H, s), 9.32 (1H, t, J=6.4 Hz), 10.35 (1H, s).

APCI-MS (m/z): 489 (M⁺+1)

Preparation 19

To a solution of 4-aminobenzonitrile (11.81 g) and triethylamine (12.14 g) in dichloromethane (300 ml) was added dropwise a solution of 4′-(trifluoromethyl)-1,1′-biphenyl-2-carbonyl chloride (31.31 g) in dichloromethane (100 ml) at 5° C. and the mixture was stirred at room temperature for 20 hours. The mixture was poured into water and the separated organic layer was washed with brine, dried over magnesium sulfate and evaporated in vacuo. The residue was purified by column chromatography on silica gel eluting with hexane:ethyl acetate (1:1) to give N-(4-cyanophenyl)-4′-(trifluoromethyl)-1,1′-biphenyl-2-carboxamide (23.12 g) as white crystals.

¹H-NMR (DMSO-d₆): δ7.5-7.8 (12H, m), 10.82 (1H, s).

APCI-MS (m/z): 367 (M⁺+1)

Preparation 20

To a solution of N-(4-cyanophenyl)-4′-(trifluoromethyl)-1,1′-biphenyl-2-carboxamide (7.33 g) in ammonia (2.0 M solution in methanol) 180 ml) was added Raney Cobalt (OFT-MS, Kawaken Fine Chemicals) (ca. 10 g) at room temperature under nitrogen and the mixture was hydrogenated at 3 atm. hydrogen pressure at 50° C. for 6 hours. The Raney Cobalt were filtered off by celite and washed with methanol. The filtrate was evaporated in vacuo and the residue was purified by column chromatography on silica gel eluting with dichloromethane:methanol (5:1) to give N-[4(aminomethyl)phenyl]-4′-(trifluoromethyl)-1,1′-biphenyl-2-carboxamide (23.12 g) as a light brown oil.

¹H-NMR (DMSO-d₆); δ3.64 (2H, s), 7.21 (2H, d, J=8.1 Hz), 7.43 (2H, d, J=8.1 Hz), 7.5-7.8 (8H, m), 10.27 (1H, s).

ESI-MS (m/z): 393 (M⁺+Na)

### EXAMPLE 53

To a suspension of 2-pyridinecarboxylic acid (616 mg) in toluene (20 ml) were added thionyl chloride (1.19 g) and N,N-dimethylformamide (5 drops) and the mixture was stirred at 100° C. for 3 hours. The mixture was evaporated in vacuo and the residue was dissolved in dichloromethane (60 ml). The acid chloride solution was added, to a solution of N-[4-(aminomethyl)phenyl)-4′-(trifluoromethyl)-1,1′-biphenyl-2-carboxamide (1.85 g) and triethylamine (1.01 g) in dichloromethane (50 ml) at 5° C. and the mixture was stirred at the same temperature for 16 hours. The mixture was poured into water and the separated organic layer was washed with brine, dried over magnesium sulfate, and evaporated in vacuo. The residue was purified by column chromatography on silica gel eluting with-ethyl acetate to give N-[4-({[4′-(trifluoromethyl)-1,1′-biphenyl-2-yl]carbonyl}amino)benzyl]-2-pyridinecarboxamide (1.34 g) as white crystals.

¹H-NMR (DMSO-d₆): δ4.43 (2H, d, J=6.3 Hz), 7.23 (2H, d, J=8.4 Hz), 7.45 (2H, d, J=8.4 Hz), 7.5-7.9 (9H, m), 7.95-8.1 (2H, m), 8.65 (2H, d, J=4.7 Hz), 9.27 (1H, t, J=6.3 Hz), 10.33 (1H, s).

APCI-MS (m/z): 476 (M⁺+1)

### EXAMPLE 54

To a solution of N-[4-(aminomethyl)phenyl]-4′-(trifluoromethyl)-1,1′-biphenyl-2-carboxamide (556 mg) and triethylamine (455 mg) in dichloromethane (30 ml) was added nicotinoyl chloride hydrochloride (267 mg) at 5° C. and the mixture was stirred at the room temperature for 20 hours. The mixture was poured into water and the separated organic layer was washed with brine, dried over magnesium sulfate, and evaporated in vacuo. The residue was purified by column chromatography on silica gel eluting with ethyl acetate to give N-[4-({[4′-(trifluoromethyl)-1,1′-biphenyl-2-yl)carbonyl}amino)benzyl]nicotinamide (517 mg) as light brown crystals.

¹H-NMR (DMSO-d₆): δ4.43 (2H, d, J=5.9 Hz), 7.24 (2H, d, J=8.5 Hz), 7.47 (2H, d, J=8.5 Hz), 7.5-7.8 (10H, m), 7.75 (2H, d, J=8.2 Hz), 8.2-8.3 (1H, m), 8.7-8.75 (1H, m), 9.18 (1H,t, J=5.9 Hz), 10.34 (1H, s).

APCI-MS (m/z): 476 (M⁺+1)

### EXAMPLE 55

N-[4-({[4′-(Trifluoromethyl)-1,1′-biphenyl-2-yl]carbonyl}amino)benzyl]isonicotinamide

The title compound was obtained from N-[4-(aminomethyl)phenyl]-4′-(trifluoromethyl)-1,1′-biphenyl-2-carboxamide and isonicotinoyl chloride hydrochloride in the same manner as in Example 54 as white crystals.

¹H-NMR (DMSO-d₆): δ4.43 (2H, d, J=5.9 Hz), 7.23 (2H, d, J=8.4 Hz), 7.47 (2H, d, J=8.45 Hz), 7.5-7.8 (10H, m), 8.73 (2H, d, J=6.0 Hz), 9.28 (1H, t, J=5.9 Hz), 10.34 (1H, s).

APCI-MS (m/z): 476 (M⁺+1)

### EXAMPLE 56

To a suspension of N-(4-aminobenzyl)-2-pyridinecarboxamide (454 mg), 4′-methyl-1,1′-biphenyl-2-carboxylic acid (424 mg) and 1-hydroxybenzotriazole (HOBT) (306 mg) in dichloromethane (40 ml) was added 1-[3-(dimethylamino)propyl]-3-ethylcarbodiimide (WSC) (310 mg) at room temperature. The resulting solution was stirred at room temperature for 20 hours and poured into water. The separated organic layer was washed with brine, dried over magnesium sulfate and evaporated in vacuo. The residue was purified by column chromatography on silica gel eluting with hexane:ethyl acetate (1:2) to give N-(4-{[(4′-methyl-1,1′-biphenyl-2-yl)carbonyl]amino}benzyl)-2-pyridinecarboxamide (524 mg) as a light brown solid.

¹H-NMR (DMSO-d₆): δ2.27 (3H, s), 4.43 (2H, d, J=6.4 Hz), 7.1-7.65 (9H, m), 7.95-8.1 (2H, m), 8.65 (1H, d, J=4.6 Hz), 9.27 (1H, t, J=6.4 Hz), 10.20 (1H, s).

APCI-MS (m/z): 422 (M⁺+1)

### EXAMPLE 57

N-(4-{[(4′-Chloro-1,1′-biphenyl-2-yl)carbonyl]amino}-benzyl)-2-pyridinecarboxamide

The title compound was obtained from N-(4-aminobenzyl)-2-pyridinecarboxamide and 4′-chloro-1,1′-biphenyl-2-carboxylic acid in the same manner as in Example 56 as a white solid.

¹H-NMR (DMSO-d₆): δ4.43 (2H, d, J=6.4 Hz), 7.24 (2H, d, J=8.5 Hz), 7.4-7.1 (11H, m), 7.9-8.1 (2H, m), 8.6-8.7 (1H, m), 8.64 (1H, t, J=6.4 Hz), 10.25 (1H, s).

APCI-MS (m/z): 442 (M⁺+1)

Preparation 21

To a solution of 4-fluoronitrobenzene (12.71 g) and 2-(2-pyridinyl)ethylamine (12.22 g) in N,N-dimethylformamide (70 ml) was added triethylamine (10.12 g) at room temperature and the mixture was stirred at 60° C. for 16 hours. The mixture was cooled to 5° C. and poured into a mixture of ethyl acetate and water. The separated organic layer was washed with water and brine, dried over magnesium sulfate, and evaporated in vacuo. The residue was triturated with diisopropyl ether, collected by filtration, washed with diisopropyl ether and dried in vacuo to give 2-[2-(4-nitroanilino)ethyl]pyridine (21.21 g) as a yellow solid.

¹H-NMR (DMSO-d₆): δ3.02 (2H, t, J=7.0 Hz), 3.55 (2H, td, J=7.0 and 5.6 Hz), 6.65 (2H, d, J=9.3 Hz), 7.24 (1H, dd, J=7.8 and 4.9 Hz), 7.31 (1H, d, J=7.8 Hz), 7.39 (1H, t, J=5.6 Hz), 7.65-7.8 (1H, m), 7.98 (1H, d, J=9.3 Hz), 8.52 (1H, d, J=4.0 Hz).

APCI-MS (m/z): 244 (M⁺+1)

Preparation 22

To a solution of 2-[2-(4-nitroanilino)ethyl]pyridine (17.87 g) in tetrahydrofuran (150 ml) were added di-tert-butyl dicarbonate (19.25 g) and triethylamine (8.92 g) at room temperature and the mixture was refluxed for 16 hours. The mixture was evaporated in vacuo and the residue was purified by column chromatography on silica gel eluting with-hexane:ethyl acetate (2:1) to give tert-butyl 4-nitrophenyl[2-(2-pyridinyl)ethyl]carbamate (18.21 g) as a yellow solid.

¹H-NMR (DMSO-d₆): δ1.37 (9H, s), 2.95 (2H, t, J=8.0 Hz), 4.09 (2H, t, J=8.0 Hz), 7.2-7.3 (2H, m), 7.52 (2H, d, J=9.1 Hz), 7.65-7.75 (1H, m), 8.17 (2H, d, J=9.1 Hz), 8.23 (1H, d, J=4.8 Hz).

APCI-MS (m/z): 344 (M⁺+1)

Preparation 23

tert-Butyl 4-aminophenyl[2-(2-pyridinyl)ethyl]carbamate

The title compound was obtained from tert-butyl 4-nitrophenyl[2-(2-pyridinyl)ethyl]carbamate in the same manner as in Preparation 16 as a light brown solid.

¹H-NMR (DMSO-d₆): δ1.29 (9H, s), 2.86 (2H,t, J=7.0 Hz), 3.78 (2H,t, J=7.0 Hz), 5.04 (2H, brs), 6.52 (2H, d, J=8.5 Hz), 6.80 (2H, d, J=8.5 Hz), 7.15-7.3 (2H, m), 7.65-7.75 (1H, m), 8.45 (1H, d, J=4.2 Hz)

APCI-MS (m/z): 314 (M⁺+1)

### EXAMPLE 58

2-[(4-{(tert-Butoxycarbonyl)[2-(2-pyridinyl)ethyl]amino}-anilino)carbonyl]-4′-(trifluoromethyl)-1,1′-biphenyl

The title compound was obtained from tert-butyl 4-aminophenyl[2-(2-pyridinyl)ethyl)carbamate and 4′-(trifluoromethyl)-1,1′-biphenyl-2-carbonyl chloride in the same manner as in Preparation 19 as a yellow solid.

¹H-NMR (DMSO-d₆): δ1.31 (9H, s), 2.88 (2H, t, J=7.6 Hz), 3.89 (2H, t, J=7.6 Hz), 7.11 (2H, d, J=8.7 Hz), 7.22 (2H, d, J=7.7 Hz), 7.45-7.8 (9H, m), 8.45 (1H, d, J=4.8 Hz), 10.40 (1H, s).

APCI-MS (m/z): 562 (M⁺+1)

### EXAMPLE 59

To a solution of 2-[(4-{(tert-butoxycarbonyl)[2-(2-pyridinyl)ethyl]amino}anilino)carbonyl]-4′-(trifluoromethyl)-1,1′-biphenyl (22.58 g) in dichloromethane (70 ml) was added trifluoroacetic acid (36.7 g) at room temperature and the mixture was stirred at room temperature for 18 hours. The mixture was evaporated in vacuo and a mixture of dichloromethane and sodium hydrogencarbonate aqueous solution was added to the residue. The separated organic layer was washed with brine, dried over magnesium sulfate, and evaporated in vacuo. The residue was purified by column chromatography on silica gel eluting with ethyl acetate and crystallized from ethyl acetate to give N-(4-{[2-(2-pyridinyl)ethyl]amino}phenyl)-4′-(trifluoromethyl)-1,1′-biphenyl-2-carboxamide (14.64 g) as white crystals.

¹H-NMR (DMSO-d₄): δ2.96 (2H, t, J=7.1 Hz), 3.34 (2H, td, J=7.1 and 5.6 Hz), 5.53 (1H, t, J=5.6 Hz), 6.50 (2H, d, J=8.8 Hz), 7.20 (2H, d, J=8.8 Hz), 7.45-7.8 (11H, m), 8.50 (1H, d, J=4.7 Hz), 9.96 (1H, s).

APCI-MS (m/z): 462 (M⁺+1)

Preparation 24

2-{2-[4-Nitro(methyl)anilino]ethyl}pyridine

The title compound was obtained from 4-fluoronitrobenzene and N-methyl-N-[2-(2-pyridinyl)ethyl]amine in the same manner as in Preparation 21 as a yellow solid.

¹H-NMR (DMSO-d₆): δ2.99 (3H, s), 3.01 (2H, t, J=7.1 Hz), 3.85 (2H, t, J=7.0 Hz), 6.78 (2H, d, J=9.5 Hz), 7.23 (1H, dd, J=7.8 and 4.9 Hz), 7.30 (1H, d, J=7.8 Hz), 7.69 (1H, ddd, J=7.8 and 4.9 and 4.0 Hz), 8.02 (2H, d, J=9.5 Hz), 8.52 (1H, d, J=4.0 Hz).

APCI-MS (m/z): 258 (M⁺+1)

Preparation 25

N¹-Methyl-N¹-[2-(2-pyridinyl) ethyl]-1,4-benzenediamine

The title compound was obtained from 2-{2-[4-nitro(methyl)anilino]ethyl}pyridine in the same manner as in Preparation 16 as a light brown oil.

¹H-NMR (DMSO-d₆): δ2.71 (3H, s), 2.85 (2H, t, J=8.0 Hz), 3.46 (2H, t, J=8.0 Hz), 4.39 (2H, brs), 6.5-6.65 (4H, m), 7.19 (1H, dd, J=8.6 and 4.9 Hz), 7.24 (1H, d, J=8.6 Hz), 7.67 (1H, ddd, J=7.8 and 4.9 and 1.9 Hz), 8.49 (1H, d, J=4.8 Hz).

APCI-MS (m/z): 228 (M⁺+1)

### EXAMPLE 60

N-(4-{Methyl[2-(2-pyridinyl)ethyl]amino}phenyl)-4′-(trifluoromethyl)-1,1′-biphenyl-2-carboxamide

The title compound was obtained from N¹-methyl-N¹-[2-(2-pyridinyl)ethyl]-1,4-benzenediamine and 4′-(trifluoromethyl)-1,1′-biphenyl-2-carbonyl chloride in the same manner as in Preparation 19 as white crystals.

¹H-NMR (DMSO-d₆): δ2.80 (3H, s), 2.89 (2H, t, J=7.1 Hz), 3.63 (2H, t, J=7.1 Hz), 6.65 (2H, d, J=9.1 Hz), 7.15-7.25 (1H, m), 7.32 (2H, d, J=9.1 Hz), 7.45-7.8 (10H, m), 8.51 (1H, d, J=4.8 Hz), 10.01 (1H, s).

APCI-MS (m/z): 476 (M⁺+1)

### EXAMPLE 61

N-(4-(Methyl[2-(2-pyridinyl)ethyl]amino}phenyl)-2-[3-(trifluoromethyl)anilino]benzamide

The title compound was obtained from N¹-methyl-N¹-[2-(2-pyridinyl)ethyl]-1,4-benzenediamine and 2-[3-(trifluoromethyl)anilino]benzoic acid in the same manner as in Example 51 as white crystals.

¹H-NMR (DMSO-d₆): δ2.83 (3H, s), 2.92 (2H, t, J=7.9 Hz), 3.66 (2H, t, J=7.9 Hz.), 6.71 (2H, d, J=9.1 Hz), 7.0-7.15 (1H, m), 7.32 (2H, d, J=9.1 Hz), 7.2-7.6 (9H, m), 7.66 (1H, dd, J=7.4 and 1.9 Hz), 7.74 (1H, d, J=7.4 Hz), 9.24 (1H, s), 10.11 (1H, s).

APCI-MS (m/z): 491 (M⁺+1)

Preparation 26

2-[(4-Nitroanilino)methyl]pyridine

The title compound was obtained from 4-fluoronitrobenzene and 2-pyridinylmethylamine in the same manner as in Preparation 21 as a yellow solid.

¹H-NMR (DMSO-d₆): δ4.52 (2H, d, J=6.1 Hz), 6.69 (2H, d, J=9.3 Hz), 7.29 (1H, dd, J=7.8 and 4.9 Hz), 7.34 (1H, d, J=7.8 Hz), 7.79 (1H, ddd, J=7.8 and 4.9 and 1.8 Hz), 7.90 (1H, t, J=6.1 Hz), 7.98 (2H, d, J=9.3 Hz), 8.55 (1H, d, J=4.8 Hz)

ESI-MS (m/z): 252 (M⁺+Na)

Preparation 27

N¹-(2-Pyridinylmethyl)-1,4-benzenediamine

The title compound was obtained from 2-[(4-nitroanilino)-methyl]pyridine in the same manner as in Preparation 16 as a light brown oil.

¹H-NMR (DMSO-d₆): δ4.21 (2H, brs), 4.23 (2H, d, J=6.2 Hz), 5.45 (2H, t, J=6.2 Hz), 6.35-6.4 (4H, m), 7.22 (1H, dd, J=7.8 and 4.9 Hz), 7.35 (1H, d, J=7.8 Hz), 7.71 (1H, ddd, J=7.8 and 4.9 and 1.8 Hz), 8.50 (1H, d, J=4.0 Hz).

ESI-MS (m/z): 222 (M⁺+Na)

### EXAMPLE 62

N-{4-[(2-Pyridinylmethyl)amino]phenyl}-4′-(trifluoromethyl)-1,1′-biphenyl-2-carboxamide

The title compound was obtained from N¹-(2-pyridinylmethyl)-1,4-benzenediamine in the same manner as in Preparation 19 as white crystals.

¹H-NMR (DMSO-d₆): δ4.32 (2H, d, J=6.1 Hz), 6.21 (2H, d, J=6.1 Hz), 6.48 (2H, d, J=8.8 Hz), 7.16 (2H, d, J=8.8 Hz), 7.2-7.8 (11H, m), 8.51 (1H, d, J=4.0 Hz), 9.20 (1H, s).

ESI-MS (m/z): 470 (M⁺+Na)

### EXAMPLE 63

tert-Butyl 2-(2-pyridinyl)ethyl[4-({2-[3-(trifluoromethyl)anilino]benzoyl}amino)phenyl]carbamate

The title compound was obtained from tert-butyl 4-aminophenyl[2-(2-pyridinyl)ethyl]carbamate and 2-[3-(trifluoromethyl)anilino]benzoic acid in the same manner as in Example 56 as a light brown solid.

¹H-NMR (DMSO-d₆): δ1.32 (9H, s), 2.89 (2H, t, J=7.8 Hz), 3.91 (2H, t, J=7.8 Hz), 7.05-7.4 (6H, m), 7.4-7.6 (5H, m), 7.7-7.9 (4H, m), 8.49 (1H, d, J=4.3 Hz), 9.03 (1H, s), 10.39 (1H, s).

ESI-MS (m/z): 599 (M⁺+Na).

### EXAMPLE 64

N-(4-{(2-(2-Pyridinyl)ethyl]amino}phenyl)-2-[3-(trifluoromethyl)anilino]benzamide

The title compound was obtained from tert-butyl 2-(2-pyridinyl)ethyl[4-({2-[3-(trifluoromethyl)anilino]benzoyl}amino)-phenyl]carbamate in the same manner as in Example 59 as white crystals.

¹H-NMR (DMSO-d₆): δ2.98 (2H, t, J=7.0 Hz), 3.36 (2H, td, J=7.0 and 5.8 Hz), 5.60 (1H, t, J=5.8 Hz), 6.57 (2H, d, J=8.8 Hz), 7.05-7.15 (1H, m), 7.2-7.6 (14H, m), 7.7-7.9 (2H, m), 8.51 (1H, d, J=4.0 Hz), 9.25 (1H, s), 10.04 (1H, s).

ESI-MS (m/z): 499 (M⁺+Na), 477 (M⁺+1)

Preparation 28

N-(4-Nitrophenyl)-4′-trifluoromethyl)-1,1′-biphenyl-2-carboxamide

The title compound was obtained from 4-aminonitrobenzene and 4′-(trifluoromethyl)-1,1′-biphenyl-2-carbonyl chloride in the same manner as in Preparation 19 as a yellow solid.

¹H-NMR (DMSO-d₆): δ7.5-7.8 (10H, m), 8.19 (2H, d, J=9.2 Hz), 10.99 (1H, s)

APCI-MS (m/z): 387 (M⁺+1)

Preparation 29

N-(4-Aminophenyl)-4′-(trifluoromethyl)-1,1′-biphenyl-2-carboxamide

The title compound was obtained from N-(4-nitrophenyl)-4′-(trifluoromethyl)-1,1′-biphenyl-2-carboxamide in the same manner as in Preparation 16 as yellow crystals.

¹H-NMR (DMSO-d₆): δ4.89 (2H, brs), 6.46 (2H, d, J=8.7 Hz), 7.11 (2H, t, J=8.7 Hz), 7.45-7.65 (4H, m), 7.63 (1H, d, J=8.2 Hz), 7.76 (2H, d, J=8.2 Hz), 9.87 (1H, s).

APCI-MS (m/z): 357 (M⁺+1)

### EXAMPLE 65

N-[4-[(2-Pyridinylacetyl)amino]phenyl}-4′-(trifluoromethyl)-1,1′-biphenyl-2-carboxamide

The title compound was obtained from N-(4-aminophenyl)-4′-(trifluoromethyl)-1,1′-biphenyl-2-carboxamide and 2-pyridinylacetic acid hydrochloride in the same manner as in Preparation 15 as white crystals.

¹H-NMR (DMSO-d₆): δ3.81 (2H, s), 7.25-7.8 (15H, m), 8.49 (1H, d, J=4.0 Hz), 10.19 (1H, s), 10.28 (1H, s).

ESI-MS (m/z): 498 (M⁺+Na), 476 (M⁺+1)

Preparation 30

To a suspension of 4-nitrothiophenol (3.10 g) in methanol (60 ml) were added 2-vinylpyridine (2.10 g) and acetic acid (1.20 g) at room temperature and the mixture was refluxed for 6 hours. The resulting solution was cooled to room temperature and evaporated in vacuo. The residue was triturated with diisopropyl ether and the yellow solid was collected by filtration, washed with diisopropyl ether, and dried to give 2-{2-[(4-nitrophenyl)sulfanyl]ethyl}pyridine (4.70 g).

¹H-NMR (DMSO-d₆): δ3.12-2H, t, J=7.4 Hz), 3.53 (2H, t, J=7.4 Hz), 7.25 (1H, dd, J=7.8 and 4.9 Hz), 7.33 (1H, d, J=7.4 Hz), 7.5-7.6 (2H, m), 7.7-7.8 (1H, m), 8.1-8.2 (2H, m), 8.52 (1H, d, J=4.0 Hz).

APCI-MS (m/z): 261 (M⁺+1)

Preparation 31

4-{[2-(2-Pyridinyl)ethyl]sulfanyl}phenylamine

The title compound was obtained from 2-{2-[(4-nitrophenyl)sulfanyl]ethyl}pyridine in the same manner as in Preparation 16 as a yellow oil.

¹H-NMR (DMSO-d₆): δ2.89 (2H, t, J=6.6 Hz), 3.04 (2H, t, J=6.6 Hz), 5.25 (2H, brs), 6.53 (2H, d, J=8.5 Hz), 7.11 (2H, d, J=8.5 Hz), 7.15-7.25 (2H, m), 7.65-7.75 (1H, m), 8.46 (1H, d, J=4.6 Hz).

APCI-MS (m/z): 231 (M⁺+1)

### EXAMPLE 66

N-(4-{[2-(2-Pyridinyl)ethyl]sulfanyl}phenyl)-4′-(trifluoromethyl)-1,1′-biphenyl-2-carboxamide

The title compound was obtained from 4-{[2-(2-pyridinyl)ethyl]sulfanyl}phenylamine and 4′-(trifluoromethyl)-1,1′-biphenyl-2-carbonyl chloride in the same manner as in Preparation 19 as a yellow solid.

¹H-NMR (DMSO-d₆): δ2.97 (2H, t, J=7.1 Hz), 3.28 (2H, t, J=7.1 Hz), 7.2-7.4 (4H, m), 7.45-7.8 (11H, m), 8.48 (1H, d, J=4.8 Hz), 10.42 (1H, s).

APCI-MS (m/z): 479 (M⁺+1)

### EXAMPLE 67

2-[(4-{(tert-Butoxycarbonyl)[2-(2-pyridinyl)ethyl]amino}-anilino)carbonyl]-4′-methyl-1,1′-biphenyl

The title compound was obtained from tert-butyl 4-aminophenyl[2-(2-pyridinyl)ethyl]carbamate and 4′-methyl-1,1′-biphenyl-2-carboxylic acid in the same manner as in Example 56 as a light yellow solid.

¹H-NMR (DMSO-₆): δ1.32 (9H, s), 2.29 (3H, s), 2.88 (2H, t, J=6.8 Hz), 3.88 (2H, t, J=6.8 Hz), 7.10 (2H, d, J=8.8 Hz), 7.15-7.3 (4H, m), 7.34 (2H, d, J=8.8 Hz), 7.4-7.7 (7H, m), 8.45 (1H, d, J=4.8 Hz), 10.29 (1H, s).

APCI-MS (m/z): 506 (M+H)⁺

### EXAMPLE 68

4′-Methyl-N-(4-{[2-(2-pyridinyl)ethyl]amino}phenyl)-1,1′-biphenyl-2-carboxamide The title compound was obtained from 2-[(4-{(tert-butoxycarbonyl)[2-(2-pyridinyl)ethyl]amino}anilino)carbonyl]-4′-methyl-1,1′-biphenyl in the same manner as in Example 59 as white crystals.

¹H-NMR (DMSO-d₆): δ2.30 (3H, s), 2.96 (2H, t, J=7.4 Hz), 3.34 (2H, td, J=7.4 and 5.8 Hz), 5.51 (1H, t, J=5.8 Hz), 6.50 (2H, d, J=8.9 Hz), 7.2-7.6 (15H, m), 7.65-7.8 (1H, m), 8.52 (1H, d, J=4.9 Hz), 9.80 (1H, s).

APCI-MS (m/z): 408 (M+H)⁺

### EXAMPLE 69

2-[(4-{(tert-Butoxycarbonyl)[2-(2-pyridinyl)ethyl]amino}-anilino)carbonyl]-4′-chloro-1,1′-biphenyl

The title compound was obtained from tert-butyl 4-aminophenyl[2-(2-pyridinyl)ethyl]carbamate and 4′-chloro-1,1′-biphenyl-2-carboxylic acid in the same manner as in Example 56 as a light yellow solid.

¹H-NMR (DMSO-d₆): δ1.32 (9H, s), 2.89 (2H, t, J=7.3 Hz), 3.89 (2H, t, J=7.3 Hz), 7.11 (2H, d, J=8.7 Hz), 7.22 (2H, d, J=8.7 Hz), 7.4-7.75 (11H, m), 8.45 (1H, d, J=4.2 Hz), 10.33 (1H, s).

ESI-MS (m/z): 550 (M+Na)⁺, 528 (M+H)⁺

### EXAMPLE 70

4′-Chloro-N-(4-{[2-(2-pyridinyl)ethyl]amino}phenyl)-1,1′-biphenyl-2-carboxamide

The title compound was obtained from 2-[(4-{(tert-butoxycarbonyl) [2-(2-pyridinyl)ethyl]amino}anilino)carbonyl]-4′-chloro-1,1′-biphenyl in the same manner as in Example 59 as white crystals.

¹H-NMR (DMSO-d₆): δ2.96 (2H, t, J=7.0 Hz), 3.33 (2H, td, J=7.0 and 5.7 Hz), 5.54 (1H, t, J=5.7 Hz), 6.51 (2H, d, J=8.9 Hz), 7.21 (2H, d, J=8.9 Hz), 7.30 (1H, d, J=7.7 Hz), 7.4-7.6 (9H, m), 7.70 (1H, ddd, J=7.7 and 7.6 and 1.9 Hz), 8.51 (1H, d, J=4.8 Hz), 9.85 (1H, s).

APCI-MS (m/z): 430, 428 (M+H)⁺

### EXAMPLE 71

4′-Methoxy-1,1′-biphenyl-2-carbonyl chloride (0.38 g) was added to a solution of tert-butyl 4-aminophenyl[2-(2-pyridinyl)ethyl]carbamate (0.4 g) and triethylamine (0.21 ml) in dichloromethane (4 ml) under ice-cooling and the mixture was stirred at ambient temperature for 20 hours. To the reaction mixture was added a solution of 10% hydrogen chloride in methanol (6 ml) and the mixture was stirred at ambient temperature for 20 hours.

The reaction mixture was poured into a mixture of ethyl acetate, tetrahydrofuran and water and the mixture was adjusted to pH 9 with 20% aqueous potassium carbonate solution. The separated organic layer was washed with water, dried over magnesium sulfate and evaporated in vacuo. The residue was triturated with a mixture of diethyl ether and diisopropyl ether to give 4′-methoxy-N-(4-{(2-(2-pyridinyl)ethyl]amino}phenyl)-1,1′-biphenyl-2-carboxamide (0.43 g).

¹H-NMR (DMSO-d₆): δ2.96 (2H, d, J=7.2 Hz), 3.27-3.42 (2H, m), 3.74 (3H, s), 5.51 (1H, t, J=5.7 Hz), 6.52 (2H, d, J=8.7 Hz), 6.94 (2H, d, J=8.7 Hz), 7.17-7.35 (4H, m), 7.35-7.53 (6H, m), 7.64-7.74 (1H, m), 8.51 (1H, d, J=4.3 Hz), 9.79 (1H, s).

APCI-MS (m/z): 424 (M+H)⁺

Preparation 32

4′-(Trifluoromethyl)-1,1′-biphenyl-2-carbonyl chloride (2.0 g) was added to a solution of 4-aminophenol (0.7 g) and N,O-bis(trimethylsilyl)acetamide (3.9 ml) in tetrahydrofuran (5 ml) under ice-cooling and the mixture was stirred at ambient temperature for 20 hours. The reaction mixture was poured into a mixture of ethyl acetate, tetrahydrofuran and water and the mixture was adjusted to pH 7 with 20% aqueous potassium carbonate solution. The separated organic layer was washed with water, dried over magnesium sulfate and evaporated in vacuo. The residue was triturated with diisopropyl ether to give N-(4-hydroxy-phenyl)-4′-(trifluoromethyl)-1,1′-biphenyl-2-carboxamide (2.21 g).

¹H-NMR (DMSO-d₆): δ6.67 (2H, d, J=8.9 Hz), 7.29 (2H, d, J=8.9 Hz), 7.47-7.67 (6H, m), 7.76 (2H, d, J=8.3 Hz), 9.22 (1H, s), 10.07 (1H, s).

### EXAMPLE 72

Diethyl azodicarboxylate (0.27 ml) was added to a mixture of N-(4-hydroxyphenyl)-4′-(trifluoromethyl)-1,1′-biphenyl-2-carboxamide (0.5 g), 2-pyridinylcarbinol (0.16 ml) and triphenylphosphine (0.44 g) in tetrahydrofuran (10 ml) under ice-cooling and the mixture was stirred under ice-cooling for 5 hours. The reaction mixture was poured into a mixture of ethyl acetate and water. The separated organic layer was washed with water, dried over magnesium sulfate and evaporated in vacuo. The residue was purified by column chromatography on silica gel eluting with ethyl acetate:hexane (1:1). The eluted fractions containing the desired product were collected and evaporated in vacuo to give N-[4-(2-pyridinylmethoxy)phenyl]-4′-(trifluoromethyl)-1,1′-biphenyl-2-carboxamide (0.25 g).

¹H-NMR (DMSO-d₆) δ5.14 (2H, s), 6.95 (2H, d, J=9.0 Hz), 7.33 (1H, dd, J=5.0 and 12.5 Hz), 7.39-7.72 (9H, m), 7.74-7.88 (1H, m), 7.76 (2H, d, J=8.5 Hz), 8.57 (1H, d, J=4.2 Hz), 10.23 (1H, s).

APCI-MS (m/z): 449 (M+H)⁺

### EXAMPLE 73

N-{4-[2-(2-Pyridinyl)ethoxy]phenyl}-4′-(trifluoromethyl)-1,1′-biphenyl-2-carboxamide

The title compound was obtained from N-(4-hydroxyphenyl)-4′-(trifluoromethyl)-1,1′-biphenyl-2-carboxamide and 2-(2-pyridinyl)ethanol in the same manner as in Example 72.

¹H-NMR (DMSO-d6): δ3.16 (2H, t, J=6.6 Hz), 4.30 (2H, t, J=6.6 Hz), 6.85 (2H, d, J=9.0 Hz), 7.24 (1H, dd, J=5.5 Hz, 12.2 Hz), 7.32-7.46 (3H, m), 7.46-7.80 (9H, m), 8.51 (1H, d, J=4.3 Hz), 10.19 (1H, s).

APCI-MS (m/z): 463 (M+H)⁺

Preparation 33

A mixture of tert-butyl 4-(2-aminoethyl)-1,3-thiazol-2-ylcarbamate (0.882 g), 1-fluoro-4-nitrobenzene (0.511 g) and triethylamine (0.76 ml) in 1,3-dimethyl-2-imidizolidinone (10 ml) was heated to 50° C. for 3 hours. The reaction mixture was cooled to room temperature, poured into water and extracted with ethyl acetate. The organic layer was washed with brine, dried over magnesium sulfate, filtered and concentrated in vacuo. The residue was purified by column chromatography on silica gel eluting with hexane:ethyl acetate (2:1) to give tert-butyl 4-[2 (4-nitroanilino)ethyl]-1,3-thiazol-2-ylcarbamate (0.763 g) as a yellow oil.

¹H-NMR (CDCl₃): δ1.54 (9H, s), 2.97 (2H, t, J=6.3 Hz), 3.47 (2H, q, J=6.3 Hz), 5.04 (1H, br s), 6.48 (2H, d, J=9.2 Hz), 6.59 (1H, s), 8.04 (2H, d, 9.2 Hz).

Preparation 34

To a solution of tert-butyl 4-[2-(4-nitroanilino)ethyl]-1,3-thiazol-2-ylcarbamate (0.749 g) and 4,4-dimethylaminopyridine (25 mg) in tetrahydrofuran (30 ml) was added di-tert-butyl dicarbonate (0.673 g) and the mixture-was heated to 50° C. for 1 hour. The reaction mixture was cooled to room temperature and concentrated in vacuo to give tert-butyl 2-{2-[(tert-butoxycarbonyl)amino]-1,3-thiazol-4-yl}ethyl(4-nitrophenyl)carbamate (0.955 g) as a yellow oil. The product was used for the next step without any purification.

Preparation 35

A solution of tert-butyl 2-(2-[(tert-butoxycarbonyl)amino]-1,3-thiazol-4-yl}ethyl(4-nitrophenyl)carbamate (0.955 g) in methanol (30 ml) was hydrogenated over 10% Pd—C at room temperature under atmospheric pressure of hydrogen for 1 hour. The reaction mixture was filtered through a pad of celite, and the filtrate was concentrated in vacuo. The residue was purified by column chromatography on silica gel eluting with hexane:ethyl acetate (2:1) to give tert-butyl 4-{2-[4-amino(tert-butoxycarbonyl)anilino]ethyl}-1,3-thiazol-2-ylcarbamate (0.709 g) as a yellow oil.

¹H-NMR (CDCl₃): δ1.51 (18H, s), 2.94 (2H, t, J=6.6 Hz), 3.38 (2H, t, J=6.6 Hz), 6.52 (2H, d, J=8.6 Hz), 6.60 (2H, d, J=8.9 Hz), 6.76 (1H, s).

### EXAMPLE 74

To a solution of tert-butyl 4-{2-[4-amino(tert-butoxycarbonyl)anilino]ethyl}-1,3-thiazol-2-ylcarbamate (0.424 g), 4′-(trifluoromethyl)-1,1′-biphenyl-2-carboxylic acid (0.259 g) and HOBT (0.158 g) in tetrahydrofuran (15 ml) was added WSC.HCl (0.224 g), followed by triethylamine (0.21 ml) at room temperature. The reaction mixture was stirred for 1 hour, quenched with water, and-extracted with ethyl acetate. The organic layer was washed with brine, dried over magnesium sulfate, filtered and concentrated in vacuo. The residue was purified by column chromatography on silica gel eluting with hexane:ethyl acetate (2:1) to give tert-butyl 2-{2-[(tert-butoxycarbonyl)amino]-1,3-thiazol-4-yl}ethyl[4-(}[4′-(trifluoromethyl)-1,1′-biphenyl-2-yl]carbonyl}amino)phenyl]carbamate (0.520 g) as a white solid.

¹H-NMR (CDCl₃): δ1.51 (18H, s), 2.93 (2H,t, J=6.6 Hz), 3.39 (2H, t, J=6.6 Hz), 6.50 (2H, d, J=8.9 Hz), 6.74 (1H, s), 6.80 (1H, s), 6.94 (2H, d, J=8.6 Hz), 7.39-7.78 (8H, m).

### EXAMPLE 75

To a solution of tert-butyl 2-{2-[(tert-butoxycarbonyl)-amino]-1,3-thiazol-4-yl}ethyl[4-({[4′-(trifluoromethyl)-1,1′-biphenyl-2-yl]carbonyl}amino)phenyl]carbamate (0.493 g) in dichloromethane (15 ml) was added trifluoroacetic acid (1.7 ml) dropwise at room temperature. The reaction mixture was stirred for 15 hours, quenched with 10% aqueous potassium carbonate solution, and extracted with dichloromethane. The organic layer was washed with brine, dried over magnesium sulfate, filtered and concentrated in vacuo. The residue was recrystallized from a mixture of ethyl acetate and diisopropyl ether to give N-(4-{[2-(2-amino-1,3-thiazol-4-yl) ethyl]amino}phenyl)-4′-(trifluoromethyl)-1,1′-biphenyl-2-carboxamide (0.277 g) as a pale brown solid.

¹H-NMR (DMSO-d₆) δ2.63 (2H, t, J=7.3 Hz), 3.19 (2H, t, J=7.3 Hz), 6.19 (1H, s), 6.47 (2H, d, J=8.9 Hz), 6.82 (2H, s), 7.18 (2H, d, J=8.9 Hz), 7.45-7.60 (6H, m), 7.62 (2H, d, J=8.2 Hz), 7.74 (2H, d, J=8.2 Hz), 9.88 (1H, s).

ESI-MS (m/z): 483 (M+H)⁺

Preparation 36

To a solution of ethyl {6-[(tert-butoxycarbonyl)amino]-2-pyridinyl}acetate (0.835 g) in tetrahydrofuran (20 ml) was added lithium borohydride (0.097 g) at room temperature. The reaction mixture was refluxed for 4 hours, cooled to room temperature, quenched with water and extracted with ethyl acetate. The organic layer was washed with brine, dried over magnesium sulfate, filtered and concentrated in vacuo. The residue was purified by column chromatography on silica gel eluting with hexane:ethyl acetate (2:1) to give tert-butyl 6-(2-hydroxyethyl)-2-pyridiny}carbamate (0.627 g) as a white solid.

¹H-NMR (CDCl₃): δ1.51 (9H, s), 2.92 (2H, t, J=5.4 Hz), 3.99 (2H, t, J=5.4 Hz), 6.80 (1H, d, J=6.9 Hz), 7.58 (1H, dd, J=8.2 and 6.9 Hz), 7.79 (1H, d, J=8.2 Hz).

Preparation 37

To a solution of tert-butyl 6-(2-hydroxyethyl)-2-pyridinylcarbamate (0.606 g), triethylamine (0.7 ml) and 4,4-dimethylaminopyridine (15 mg) in 1,2-dichloroethane (25 ml) was added p-toluenesulfonyl chloride (0.582 g) portionwise at room temperature. The reaction mixture was stirred for 15 hours, quenched with water and extracted with 1,2-dichloroethane. The organic layer was washed with brine, dried over magnesium sulfate, filtered and concentrated in vacuo. The residue was purified by column chromatography on silica gel eluting with hexane:ethyl acetate (4:1) to give 2-{6-[(tert-butoxycarbonyl)amino]-2-pyridinyl}ethyl 4-methylbenzenesulfonate (0.785 g) as a clear oil.

¹H-NMR (CDCl₃): δ1.52 (9H, s), 2.43 (3H, s), 2.96 (2H, t, J=6.6 Hz), 4.37 (2H, t, J=6.6 Hz), 6.76 (1H, d, J=7.2 Hz), 7.00 (1H, br s), 7.26 (2H, d, J=7.9 Hz), 7.52 (1H, dd, J=8.2 and 7.2 Hz), 7.65 (2H, d, J=7.9 Hz), 7.73 (1H, d, J=8.2 Hz).

Preparation 38

A mixture of 2-(6-[(tert-butoxycarbonyl)amino]-2-pyridinyl}ethyl 4-methylbenzenesulfonate (1.342 g) and sodium azide (0.444 g) in N,N-dimethylformamide (20 ml) was stirred at room temperature for 15 hours. The solvent was evaporated. The residue was dissolved in a mixture of ethyl acetate and water, and extracted with ethyl acetate. The organic layer was washed with water and brine, dried over magnesium sulfate, filtered and concentrated in vacuo to give tert-butyl 6-(2-azidoethyl)-2-pyridinylcarbamate (0.880 g) as a yellow oil. The product was used for the next step without any purification.

¹H-NMR (CDCl₃): δ1.52 (9H, s), 2.93 (2H, t, J=6.9 Hz), 3.64 (2H, t, J=6.9 Hz), 6.84 (1H, d, J=6.6 Hz), 7.59 (1H, dd, J=8.2 and 6.6 Hz), 7.78 (1H, d, J=8.2 Hz).

Preparation 39

A solution of tert-butyl 6-(2-azidoethyl)-2-pyridinylcarbamate (0.88 g) in methanol (35 ml) was hydrogenated over 10% Pd—C at room temperature under atmospheric pressure of hydrogen for 1 hour. The reaction mixture was filtered through a pad of celite, and the filtrate was concentrated in vacuo to give tert-butyl 6-(2-aminoethyl)-2-pyridinylcarbamate (0.776 g) as a yellow oil. The product was used for the next step without any purification.

¹H-NMR (CDCl₃): δ1.51 (9H, s), 2.79 (2H, t, J=6.3 Hz), 3,05 (2H, t, J=6.3 Hz), 6.81 (1H, d, J=7.3 Hz), 7.57 (1H, dd, J=8.2 and 7.3 Hz).

Preparation 40

A mixture of tert-butyl 6-(2-aminoethyl)-2-pyridinylcarbamate (0.776 g), 1-fluoro-4-nitrobenzene (0.462 g) and triethylamine (0.69 ml) in 1,3-dimethyl-2-imidizolidinone (10 ml) was heated to 50° C. for 3.5 hours. The reaction mixture was cooled to room temperature, poured into water and extracted with ethyl acetate. The organic layer was washed with brine, dried over magnesium sulfate, filtered and concentrated in vacuo. The residue was purified by column chromatography on silica gel eluting with hexane:ethyl acetate (3:2) to give tert-butyl 6-[2-(4-nitroanilino)ethyl]-2-pyridinylcarbamate (0.666 g) as a yellow oil.

¹H-NMR (CDCl₃): δ1.53 (9H, s), 2.99 (2H, t, J=6.6 Hz), 3.57 (2H, dd, J=12.2 and 6.2 Hz), 5.21 (1H, br s), 6.53 (2H, d, J=9.2 Hz), 6.82 (1H, dd, J=7.6 and 0.7 Hz), 7.30 (1H, br s), 7.59 (1H, d, J=7.8 Hz), 7.95 (1H, d, J=7.9 Hz), 8.05 (2H, d, J=8.9 Hz).

Preparation 41

To a solution of tert-butyl 6-[2-(4-nitroanilino)ethyl]-2-pyridinylcarbamate (0.666 g) and 4,4-dimethylaminopyridine (23 mg) in tetrahydrofuran (25 ml) was added di-tert-butyl dicarbonate (0.608 g) and the mixture was heated to 50° C. for 1 hour. The reaction mixture was cooled to room temperature and concentrated in vacuo. The residue was dissolved in ethyl acetate and water, and extracted with ethyl acetate. The organic layer was washed with brine, dried over magnesium sulfate, filtered and concentrated in vacuo to give tert-butyl 2-{6-[(tert-butoxycarbonyl)amino]-2-pyridinyl}ethyl(4-nitrophenyl)carbamate (0.851 g). The product was used for the next step without any purification.

Preparation 42

A solution of tert-butyl 2-{6-[(tert-butoxycarbonyl)amino]-2-pyridinyl}ethyl(4-nitrophenyl)carbamate (0.851 g) in methanol (30 ml) was hydrogenated over 10% Pd—C at room temperature under atmospheric pressure of hydrogen for 1 hour. The reaction mixture was filtered through a pad of celite, and the filtrate was concentrated in vacuo. The residue was purified by column chromatography on silica gel eluting with hexane:ethyl acetate (3:2) to give tert-butyl 6-{2-[4-amino(tert-butoxycarbonyl)-anilino]ethyl}-2-pyridinylcarbamate (0.701 g) as a yellow oil.

### EXAMPLE 76

To a solution of tert-butyl 6-{2-[4-amino(tert-butoxycarbonyl)anilino]ethyl}-2-pyridinylcarbamate (0.242 g), 4′-(trifluoromethyl)-1,1′-biphenyl-2-carboxylic acid (0.150 g) and HOBT (92 mg) in N,N-dimethylformamide (10 ml) was added WSC.HCl (0.130 g), followed by triethylamine (0.12 ml) at room temperature. The reaction mixture was stirred at 50° C. for 15 hours, quenched with water and extracted with ethyl acetate. The organic layer was washed with brine, dried over magnesium sulfate, filtered and concentrated in vacuo. The residue was purified by column chromatography on silica gel eluting with hexane:ethyl acetate (2:1) to give tert-butyl 2-{6-[(tert-butoxycarbonyl)amino]-2-pyridinyl}ethyl[4-({[4′-(trifluoromethyl)-1,1′-biphenyl-2-yl]carbonyl}amino)phenyl]-carbamate (0.279 g) as a yellow oil.

### EXAMPLE 77

To a solution of tert-butyl 2-{6-[(tert-butoxycarbonyl)-amino]-2-pyridinyl}ethyl[4-({[4′-(trifluoromethyl)-1,1′-biphenyl-2-yl]carbonyl}amino)phenyl]carbamate (0.279 g) in dichloroethane (10 ml) was added trifluoroacetic acid (0.95 ml) dropwise. The reaction mixture was stirred for 15 hours, quenched with 10% aqueous potassium carbonate solution, and extracted with dichloromethane. The organic layer was washed with brine, dried over magnesium sulfate, filtered and concentrated in vacuo. The residue was recrystallized from a mixture of ethyl acetate and diisopropyl ether to give N-(4-{[2-(6-amino-2-pyridinyl)ethyl]amino}phenyl)-4′-(trifluoromethyl)-1,1′-biphenyl-2-carboxamide (96 mg) as a white solid.

¹H-NMR (DMSO-d₆): δ2.71 (2H, t, J=7.2 Hz), 3.25 (2H, t, J=7.2 Hz), 5.81 (2H, s), 6.27 (1H, d, J=8.2 Hz), 6.38 (1H, d, J=6.6 Hz), 6.49 (2H, d, J=8.9 Hz), 7.20 (2H, d, J=8.9 Hz), 7.24-7.30 (1H, m), 7.47-7.65 (6H, m), 7.76 (2H, d, J=7.9 Hz), 9.90 (1H, s).

ESI-MS (m/z): 477 (M+H)⁺

### EXAMPLE 78

To a suspension of sodium hydride (60% in oil dispersion, 16 mg) in tetrahydrofuran (5 ml) was added N-(4-hydroxyphenyl)-4′-(trifluoromethyl)-1,1′-biphenyl-2-carboxamide (0.118 g) in tetrahydrofuran (4 ml) dropwise at 0° C. After stirring for 20 minutes, 2-{2-[(tert-butoxycarbonyl)amino]-1,3-thiazol-4-yl}ethyl 4-methylbenzenesulfonate (0.132 g) in tetrahydrofuran (2 ml) was added dropwise. The reaction mixture was stirred at 0° C. for 5 hours and heated to 50° C. for 15 hours. After cooling, the reaction mixture was extracted with ethyl acetate. The organic layer was washed with brine, dried over magnesium sulfate, filtered and concentrated in vacuo. The residue was purified by column chromatography on silica gel eluting with hexane:ethyl acetate (2:1) to give tert-butyl 4-{2-(4-({[4′-(trifluoromethyl)-1,1′-biphenyl-2-yl]carbonyl}amino)phenoxy]ethyl}-1,3-thiazol-2-ylcarbamate (0.128 g) as a yellow oil.

¹H-NMR (DMSO-d₆): δ1.47 (9H, s), 2.98 (2H, t, J=6.9 Hz), 4.18 (2H, t, J=6.9 Hz), 6.84 (1H, s), 6.86 (1H, s), 7.39-7.77 (12H, m), 10.18 (1H, s).

ESI-MS (m/z): 584 (M+H)⁺

### EXAMPLE 79

To a solution of tert-butyl 4-{2-[4-({[4′-(trifluoromethyl)-1,1′-biphenyl-2-yl]carbonyl}amino)phenoxy]-ethyl}-1,3-thiazol-2-ylcarbamate (0.124 g) in dichloromethane (10 ml) was added trifluoroacetic acid (0.5 ml) dropwise. The reaction mixture was stirred at room temperature for 15 hours, quenched with 10% aqueous potassium carbonate solution and extracted with dichloromethane. The organic layer was washed with brine, dried over magnesium sulfate, filtered and concentrated in vacuo. The residue was recrystallized from a mixture of ethyl acetate and diisopropyl ether to give N-{4-[2-(2-amino-1,3-thiazol-4-yl)ethoxy]phenyl}-4′-(trifluoromethyl)-1,1′-biphenyl-2-carboxamide (45 mg) as a white solid.

¹H-NMR (DMSO-d₆): δ2.83 (2H, d, J=6.9 Hz), 4.14 (2H, d, J=6.9 Hz), 6.4 (1H, s), 6.83-6.86 (4H, m), 7.40 (2H, d, J=9.2 Hz), 7.49-7.64 (6H, m), 10.18 (1H, s).

ESI-MS (m/z): 484 (M+H)⁺

### EXAMPLE 80

tert-Butyl 6-{2-[4-({[4′-(trifluoromethyl)-1,1′-biphenyl-2-yl]carbonyl}amino)phenoxy]ethyl)-2-pyridinylcarbamate

The title compound was obtained from N-(4-hydroxyphenyl)-4′-(trifluoromethyl)-1,1′-biphenyl-2-carboxamide and 2-{6-[(tert-butoxycarbonyl)amino]-2-pyridinyl}ethyl 4-methylbenzenesulfonate in the same manner as in Example 78 as a white solid.

¹H-NMR (CDCl₃): δ1.51 (9H, s), 3.09 (2H, t, J=6.9 Hz), 4.25 (2H, t, J=6.9 Hz), 6.28 (2H, d, J=6.9 Hz), 6.58 (2H, d, J=8.9 Hz), 7.05 (2H, d, J=8.9 Hz), 7.40-7.79 (12H, m).

### EXAMPLE 81

To a solution of tert-butyl 6-{2-[4-({[4′-(trifluoromethyl)-1,1′-biphenyl-2-yl]carbonyl}amino)phenoxy]-ethyl}-2-pyridinylcarbamate (0.466 g) in dichloromethane (20 ml) was added trifluoroacetic acid (1.5 ml) dropwise. The reaction mixture was stirred at room temperature for 15 hours, quenched with 10% aqueous potassium carbonate solution, and extracted with dichloromethane. The organic layer was washed with brine, dried over magnesium sulfate, filtered and concentrated in vacuo. The residue was purified by column chromatography on silica gel eluting with hexane:ethyl acetate (1:3) to give N-{4-[2-(6-amino-2-pyridinyl)ethoxy]phenyl}-4′-(trifluoromethyl)-1,1′-biphenyl-2-carboxamide (0.314 g) as a white solid.

¹H-NMR (DMSO-d₆): δ2.89 (2H, t, J=6.9 Hz), 4.04 (2H, t, J=6.9 Hz), 5.81 (2H, s), 6.28 (1H, d, J=7.9 Hz), 6.43 (1H, d, J=6.9 Hz), 6.84 (1H, d, J=7.3 Hz), 6.85 (1H, d, J=6.9 Hz), 7.25-7.76 (11H, m), 10.17 (1H, s).

ESI-MS (m/z): 478 (M+H1)⁺

Preparation 43

4-[2-(4-Nitroanilino)ethyl]-1,3-thiazole

The title compound was obtained from 4-(2-aminoethyl)-1,3-thiazole and 1-fluoro-4-nitrobenzene in the same manner as in Preparation 33 as a brown oil.

¹H-NMR (CDCl₃): δ3.17 (2H, t, J=6.4 Hz), 3.60 (2H, q, J=6.1 Hz), 6.53-8.09 (4H, AaBb), 7.08 (1H, d, J=2.0 Hz), 8.8 (1H, s, J=2.0 Hz).

Preparation.44

tert-Butyl 4-nitrophenyl[2-(1,3-thiazol-4-yl)ethyl]-carbamate

The title compound was obtained from 4-[2-(4-nitroanilino)-ethyl]-1,3-thiazole in the same manner as in Preparation 34 as a yellow oil.

¹H-NMR (CDCl₃): δ1.46 (9H, s) 3.14 (2H, t, J=6.8 Hz), 4.11 (2H, t, J=7.1 Hz), 7.01 (1H, d, J=2.0 Hz), 7.26-8.16 (4H, AaBb), 8.69 (1H, d, J=2.0 Hz).

Preparation 45

tert-Butyl 4-aminophenyl[2-(1,3-thiazol-4-yl)ethyl]-carbamate

The title compound was obtained from tert-butyl 4-nitrophenyl[2-(1,3-thiazol-4-yl)ethyl]carbamate in the same manner as in Preparation 35 as an orange oil.

¹H-NMR (CDCl₃): δ1.39 (9H, s) 3.07 (2H, t, J=7.4 Hz), 3.93 (2H, t, J=7.4 Hz), 6.11 (2H, d, J=8.6 Hz), 6.9 (2H, brs), 7.0 (1H, brs), 8.7 (1H, d, J=2.0 Hz).

### EXAMPLE 82

2-[(4-{(tert-Butoxycarbonyl)[2-(1,3-thiazol-4-yl)ethyl]amino}anilino)carbonyl]-4′-(trifluoromethyl)-1,1′-biphenyl

The title compound was obtained from tert-butyl 4-aminophenyl[2-(1,3-thiazol-4-yl)ethyl]carbamate and 4′-(trifluoromethyl)-1,1′-biphenyl-2-carboxylic acid in the same manner as in Example 74 as a yellow oil.

¹H-NMR (CDCl₃): δ1.39 (9H, s) 3.06 (2H, t, J=7.3 Hz), 3.96 (2H, t, J=7.3 Hz), 6.94-7.83 (13H, m), 8.69 (1H, s).

### EXAMPLE 83

N-(4-{[2-(1,3-Thiazol-4-yl)ethyl]amino}phenyl)-4′-(trifluoromethyl)-1,1′-biphenyl-2-carboxamide

The title compound was obtained from 2-[(4-{(tert-butoxycarbonyl)[2-(1,3-thiazol-4-yl)ethyl]amino}anilino)-carbonyl]-4′-(trifluoromethyl)-1,1′-biphenyl in the same manner as in Example 75 as a yellow solid.

¹H-NMR (CDCl₃): δ3.10 (2H, t, J=6.4 Hz), 3.46 (2H, t, J=6.6 Hz), 6.50-6.96 (4H, AaBb), 6.76 (1H, brs), 7.01 (1H, d, J=1.4 Hz), 7.40-7.80 (8H, m), 8.77 (1H, d, J=2.0 Hz).

ESI-MS (m/z): 490 (M+Na)⁺, 468 (M+H)⁺

### EXAMPLE 84

2-[(4-{(tert-Butoxycarbonyl)[2-(2-pyridinyl)ethyl]amino}anilino)carbonyl]-1,1′-biphenyl

The title compound was obtained from tert-butyl 4-aminophenyl[2-(2-pyridinyl)ethyl]carbamate and 1,1′-biphenyl-2-carboxylic acid in the same manner as in Example 56 as a light yellow solid.

¹H-NMR (DMSO-d₆): δ1.26 (9H, s), 2.75 (2H, t, J=7.5 Hz), 3.84 (2H, t, J=7.5 Hz), 6.46 (2H, d, J=8.6 Hz), 6.95 (2H, d, J=8.6 Hz), 7.05-7.5 (7.6-7.7 (1H, m), 8.43 (1H, d, J=4.7 Hz), 10.27 (1H, s)

APCI-MS (m/z): 494 (M+H)⁺

### EXAMPLE 85

N-(4-{[2-(2-Pyridinyl)ethyl]amino}phenyl)-1,1′-biphenyl-2-carboxamide

The title compound was obtained from 2-[(4-{(tert-butoxycarbonyl)[2-(2-pyridinyl)ethyl]amino}anilino)carbonyl]-1,1′-biphenyl in the same manner as in Example 59 as white crystals.

¹H-NMR (DMSO-d₆): δ2.96 (2H, t, J=7.0 Hz), 3.33 (2H, td, J=7.0 and 5.7 Hz), 5.51 (1H, t, J=5.7 Hz), 6.49 (2H, d, J=8.8 Hz), 7.1 (2H, d, J=8.8 Hz), 7.3-7.6 (11H, m), 7.65-7.8 (1H, m), 8.50 (1H, d, J=4.09 Hz), 9.78 (1H, s)

ESI-MS (m/z): 416 (M+Na)⁺, 394 (M+H)⁺

### EXAMPLE 86

2-[(4-{(tert-Butoxycarbonyl)[2-(2-pyridinyl)ethyl]amino}anilino)carbonyl]-4′-fluoro-1,1′-biphenyl

The title compound was obtained from tert-butyl 4-aminophenyl[2-(2-pyridinyl)ethyl]carbamate and 4′-fluoro-1,1′-biphenyl-2-carboxylic acid in the same manner as in Example 56 as a light yellow solid.

¹H-NMR (DMSO-d₆): δ1.33 (9H, m), 2.90 (2H, t, J=7.0 Hz), 3.88 (2H, t, J=7.0 Hz), 7.1-8.0 (15H, m), 8.4-8.5 (1H, m), 10.45 (1H, s)

APCI-MS (m/z): 512 (M+H)⁺

### EXAMPLE 87

4′-Fluoro-N-(4-{[2-(2-pyridinyl)ethyl]amino}phenyl)-1,1′-biphenyl-2-carboxamide

The title compound was obtained from 2-[(4-{(tert-butoxycarbonyl)[2-(2-pyridinyl)ethyl]amino}anilino)carbonyl]-4′-fluoro-1,1′-biphenyl in the same manner as in Example 59 as white crystals.

¹H-NMR (DMSO-d₆): δ2.96 (2H, t, J=7.1 Hz), 3.33 (2H, td, J=7.1 and 5.8 Hz), 5.53 (1H, t, J=5.8 Hz), 6.50 (2H, d, J=8.9 Hz), 7.2-7.4 (5H, m), 7.45-7.65 (6H, m), 7.70 (1H, ddd, J=8.0 and 8.2 and 1.9 Hz), 8.50 (1H, d, J=4.0 Hz), 9.81 (1H, s)

APCI-MS (m/z): 412 (M+H)⁺

### EXAMPLE 88

4-Bromo-2′-[(4-{(tert-butoxycarbonyl)[2-(2-pyridinyl)ethyl]amino}anilino)carbonyl]-1,1′-biphenyl

The title compound was obtained from tert-butyl 4-aminophenyl[2-(2-pyridinyl)ethyl]carbamate and 4′-bromo-1,1′-biphenyl-2-carboxylic acid in the same manner as in Example 56 as a light yellow solid.

¹H-NMR (DMSO-d₆): δ1.32 (9H, s), 2.89 (2H, t, J=6.7 Hz), 3.89 (2H, t, J=6.7 Hz), 7.1-7.85 (15H, m), 8.45 (1H, d, J=4.0 Hz), 10.35 (1H, s)

APCI-MS (m/z): 574, 572 (M+H)⁺

### EXAMPLE 89

4′-Bromo-N-(4-{[2-(2-pyridinyl)ethyl]amino}phenyl)-1,1′-biphenyl-2-carboxamide

The title compound was obtained from 4-bromo-2′-[(4-{(tert-butoxycarbonyl)[2-(2-pyridinyl)ethyl]amino}anilino)carbonyl]-1,1′-biphenyl in the same manner as in Example 59 as white crystals.

¹H-NMR (DMSO-d₆): δ2.96 (2H, t, J=7.0 Hz), 3.33 (2H, td, J=7.0 and 5.7 Hz), 5.52 (1H, t, J=5.7 Hz), 6.50 (2H, d, J=8.8 Hz), 7.15-7.75 (11H, m), 7.65-7.8 (1H, m), 8.51 (1H, d, J=4.8 Hz), 9.80 (1H, s)

APCI-MS (m/z): 474, 472 (M+H)⁺

Preparation 46

tert-Butyl-4-[(2-iodobenzoyl)amino]phenyl[2-(2-pyridinyl)ethyl]carbamate

The title compound was obtained from tert-butyl 4-aminophenyl[2-(2-pyridinyl)ethyl]carbamate and 2-iodobenzoyl chloride in the same manner as in Preparation 19 as a light-brown amorphous solid. The obtained product was used for the next step without further purification.

Preparation 47

2-Iodo-N-(4-{[2-(2-pyridinyl)ethyl]amino}phenyl)benzamide

The title compound was obtained from tert-butyl 4-[(2-iodobenzoyl)amino]phenyl[2-(2-pyridinyl)ethyl]carbamate in the same manner as in Example 59 as white crystals.

¹H-NMR (DMSO-d₆): δ2.98 (2H, t, J=7.14 Hz), 3.34 (2H, td, J=7.1 and 5.6 Hz), 6.58 (2H, d, J=8.8 Hz), 7.2-7.6 (7H, m), 7.65-7.8 (1H, m), 7.71 (1H, ddd, J=7.9 and 7.7 and 1.8 Hz), 8.52 (1H, d, J=4.0 Hz), 9.99 (1H, s)

ESI-MS (m/z): 466 (M+Na)⁺, 444 (M+H)⁺

### EXAMPLE 90

To a solution of 2-iodo-N-(4-{[2-(2-pyridinyl)ethyl]amino}phenyl)benzamide (1.87 g) and 3-methylphenylboronic acid (651 mg) in N,N-dimethylformamide (30 ml) were added triethylamine (1.12 g) and teterakis(triphenylphosphine)palladium (213 mg) and the mixture was stirred at 110° C. under nitrogen for 72 hours. The mixture was poured into a mixture of ethyl acetate and ice water, and the separated organic layer was washed with water and brine, and evaporated in vacuo. The residue was purified by column chromatography on silica gel to give 3′-methyl-N-(4-{[2-(2-pyridinyl)ethyl]amino}phenyl)-1,1′-biphenyl-2-carboxamide (827 mg) as a white amorphous solid.

¹H-NMR (DMSO-d₆): δ2.29 (3H, s), 2.96 (2H, t, J=7.0 Hz), 3.32 (2H, td, J=7.0 and 5.8 Hz), 5.51 (1H, t, J=5.8 Hz), 6.50 (2H, d, J=8.8 Hz), 7.15-7.35 (8H, m), 7.4-7.55 (1H, m), 7.65-7.8 (1H, m), 8.51 (1H, d, J=4.8 Hz), 9.75 (1H, s)

APCI-MS (m/z): 408 (M+H)⁺

### EXAMPLE 91

4′-Methoxy-N-[4-(2-pyridinylmethyl)phenyl]-1,1′-biphenyl-2-carboxamide

The title compound was obtained from 4-(2pyridinylmethyl)phenylamine and 4′-methoxy-1,1′-biphenyl-2-carbonyl chloride in the same manner as in Preparation 19.

¹H-NMR (DMSO-d₆): δ3.73 (3H, s), 4.01 (2H, s), 6.92 (2H, d, J=8.7 Hz), 7.12-7.28 (4H, m), 7.32-7.56 (8H, m), 7.62-7.74 (1H, m), 8.47 (1H, d, J=4.1 Hz), 10.17 (1H, s)

(+)APCI-MS (m/z): 395 (M+H)⁺

### EXAMPLE 92

N-[4-(2-Pyridinylmethyl)phenyl]-4′-(trifluoromethoxy)-1,1′-biphenyl-2-carboxamide

The title compound was obtained from 4-(2-pyridinylmethyl)phenylamine and 4′-(trifluoromethoxy)-1,1′-biphenyl-2-carbonyl chloride in the same manner as in Preparation 19.

¹H-NMR (DMSO-d₆): δ4.01 (2H, s), 7.12-7.26 (4H,m), 7.35-7.43(4H, m), 7.43-7.61 (6H, m), 7.61-7.74 (1H, m), 8.47 (1H, d, J=4.4 Hz), 10.22 (1H, s)

(+)APCI-MS (m/z): 449 (M+H)⁺

### EXAMPLE 93

2-[(4-{(tert-Butoxycarbonyl)[2-(2-pyridinyl)ethyl]amino}anilino)carbonyl]-4′-(trifluoromethoxy)-1,1′-biphenyl

The title compound was obtained from tert-butyl 4-aminophenyl[2-(2-pyridinyl)ethyl]carbamate and 4′-(trifluoromethoxy)-1,1′-biphenyl-2-carboxylic acid in the same manner as in Example 49.

¹H-NMR (DMSO-d₆): δ1.29 (9H, s), 2.94 (2H, t, J=7.0 Hz), 3.91 (2H, t, J=7.0), 7.11 (2H, d, J=8.7 Hz), 7.30-7.64 (12H, m), 7.85 (1H, t, J=7.0 Hz), 8.53 (1H, d, J=4.5), 10.32 (1H, s)

(+)APCI-MS (m/z): 578 (M+H)⁺

### EXAMPLE 94

N-(4-{[2-(2-Pyridinyl)ethyl]amino}phenyl)-4′-(trifluoromethoxy)-1,1′-biphenyl-2-carboxamide

The title compound was obtained from 2-[(4-{(tert-butoxycarbonyl)[2-(2-pyridinyl)ethyl]amino}anilino)carbonyl]-4′-(trifluoromethoxy)-1,1′-biphenyl in the same manner as in Example 59.

¹H-NMR (DMSO-d₆): δ2.96 (2H, d, J=7.2 Hz), 3.27-3.45 (2H, m), 5.54 (1H, t, J=5.7 Hz), 6.50 (2H, d, J=8.8 Hz), 7.18 (2H, d, J=8.8 Hz), 7.17-7.55 (10H, m), 7.68 (1H, dt, J=1.8 Hz 7.6 Hz), 8.51 (1H, d, J=4.0 Hz), 9.83 (1H, s)

(+)APCI-MS: 478 (M+H)⁺

### EXAMPLE 95

4′-(Methylthio)-N-[4-(2-pyridinylmethyl)phenyl]-1,1′-biphenyl-2-carboxamide

The title compound was obtained from 4-(2-pyridinylmethyl)phenylamine and 4′-(methylthio)-1,1′-biphenyl-2-carbonyl chloride in the same manner as in Preparation 19.

¹H-NMR (DMSO-d₆): δ2.44 (3H, s), 4.01 (2H, s), 7.13-7.60 (14H, m), 7.68 (1H, dt, J=1.7 Hz, 7.7 Hz), 8.47 (1H, d, J=4.2 Hz), 10.24 (1H, s)

(+)APCI-MS (m/z): 411 (M+H)⁺

### EXAMPLE 96

2-[(4-[(tert-Butoxycarbonyl)[2-(2-pyridinyl)ethyl]amino}anilino)carbonyl]-4′-(methylthio)-1,1′-biphenyl

The title compound was obtained from tert-butyl 4-aminophenyl[2-(2-pyridinyl)ethyl]carbamate and 4′-(methylthio)-1,1′-biphenyl-2-carbonyl chloride in the same manner as in Preparation 19.

¹H-NMR (DMSO-d₆): δ1.30 (9H, s), 2.46 (3H, s), 2.95 (2H, t, J=7.0 Hz), 3.92 (2H, t, J=7.0), 7.12 (2H, d, J=8.7 Hz), 7.23-7.70 (12H, m), 7.86 (1H, t, J=7.5 Hz), 8.54 (1H, d, J=4.3), 10.35 (1H, s)

### EXAMPLE 97

4′-(Methylthio)-N-(4-{[2-(2-pyridinyl)ethyl]amino}phenyl)-1,1′-biphenyl-2-carboxamide

The title compound was obtained from 2-[(4-{(tert-butoxycarbonyl)[2-(2-pyridinyl)ethyl]amino}anilino)carbonyl]-4′-(methylthio)-1,1′-biphenyl in the same manner as in Example 59.

¹H-NMR (DMSO-d₆): δ2.47 (3H, s), 2.96 (2H, t, J=7.2 Hz), 3.27-3.40 (2H, m), 5.52 (1H, s), 6.51 (2H, d, J=8.8 Hz), 7.17-7.55 (12H, m), 7.70 (1H, dt, J=1.8 Hz 7.6 Hz), 8.50 (1H, d, J=4.8 Hz), 9.84 (1H, s)

(+)APCI-MS (m/z): 440 (M+H)⁺

### EXAMPLE 98

4′-(Methylsulfonyl)-N-[4-(2-pyridinylmethyl)phenyl]-1,1′-biphenyl-2-carboxamide

The title compound was obtained from 4-(2-pyridinylmethyl)phenylamine and 4′-(methylsulfonyl)-1,1′-biphenyl-2-carbonyl chloride in the same manner as in Preparation 19.

¹H-NMR (DMSO-d₆): δ3.21 (3H, s), 4.01 (2H, s), 7.14-7.26 (4H, m), 7.40-7.73 (9H, m), 7.92 (1H, d, J=8.4 Hz), 8.46 (1H, d, J=4.0 Hz), 10.33 (1H, s)

(+)APCI-MS (m/z): 443 (M+H)⁺

### EXAMPLE 99

2-[(4-{(tert-Butoxycarbonyl)[2-(2-pyridinyl)ethyl]amino}anilino)carbonyl]-4′-(methylsulfonyl)-1,1′-biphenyl

The title compound was obtained from tert-butyl 4-aminophenyl[2-(2-pyridinyl)ethyl]carbamate and 4′-(methylsulfonyl)-1,1′-biphenyl-2-carbonyl chloride in the same manner as in Preparation 19.

¹H-NMR (DMSO-d₆): δ1.32 (9H, s), 2.88 (2H, t, J=7.2 Hz), 3.23 (3H, s), 3.89 (2H, t, J=7.2), 7.12 (2H, d, J=8.7 Hz), 7.16-7.26 (2H, m), 7.46-7.73 (9H, m), 7.95 (2H, d, J=8.4 Hz), 8.46 (1H, d, J=4.0), 10.44 (1H, s)

### EXAMPLE 100

4′-(Methylsulfonyl)-N-(4-{[2-(2-pyridinyl)ethyl]amino}phenyl)-1,1′-biphenyl-2-carboxamide

The title compound was obtained from 2-[(4-{(tert-butoxycarbonyl)[2-(2-pyridinyl)ethyl]amino}anilino)carbonyl]-4′-(methylsulfonyl)-1,1′-biphenyl in the same manner as in Example 59.

¹H-NMR (DMSO-d₆): δ2.96 (2H, t, J=7.2 Hz), 3.24 (3H, s), 3.28-3.40 (2H, m), 5.55 (1H, t, J=5.7 Hz), 6.52 (2H, d, J=8.8 Hz), 7.08-7.33 (2H, m), 7.12 (2H, d, J=8.8 Hz), 7.45-7.75 (7H, m), 7.94 (2H, d, J=8.4 Hz), 8.51 (1H, d, J=4.0 Hz), 9.96 (1H, s)

(+)APCI-MS (m/z): 472 (M+H)⁺

### EXAMPLE 101

WSC (0.17 g) was added to a solution of tert-butyl 4-aminophenyl[2-(2-pyridinyl)ethyl]carbamate (0.31 g), 4′-(isopropylthio)-1,1′-biphenyl-2-carboxylic acid 40.3 g), HOBT (0.17 g) and 4-dimethylaminopyridine (2.4 mg) in dichloromethane (3 ml) under ice-cooling and the mixture was stirred at ambient temperature for 20 hours. To the reaction mixture was added a solution of 10% hydrogen chloride in methanol (9 ml) and the mixture was stirred at ambient temperature for 22 hours.

The reaction mixture was poured into a mixture of ethyl acetate, tetrahydrofuran and water and the mixture was adjusted to pH 9 with 20% aqueous potassium carbonate solution. The separated organic layer was washed with water, dried over magnesium sulfate and evaporated in vacuo. The residue was triturated with diethyl ether to give 4′-(isopropylthio)-N-(4-{[2-(2-pyridinyl)ethyl]amino}phenyl)-1,1′-biphenyl-2-carboxamide (0.30 g).

¹H-NMR (DMSO-d₆): δ1.22 (6H, d, J=6.6 Hz), 2.96 (2H, d, J=7.2 Hz), 3.27-3.40 (2H, m), 3.43-3.57 (1H, m), 5.52 (1H, t, J=5.7 Hz), 6.49 (2H, d, J=8.8 Hz), 7.14-7.57 (10H, m), 7.18 (2H, d, J=8.8 Hz), 7.70 (1H, dt, J=1.7 Hz, 7.6 Hz), 8.51 (1H, d, 4.7 Hz), 9.74 (1H, s)

(+)APCI-MS (m/z): 468 (M+H)⁺

### EXAMPLE 102

4′-(Isopropylsulfonyl)-N-(4-{[2-(2-pyridinyl)ethyl]amino}phenyl)-1,1′-biphenyl-2-carboxamide

The title compound was obtained from tert-butyl 4-aminophenyl[2-(2-pyridinyl)ethyl]carbamate and 4′-(isopropylsulfonyl)-1,1′-biphenyl-2-carboxylic acid in the same manner as in Example 101.

¹H-NMR (DMSO-d₆): δ1.10 (6H, d, J=6.7 Hz), 2.95 (2H, d, J=7.0 Hz), 3.23-3.46 (3H, m), 5.53 (1H, t, J=5.7 Hz), 6.48 (2H, d, J=8.5 Hz), 7.08-7.37 (2H, m), 7.14 (2H, d, J=8.5 Hz), 7.47-7.78(7H, m), 7.85 (2H, d, J=8.1 Hz), 8.50 (1H, d, J=3.9 Hz), 9.77 (1H, s)

(+)APCI-MS (m/z): 500 (M+H)⁺

### EXAMPLE 103

4′-Iodo-N-(4-{[2-(2-pyridinyl)ethyl]amino}phenyl)-1,1′-biphenyl-2-carboxamide

The title compound was obtained from tert-butyl 4-aminophenyl[2-(2-pyridinyl)ethyl]carbamate and 4′-iodo-1,1′-biphenyl-2-carboxylic acid in the same manner as in Example 101.

¹H-NMR (DMSO-d₆): δ2.97 (2H, d, J=7.2 Hz), 3.28-3.40 (2H, m), 5.54 (1H, t, J=5.7 Hz), 6.52 (2H, d, J=8.8 Hz), 7.17-7.37 (6H, m), 7.39-7.60 (4H, m), 7.66-7.80 (3H, m), 8.51 (1H, d, J=4.0 Hz), 9.87 (1H, s)

(+)APCI-MS (m/z): 520 (M+H)⁺

### EXAMPLE 104

A mixture of potassium cyanide (75.2 mg) and zinc powder (44.1 mg) in N,N-dimethylformamide (5 ml) was stirred at ambient temperature for 10 hours. To the mixture was added a mixture of 4′-iodo-N-(4-{[2-(2-pyridinyl)ethyl]amino}phenyl)-1,1′-biphenyl-2-carboxamide (0.5 g), triethylamine (0.16 ml) and [1,1′-bis(diphenylphosphino)ferrocene]dichloropalladium (II), complex with dichloromethane (1:1) (78.6 mg) and the mixture was stirred at 60° C. for 3 hours. The reaction mixture was poured into a mixture of ethyl acetate and water and the mixture was adjusted to pH 2 with 6N-hydrochloric acid. The separated aqueous layer was adjusted to pH 9 with 20% aqueous potassium carbonate solution and extracted with a mixture of ethyl acetate and tetrahydrofuran. The extract was washed with water, dried over magnesium sulfate and evaporated in vacuo. The residue was purified by column chromatography on silica gel using an ethyl acetate as an eluent. The eluted fractions containing the desired product were collected and evaporated in vacuo to give 4′-cyano-N-(4-{[2-(2-pyridinyl)ethyl]amino}phenyl)-1,1′-biphenyl-2-carboxamide (0.27 g).

¹H-NMR (DMSO-d₆): δ2.96 (2H, d, J=7.2 Hz), 3.27-3.41 (2H, m), 5.55 (1H, t, J=5.8 Hz), 6.51 (2H, d, J=8.8 Hz), 7.17-7.37 (4H, m), 7.42-7.64 (6H, m), 7.70 (1H, dt, J=1.8 Hz, 7.6 Hz), 7.87 (2H, d, J=8.2 Hz), 8.51 (1H, d, J=4.8 Hz), 9.93 (1H, s)

(+)APCI-MS (m/z): 419 (M+H)⁺

### EXAMPLE 105

Methyl 2′-[(4-{[2-(2-pyridinyl)ethyl]amino}anilino)-carbonyl]-1,1′-biphenyl-4-carboxylate

The title compound was obtained from tert-butyl 4-aminophenyl[2-(2-pyridinyl)ethyl]carbamate and 4′-(methoxycarbonyl)-1,1′-biphenyl-2-carboxylic acid in the same manner as in Example 101.

¹H-NMR (DMSO-d₆): δ2.96 (2H, t, J=7.2 Hz), 3.28-3.39 (2H, m), 3.84 (3H, s), 5.55 (1H, s), 6.51 (2H, d, J=8.8 Hz), 7.17-7.33 (4H, m), 7.45-7.63 (6H, m), 7.70 (1H, dt, J=1.8 Hz, 7.6 Hz), 7.96 (2H, d, J=8.3 Hz), 8.51 (1H, d, J=4.4), 9.87 (1H, s)

(+)APCI-MS (m/z): 452 (M+H)⁺

### EXAMPLE 106

2-[(4-{(tert-Butoxycarbonyl)[2-(2-pyridinyl)ethyl]amino}anilino)carbonyl]-4′-nitro-1,1′-biphenyl

The title compound was obtained from tert-butyl 4-aminophenyl[2-(2-pyridinyl)ethyl]carbamate and 4′-nitro-1,1′-biphenyl-2-carbonyl chloride in the same manner as in Preparation 19.

¹H-NMR (DMSO-d₆): δ1.31 (9H, s), 2.88 (2H, t, J=7.3 Hz), 3.89 (2H, t, J=7.3), 7.10-7.25 (2H, m), 7.12(2H, d, J=8.7 Hz), 7.46-7.75 (7H, m), 7.51 (2H, d, J=8.7), 8.27 (2H, d, J=8.7), 8.45 (1H, d, J=4.6 Hz), 10.45 (1H, s)

### EXAMPLE 107

4′-Nitro-N-(4-{[2-(2-pyridinyl)ethyl]amino}phenyl)-1,1′-biphenyl-2-carboxamide

The title compound-was obtained from 2-[(4-{(tert-butoxycarbonyl)[2-(2-pyridinyl)ethyl]amino}anilino)carbonyl]-4′-nitro-1,1′-biphenyl in the same manner as in Example 59.

¹H-NMR (DMSO-d₆): δ2.96 (2H, t, J=7.2 Hz), 3.27-3.40 (2H, m), 5.55 (1H, t, J=5.7 Hz), 6.51 (2H, d, J=8.8 Hz), 7.17-7.33 (4H, m), 7.47-7.75 (7H, m), 8.26 (2H, d, J=8.8 Hz), 8.50 (1H, d, J=4.1 Hz), 9.96 (1H, s)

### EXAMPLE 108

To a solution of 4′-nitro-N-(4-{[2-(2-pyridinyl)ethyl]amino}phenyl)-1,1′-biphenyl-2-carboxamide (0.4 g) in a mixture of methanol (8 ml) and tetrahydrofuran (8 ml) was added 10% palladium on carbon (0.4 g, 50% wet). The reaction mixture was stirred at ambient temperature for 4 hours under a hydrogen atmosphere. The catalyst was filtered off and the solvent was removed by evaporation. The residue was triturated with diethyl ether to give 4′-amino-N-(4-{[2-(2pyridinyl)ethyl]amino}phenyl)-1,1′-biphenyl-2-carboxamide (0.23 g).

¹H-NMR (DMSO-d₆): δ2.97 (2H, t, J=7.2 Hz), 3.28-3.44 (2H, m), 5.13 (2H, s), 5.50 (1H, t, J=5.7 Hz), 6.51 (2H, d, J=8.7 Hz), 6.54 (2H, d, J=8.3 Hz), 7.11-7.49 (8H, m), 7.14 (2H, d, J=8.3 Hz), 7.70 (1H, dt, J=1.8 Hz, 7.6 Hz), 8.51 (1H, d, J=4.1), 9.66 (1H, s)

(+)APCI-MS (m/z): 409 (M+H)⁺

### EXAMPLE 109

To a mixture of 2-[(4-{(tert-butoxycarbonyl)[2-(2pyridinyl)ethyl]amino}anilino)carbonyl]-4′-nitro-1,1′-biphenyl (0.6 g) and 37% aqueous formaldehyde (1.7 ml) in a mixture of methanol (4 ml) and tetrahydrofuran (4 ml) was added 10% palladium on carbon (0.6 g, 50% wet). The reaction mixture was stirred at ambient temperature for 8 hours under a hydrogen atmosphere.

The catalyst was filtered off and the solvent was removed by evaporation. The residue was triturated with a mixture of diethyl ether and diisopropyl ether to give 2-[(4-{(tert-butoxycarbonyl)[2-(2-pyridinyl)ethyl]amino}anilino)carbonyl]-4′-(dimethylamino)-1,1′-biphenyl (0.53 g).

¹H-NMR (DMSO-d₆): δ1.32 (9H, s), 2.88 (2H, t, J=7.2 Hz), 2.89 (6H, s), 3.89 (2H, t, J=7.2 Hz), 6.72 (2H, d, J=8.8 Hz), 7.11 (2H, d, J=8.8 Hz), 7.15-7.58 (10H, m), 7.68 (1H, dt, J=1.8 Hz, 7.6 Hz), 8.46 (1H, d, J=4.4), 10.25 (1H, s)

### EXAMPLE 110

4′-(Dimethylamino)-N-(4-{[2-(2-pyridinyl)ethyl]amino}-phenyl)-1,1′-biphenyl-2-carboxamide

The title compound was obtained from 2-[(4-{(tert-butoxycarbonyl)[2-(2-pyridinyl)ethyl]amino}anilino)carbonyl]-4′-(dimethylamino)-1,1′-biphenyl in the same manner as in Example 59.

¹H-NMR (DMSO-d₆): δ2.89 (6H, s), 2.96 (2H, t, J=7.2 Hz), 3.28-3.40 (2H, m), 5.51 (1H, t, J=5.7 Hz), 6.52 (2H, d, J=8.8 Hz), 6.71 (2H, d, J=8.8 Hz), 7.17-7.51 (10H, m), 7.70 (1H, dt, J=1.1 Hz, 7.6 Hz), 8.51 (1H, d, J=4.7 Hz), 9.77 (1H, s)

(+)APCI-MS (m/z): 437 (M+H)⁺

### EXAMPLE 111

4-Amino-2′-[(4-{(tert-butoxycarbonyl)[2-(2-pyridinyl)ethyl]amino}anilino)carbonyl]-1,1′-biphenyl

The title compound was obtained from 2-[(4-{(tert-butoxycarbonyl)[2-(2-pyridinyl)ethyl]amino}anilino)carbonyl]-4′-nitro-1,1′-biphenyl in the same manner as in Example 108.

¹H-NMR (DMSO-d₆): δ1.32 (9H, s), 2.89 (2H, t, J=7.2 Hz), 3.89 (2H, t, J=7.2 Hz), 5.15 (2H, s), 6.55 (2H, d, J=8.4 Hz), 7.05-7.28 (6H, m), 7.30-7.68 (6H, m), 7.68 (1H, dt, J=1.8 Hz, 7.6 Hz), 8.46 (1H, d, J=4.5 Hz), 10.16 (1H, s)

### EXAMPLE 112

Acetyl chloride (0.09 ml) was added to a solution of 4-amino-2′-[(4-{(tert-butoxycarbonyl)[2-(2-pyridinyl)ethyl]amino}anilino)carbonyl]-1,1′-biphenyl (0.51 g) and triethylamine (0.17 ml) in tetrahydrofuran (5 ml) under ice-cooling and the mixture was stirred at ambient temperature for 20 hours.

The reaction mixture was poured into a mixture of ethyl acetate, tetrahydrofuran and water and the mixture was adjusted to pH 8 with 20% aqueous potassium carbonate solution. The separated organic layer was washed with water, dried over magnesium sulfate and evaporated in vacuo. The residue was triturated with diisopropyl ether to give 4-(acetylamino)-2′-[(4-(tert-butoxycarbonyl)[2-(2-pyridinyl)ethyl]amino}anilino)carbonyl]-1,1′-biphenyl (0.52 g)

¹H-NMR (DMSO-d₆): δ1.32 (9H, s), 2.02 (3H, s), 2.88 (2H, t, J=7.2 Hz), 3.89 (2H, t, J=7.2 Hz), 7.10 (2H, d, J=8.7 Hz), 7.16-7.26 (2H, m), 7.34-7.62 (10H, m), 7.68 (1H, dt, J=1.6 Hz, 7.6 Hz), 8.45 (1H, d, J=4.7 Hz), 9.96 (1H, s), 10.26 (1H, s)

### EXAMPLE 113

4′-(Acetylamino)-N-(4-{[2-(2-pyridinyl)ethyl]amino}phenyl)-1,1′-biphenyl-2-carboxamide

The title compound was obtained from 4-(acetylamino)-2′-[(4-{(tert-butoxycarbonyl)[2-(2-pyridinyl)ethyl]amino}anilino)-carbonyl]-1,1′-biphenyl in the same manner as in Example 59.

¹H-NMR (DMSO-d₆): δ2.03 (3H, s), 2.96 (2H, t, J=7.2 Hz), 3.27-3.40 (2H, m), 5.52 (1H, t, J=5.7 Hz), 6.50 (2H, d, J=8.8 Hz), 7.18-7.60 (12H, m), 7.70 (1H, dt, J=1.8 Hz, 7.6 Hz), 8.51 (1H, d, J=4.7 Hz), 9.77 (1H, s), 9.96 (1H, s)

(+)APCI-MS (m/z): 451 (M+H)⁺

### EXAMPLE 114

2-(4-Pyridinyl)-N-[4-(2-pyridinylmethyl)phenyl]benzamide

The title compound was obtained from 4-(2-pyridinylmethyl)phenylamine and 2-(4-pyridinyl)benzoic acid in the same manner as in Example 56.

¹H-NMR (DMSO-d₆): δ4.03 (2H, s), 7.16-7.27 (2H, m), 7.18 (2H, d, J=8.4 Hz), 7.40-7.68 (8H, m), 7.69 (1H, dt, J=1.9 Hz, 7.6 Hz), 8.47 (1H, dd, J=0.9 Hz, 3.9 Hz), 8.55 (2H, dd, J=1.6 Hz, 4.5 Hz), 10.31 (1H, s)

(+)APCI-MS (m/z): 366 (M+H)⁺

### EXAMPLE 115

2-(4-Pyridinyl)-N-(4-{[2-(2-pyridinyl)ethyl]amino}phenyl)-benzamide

The title compound was obtained from tert-butyl 4-aminophenyl[2-(2-pyridinyl)ethyl]carbamate and 2-(4pyridinyl)benzoic acid in the same manner as in Example 101.

¹H-NMR (DMSO-d₆): δ2.96 (2H, t, J=7.3 Hz), 3.28-3.44 (2H, m), 5.55 (1H, t, J=5.7 Hz), 6.52 (2H, d, J=8.8 Hz), 7.18-7.38 (4H, m), 7.46 (2H, dd, J=1.4 Hz, 10.2 Hz), 7.49-7.68 (4H, m), 7.70 (1H, dt, J=1.8 Hz, 7.6 Hz), 8.51 (1H, d, J=4.1), 8.57 (2H, d, J=6.0 Hz), 9.94 (1H, s)

(+)APCI-MS (m/z): 395 (M+H)⁺

Preparation 48

N-(4-Hydroxy-3-nitrophenyl)-4′-(trifluoromethyl)-1,1′-biphenyl-2-carboxamide

The title compound was obtained from 4′-(trifluoromethyl)-1,1′-biphenyl-2-carbonyl chloride and 4-amino-2-nitrophenol in the same manner as in Preparation 32.

¹H-NMR (DMSO-d₆): δ7.09 (1H, d, J=9.0 Hz), 7.50-7.68 (7H, m), 7.77 (2H, d, J=8.3 Hz), 8.23 (1H, d, J=2.6 Hz), 10.51 (1H, s), 10.74 (1H, br-s)

(+)APCI-MS (m/z): 403 (M+H)⁺

### EXAMPLE 116

N-{3-Nitro-4-[2-(2-pyridinyl)ethoxy]phenyl}-4′-(trifluoromethyl)-1,1′-biphenyl-2-carboxamide

The title compound was obtained from N-(4-hydroxy-3-nitrophenyl)-4′-(trifluoromethyl)-1,1′-biphenyl-2-carboxamide in the same manner as in Example 72.

¹H-NMR (DMSO-d₆): δ3.18 (2H, t, J=6.5 Hz), 4.49 (2H, t, J=6.5 Hz), 7.20-7.27 (1H, m), 7.32-7.42 (2H, m), 7.50-7.80 (10H, m), 8.11 (1H, d, J=2.6 Hz), 8.49 (1H, d, J=4.0 Hz), 10.58 (1H, s)

(+)APCI-MS (m/z): 508 (M+H)⁺

### EXAMPLE 117

N-{3-Amino-4-[2-(2-pyridinyl)ethoxy]phenyl}-4′-(trifluoromethyl)-1,1′-biphenyl-2-carboxamide

The title compound was obtained from N-{3-nitro-4-[2-(2pyridinyl)ethoxy]phenyl}-4′-(trifluoromethyl)-1,1′-biphenyl-2-carboxamide in the same manner as in Example 108.

¹H-NMR (DMSO-d₆): δ3.17 (2H, t, J=6.5 Hz), 4.23 (2H, t, J=6.5 Hz), 4.65 (2H, s), 6.61 (1H, dd, J=2.3 Hz, 8.6 Hz), 6.71 (1H, d, J=8.6 Hz), 6.97 (1H, d, J=2.3 Hz), 7.21-7.27 (1H, m), 7.39 (1H, d, J=7.8 Hz), 7.44-7.80 (9H, m), 8.51 (1H, dd, J=0.9 Hz, 4.8 Hz), 10.00 (1H, s)

(+)APCI-MS (m/z): 478 (M+H)⁺

Preparation 49

N-(4-Fluoro-3-nitrophenyl)-4′-(trifluoromethoxy)-1,1′-biphenyl-2-carboxamide

The title compound was obtained from 4-fluoro-3-nitrophenylamine and 4′-(trifluoromethoxy)-1,1′-biphenyl-2-carbonyl chloride in the same manner as in Preparation 19.

¹H-NMR (DMSO-d₆): δ7.38 (2H, d, J=8.2 Hz), 7.47-7.60 (7H, m), 7.73-7.85 (1H, m), 8.44 (1H, dd, J=2.6 Hz, 6.9 Hz), 10.75 (1H, s)

### EXAMPLE 118

A mixture of 1-(4-fluoro-3-nitrophenyl)-4′-(trifluoromethoxy)-1,1′-biphenyl-2-carboxamide (0.45 g), 2-(2-aminoethyl)pyridine (0.26 ml) and triethylamine (0.3 ml) in N,N-dimethylformamide (4.5 ml) was stirred at 60° C. for 3 hours. The reaction mixture was poured into water and the mixture was extracted with a mixture of ethyl acetate and tetrahydrofuran. The extract was washed with water, dried over magnesium sulfate and evaporated in vacuo to give N-(3-nitro-4-([2-(2-pyridinyl)ethyl]amino}phenyl)-4′-(trifluoromethoxy)-1,1′-biphenyl-2-carboxamide (0.54 g).

¹H-NMR (DMSO-d₆): δ3.10 (2H, t, J=6.8 Hz), 3.65-3.77 (2H, m), 7.09 (1H, d, J=9.4 Hz), 7.21-7.29 (1H, m), 7.31-7.46 (3H, m), 7.48-7.78 (8H, m), 8.29 (1H, t, J=5.4 Hz), 8.40 (1H, d, J=2.5 Hz), 8.53 (1H, d, J=4.0 Hz), 10.28 (1H, s)

(+)APCI-MS (m/z): 523 (M+H)⁺

### EXAMPLE 119

N-(3-Amino-4-{[2-(2-pyridinyl)ethyl]amino}phenyl)-4′-(trifluoromethoxy)-1,1′-biphenyl-2-carboxamide

The title compound was obtained from N-(3-nitro-4-{[2-(2-pyridinyl)ethyl]amino}phenyl)-4′-(trifluoromethoxy)-1,1′-biphenyl-2-carboxamide in the same manner as in Example 108.

¹H-NMR (DMSO-d₆): δ3.01 (2H, t, J=7.2 Hz), 4.47 (1H, s), 4.52 (2H, s), 6.39 (1H, d, J=8.5 Hz), 6.59 (1H, dd, J=2.1 Hz, 8.4 Hz), 6.88 (1H, d, J=2.1 Hz), 7.22 (1H, dd, J=5.6 Hz, 7.6 Hz), 7.28-7.6 (9H, m), 7.70 (1H, dt, J=1.8 Hz, 7.6 Hz), 8.51 (1H, d, J=4.0 Hz), 9.76 (1H, s)

### EXAMPLE 120

To a mixture of 4′-ethoxy-1,1′-biphenyl-2-carboxylic acid (420 mg), tert-butyl 4-aminophenyl[2-(2-pyridinyl)ethyl]carbamate (543 mg) and HOBT.H₂O (344 mg) in tetrahydrofuran (8.5 ml) was added WSC (0.473 ml) dropwise under a nitrogen atmosphere and the solution was refluxed for 17 hours. After the reaction mixture was cooled down to ambient temperature, the solvent was evaporated in vacuo. The residue was dissolved in ethyl acetate and washed with water three times and then brine, dried over magnesium sulfate and evaporated in vacuo. The residue was chromatographed on silica gel eluting with hexane-ethyl acetate (from hexane-ethyl acetate 2:1 to 2:3). The eluate was concentrated in vacuo to give 2-[(4-{(tert-butoxycarbonyl)[2-(2-pyridinyl)ethyl]amino}anilino)carbonyl]-4′-ethoxy-1,1′-biphenyl (718 mg) as a white amorphous.

¹H-NMR (DMSO-d₆): δ1.30 (3H, t, J=7.0 Hz), 1.32 (9H, s), 2.85-2.92 (2H, m), 3.85-3.92 (2H, m), 4.01 (2H, q, J=7.0 Hz), 6.93 (2H, d, J=8.7 Hz), 7.10 (2H, d, J=8.7 Hz), 7.17-7.25 (2H, m), 7.34-7.58 (8H, m), 7.68 (1H, td, J=7.7 and 1.8 Hz), 8.46 (1H, d, J=4.6 Hz), 10.25 (1H, s)

APCI-MS (m/z): 538 (M+H)⁺

### EXAMPLE 121

To a solution of 2-[(4-{(tert-butoxycarbonyl)[2-(2-pyridinyl)ethyl]amino}anilino)carbonyl]-4′-ethoxy-1,1′-biphenyl (710 mg) in dichloromethane (14 ml) was added trifluoroacetic acid (0.7 ml) dropwise. After the mixture was stirred for 18 hours, the solvent was evaporated in vacuo. The resultant residue was dissolved in ethyl acetate and the pH of the solution was adjusted to 8.0 with saturated aqueous sodium hydrogencarbonate solution. The separated organic layer was washed with water and brine, dried over magnesium sulfate and evaporated in vacuo. The crystallization of the residue was induced by scratching the flask and the resulting crystals were washed with ether and dried in vacuo to give 4′-ethoxy-N-(4-{[2-(2-pyridinyl)ethyl]amino}phenyl)-1,1′-biphenyl-2-carboxamide (495 mg) as white crystals.

¹H-NMR (DMSO-d₆): δ1.31 (3H, t, J=7.0 Hz), 2.96 (2H, t, J=7.4 Hz), 3.28-3.39 (2H, m), 4.01 (2H, q, J=7.0 Hz), 5.12 (1H, t, J=5.8 Hz), 6.51 (2H, d, J=8.8 Hz), 6.92 (2H, d, J=8.7 Hz), 7.19-7.53 (10H, m), 7.70 (1H, td, J=7.6 and 1.8 Hz), 8.51 (1H, d, J=4.1 Hz), 9.76 (1H, s)

APCI-MS (m/z): 438 (M+H)⁺

### EXAMPLE 122

2-[(4-{(tert-Butoxycarbonyl)[2-(2-pyridinyl)ethyl]amino}anilino)carbonyl]-4′-isopropoxy-1,1′-biphenyl

The title compound was obtained from 4′-isopropoxy-1,1′-biphenyl-2-carboxylic acid in the same manner as in Example 120.

¹H-NMR (DMSO-d₆): δ1.23 (6H, d, J=6.0 Hz), 1.31 (9H, s), 2.84-2.92 (2H, m), 3.84-3.92 (2H, m), 4.61 (1H, septet, J=6.0 Hz), 6.91 (2H, d, J=8.8 Hz), 7.10 (2H, d, J=8.8 Hz), 7.21-7.24 (2H, m), 7.33-7.57 (8H, m), 7.64-7.73 (1H, m), 8.44-8.47 (1H, m), 10.21 (1H, s)

(−)APCI-MS (m/z): 550 (M−H)⁻

### EXAMPLE 123

4′-Isopropoxy-N-(4-{[2-(2-pyridinyl)ethyl]amino}phenyl)1,1′-biphenyl-2-carboxamide

The title compound was obtained from 2-[(4-{(tert-butoxycarbonyl)[2-(2-pyridinyl)ethyl]amino}anilino)carbonyl]-4′-isopropoxy-1,1′-biphenyl in the same manner as in Example 121.

¹H-NMR (DMSO-d₆): δ1.25 (6H, d, J=6.0 Hz), 2.92-3.00 (2H, m), 3.28-3.39 (2H, m), 4.61 (1H, septet, J=6.0 Hz), 5.49-5.55 (2H, m), 6.50 (2H, d, J=8.8 Hz), 6.91 (2H, d, J=8.6 Hz), 7.20 (2H, d J=8.6 Hz), 7.26 (1H, d, J=7.1 Hz), 7.33-7.53 (8H, m), 7.70 (1H, td, J=7.6 and 1.8 Hz), 8.51 (1H, d, J=4.8 Hz), 9.73 (1H, s)

APCI-MS (m/z): 452 (M+H)⁺

### EXAMPLE 124

2-[(4-{(tert-Butoxycarbonyl)[2-(2-pyridinyl)ethyl]amino}-anilino)carbonyl]-4′-(cyclohexyloxy)-1,1′-biphenyl

The title compound was obtained from 4′-(cyclohexyloxy)-1,1′-biphenyl-2-carboxylic acid in the same manner as in Example 120.

¹H-NMR (DMSO-d₆): δ1.24-1.95 (10H, m), 1.32 (9H, s), 2.84-2.92 (2H, m), 3.84-3.92 (2H, m), 4.29-4.37 (1H, m), 6.93 (2H, d, J=8.7 Hz), 7.10 (2H, d, J=8.7 Hz), 7.17-7.254 (2H, m), 7.35 (2H, d, J=8.6 Hz), 7.43-7.55 (6H, m), 7.68 (1H, td, J=7.7 and 1.8 Hz), 8.45 (1H, d, J=4.8 Hz), 10.21 (1H, s)

APCI-MS (m/z): 592 (M+H)⁺

### EXAMPLE 125

4′-(Cyclohexyloxy)-N-(4-{[2-(2-pyridinyl)ethyl]amino}-phenyl)-1,1′-biphenyl-2-carboxamide

The title compound was obtained from 2-[(4-{(tert-butoxycarbonyl)[2-(2-pyridinyl)ethyl]amino}anilino)carbonyl]-4′-(cyclohexyloxy)-1,1′-biphenyl in the same manner as in Example 121.

¹H-NMR (DMSO-d₆): δ1.27-1.99 (10H, m), 2.92-3.00 (2H, m), 3.28-3.39 (2H, m), 4.31-4.35 (1H, m), 5.48-5.54 (1H, m), 6.50 (2H, d, J=8.8 Hz), 6.92 (2H, d, J=8.7 Hz), 7.17-7.49 (10H, m), 7.65-7.76 (1H, m), 8.49-8.52 (1H, m), 9.70 (1H, s)

APCI-MS (m/z): 492 (M+H)⁺

### EXAMPLE 126

2-[(4-{(tert-Butoxycarbonyl)[2-(2-pyridinyl)ethyl]amino}anilino)carbonyl]-4′-(2,2,2-trifluoroethoxy)-1,1′-biphenyl

The title compound was obtained from 4′-(2,2,2-trifluoroethoxy)-1,1′-biphenyl-2-carboxylic acid in the same manner as in Example 120.

¹H-NMR (DMSO-d₆): δ1.31 (9H, s), 2.84-2.92 (2H, m), 3.85-3.92 (2H, m), 4.75 (2H, q, J=8.9 Hz), 7.08 (2H, d, J=8.7 Hz), 7.11 (2H, d, J=8.5 Hz), 7.17-7.25 (2H, m), 7.39-7.57 (8H, m), 7.69 (1H, td, J=7.6 and 1.8 Hz), 8.45 (1H, d, J=4.7 Hz), 10.31 (1H, s)

APCI-MS (m/z): 592 (M+H)⁺

### EXAMPLE 127

N-(4-{[2-(2-Pyridinyl)ethyl]amino}phenyl)-4′-(2,2,2trifluoroethoxy)-1,1′-biphenyl-2-carboxamide

The title compound was obtained from 2-[(4-{(tert-butoxycarbonyl)[2-(2-pyridinyl)ethyl]amino}anilino)carbonyl]-4′-(2,2,2-trifluoroethoxy)-1,1′-biphenyl in the same manner as in Example 121.

¹H-NMR (DMSO-d₆): δ2.92-3.00 (2H, m), 3.29-3.39 (2H, m), 4.76 (2H, q, J=8.9 Hz), 5.52 (1H, t, J=5.8 Hz), 6.51 (2H, d, J=8.8 Hz), 7.07 (2H, d, J=8.8 Hz), 7.21-7.55 (10H, m), 7.70 (1H, td, J=7.6 and 1.8 Hz), 8.50 (1H, d, J=4.8 Hz), 9.82 (1H, s)

APCI-MS (m/z) : 492 (M+H)⁺

### EXAMPLE 128

2-1 (4-{(tert-Butoxycarbonyl) [2-(2-pyridinyl)ethyl]amino}anilino)carbonyl]-4′-(2,2,3,3-tetrafluoropropoxy)-1,1′-biphenyl

The title compound was obtained from 4′-(2,2,3,3-tetrafluoropropoxy)-1,1′-biphenyl-2-carboxylic acid in the same manner as in Example 120.

¹H-NMR (DMSO-d₆): δ1.31 (9H, s), 2.84-2.92 (2H, m), 3.84-3.92 (2H, m), 4.58 (2H, t, J=13.3 Hz), 6.66 (1H, tt, J=51.9 and 5.6 Hz), 7.05-7.13 (4H, m), 7.18-7.25 (2H, m), 7.39-7.57 (8H, m), 7.65-7.73 (1H, m), 8.46 (1H, d, J=4.3 Hz), 10.31 (1H, s)

APCI-MS (m/z): 624 (M+H)⁺

### EXAMPLE 129

N-(4-{[2-(2-Pyridinyl)ethyl]amino}phenyl)-4′-(2,2,3,3-tetrafluoropropoxy)-1,1′-biphenyl-2-carboxamide

The title compound was obtained from tert-butyl 2-[(4-{(tert-butoxycarbonyl)[2-(2-pyridinyl)ethyl]amino}anilino)-carbonyl]-4′-(2,2,3,3-tetrafluoropropoxy)-1,1′-biphenyl in the same manner as in Example 121.

¹H-NMR (DMSO-d₆): δ2.92-3.00 (2H, m); 3.29-3.40 (2H, m), 4.59 (2H, t, J=13.4 Hz), 5.52 (1H, t, J=5.7 Hz), 6.51 (2H, d, J=8.8 Hz), 6.68 (1H, tt, J=51.9 and 5.6 Hz), 7.06 (2H, d, J=8.7 Hz), 7.19-7.32 (4H, m), 7.39-7.55 (6H, m), 7.70 (1H, td, J=7.8 and 1.8 Hz), 8.50 (1H, d, J=4.8 Hz), 9.81 (1H, s)

APCI-MS (m/z): 524 (M+H)⁺

### EXAMPLE 130

2′-[(4-{(tert-Butoxycarbonyl)[2-(2-pyridinyl)ethyl]amino}anilino)carbonyl]-1,1′-biphenyl-4-yl 4-methylbenzenesulfonate

The title compound was obtained from 4′-{[(4-methylphenyl)sulfonyl]oxy}-1,1′-biphenyl-2-carboxylic acid in the same manner as in Example 120.

¹H-NMR (DMSO-d₆): δ1.30 (9H, s), 2.37 (3H,s), 2.84-2.91 (2H, m), 3.85-3.92 (2H, m), 7.01 (2H, d, J=8.6 Hz), 7.12-7.22 (4H, m), 7.33-7.71 (13H, m), 8.43-8.46 (1H, m), 10.21 (1H, s)

APCI-MS (m/z): 663 (M⁺)

### EXAMPLE 131

2′-[(4-{[2-(2-Pyridinyl)ethyl]amino}anilino)carbonyl]-1,1′-biphenyl-4-yl 4-methylbenzenesulfonate

The title compound was obtained from 2′-[(4-{(tert-butoxycarbonyl)[2-(2-pyridinyl)ethyl]amino}anilino)carbonyl]-1,1′-biphenyl-4-yl 4-methylbenzenesulfonate in the same manner as in Example 121.

¹H-NMR (DMSO-d₆): δ2.37 (3H, s), 2.93-3.00 (2H, m), 3.30-3.40 (2H, m), 5.53-5.59 (1H, m), 7.00 (2H, d, J=8.7 Hz), 7.17-7.53 (12H, m), 7.62-7.69 (3H, m), 8.49-8.51 (1H, m), 9.72 (1H, s)

APCI-MS (m/z): 564 (M+H)⁺

### EXAMPLE 132

To a mixture of 4′-(benzyloxy)-1,1′-biphenyl-2-carboxylic acid (1.24 g) and N,N-dimethylformamide (0.0158 ml) in toluene (13 ml) was added thionyl chloride (0.939 ml) dropwise under a nitrogen atmosphere and the solution was stirred at 100° C. for 2 hours. The resultant mixture was cooled down to ambient temperature, and then the solvent was evaporated in vacuo. The excess thionyl chloride was removed as the toluene azeotrope twice. The residue was dissolved in tetrahydrofuran (25 ml) and the solution was cooled to 5° C. with ice bath. tert-Butyl 4-aminophenyl[2-(2-pyridinyl)ethyl]carbamate (1.28 g) was added portionwise to the above solution at 5° C. under a nitrogen atmosphere, and then diisopropylethylamine (0.85 ml) was added dropwise. The solution was allowed to warm to ambient temperature with stirring for 15 minutes, and the solvent was removed under reduced pressure. The resultant residue was dissolved in ethyl acetate, washed with water and brine, dried over magnesium sulfate and evaporated in vacuo. The residue was chromatographed on silica gel eluting with hexane-ethyl acetate (from hexane-ethyl acetate 3:1 to 3:2). The eluate was concentrated in vacuo to give 4-benzyloxy-2′-[(4-{(tert-butoxycarbonyl)[2-(2-pyridinyl)ethyl]amino}anilino)carbonyl]-1,1′-biphenyl (2.31 g) as a white amorphous.

¹H-NMR (DMSO-d₆): δ1.32 (9H, s), 2.85-2.92 (2H, m), 3.85-3.92 (2H, m), 5.09 (2H, s), 7.03 (2H, d, J=8.7 Hz), 7.11 (2H, d, J=8.7 Hz), 7.17-7.25 (2H, m), 7.31-7.53 (13H, m), 7.68 (1H, td, J=7.6 and 1.8 Hz), 8.46 (1H, d, J=4.7 Hz), 10.28 (1H, s)

APCI-MS (m/z): 600 (M+H)⁺

### EXAMPLE 133

4′-Benzyloxy-N-(4-{[2-(2-pyridinyl)ethyl]amino}phenyl)-1,1′-biphenyl-2-carboxamide

The title compound was obtained from 4-benzyloxy-2′-[(4-(tert-butoxycarbonyl){(2-(2-pyridinyl)ethyl]amino}anilino)-carbonyl]-1,1′-biphenyl in the same manner as in Example 121.

¹H-NMR (DMSO-d₆): δ2.93-3.00 (2H, m), 3.29-3.40 (2H, m), 5.10 (1H, s), 5.53 (1H, t, J=5.7 Hz), 6.51 (2H, d, J=8.8 Hz), 7.02 (2H, d, J=8.6 Hz), 7.19-7.54 (15H, m), 7.70 (1H, td 7.6 and 1.7 Hz), 8.51 (1H, d, J=4.9 Hz), 9.78 (1H, s)

APCI-MS (m/z): 500 (M+H)⁺

### EXAMPLE 134

To a solution of 4-benzyloxy-2′-[(4-[(tert-butoxycarbonyl)-[2-(2-pyridinyl)ethyl]amino}anilino)carbonyl]-1,1′-biphenyl (11.5 g) in methanol (115 ml) was added 10% palladium on carbon (50% wet, 2.3 g). The mixture was reduced under a medium pressured hydrogen gas with vigorous stirring for 17 hours. The catalyst was removed by filtration, washed with methanol, and then the filtrate was evaporated in vacuo. The residue was chromatographed on silica gel eluting with hexane-ethyl acetate (from hexane-ethyl acetate 1:1 to ethyl acetate only). The eluate was concentrated in vacuo to give 2-[(4-{(tert-butoxycarbonyl)[2-(2-pyridinyl)ethyl]amino}anilino)carbonyl]-4′-hydroxy-1,1′-biphenyl (8.53 g) as a white solid.

¹H-NMR (DMSO-d₆): δ1.32 (9H, s), 2.85-2.92 (2H, m), 3.85-3.92 (2H, m), 6.76 (2H, d, J=8.6 Hz), 7.10 (2H, d, J=8.7 Hz), 7.17-7.29 (4H, m), 7.40-7.56 (6H, m), 7.68 (1H, td, J=7.6 and 1.8 Hz), 8.46 (1H, d, J=4.0 Hz), 9.47 (1H, brs), 10.28 (1H, s)

APCI-MS (m/z): 510 (M+H)⁺

### EXAMPLE 135

To a solution of 2-[(4-{(tert-butoxycarbonyl)[2-(2-pyridinyl)ethyl]amino}anilino)carbonyl]-4′-hydroxy-1,1′-biphenyl (300 mg) dissolved in N,N-dimethylformamide (6.0 ml) was added potassium carbonate (309 mg) under a nitrogen atmosphere and the solution was stirred at 65° C. for 30 minutes. After 2-(dimethylamino)ethyl chloride hydrochloride (255 mg) was added, the mixture was stirred at 65° C. for 5 hours. The reaction mixture was cooled down to ambient temperature and diluted with ethyl acetate. The solution was washed with water three times and brine, dried over magnesium sulfate and evaporated in vacuo. The residue was chromatographed on silica gel eluting with dichloromethane-methanol (from dichloromethane only to dichloromethane-methanol 100:8). The eluate was concentrated in vacuo to give 2-[(4-{(tert-butoxycarbonyl)[2-(2-pyridinyl)ethyl]amino}anilino)-carbonyl]-4′-[2-(dimethylamino)ethoxy]-1,1′-biphenyl (139 mg) as a white amorphous.

¹H-NMR (DMSO-d₆): δ1.31 (9H, s), 2.18 (6H, s), 2.59 (2H, t, J=5.8 Hz), 2.85-2.92 (2H, m), 3.85-3.92 (2H, m), 4.03 (2H, t, J=5.8 Hz), 6.95 (2H, d, J=8.6 Hz), 7.10 (2H, d, J=8.7 Hz), 7.17-7.25 (2H, m), 7.34-7.52 (8H, m), 7.63-7.74 (1H, m), 8.45 (1H, d, J=4.8 Hz), 10.25 (1H, s)

APCI-MS (m/z): 581 (M+H)⁺

### EXAMPLE 136

4′-[2-(Dimethylamino)ethoxy]-N-(4-{[2-(2-pyridinyl)ethyl]amino}phenyl)-1,1′-biphenyl-2-carboxamide

The title compound was obtained from 2-[(4-{(tert-butoxycarbonyl)[2-(2-pyridinyl)ethyl]amino}anilino)carbonyl]-4′-[2-(dimethylamino)ethoxy]-1,1′-biphenyl in the same manner as in Example 121.

¹H-NMR (DMSO-d₆): δ2.20 (6H, s), 2.61 (2H, t, J=5.8 Hz), 2.92-2.99 (2H, m), 3.32-3.40 (2H, m), 4.04 (2H, m, J=5.8 Hz), 5.48-5.54 (1H, m), 6.50 (2H, d, J=8.9 Hz), 6.94 (2H, d, J=8.8 Hz), 7.19-7.49 (10H, m), 7.70 (1H, td, J=7.6 and 1.9 Hz), 8.49-8.52 (1H, m), 9.75 (1H, s)

APCI-MS (m/z): 481 (M+H)⁺

### EXAMPLE 137

2-[(4-{(tert-Butoxycarbonyl)[2-(2-pyridinyl)ethyl]amino}anilino)carbonyl]-4′-(2-methoxyethoxy)-1,1′-biphenyl

The title compound was obtained from 2-[(4-{(tert-butoxycarbonyl)[2-(2-pyridinyl)ethyl]amino}anilino)carbonyl]-4′-hydroxy-1,1′-biphenyl in the same manner as in Example 135.

¹H-NMR (DMSO-d₆): δ1.41 (9H, s), 2.84-2.92 (2H, m), 3.28 (3H, s), 3.61-3.65 (2H, m), 3.84-3.92 (2H, m), 4.06-4.10 (2H, m), 6.95 (2H, d, J=8.7 Hz), 7.10 (2H, d, J=8.8 Hz), 7.17-7.25 (2H, m), 7.35-7.58 (8H, m), 7.68 (1H, td, J=7.6 and 1.8 Hz), 8.46 (1H, d, J=4.0 Hz), 10.26 (1H, s)

APCI-MS (m/z): 568 (M+H)⁺

### EXAMPLE 138

4′-(2-methoxyethoxy)-N-(4-{[2-(2-pyridinyl)ethyl]amino}phenyl)-1,1′-biphenyl-2-carboxamide

The title compound was obtained from 2-[(4-{(tert-butoxycarbonyl)[2-(2-pyridinyl)ethyl]amino}anilino)carbonyl]-4′-(2-methoxyethoxy)-1,1′-biphenyl in the same manner as in Example 121.

¹H-NMR (DMSO-d₆): δ2.92-3.00 (2H, m), 3.19-3.42 (2H, m), 3.29 (3H, s), 3.62-3.66 (2H, m), 4.06-4.11 (2H, m), 5.52 (2H, t, J=5.7 Hz), 6.51 (2H, d, J=8.8 Hz), 6.94 (2H, d, J=8.7 Hz), 7.20-7.50 (10H, m), 7.70 (1H, td, J=7.6 and 1.9 Hz), 8.51 (1H, d, J=4.8 Hz), 9.77 (1H, s)

APCI-MS (m/z): 468 (M+H)⁺

### EXAMPLE 139

To a solution of 2-[(4-{(tert-butoxycarbonyl)[2-(2pyridinyl)ethyl]amino}anilino)carbonyl]-4′-hydroxy-1,1′-biphenyl (4.0 g) dissolved in N,N-dimethylformamide (80 ml) was added potassium carbonate (1.30 g) under a nitrogen atmosphere and the solution was stirred at 65° C. for 1 hour. After the solution was cooled down to ambient temperature, ethyl bromoacetate (2.61 ml) was added dropwise and the mixture was stirred for 4 hours. The resultant reaction mixture was poured into saturated aqueous ammonium chloride solution and extracted with ethyl acetate. The extract was washed with water three times and brine, dried over magnesium sulfate and evaporated in vacuo. The residue was chromatographed on silica gel eluting with hexane-ethyl acetate (from hexane-ethyl acetate 3:1 to 1:2). The eluate was concentrated in vacuo to give ethyl ({2′-[(4-{(tert-butoxycarbonyl)[2-(2-pyridinyl)ethyl]amino}anilino)carbonyl]-1,1′-biphenyl-4-yl)oxy)acetate (1.91 g) as a white amorphous. The title compound 1.18 (3H, t, J=7.1 Hz), 1.32 (9H, s), 2.85-2.92 (2H, m), 3.85-3.92 (2H, m), 4.14 (1H, q, J=7.1 Hz), 4.77 (2H, s), 6.94 (2H, d, J=8.7 Hz), 7.10 (2H, d, J=8.8 Hz), 7.18-7.25 (2H, m), 7.35-7.56 (8H, m), 7.69 (1H, td, J=7.7 and 1.8 Hz), 8.46 (1H, d, J=4.8 Hz), 10.28 (1H, s)

APCI-MS (m/z): 596 (M+H)⁺

### EXAMPLE 140

Ethyl ({2′-[(4-{[2-(2-pyridinyl)ethyl]amino}anilino)-carbonyl]-1,1′-biphenyl-4-yl}oxy)acetate

The title compound was obtained from ethyl ({2′-[(4-{(tert-butoxycarbonyl)[2-(2-pyridinyl)ethyl]amino}anilino)carbonyl]-1,1′-biphenyl-4-yl}oxy)acetate in the same manner as in Example 121.

¹H-NMR (DMSO-d₆): δ1.19 (3H, t, J=7.1 Hz), 2.92-3.00 (2H, m), 3.32-3.40 (2H, m), 4.15 (2H, q, J=7.1 Hz), 4.77 (2H, s), 5.49-5.54 (1H, m), 6.51 (2H, d, J=8.9 Hz), 6.93 (2H, d, J=8.8 Hz), 7.19-7.25 (3H, m), 7.28-7.50 (7H, m), 7.70 (1H, td, J=7.7 and 1.9 Hz), 8.49-8.52 (1H, m), 9.77 (1H, s)

APCI-MS (m/z): 496 (M+H)⁺

### EXAMPLE 141

To a solution of 2-[(4-{(tert-butoxycarbonyl)[2-(2pyridinyl)ethyl]amino}anilino)carbonyl]-4′-hydroxy-1,1′-biphenyl (400 mg) and triphenylphosphine (309 mg) dissolved in tetrahydrofuran (4.0 ml) was added diethyl azodicarboxylate (0.186 ml) over a period of 1 minutes. After stirring for 20 minutes, a solution of 3-dimethylamino-1-propanol (0.242 ml) in tetrahydrofuran (5.0 ml) was added. The mixture was stirred overnight and then the solvent was evaporated in vacuo. A residue was dissolved in ethyl acetate and washed with water and brine, dried over magnesium sulfate and evaporated in vacuo. The resultant residue was chromatographed on silica gel eluting with dichloromethane-methanol (from dichloromethane-methanol 100:1 to 10:1). The eluate was concentrated in-vacuo to give 2-[(4-{(tert-butoxycarbonyl)[2-(2-pyridinyl)ethyl]amino}anilino)carbonyl]-4′-[3-(dimethylamino)propoxy]-1,1′-biphenyl (210 mg) as a white amorphous.

¹H-NMR (DMSO-d₆): δ1.32 (9H, s), 1.75-1.89 (2H, m), 2.14 (6H, s), 2.32-2.39 (2H, m), 2.85-2.93 (2H, m), 3.86-4.10 (4H, m), 6.93 (2H, d, J=8.7 Hz), 7.10 (2H, d, J=8.7 Hz), 7.19-7.25 (2H, m), 7.34-7.60 (8H, m), 7.67-7.76 (1H, m), 8.48-9.51 (1H, m), 10.25 (1H, s)

APCI-MS (m/z): 595 (M+H)⁺

### EXAMPLE 142

4′-[3-(Dimethylamino)propoxy)-N-(4-{[2-(2pyridinyl)ethyl]amino}phenyl)-1,1′-biphenyl-2-carboxamide

The title compound was obtained from 2-[(4-{(tert-butoxycarbonyl)[2-(2-pyridinyl)ethyl]amino}anilino)carbonyl]-4′-[3-(dimethylamino)propoxyl-1,1′-biphenyl in the same manner as in Example 121.

¹H-NMR (DMSO-d₆): δ1.75-1.89 (2H, m), 2.13 (6H, s), 2.34 (2H, t, J=7.1 Hz), 2.92-3.00 (2H, m), 3.29-3.39 (2H, m), 3.95-4.01 (2H, m) 5.52 (1H, t, J=5.7 Hz), 6.51 (2H, d, J=8.89 Hz), 6.92 (2H, d, J=8.6 Hz), 7.19-7.54 (10H, m), 7.70 (1H, td, J=7.7 and 1.8 Hz), 8.51 (1H, d, J=4.8 Hz), 9.76 (1H, s)

APCI-MS (m/z): 495 (M+H)⁺

### EXAMPLE 143

Ethyl ({2′-[(4-{(tert-butoxycarbonyl)[2-(2-pyridinyl)ethyl]amino}anilino)carbonyl]-1,1′-biphenyl-4-yl}oxy)acetate (1.90 g) was dissolved in tetrahydrofuran-methanol 111) (38 ml) and the mixture was cooled to 5° C. with ice bath. 1N Aqueous lithium hydroxide solution (9.57 ml) was added dropwise at 5° C. After stirring for 2 hours, the pH of the solution was adjusted to 4.0 with 5% aqueous potassium hydrogensulfate solution-and the solvent was removed under reduced pressure. The resultant aqueous suspension was extracted with ethyl acetate-tetrahydrofuran (1:1), and then the extract was washed with brine, dried over magnesium sulfate and evaporated in vacuo. The crystallization of the residue was induced by scratching the flask and the resulting crystals were washed with ether and dried in vacuo to give ({2′-[(4-{(tert-butoxycarbonyl)[2-(2-pyridinyl)ethyl]amino}anilino)carbonyl]-1,1′-biphenyl-4-yl}oxy)acetic acid (1.56g) as white crystals.

¹H-NMR (DMSO-d₆): δ1.32 (9H, s), 2.85-2.92 (2H, m), 3.85-3.92 (2H, m), 4.64 (2H, s), 6.94 (2H, d, J=8.7 Hz), 7.11 (2H, d, J=8.7 Hz), 7.18-7.25 (2H, m), 7.35-7.56 (8H, m), 7.69 (1H, td, J=7.6 and 1.8 Hz), 8.46 (1H, d, J=4.1 Hz), 10.30 (1H, s)

APCI-MS (m/z): 566 (M+H)⁺

### EXAMPLE 144

To a mixture of ({2′-[(4-{(tert-butoxycarbonyl)[2-(2-pyridinyl)ethyl]amino}anilino)carbonyl]-1,1′-biphenyl-4-yl}oxy)acetic acid (200 mg) and HOBT.H₂O (70.1 mg) in dichloromethane (4.0 ml) was added WSC.HCl (101 mg) portionwise under a nitrogen atmosphere. After stirring for 20 minutes, 28% aqueous ammonia solution was added dropwise and the mixture was stirred for 2 hours. The reaction mixture was diluted with dichloromethane, washed with water three times and then brine, dried over magnesium sulfate and evaporated in vacuo. The residue was chromatographed on silica gel eluting with ethyl acetate-methanol (10:1). The eluate was concentrated in vacuo to give 4-(2-amino-2-oxoethoxy)-2′-[(4-{(tert-butoxycarbonyl)[2-(2-pyridinyl)ethyl]amino}anilino)carbonyl]-1,1′-biphenyl (187 mg) as white crystals.

### EXAMPLE 145

4′-(2-Amino-2-oxoethoxy)-N-(4-{[2-(2pyridinyl)-ethyl]amino}phenyl)-1,1′-biphenyl-2-carboxamide

The title compound was obtained from 4-(2-amino-2-oxoethoxy)-2′-[(4-{(tert-butoxycarbonyl)[2-(2-pyridinyl)ethyl]amino}anilino)carbonyl]-1,1′-biphenyl in the same manner as in Example 121.

¹H-NMR (DMSO-d₆): δ2.93-3.00 (2H, m), 3.29-3.39 (2H, m), 4.42 (2H, s), 5.49-5.54 (1H, m), 6.51 (2H, d, J=8.8 Hz), 6.96 (2H, d, J=8.7 Hz), 7.19-7.52 (12H, m), 7.70 (1H, td, J=7.6 and 1.8 Hz), 8.51 (1H, d, J=4.0 Hz), 9.80 (1H, s)

APCI-MS (m/z): 467 (M+H)⁺

### EXAMPLE 146

2-[(4-{(tert-Butoxycarbonyl)[2-(2pyridinyl)ethyl]amino}anilino)carbonyl]-4′-[2-(methylamino)-2-oxoethoxy]-1,1′-biphenyl

The title compound was obtained from ({2′-[(4-{(tert-butoxycarbonyl)[2-(2-pyridinyl)ethyl]amino}anilino)carbonyl]-1,1′-biphenyl-4-yl}oxy)acetic acid in the same manner as in Example 144.

### EXAMPLE 147

4′-[2-(Methylamino)-2-oxoethoxy]-N-(4-{[2-(2-pyridinyl)ethyl]amino}phenyl)-1,1′-biphenyl-2-carboxamide

The title compound was obtained from 2-[(4-{(tert-butoxycarbonyl)[2-(2-pyridinyl)ethyl]amino}anilino)carbonyl]-4′-[2-(methylamino)-2-oxoethoxy]-1,1′-biphenyl in the same manner as in Example 121.

¹H-NMR (DMSO-d₆): δ2.65 (3H, d, J=4.6 Hz), 2.92-3.00 (2H, m), 3.29-3.39 (2H, m), 4.45 (2H, s), 5.52 (1H, t, J=5.7 Hz), 6.51 (2H, d, J=8.8 Hz), 6.97 (2H, d, J=8.7 Hz), 7.20-7.54 (10H, m), 7.70 (1H, td, J=7.6 and 1.8 Hz), 8.02 (1H, d, J=4.6 Hz), 8.51 (1H, d, J=4.0 Hz), 9.79 (1H, s)

APCI-MS (m/z): 481 (M+H)⁺

### EXAMPLE 148

4-(2-Anilino-2-oxoethoxy)-2′-[(4-{(tert-butoxycarbonyl)[2-(2-pyridinyl)ethyl]amino}anilino)carbonyl]-1,1′-biphenyl

The title-compound was obtained from ({2′-[(4-{(tert-butoxycarbonyl)[2-(2-pyridinyl)ethyl]amino}anilino)carbonyl]-1,1′-biphenyl-4-yl}oxy)acetic acid in the same manner as in Example 144.

### EXAMPLE 149

4′-(2-Anilino-2-oxoethoxy)-N-(4-{[2-(2-pyridinyl)ethyl]amino}phenyl)-1,1′-biphenyl-2-carboxamide

The title compound was obtained from 4-(?-anilino-2-oxoethoxy)-2′-[(4-{(tert-butoxycarbonyl)[2-(2-pyridinyl)ethyl]amino}anilino)carbonyl]-1,1′-biphenyl in the same manner as in Example 121.

¹H-NMR (DMSO-d₆): δ2.93-3.00 (2H, m), 3.33-3.44 (2H, m), 4.70 (2H, s), 5.51 (1H, t, J=5.6 Hz), 6.50 (2H, d, J=8.8 Hz), 7.01 (2H, d, J=8.7 Hz), 7.08 (1H, d, J=7.3 Hz), 7.20-7.50 (12H, m), 7.61-7.66 (1H, m), 7.70 (1H, td, J=7.6 and 1.7 Hz), 8.51 (1H, d, J=4.2 Hz), 9.79 (1H, s), 10.07 (1H, s)

APCI-MS (m/z): 543 (M+H)⁺

### EXAMPLE 150

A solution of ({2′-[(4-{(tert-butoxycarbonyl)[2-(2pyridinyl)ethyl]amino}anilino)carbonyl]-1,1′-biphenyl-4-yl}oxy)acetic acid (200 mg), methanesulfonamide (40.2 mg), WSC.HCl (101 mg) and 4-(dimethylamino)pyridine (64.5 mg) dissolved in dichloromethane (4.0 ml) was stirred for 3 days. The pH of the reaction mixture was adjusted to 3.0 with 5% aqueous potassium hydrogensulfate solution and the mixture was extracted with ethyl acetate. The extract was washed with water twice and brine, dried over magnesium sulfate and evaporated in vacuo. The crystallization of the residue was induced by scratching the flask and the resulting crystals were washed with ether and dried in vacuo to give 2-[(4-{(tert-butoxycarbonyl)[2-(2pyridinyl)ethyl]amino}anilino)carbonyl]-4′-{2-[(methylsulfonyl)amino]-2-oxoethoxy}-1,1′-biphenyl (190 mg) as beige crystals.

¹H-NMR (DMSO-d₆): δ1.32 (9H, s), 2.85-2.92 (2H, m), 3.24 (3H, s), 3.85-3.92 (2H, m), 4.69 (2H, s), 6.94 (2H, d, J=8.8 Hz), 7.11 (2H, d, J=8.7 Hz), 7.19-7.26 (2H, m), 7.35-7.56 (9H, m), 7.70 (1H, td, J=7.6 and 1.8 Hz), 8.46 (1H, d, J=4.0 Hz), 10.31 (1H, s)

(−)APCI-MS (m/z): 643 (M−H)⁻

### EXAMPLE 151

2-[(4-{(tert-Butoxycarbonyl)[2-(2-pyridinyl)ethyl]amino}anilino)carbonyl]-4′-{2-[(methylsulfonyl)amino]-2-oxoethoxy}-1,1′-biphenyl (190 mg) was dissolved in trifluoroacetic acid (0.95 ml). After the solution was stirred for 5 hours, trifluoroacetic acid was removed under reduced pressure. The residue was dissolved in ethyl acetate-tetrahydrofuran (1:1) and the pH of the solution was adjusted to 4.0 with saturated aqueous sodium hydrogencarbonate solution. The separated organic layer was washed with brine twice, dried over magnesium sulfate and evaporated in vacuo. The resultant residue was chromatographed on silica gel eluting with dichloromethane-methanol (from dichloromethane only to 10:1). The eluate was concentrated in vacuo to give 4′-{2-[(methylsulfonyl)amino]-2-oxoethoxy}-N-(4-{[2-(2-pyridinyl)ethyl]amino}phenyl)-1,1′-biphenyl-2-carboxamide (131 mg) as an yellow solid.

¹H-NMR (DMSO-d₆): δ2.91 (s, 3H), 2.94-3.00 (m, 2H), 3.17 (1H, brs), 3.30-3.38 (2H, m), 4.41 (2H, s), 6.51 (2H, d, J=8.8 Hz), 6.87 (2H, d, J=8.7 Hz), 7.20-7.53 (10H, m), 7.70 (1H, td, J=7.6 and 1.8 Hz), 8.51 (1H, d, J=4.0 Hz), 9.78 (1H, s)

APCI-MS (m/z): 545 (M+H)⁺

### EXAMPLE 152

2-[(4-{(tert-Butoxycarbonyl)[2-(2pyridinyl)ethyl]amino}anilino)carbonyl]-4′-{2-oxo-2-[(phenylsulfonyl)amino]ethoxy}-1,1′-biphenyl

The title compound was obtained from ({2′-[(4-{(tert-butoxycarbonyl)[2-(2-pyridinyl)ethyl]amino}anilino)carbonyl]-1,1′-biphenyl-4-yl}oxy)acetic acid in the same manner as in Example 150.

¹H-NMR (DMSO-d₆): δ1.31 (9H, s), 2.85-2.92 (2H, m), 3.85-3.92 (2H, m), 4.58 (2H, s), 6.80 (2H, d, J=8.7 Hz), 7.11 (2H, d, J=8.7 Hz), 7.18-7.25 (2H, m), 7.32 (2H, d, J=8.7 Hz), 7.40-7.65 (10H, m), 7.69 (1H, td, J=7.7 and 1.8 Hz), 7.87-7.91 (1H, m), 8.46 (1H, d, J=4.8 Hz), 10.30 (1H, s)

APCI-MS (m/z): 706 (M⁺)

### EXAMPLE 153

4′-{2-Oxo-2-[(phenylsulfonyl)amino]ethoxy}-N-(4-{[2-(2-pyridinyl)ethyl]amino}phenyl)-1,1′-biphenyl-2-carboxamide

The title compound was obtained from 2-[(4-{(tert-butoxycarbonyl)[2-(2-pyridinyl)ethyl]amino}anilino)carbonyl]-4′-(2-oxo-2-[(phenylsulfonyl)amino]ethoxy}-1,1′-biphenyl in the same manner as in Example 151.

¹H-NMR (DMSO-d₆): δ2.93-3.00 (2H, m), 3.17 (3H, s), 3.32-3.40 (2H, m), 4.52 (2H, s), 6.52 (2H, d, J=8.9 Hz), 6.78 (2H, d, J=8.8 Hz), 7.20-7.61 (14H, m), 7.71 (1H, td, J=7.6 and 1.8 Hz), 7.87 (2H, dd, J=1.8 and 8.2 Hz), 8.51 (1H, d, J=4.1 Hz), 9.79 (1H, s)

(−)APCI-MS (m/z): 605 (M−1)⁻

### EXAMPLE 154

To a solution of 2-[(4-{(tert-butoxycarbonyl)[2-(2-pyridinyl)ethyl]amino}anilino)carbonyl]-4′-methoxy-1,1′-biphenyl (265 mg) dissolved in acetic acid (4.0 ml) was added dropwise 47% aqueous hydrobromic acid (0.589 ml). The mixture was refluxed overnight. After the reaction mixture was cooled down to ambient temperature, the solvent was removed under reduced pressure. The residue was diluted with ethyl acetate and washed with saturated aqueous sodium hydrogencarbonate solution. The separated organic layer was washed with water and brine, dried over magnesium sulfate and evaporated in vacuo. The resultant residue was chromatographed on silica gel eluting with hexane-ethyl acetate (from hexane-ethyl acetate 2:1 to 1:2). The eluate was concentrated in vacuo and the crystallization of the residue was induced by scratching the flask. The resulting crystals were washed with diisopropyl ether and dried in vacuo to give 4′-hydroxy-N-(4-{[2-(2-pyridinyl)ethyl]amino}phenyl)-1,1′-biphenyl-2-carboxamide (54 mg).

¹H-NMR (DMSO-d₆): δ2.93-3.00 (2H, m), 3.30-3.39 (2H, m), 5.49-5.55 (10H, m), 7.70 (1H, td, J=7.6 and 1.8 Hz), 8.51 (1H, d, J=4.5 Hz), 9.45 (1H, s), 9.70 (1H, s)

APCI-MS (m/z): 410 (M+H)⁺

### EXAMPLE 155

To a solution of ethyl({2′-[(4-{[2-(2-pyridinyl)ethyl]amino}anilino)carbonyl]-1,1′-biphenyl-4-yl}oxy)acetate (120 mg) dissolved in tetrahydrofuran (4.8 ml) was added lithium borohydride (10.5 mg) portionwise, and then methanol (0.024 ml) dropwise. After stirring at ambient temperature for 1.5 hours, 1N aqueous HCl (3.0 ml) was added dropwise and the mixture was stirred for 30 minutes. The pH of the mixture was adjusted to 7.0 with saturated aqueous sodium hydrogencarbonate solution and the solvent was evaporated in vacuo. The residue was dissolved in ethyl acetate and the separated organic layer was washed with water and brine, dried over magnesium sulfate and evaporated in vacuo. The resultant residue was chromatographed on silica gel eluting with ethyl acetate-ethanol (from ethyl acetate only to ethyl acetate-ethanol 25:1) The eluate was concentrated in vacuo and the crystallization of the residue was induced by scratching the flask. The resulting crystals were washed with diisopropyl ether and dried in vacuo to give 4′-(2-hydroxyethoxy)-N-(4-{[2-(2-pyridinyl)ethyl]amino}phenyl)-1,1′-biphenyl-2-carboxamide (28 mg).

¹H-NMR (DMSO-d₆): δ2.92-3.00 (2H, m), 3.28-3.38 (2H, m); 3.66-3.73 (2H, m), 3.95-4.00 (2H, m), 4.85 (2H, t, J=5.5 Hz), 5.51 (2H, t, J=5.7 Hz), 6.51 (2H, d, J=8.8 Hz), 6.94 (2H, d, J=8.7 Hz), 7.19-7.54 (10H, m), 7.70 (1H, td, J=7.6 and 1.8 Hz), 8.51 (1H, d, J=4.7 Hz), 9.77 (1H, s)

APCI-MS (m/z): 454 (M+H)⁺

### EXAMPLE 156

4-Amino-2′-[(4-{(tert-butoxycarbonyl)[2-(2-pyridinyl)ethyl]amino}anilino)carbonyl]-1,1′-biphenyl (400 mg) was dissolved in dichloromethane (8.0 ml) and the mixture was cooled to 5° C. with ice bath under a nitrogen atmosphere. Triethylamine (1.10 ml) was added portionwise to the above solution at 5° C., and then methyl chloroformate (0.607 ml) was added dropwise. After the mixture was stirred at 5° C. for 1 hour, water and dichloromethane were poured into the reaction mixture. The pH of the mixture was adjusted to 5.0 with 5% aqueous potassium hydrogensulfate solution. The separated organic layer was washed with water and brine, dried over magnesium sulfate and evaporated in vacuo. The residue was chromatographed on silica gel eluting with hexane-ethyl acetate (from hexane-ethyl acetate 3:1 to 1:2). The eluate was concentrated in vacuo to give 2-[(4-{(tert-butoxycarbonyl)[2-(2-pyridinyl)ethyl]amino}anilino)-carbonyl]-4′-[(methoxycarbonyl)amino]-1,1′-biphenyl (317 mg) as a white solid.

¹H-NMR (DMSO-d₆): δ1.31 (1H, s), 2.85-2.92 (2H, m), 3.65 (3H, s), 3.85-3.92 (2H, m), 7.10 (2H, d, J=8.8 Hz), 7.17-7.25 (2H, m), 7.34-7.56 (2H, m), 7.68 (1H, td, J=7.7 and 1.8 Hz), 8.45 (1H, d, J=4.0 Hz), 9.68 (1H, s), 10.27 (1H, s)

APCI-MS (m/z): 567 (M+H)⁺

### EXAMPLE 157

Methyl 2′-[(4-{[2-(2-pyridinyl)ethyl]amino}anilino)-carbonyl]-1,1′-biphenyl-4-ylcarbamate

The title compound was obtained from 2-[(4-{(tert-butoxycarbonyl)[2-(2-pyridinyl)ethyl]amino}anilino)carbonyl]-4′-[(methoxycarbonyl)amino]-1,1′-biphenyl in the same manner as in Example 151.

¹H-NMR (DMSO-d₆): δ2.93-3.00 (2H, m), 3.29-3.39 (2H, m), 3.66 (3H, s), 5.52 (1H, t, J=5.8 Hz), 6.51 (2H, d, J=8.8 Hz), 7.19-7.54 (12H, m), 7.70 (1H, td, J=7.7 and 1.8 Hz), 8.51 (1H, d, J=4.7 Hz), 9.69 (1H, s), 9.78 (1H, s)

APCI-MS (m/z): 467 (M+H)⁺

### EXAMPLE 158

4-Amino-2′-[(4-{(tert-butoxycarbonyl)[2-(2-pyridinyl)ethyl]amino}anilino)carbonyl]-1,1′-biphenyl (400 mg) was dissolved in dichloromethane (8.0 ml) and the mixture was cooled to 5° C. with, ice bath under a nitrogen atmosphere. Triethylamine (1.14 ml) was added portionwise to the above solution at 5° C., and then methanesulfonyl chloride (0.675 ml) was added dropwise. The mixture was allowed to warm to ambient temperature and stirred for 1 hour. After water and ethyl acetate were poured into the reaction mixture, the pH of the mixture was adjusted to 4.0 with 5% aqueous potassium hydrogensulfate solution. The separated organic layer was washed with water and brine, dried over magnesium sulfate and evaporated in vacuo. The residue was chromatographed on silica gel eluting with hexane-ethyl acetate (from hexane-ethyl acetate 3:1 to 1:2). The eluate was concentrated in vacuo to give 4-[bis(methylsulfonyl)amino]-2′-[(4-{(tert-butoxycarbonyl)[2-(2-pyridinyl)ethyl]amino}anilino)carbonyl]-1,1′-biphenyl (285 mg) as a light yellow amorphous.

¹H-NMR (DMSO-d₆): δ1.32 (1H, s), 2.85-2.92 (2H, m), 3.51 (6H, s), 3.85-3.92 (2H, m), 7.11 (2H, d, J=8.8 Hz), 7.18-7.25(2H, m), 7.42-7.66 (10H, m), 7.69 (1H, dt, J=7.6 and 1.8 Hz), 8.46 (1H, d, J=4.7 Hz), 10.29 (1H, s)

APCI-MS (m/z): 665 (M+H)⁺

### EXAMPLE 159

4′-[Bis(methylsulfonyl)amino]-N-(4-{[2-(2-pyridinyl)ethyl]amino}phenyl)-1,1′-biphenyl-2-carboxamide

The title compound was obtained from 4-[bis(methyl-sulfonyl)amino]-2′-[(4-{(tert-butoxycarbonyl)[2-(2-pyridinyl)ethyl]amino}anilino)carbonyl]-1,1′-biphenyl in the same manner as in Example 151.

¹H-NMR (DMSO-d₆): δ2.92-2.99 (2H, m), 3.29-3.39 (2H, m), 3.52 (6H, s), 5.53 (1H, t, J=5.6 Hz), 6.50 (2H, d, J=8.8 Hz), 7.11 (1H, d, J=8.7 Hz), 7.19-7.25 (1H, m), 7.70 (1H, td, J=7.7 and 1.8 Hz), 8.51 (1H, d, J=4.0 Hz), 9.75 (1H, s)

APCI-MS (m/z): 565 (M+H)⁺

### EXAMPLE 160

To a solution of 4′-[bis(methylsulfonyl)amino]-N-(4-{[2-(2pyridinyl)ethyl]amino}phenyl)-1,1′-biphenyl-2-carboxamide (190 mg) dissolved in tetrahydrofuran (0.95 ml) and methanol (0.95 ml) was added dropwise 1N aqueous sodium hydroxide solution (0.668 ml). The mixture was stirred for 2 hours and evaporated in vacuo. The residue was dissolved in ethyl acetate and the solution was washed with water and brine, dried over magnesium sulfate and evaporated in vacuo. The crystallization of the residue was induced by scratching the flask and the resulting crystals were washed with ethyl acetate and dried in vacuo to give 4′-[(methylsulfonyl)amino]-N-(4-{[2-(2-pyridinyl)ethyl]amino}-phenyl)-1,1′-biphenyl-2-carboxamide (104 mg) as a white solid.

¹H-NMR (DMSO-d₆): δ2.93-3.00 (2H, m), 2.96 (3H, s), 3.29-3.39 (2H, m), 5.52 (1H, t, J=5.7 Hz), 6.50 (2H, d, J=8.8 Hz), 7.18-7.32 (5H, m), 7.38-7.56 (7H, m), 7.70 (1H, td, J=7.6 and 1.9 Hz), 8.51 (1H, d, J=4.0 Hz), 9.75 (1H, s), 9.80 (1H, s)

APCI-MS (m/z): 487 (M+H)⁺

### EXAMPLE 161

4-[Bis(benzylsulfonyl)amino]-2′-[(4-{(tert-butoxycarbonyl)[2-(2-pyridinyl)ethyl]amino}anilino)carbonyl]-1,1′-biphenyl

The title compound was obtained from 4-amino-2′-[(4-{(tert-butoxycarbonyl)[2-(2-pyridinyl)ethyl]amino}anilino)carbonyl]-1,1′-biphenyl in the same manner as in Example 158.

¹H-NMR (DMSO-d₆): δ1.31 (1H, s), 2.84-2.92 (2H, m), 3.85-3.93 (2H, m), 5.01 (4H, s), 6.65 (2H, d, J=8.4 Hz), 7.14-7.20 (4H, m), 7.27 (2H, d, J=8.4 Hz), 7.38 (10H, s), 7.42-7.62 (6H, m), 7.66 (1H, td, J=7.6 and 1.9 Hz), 8.44-8.47 (1H, m), 10.28 (1H, s)

(−)APCI-MS (m/z): 815 (M−H)⁺

### EXAMPLE 162

4′-Bis(benzylsulfonyl)amino]-N-(4-([2-(2-pyridinyl)ethyl]amino}phenyl)-1,1′-biphenyl-2-carboxamide

The title compound was obtained from 4-(bis(benzylsulfonyl)amino]-2′-(4-{(tert-butoxycarbonyl)(2-(2pyridinyl)ethyl]amino}anilino)carbonyl]-1,1′-biphenyl in the same manner as in Example 151.

¹H-NMR (DMSO-d₆): δ2.93-3.00 (2H, m), 3.23-3.35 (2H, m), 5.01 (4H, s), 5.57 (1H, t, J=5.4 Hz), 6,54 (2H, d, J=8.8 Hz), 6.70 (2H, d, J=8.5 Hz), 7.14 (2H, d, J=8.8 Hz), 7.19-7.25 (2H, m), 7.28(2H, d, J=8.5 Hz), 7.39 (10H, s), 7.43-7.58 (4H, m), 7.69 (1H, td, J=7.6 and 1.8 Hz), 8.50 (1H, d, J=4.0 Hz), 9.73 (1H, s)

APCI-MS (m/z): 717 (M+H)⁺

### EXAMPLE 163

4′-[(Benzylsulfonyl)amino]-N-(4-{[2-(2-pyridinyl)ethyl]amino}phenyl)-1,1′-biphenyl-2-carboxamide

The title compound was obtained from 4′-(bis(methylsulfonyl)amino]-N-(4-{[2-(2-pyridinyl)ethyl]amino}phenyl)-1,1′-biphenyl-2-carboxamide in the same manner as in Example 160.

¹H-NMR (DMSO-d₆): δ2.89-2.96 (2H, m), 3.23-3.32 (2H, m), 4.41 (2H, s), 5.49 (1H, t, J=5.9 Hz), 6.48 (2H, d, J=8.9 Hz) 7.15-7.57 (17H, m), 7.69 (1H, td, J=7.6 and 1.9 Hz), 8.50 (1H, d, J=4.0 Hz), 9.74 (1H, s), 9.89 (1H, brs)

APCI-MS (m/z): 563 (M+H)⁺

Preparation 50

Ethyl 2-(4-nitroanilino)-3-(2-pyridinyl)propanoate

The title compound was obtained from ethyl 2-amino-3-(2-pyridinyl)propanoate dihydrochloride in the same manner as in Preparation 33.

¹H-NMR (DMSO-d₆): δ1.10 (3H, t, J=7.1 Hz), 3.15-3.29 (2H, m), 4.08 (2H, q, J=7.1 Hz), 4.65-4.77 (1H, m), 6.68(2H, d, J=9.3 Hz), 7.20-7.26 (1H, m), 7.34 (1H, d, J=7.7 Hz), 7.60 (1H, d, J=8.3 Hz), 7.72 (1H, td, J=7.7 and 1.7 Hz), 7.98 (2H, d, J=9.2 Hz), 8.49 (1H, d, J=4.8 Hz)

APCI-MS (m/z): 316 (M+H)⁺

Preparation 51

To a solution of ethyl 2-(4-nitroanilino)-3-(2-pyridinyl)propanoate (10.5 g) in tetrahydrofuran (210 ml) under a nitrogen atmosphere was added di-tert-butyl dicarbonate (9.45 g) followed by addition of 4-(dimethylamino)pyridine (404 mg). After the solution was refluxed for 16 hours, the reaction mixture was cooled down to ambient temperature and the solvent was removed under reduced pressure. The residue was dissolved in ethyl acetate and the solution was washed with saturated aqueous sodium hydrogencarbonate solution, water and brine, dried over magnesium sulfate and evaporated in vacuo.

The residue was chromatographed on silica gel eluting with hexane-ethyl acetate (from hexane-ethyl acetate 10:1 to 2:1). The eluate was concentrated in vacuo to give a solid. The solid was washed with diisopropyl ether to give ethyl 2-[(tert-butoxycarbonyl)-4-nitroanilino]-3-(2-pyridinyl)propanoate (11.7 g) as a solid.

¹H-NMR (DMSO-d₆): δ1.23 (3H, t, J=7.1 Hz), 1.40 (9H, s), 3.42-3.46 (2H, m), 4.19 (2H, qd, J=7.1 and 2.8 Hz), 5.03-5.10 (1H, m), 7.11 (2H, d, J=9.0 Hz), 7.18-7.21 (2H, m), 7.67(1H, td, J=7.6 and 1.7 Hz), 8.08 (2H, d, J=9.0 Hz), 8.34-8.36 (1H, m)

APCI-MS (m/z): 416 (M+H)⁺

Preparation 52

To a solution of ethyl 2-[(tert-butoxycarbonyl)-4-nitroanilino]-3-(2-pyridinyl)propanoate (1.32 g) dissolved in ethanol (40 ml) and water (5.3 ml) was added iron powder (888 mg) followed by addition of ammonium chloride (170 mg). The mixture was refluxed for 50 minutes and then cooled down to ambient temperature. The reaction mixture was filtered through celite and washed with ethanol and the filtrate was evaporated in vacuo.

The resultant-residue was dissolved in ethyl acetate and the solution was washed with water and brine, dried over magnesium sulfate and evaporated in vacuo to give ethyl 2-[4-amino(tert-butoxycarbonyl)anilino]-3-(2-pyridinyl)propanoate (1.22 g) as a yellow oil.

¹H-NMR (DMSO-d₆): δ1.26 (9H, s), 1.40 (3H, s), 3.23-3.27 (2H, m), 4.05-4.11 (2H, m), 4.74-4.87 (1H, m), 5.01 (2H, s), 7.17-7.27 (2H, m), 7.68-7.76 (1H, m), 8.43 (1H, d, J=4.4 Hz)

APCI-MS (m/z): 385 (M⁺)

### EXAMPLE 164

To a mixture of 4′-(trifluoromethyl)-1,1′-biphenyl-2-carboxylic acid (828 mg), ethyl 2-[4-amino(tert-butoxycarbonyl)anilino)-3-(2-pyridinyl)propanoate (1.20 g), HOBT.H₂O (620 mg) and 4-(dimethylamino)pyridine (60 mg) in tetrahydrofuran (24 ml) was added WSC (0.851 ml) dropwise under a nitrogen atmosphere. The solution was stirred at 65° C. for 23 hours and then cooled down to ambient tempepature. The solvent was evaporated in vacuo. The residue was disssolved in ethyl acetate, washed with saturated aqueous sodium hydrogencarbonate solution, water and brine, dried over magnesium sulfate and then evaporated in vacuo.

The resultant residue was chromatographed on silica gel eluting with hexane-ethyl acetate (from hexane-ethyl acetate 5:1 to 2:1). The eluate was concentrated in vacuo to give ethyl 2-[(tert-butoxycarbonyl)-4-({[4′-(trifluoromethyl)-1,1′-biphenyl-2-yl]carbonyl}amino)anilino]-3-(2-pyridinyl)propanoate (1.39 g) as a yellow amorphous.

¹H-NMR (DMSO-d₆): δ1.21 (3H, t, J=7.0 Hz), 1.35 (9H, brs), 3.33 (2H, s), 4.15 (2H, q, J=7.1 Hz), 4.79-4.86 (1H, m), 6.70 (2H, d, J=8.7 Hz), 7.17-7.25 (2H, m), 7.35 (2H, d, J=8.7 Hz), 7.50-7.78 (9H, m), 8.42 (1H, d, J=4.6 Hz), 10.34 (1H, s)

APCI-MS (m/z): 634 (M+H)⁺

### EXAMPLE 165

To a solution of ethyl 2-[(tert-butoxycarbonyl)-4-({[4′-(trifluoromethyl)-1,1′-biphenyl-2-yl]carbonyl}amino)anilino]-3-(2-pyridinyl)propanoate (200 mg) in ethyl acetate (4.0 ml) was added dropwise 4N hydrogen chloride in dioxane (1.25 ml) under a nitrogen atmosphere. After stirring overnight, the pH of the solution was adjusted to 8.0 with saturated aqueous sodium hydrogencarbonate solution. The separeted organic layer was washed with water and brine, dried over magnesium sulfate and evaporated in vacuo to give a solid. The solid was washed with ether and dried in vacuo to give ethyl 3-(2-pyridinyl)-2-[4-({[4′-(trifluoromethyl)-1,1′-biphenyl-2-yl]carbonyl}amino)anilino]propanoate (158 mg) as a solid.

¹H-NMR (DMSO-d₆): δ1.05 (3H, t, J=7.1 Hz), 3.15 (2H, d, J=7.1 Hz), 4.01 (2H, q, J=7.1 Hz), 4.34-4.45 (1H, m), 5.97 (1H, d, J=9.2 Hz), 6.48 (2H, d, J=8.8 Hz), 7.18 (2H, d, J=8.7 Hz), 7.20-7.22 (1H, m), 7.32 (1H, d, J=7.8 Hz), 7.45-7.67 (6H, m), 7.70-7.77 (3H, m), 8.49 (1H, dd, J=4.8 and 0.9 Hz), 9.94 (1H, s)

APCI-MS (m/z): 534 (M+H)⁺

### EXAMPLE 166

To a solution of ethyl 2-[(tert-butoxycarbonyl)-4-({[4′-(trifluoromethyl)-1,1′-biphenyl-2-yl]carbonyl}amino)anilino]-3-(2-pyridinyl)propanoate (969 mg) dissolved in tetrahydrofuran-ethanol (1:1) (20 ml) was added portionwise lithium borohydride (99.9 mg) under a nitrogen atmosphere. The mixture was refluxed for 1 hour and then cooled down to ambient temperature. The reaction mixture was poured into saturated aqueous ammonium chloride solution and the solvent was removed under reduced pressure. The resultant suspension was extracted with ethyl acetate and the solution was washed with water and brine, dried over magnesium sulfate and evaporated in vacuo.

The resultant residue was chromatographed on silica gel eluting with hexane-ethyl acetate (from hexane-ethylacetate 3:1 to ethyl acetate only). The eluate was concentrated in vacuo to give 2-[(4-{(tert-butoxycarbonyl)[2-hydroxy-1-(2-pyridinylmethyl)ethyl]amino}anilino)carbonyl]-4′-(trifluoromethyl)-1,1′-biphenyl (318 mg) as a white amorphous.

¹H-NMR (DMSO-d₆): δ1.25 (s, 9H), 2.79-3.00 (m, 2H), 3.40-3.59 (m, 2H), 4.32-4.48 (m, 1H), 4.88 (t, 1H, J=5.2 Hz), 6.90 (d, 2H, J=8.6 Hz), 7.19-7.27 (m, 2H), 7.40 (d, 2H, J=8.7 Hz), 7.50-7.78 (m, 9H), 8.47 (d, 1H, J=4.7 Hz), 10.35 (s, 1H)

APCI-MS (m/z): 590 (M+H)⁺

### EXAMPLE 167

N-(4-{(2-Hydroxy-1-(2-pyridinylmethyl)ethyl]amino}phenyl)-4′-(trifluoromethyl)-1,1′-biphenyl-2-carboxamide

The title compound was obtained from 2-[(4-{(tert-butoxycarbonyl)[2-hydroxy-1-(2-pyridinylmethyl)ethyl]amino}-anilino)carbonyl]-4′-(trifluoromethyl)-1,1′-biphenyl in the same manner as in-Example 121.

¹H-NMR (DMSO-d₆): δ2.76-2.87 (1H, m), 2.94-3.04 (1H, m), 3.32-3.47 (2H, m), 3.64-3.79 (1H, m), 4.74 (1H, t, J=5.7 Hz), 5.29 (1H, d, J=8.5 Hz), 6.50 (2H, d, J=8.8 Hz), 7.12-7.21 (1H, m), 7.15 (2H, d, J=8.7 Hz), 7.28 (1H, d, J=7.8 Hz), 7.46-7.78 (9H, m), 8.49 (1H, d, J=3.9 Hz), 9.89 (1H, s)

APCI-MS (m/z): 492 (M+H)⁺

Preparation 53

To a solution of 4-fluoro-3-methylnitrobenzene (3.0 g) and triethylamine (4.04 ml) dissolved in 1,3-dimethyl-2-imidazolidinone (9 ml) was added 2-(2-aminoethyl)pyridine (2.77 ml) under a nitrogen atmosphere. Afetr stirring at 120° C. for 3 hours, the reaction mixture was cooled down to ambient temperature and poured into water. The resultant solid was collected by filtration, washed with water and dried in vacuo to give 2-[2-(2-methyl-4-nitroanilino)ethyl]pyridine (4.67 mg) as a yellow solid.

¹H-NMR (DMSO-d₆): δ2.16 (3H, s), 3.13-3.19 (2H, m), 3.58-3.67 (2H, m), 5.78 (1H, brs), 6.53 (1H, d, J=9.0 Hz), 7.17-7.23 (1H, m), 7.65 (1H, td, J=7.7 and 1.8 Hz), 7.94 (1H, dd, J=2.6 and 0.7 Hz), 8.04 (1H, dd, J=9.0 and 2.6 Hz), 8.55-8.58 (1H, m)

APCI-MS (m/z): 258 (M+H)⁺

Preparation 54

To a solution of 2-[2-(2-methyl-4nitroanilino)ethyl]pyridine (2.0 g) dissolved in 98% formic acid (10 ml) was added dropwise acetic anhydride (4.0 ml). The solution was stirred at 60° C. for 1.5 hours and then cooled down to ambient temrepature. The solvent was removed under reduced pressure and the residue was diluted with ethyl acetate. The organic layer was washed with saturated aqueous sodium hydrogencarbonate solution, water and brine, dried over magnesium sulfate and evaporated in vacuo to give a white solid. The solid was washed with isopropyl alcohol and dried in vacuo to give 2-methyl-4-nitrophenyl[2-(2-pyridinyl)ethyl]formamide (1.98 g) as a white solid.

¹H-NMR (DMSO-d₆): δ2.21 and 2.33 (total 3H, s), 2.93-3.13 (2H, m), 4.13-4.22 (2H, m), 7.06-7.33 (3H, m), 7.55-7.65 (1H, m), 8.01-8.15 (2H, m), 8.13 and 8.30 (total 1H, s), 8.40-8.56 (1H, m)

APCI-MS (m/z): 286 (M+H)⁺

Preparation 55

To a suspension of 2-methyl-4-nitrophenyl[2-(2pyridinyl)ethyl]formamide (1.70 g), iron(III) chloride (19.3 mg) and activated carbon (1.70 g) in ethanol (34 ml) was added dropwise hydrazine monohydrate (1.16 ml) at 80° C. under a nitrogen atmosphere. After stirring at 80° C. for 1.5 hours, the reaction mixture was cooled down to ambient temperature. The resultant suspension was filtered through celite and washed with ethanol. The filtrate was evaporated in vacuo and the residue was diluted with ethyl acetate. The solution was washed with water and brine, dried over magnesium sulfate and evaporated in vacuo to give a white solid. The solid was washed with ether and dried in vacuo to give 4-amino-2-methylphenyl[2-(2-pyridinyl)ethyl]formamide (1.02 g) as a white solid.

¹H-NMR (DMSO-d₆): δ1.88 and 2.01 (total 3H, s), 2.84-2.91 (2H, m), 3.79-3.87 (2H, m), 5.08 and 5.19 (total 2H, s), 6.36-6.47 (2H, s), 6.76-6.80 (1H, m), 7.17-7.31 (2H, m), 7.64-7.76 (2H, m), 7.94 and 8.12 (total 1H, s), 8.43-8.51 (1H, m)

APCI-MS (m/z): 256 (M+H)⁺

### EXAMPLE 168

To a mixture of 4′-(trifluoromethyl)-1,1′-biphenyl-2carboxylic acid (1.04 g), 4-amino-2-methylphenyl[2-(2-pyridinyl)ethyl]formamide (1.00 g), HOBT.H₂O (689 mg) and 4-(dimethylamino)pyridine (50 mg) in N,N-dimethylformamide (20 ml) was added WSC.HCl (1.13 g) portionwise under a nitrogen atmosphere. The solution was stirred for 3 days. The reaction mixture was poured into water and extracted with ethyl acetate. The extract was washed with water three times and brine, dried over magnesium sulfate and evaporated in vacuo. The residue was chromatographed on silica gel eluting with hexane-ethyl acetate (from hexane-ethyl acetate 1:2 to ethyl acetate only). The eluate was concentrated in vacuo to give N-(4-{formyl[2-(2pyridinyl)ethyl]amino}-3-methylphenyl)-4′-(trifluoromethyl)-1,1′-biphenyl-2-carboxamide (1.55 g).

¹H-NMR (DMSO-d₆): δ2.05 and 2.14 (total 3H, s), 2.90-3.06 (2H, m), 3.95-4.08 (2H, m), 6.84-7.22 (6H, m), 7.42-7.82 (8H, m), 8.03 and 8.20 (total 1H, s), 8.43-8.56 (1H, m)

APCI-MS (m/z): 504 (M+H)⁺

### EXAMPLE 169

To a solution of N-(4-{formyl[2-(2-pyridinyl)ethyl]amino}-3-methylphenyl)-4′-(trifluoromethyl)-1,1′-biphenyl-2-carboxamide (1.50 g) dissolved in methanol was added dropwise conc. hydrochloric acid (2.48 ml) at ambient temperature. After stirring at 50° C. for 3.5 hours, the reaction mixture was cooled down to ambient temperature. The solvent was removed under reduced pressure and the residue was dissolved in ethyl acetate. The solution was washed with saturated aqueous sodium hydrogencarbonate solution, water and brine, dried over magnesium sulfate and evaporated in vacuo to give a white solid. The solid was washed with ether and dried in vacuo to give N-(3-methyl-4-{[2-(2-pyridinyl)ethyl]amino}phenyl)-4′-(trifluoromethyl)-1,1′-biphenyl-2-carboxamide (1.26 g) as a white solid.

¹H-NMR (DMSO-d₆): δ2.00 (3H, s), 3.04 (2H, t, J=7.0 Hz), 3.40 (2H, t, J=7.0 Hz), 6.52 (1H, d, J=8.6 Hz), 7.10-7.18 (2H, m), 7.28 (1H, dd, J=7.4 and 5.0 Hz), 7.36 (1H, d, J=7.8 Hz), 7.46-7.65 (6H, m), 7.74-7.82 (3H, m), 8.54 (1H, d, J=4.0 Hz), 9.88 (1H, s)

APCI-MS (m/z): 476 (M+H)⁺

Preparation 56

To a solution of 3-chloro-4-fluoronitrobenzene (3.0 g) and triethylamine (3.58 ml) dissolved in N,N-dimethylformamide (15 ml) was added 2-(2-aminoethyl)pyridine (2.46 ml) under a nitrogen atmosphere and the mixture was stirred for 4 hours. The reaction mixture was poured into water and the resultant percipitate was collected by filtration, washed with water and dried in vacuo to give 2-[2-(2-chloro-4-nitroanilino)ethyl]pyridine (4.69 mg) as a yellow solid.

¹H-NMR (DMSO-d₆): δ3.13-3.20 (2H, m), 3.63-3.77 (2H, m), 5.78 (1H, brs), 6.21 (1H, brs), 6.63 (1H, d, J=9.1 Hz), 7.12-7.23 (1H, m), 7.65 (1H, td, J=7.7 and 1.8 Hz), 8.05 (1H, dd, J=9.1 and 2.5 Hz), 8.18 (1H, d, J=2.5 Hz), 8.57-8.60 (1H, m)

APCI-MS (m/z): 278 (M+H)⁺

Preparation 57

To a solution of 2-[2-(2-chloro-4nitroanilino)ethyl]pyridine (2.0 g) dissolved in 98% formic acid (10 ml) was added dropwise acetic anhydride (4.0 ml). The solution was refluxed under stirring for 12 hours and then cooled down to ambient temrepature. The solvent was removed under reduced pressure and the residue was diluted with ethyl acetate. The solution was washed with saturated aqueous sodium hydrogencarbonate solution, water and brine, dried over magnesium sulfate and evaporated in vacuo to give a light yellow solid. The solid was washed with isopropyl alcohol and dried in vacuo to give 2-chloro-4-nitrophenyl[2-(2-pyridinyl)ethyl]formamide (1.79 g) as a light yellow solid.

¹H-NMR (DMSO-d₆): δ2.94-3.13 (2H, m), 4.19-4.29 (2H, m), 7.07-7.22 (2H, m), 7.27 and 7.41 (total 1H, d, J=8.7 Hz), 7.54-7.63 (1H, m), 8.08-8.24 (2H, m), 8.33-8.53 (2H, m)

APCI-MS (m/z): 306 (M+H)⁺

Preparation 58

To a suspension of 2-chloro-4-nitrophenyl[2-(2-pyridinyl)ethyl]formamide (1.81g), iron(III) chloride (19.2 mg) and activated carbon (1.80 g) in ethanol (64 ml) was added dropwise hydrazine monohydrate (1.15 ml) at 80° C. under a nitrogen atmosphere. After stirring at 80° C. for 1.5 hours, the reaction mixture was cooled down to ambient temperature. The resultant suspension was filtered through celite and washed with ethanol. The filtrate was evaporated in vacuo and diluted with ethyl acetate. The solution was washed with water and brine and dried over magnesium sulfate. Under a nitrogen atmosphere, 4N hydrogen chloride in dioxane (2.96 ml) was added dropwise to the above solution and the suspension was stirred for 10 minutes. The resultant precipitate was filtrated, washed with ethyl acetate and dried in vacuo to give 4-amino-2-chlorophenyl[2-(2-pyridinyl)ethyl]formamide dihydrochloride (1.64 g) as a white solid.

¹H-NMR (DMSO-d₆): δ3.22-3.29 (2H, m), 4.01-4.06 (2H), m, 6.83 (1H, dd, J=8.5 and 2.4 Hz), 7.01 and 7.04 (total 1H, d, J=2.4 Hz), 7.17-7.29 (2H, m), 7.86-8.04 (2H, m), 7.98 and 8.37 (total 1H, s), 8.47 (1H, td, J=7.9 and 1.6 Hz), 8.77-8.79 (1H, m)

APCI-MS (m/z): 276 (M+H)⁺

### EXAMPLE 170

To a mixture of 4′-(trifluoromethyl)-1,1′-biphenyl-2-carboxylic acid (1.19 g), 4-amino-2-chlorophenyl[2-(2pyridinyl)ethyl]formamide dihydrochloride (1.55 g), HOBT.H₂O (785 mg), WSC (1.22 ml) and 4-(dimethylamino)pyridine (77.5 mg) in N,N-dimethylformamide (25 ml) was added triethylamine (0.623 ml) dropwise under a nitrogen atmosphere. The solution was stirred at 120° C. for 2 days. The reaction mixture was cooled down to ambient temperature, poured into water and extracted with ethyl acetate. The extract was washed with water three times and brine, dried over magnesium sulfate and evaporated in vacuo. The residue was chromatographed on silica gel eluting with ethyl acetate-mathanol (from ethyl acetate only to ethyl acetate-methanol 30:1). The eluate was concentrated in vacuo to give N-(3-chloro-4-{formyl[2-(2-pyridinyl)ethyl]amino}phenyl)-4′-(trifluoromethyl)-1,1′-biphenyl-2-carboxamide (188 mg).

¹H-NMR (DMSO-d₆): δ2.89-3.06 (2H, m), 3.99-4.11 (2H, m), 7.07-7.82 (14H, m), 8.05-8.16 (1H, m), 8.42-8.56 (1H, m)

APCI-MS (m/z): 524 (M+H)⁺

### EXAMPLE 171

N-(3-Chloro-4-{[2-(2-pyridinyl)ethyl]amino}phenyl)-4′-(trifluoromethyl)-1,1′-biphenyl-2-carboxamide

The title compound was obtained from N-(3-chloro-4-{formyl[2-(2-pyridinyl)ethyl]amino}phenyl)-4′-(trifluoromethyl)-1,1′-biphenyl-2-carboxamide in the same manner as in Example 169.

¹H-NMR (DMSO-d₆): δ3.02 (2H, t, J=6.9 Hz), 3.45 (2H, t, J=6.9 Hz), 5.49 (1H, brs), 6.71 (1H, d, J=8.9 Hz), 7.20-7.26 (2H, m), 7.32 (1H, d, J=7.8 Hz), 7.48-7.78 (10H, m), 8.52 (1H, dd, J=4.8 and 0.8 Hz), 10.12 (1H, s)

APCI-MS (m/z): 496 (M+H)⁺

Preparation 59

2-Methyl-N¹-(2-(2-pyridinyl)ethyl]-1,4-benzenediamine

The title compound was obtained from 2-[2-(2-methyl-4-nitroanilino)ethyl]pyridine in the same manner as in Preparation 55.

¹H-NMR (DMSO-d₆): δ1.95 (3H, s), 2.96-3.03 (2H, m), 3.23-3.30 (2H, m), 4.18 (2H, s), 4.23 (1H, s), 6.29-6.41 (3H, m), 7.18-7.25 (1H, m), 7.30 (1H, d, J=7.8 Hz), 7.71 (1H, td, J=7.6 and 1.9 Hz), 8.49-8.52 (1H, m)

APCI-MS (m/z): 228 (M+H)⁺

### EXAMPLE 172

4′-Methoxy-N-(3-methyl-4-{[2-(2-pyridinyl)ethyl]amino}phenyl)-1,1′-biphenyl-2-carboxamide

The title compound was obtained from 2-methyl-N¹-[2-(2-pyridinyl)ethyl]-1,4-benzenediamine in the same manner as in Example 120.

¹H-NMR (DMSO-d₆): δ2.01 (3H, s), 2.98-3.05 (2H, m), 3.33-3.43 (2H, m), 3.75 (3H, s), 4.88 (1H, t, J=5.4 Hz), 6.51 (1H, d, J=9.3 Hz), 6.94 (2H, d, J=8.7 Hz), 7.15-7.55 (10H, m), 7.71 (1H, td, J=7.6 and 1.8 Hz), 8.52 (1H, d, J=4.1 Hz), 9.75 (1H, s)

APCI-MS (m/z): 438 (M+H)⁺

Preparation 60

2-Chloro-N¹-[2-(2-pyridinyl)ethyl]-1,4-benzenediamine

The title compound was obtained from 2-[2-(2-chloro-4nitroanilino)ethyl]pyridine in the same manner as in Preparation 55.

¹H-NMR (DMSO-d₆): δ2.95-3.06 (2H, m), 3.30-3.42 (2H, m), 4.54-4.62 (3H, m), 6.46 (1H, dd, J=8.6 and 2.4 Hz), 6.55-6.60 (2H, m), 7.19-7.35 (2H, m), 7.67-7.75 (1H, m), 8.51 (1H, d, J=4.4 Hz)

APCI-MS (m/z): 248 (M+H)⁺

### EXAMPLE 173

N-(3-Chloro-4-{[2-(2-pyridinyl)ethyl]amino}phenyl)-4′-methoxy-1,1′-biphenyl-2-carboxamide

The title compound was obtained from 2-chloro-N¹-[2-(2-pyridinyl)ethyl]-1,4-benzenediamine in the same manner as in Example 120.

¹H-NMR (DMSO-d₆): δ2.98-3.05 (2H, m), 3.40-3.50 (2H, m), 3.75 (3H, s), 5.35 (1H, t, J=5.7 Hz), 6.71 (1H, d, J=8.8 Hz), 6.94 (2H, d, J=8.6 Hz), 7.21-7.56 (10H, m), 7.72 (1H, td, J=7.7 and 1.7 Hz), 8.52 (1H, d, J=4.8 Hz), 9.99 (1H, s)

APCI-MS (m/z): 458 (M+H)⁺

### EXAMPLE 174

To a solution of N-(4-aminophenyl)-4′-(trifluoromethyl)-1,1′-biphenyl-2-carboxamide (0.697 g), (6-[(tert-butoxycarbonyl)amino]-2-pyridinyl)acetic acid (0.494 g) and HOBT (0.317 g) in N,N-dimethylformamide (15 ml) was added WSC.HCl (0.450 g), followed by addition of triethylamine (0.41 ml) at room temperature. The reaction mixture was stirred at 50° C. for 12 hours and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane:ethyl acetate (1:1) to give tert-butyl 6-(2-oxo-2-[4-({[4′-(trifluoromethyl)-1,1′-biphenyl-2-yl]carbonyl}amino)anilino]ethyl}-2-pyridinylcarbamate (0.809 g) as a white soild.

¹H-NMR (DMSO-d₆): δ1.46 (9H, s), 3.72 (2H, s), 7.01 (1H, d, J=7.0 Hz), 7.41-7.76 (14H, m), 9.69 (1H, s), 10.12 (1H, s), 10.26 (1H, s)

### EXAMPLE 175

To a solution of tert-butyl 6-{2-oxo-2-[4-({[4′-(trifluoromethyl}-1,1′-biphenyl-2-yl]carbonyl}amino)anilino]-ethyl}-2-pyridinylcarbamate (0.792 g) in dichloromethane (30 ml) was added trifluoroacetic acid (3.0 ml) dropwise. The reaction mixture was stirred for 15 hours, quenched with 10% aqueous potassium carbonate solution, and extracted with dichloromethane. The organic layer was washed with brine, dried over magnesium sulfate, filtered and concentrated in vacuo. The residue was recrystallized from ethyl acetate-diisopropyl ether to give N-(4-{[(6-amino-2-pyridinyl)acetyl]amino}phenyl)-4′-(trifluoromethyl)-1,1′-biphenyl-2-carboxamide (0.412 g) as a white solid.

¹H-NMR (DMSO-d₆): δ3.54 (2H, s), 5.89 (2H, s), 6.31 (2H, d, J=7.6 Hz), 6.46 (2H, d, J=6.9 Hz), 7.28-7.76 (13H, m), 10.15 (1H, s), 10.26 (1H, s)

Preparation 61

To a solution of 4′-ethyl-1,1′-biphenyl-2-carboxylic acid (1.001 g), 1,4-benzenediamine (1.493 g) and HOBT (0.758 g) in N,N-dimethylformamide (35 ml) was added WSC.HCl (0.949 g), followed by addition of triethylamine (0.541 g) at room temperature. The mixture was stirred at 40° C. for 12 hours. N,N-Dimethylformamide was removed under reduced pressure, then ethyl acetate (20 ml) and water (20 ml) were added to the residue. The separated organic layer was washed with brine, dried over magnesium sulfate and concentrated in vacuo. The residue was purified by column chromatography on silica gel eluting with chloroform:methanol (19:1) to give N-(4-aminophenyl)-4′-ethyl-1,1′-biphenyl-2-carboxamide (1.400 g) as a yellow foamy solid.

¹H-NMR (CDCl₃): δ1.27 (3H, t, J=7.6 Hz), 2.70 (2H, q, J=7.6 Hz), 6.54 (2H, d, J=8.8 Hz), 6.69 (1H, brs), 6.85 (2H, d, J=8.8 Hz), 7.27 (2H, d, J=7.9 Hz), 7.37-7.54 (5H, m), 7.87 (1H, d, J=7.3 Hz).

### EXAMPLE 176

To a solution of N-(4-aminophenyl)-4′-ethyl-1,1′-biphenyl-2-carboxamide (99 mg), 2-pyridinylacetic acid hydrochloride (54 mg) and HOBT (53 mg) in N,N-dimethylformamide (5 ml) was added WSC.HCl (67 mg), followed by addition of triethylamine (77 mg) at room temperature. The mixture was stirred at 40° C. for 18 hours. N,N-Dimethylformamide was removed under reduced pressure, then ethyl acetate (20 ml) and water (20 ml) were added to the residue. The separated organic layer was washed with brine, dried over magnesium sulfate and concentrated in vacuo. The residue was purified by column chromatography on silica gel eluting with chloroform:methanol (19:1) to give 4′-ethyl-N-[4-[(2-pyridinylacetyl)amino]phenyl)-1,1′-biphenyl-2-carboxamide (64 mg) as orange crystals.

¹H-NMR (CDCl₃): δ1.26 (3H, t, J=7.6 Hz), 2.69 (2H, q, J=7.6 Hz), 3.84 (2H, s), 6.85 (1H, brs), 7.02 (2H, d, J=8.9 Hz), 7.23-7.31 (3H, m), 7.36-7.53 (8H, m), 7.70 (1H, dt, J=2.0 Hz and 7.6 Hz), 7.90 (1H, d, J=6.0 Hz), 8.60 (1H, d, J=4.0 Hz), 9.75 (1H, brs).

### Preparation 62

4′-Acetyl-N-(4-aminophenyl)-1,1′-biphenyl-2-carboxamide

The title compound was obtained from 4′-acetyl-1,1′-biphenyl-2-carboxylic acid in the same manner as in Preparation 61 as a dark greenish foamy solid.

¹H-NMR (CDCl₃): δ2.61 (3H, s), 6.56 (2H, d, J=8.9 Hz), 6.81 (1H, brs), 6.96 (2H, d, J=8.9 Hz), 7.38-7.60 (5H, m), 7.79 (1H, d, J=7.3 Hz), 8.01 (2H, d, J=8.6 Hz).

### EXAMPLE 177

To a solution of 4′-acetyl-N-(4-aminophenyl)-1,1′-biphenyl-2-carboxamide (228 mg), {6-[(tert-butoxycarbonyl)amino]-2-pyridinyl}acetic acid (174 mg) and HOBT (116 mg) in N,N-dimethylformamide (10 ml) was added WSC.HCl (146 mg), followed by addition of triethylamine (84 mg) at room temperature. The mixture was stirred at 40° C. for 14 hours. N,N-Dimethylformamide was removed under reduced pressure, then ethyl acetate (10 ml) and water (10 ml) were added. The separated organic layer was washed with brine, dried over magnesium sulfate and concentrated in vacuo. The residue was purified by column chromatography on silica gel eluting with chloroform:methanol (39:1) to give tert-butyl 6-[2-(4-≡[(4′-acetyl-1,1′-biphenyl-2-yl)carbonyl]amino}anilino)-2-oxoethyl]-2-pyridinylcarbamate (390 mg) as a brown tar.

¹H-NMR (CDCl₃): δ1.55 (9H, s), 2.60 (3H, s), 3.72 (2H, s), 6.93-8.01 (17H, m), 9.00 (1H, brs).

### EXAMPLE 178

To a solution of tert-butyl 6-[2-(4-{[(4′-acetyl-1,1′-biphenyl-2-yl)carbonyl]amino}anilino)-2-oxoethyl]-2-pyridinylcarbamate (390 mg) in dichloromethane (10 ml) was added trifluoroacetic acid (1.48 g) at room temperature and the reaction mixture was stirred for 18 hours. The mixture was basified with 10% aqueous potassium carbonate solution and the separated organic layer was washed with brine, dried over magnesium sulfate and concentrated in vacuo. The residue was purified by column chromatography on silica gel eluting with chloroform:methanol (19:1). The solid obtained was recrystallized from ethyl acetate-diisopropyl ether to give 4′-acetyl-N-(4-{[(6amino-2-pyridinyl)acetyl]amino}phenyl)-1,1′-biphenyl-2-carboxamide (122 mg) as pale yellow crystals.

¹H-NMR (DMSO-d₆): δ2.56 (3H, s), 3.53 (2H, s), 5.89 (2H, brs), 6.31 (1H, d, J=7.6 Hz), 6.45 (1H, d, J=6.6 Hz), 7.28-7.34 (1H, m), 7.43-7.68 (10H, m), 7.96 (2H, d, J=8.6 Hz), 10.14 (1H, brs), 10.26 (1H, brs).

ESI-MS (m/z): 487 (M+Na)⁺

### EXAMPLE 179

2-[(4-{(tert-Butoxycarbonyl)[2-(2-pyridinyl)ethyl]amino}anilino)carbonyl]-4′-ethyl-1,1′-biphenyl

The title compound was obtained from tert-butyl 4-aminophenyl[2-(2-pyridinyl)ethyl]carbamate and 4′-ethyl-1,1′-biphenyl-2-carboxylic acid in the same manner as in Example 51 as a yellow foamy solid.

¹H-NMR (CDCl₃): δ1.26 (3H, t, J=7.6 Hz), 1.37 (9H, s), 2.70 (2H, q, J=7.6 Hz), 3.03 (2H, t, J=7.2 Hz), 3.96 (2H, t, J=7.2 Hz), 6.92-7.64 (15H, m), 7.84-7.92 (1H, m), 8.47 (1H, d, J=4.0 Hz).

### EXAMPLE 180

4′-Ethyl-N-(4-{[2-(2-pyridinyl)ethyl]amino}phenyl)-1,1′-biphenyl-2-carboxamide

The title compound was obtained from 2-[(4-{(tert-butoxycarbonyl)[2-(2-pyridinyl)ethyl]amino}anilino)carbonyl]-4′-ethyl-1,1′-biphenyl in the same manner as in Example 59 as a white solid.

¹H-NMR (CDCl₃): δ1.27 (3H, t, J=7.6 Hz), 2.70 (2H, q, J=7.6 Hz), 3.05 (2H, t, J=6.6 Hz), 3.48 (2H, t, J=6.6 Hz), 6.49 (2H, d, J=8.9 Hz), 6.69 (1H, brs), 6.87 (2H, d, J=8.9 Hz), 7.12-7.16 (2H, m), 7.26-7.29 (2H, m), 7.37-7.63 (6H, m), 7.86-7.89 (1H, m), 8.54-8.56 (1H, m).

FAB-MS (m/z): 422 (M+H)⁺

### EXAMPLE 181

tert-Butyl 4-{[(3′-acetyl-1,1′-biphenyl-2-yl)carbonyl]amino}phenyl[2-(2-pyridinyl)ethyl]carbamate

The title compound was obtained from tert-butyl 4-aminophenyl[2-(2-pyridinyl)ethyl]carbamate and 3′-acetyl-1,1′-biphenyl-2-carboxylic acid in the same manner as in Example 51 as an orange oil.

¹H-NMR (CDCl₃): δ1.36 (9H, s), 2.57 (3H, s), 3.04 (2H, t, J=7.3 Hz), 3.97 (2H, t, J=7.4 Hz), 6.90-7.68 (12H, m), 7.81 (2H, d, J=8.0 Hz), 7.94 (1H, d, J=7.6 Hz), 8.08 (1H, s), 8.46 (1H, d, J=4.3 Hz)

### EXAMPLE 182

3′-Acetyl-N-(4-{[2-(2-pyridinyl)ethylπamino}phenyl)-1,1′-biphenyl-2-carboxamide

The title compound was obtained from tert-butyl 4-{[(3′-acetyl-1,1′-biphenyl-2-yl)carbonyl]amino}phenyl[2-2-pyridinyl)ethyl]carbamate in the same manner as in Example 59 as faintly brown crystals.

¹H-NMR (CDCl₃): δ2.57 (3H, s), 3.05 (2H, t, J=6.6 Hz), 3.48 (2H, t, J=6.6 Hz), 6.50 (2H, d, J=8.9 Hz), 6.75 (1H, brs), 6.95 (2H, d, J=8.6 Hz), 7.12-7.17 (2H, m), 7.44-7.64 (5H, m), 7.70 (1H, d, J=7.9 Hz), 7.80 (1H, d, J=7.6 Hz), 7.97 (1H, d, J=7.9 Hz), 8.09 (1H, brs), 8.55 (1H, d, J=4.0 Hz).

ESI-MS (m/z): 458(M+Na)⁺

### EXAMPLE 183

3′-(1-Hydroxyethyl)-N-(4-{[2-(2-pyridinyl)ethyl]amino}phenyl)-1,1′-biphenyl-2-carboxamide

The title compound was obtained from 3′-acetyl-N-(4-[2-(2pyridinyl)ethyl]amino}phenyl)-1,1′-biphenyl-2-carboxamide in the same manner as in Example 30 as white crystals.

¹H-NMR (CDCl₃): δ1.42 (3H, d, J=6.6 Hz), 3.04 (2H, t, J=6.6 Hz), 3.47 (2H, t, J=6.6 Hz), 4.86 (1H, q, J=6.3 Hz), 6.49 (2H, d, J=8.9 Hz), 6.69 (1H, brs), 6.90 (2H, d, J=8.9 Hz), 7.13-7.16 (2H, m), 7.41-7.63 (8H, m), 7.83-7.86 (1H, m), 8.53-8.55 (1H, m).

ESI-MS (m/z): 438 (M+H)⁺

### EXAMPLE 184

Tert-Butyl 4-{(3′-isopropyl-1,1′-biphenyl-2yl)carbonyl]amino}phenyl[2-(2-pyridinyl)ethyl]carbamate

The title compound was obtained from tert-butyl 4-aminophenyl[2-(2-pyridinyl)ethyl]carbamate and 3′-isopropyl-1,1′-biphenyl-2-carboxylic acid in the same manner as in Example 51 as a yellow oil.

¹H-NMR (CDCl₃): δ1.16 (6H, d, J=6.9 Hz), 1.36 (9H, s), 2.84-2.93 (1H, m), 2.99 (2H, t, J=7.4 Hz), 3.95 (2H, t, J=7.4 Hz), 6.87 (1H, brs), 6.98-7.15 (6H, m), 7.26-7.31 (3H, m), 7.36-7.60 (5H, m), 7.92 (1H, d, J=7.3 Hz), 8.47 (1H, d, J=4.9 Hz)

### EXAMPLE 185

3′-Isopropyl-N-(4-{[2-(2-pyridinyl)ethyl]amino}phenyl)-1,1′-biphenyl-2-carboxamide

The title compound was obtained from tert-butyl 4-{[(3′-isopropyl-1,1′-biphenyl-2-yl)carbonyl]amino}phenyl[2(2-pyridinyl)ethyl]carbamate in the same manner as in Example 59 as white crystals.

¹H-NMR (CDCl₃): δ1.19 (6H, d, J=6.9 Hz), 2.84-2.95 (1H, m), 3.04 (2H, t, J=6.6 Hz), 3.47 (2H, t, J=6.6 Hz), 6.48 (2H, d, J=8.9 Hz), 6.67 (1H, brs), 6.86 (2H, d, J=8.6 Hz), 7.12-7.16 (2H, m), 7.24-7.63 (8H, m), 7.88-7.91 (1H, m), 8.54-8.56 (1H, m).

ESI-MS (m/z): 436 (M+H)⁺

### EXAMPLE 186

tert-Butyl 2-{6-[(tert-butoxycarbonyl)amino]-2-pyridinyl}ethyl(4-{[(4′-ethyl-1,1′-biphenyl-2-yl)carbonyl]amino}phenyl)carbamate

The title compound was obtained from tert-butyl 4-aminophenyl(2-{6-[(tert-butoxycarbonyl)amino]-2-pyridinyl}ethyl)carbamate and 4′-ethyl 1,1′-biphenyl-2-carboxylic acid in the same manner as in Example 76 as a pale brown oil.

¹H-NMR (CDCl³): δ1.26 (3H, t, J=7.6 Hz), 1.42 (18H, s), 2.70 (2H, q, J=7.6 Hz), 3.00 (2H, t, J=7.6 Hz), 3.89 (2H, t, J=7.9 Hz), 6.77-7.70 (16H, m), 7.91 (1H, d, J=7.6 Hz).

### EXAMPLE 187

N-(4-{[2-(6-Amino-2-pyridinyl)ethyl]amino}phenyl)-4′-ethyl-1,1′-biphenyl-2-carboxamide

The title compound was obtained from tert-butyl 2-{6[(tert-butoxycarbonyl)amino]-2-pyridinyl}ethyl(4-{[(4′-ethyl-1,1′-biphenyl-2-yl)carbonyl]amino}phenyl)carbamate in the same manner as in Example 77 as a pale brown foamy solid.

¹H-NMR (CDCl₃): δ1.27 (3H, t, J=7.6 Hz), 2.70 (2H, q, J=7.6 Hz), 2.86 (2H, t, J=6.6 Hz), 3.41 (2H, t, J=6.6 Hz), 4.52 (2H, brs), 6.35 (1H, d, J=8.2 Hz), 6.37-6.51 (3H, m), 6.86 (2H, d, J=8.9 Hz), 7.26-7.50 (8H, m) 7.88 (1H, d, J=7.3 Hz).

### EXAMPLE 188

tert-Butyl 2-{6-[(tert-butoxycarbonyl)amino]-2-pyridinyl}ethyl(4-{[(4′-methyl-1,1′-biphenyl-2-yl)carbonyl]amino}phenyl)carbamate

The title compound was obtained from tert-butyl 4-aminophenyl(2-{6-[(tert-butoxycarbonyl)amino]-2-pyridinyl}ethyl)carbamate and 4′-methyl-1,1′-biphenyl-2-carboxylic acid in the same manner as in Example 76 as a pale yellow oil.

¹H-NMR (CDCl₃): δ1.41 (18H, s), 2.40 (3H, s), 3.01 (2H, t, J=7.6 Hz), 3.89 (2H, t, J=7.6 Hz), 6.77-7.90 (16H, m)

### EXAMPLE 189

N-(4-{[2-(6-Amino-2-pyridinyl)ethyl]amino}phenyl)-4′-methyl-1,1′-biphenyl-2-carboxamide

The title compound was obtained from tert-butyl 2-{6-[(tert-butoxycarbonyl)amino]-2-pyridinyl}ethyl(4-{[(4′-methyl-1,1′-biphenyl-2-yl)carbonyl]amino}phenyl)carboxamide in the same manner as in Example 77 as a pale yellow foam.

¹H-NMR (CDCl₃): δ2.39 (3H, s), 2.87 (2H, t, J=6.6 Hz), 3.41 (2H, t, J=6.6 Hz), 4.46 (2H, brs), 6.36 (1H, d, J=8.3 Hz), 6.48-6.51 (3H, m), 6.73 (1H, brs), 6.91 (2H, d, J=8.9 Hz), 7.22-7.52 (8H, m), 7.85 (1H, dd, J=1.3 and 7.3 Hz).

ESI-Ms (m/z): 423 (M+H)⁺

### EXAMPLE 190

tert-Butyl 2-{6-[(tert-butoxycarbonyl)amino]-2-pyridinyl}ethyl(4-{[(4′-methoxy-1,1′-biphenyl-2-yl)carbonyl]amino}phenyl)carbamate

The title compound was obtained from tert-butyl 4-aminophenyl(2-{6-[(tert-butoxycarbonyl)amino]-2-pyridinyl}ethyl)carbamate and 4′-methoxy-1,1′-biphenyl-2-carboxylic acid in the same manner as in Example 76 as a yellow oil.

### EXAMPLE 191

N-(4-{[2-(6-Amino-2-pyridinyl)ethyl]amino}phenyl)-4′-methoxy-1,1′-biphenyl-2-carboxamide

The title compound was obtained from tert-butyl 2-{6-[(tert-butoxycarbonyl)amino]-2-pyridinyl}ethyl(4-{[(4′-1,1′-biphenyl-2-yl)carbonyl]amino}phenyl)carbamate in the same manner as in Example 77 as a pale yellow foam.

¹H-NMR (CDCl₃): δ2.86 (2H, t, J=6.6 Hz), 3.41 (2H, t, J=6.6 Hz), 3.83 (3H, s), 4.50 (2H, brs), 6.35 (1H, d, J=8.2 Hz), 6.50 (3H, d, J=8.9 Hz), 6.78 (1H, brs), 6.93-6.98 (4H, m), 7.32-7.52 (6H, m), 7.83 (1H, d, J=7.3 Hz).

ESI-MS (m/z): 439 (M+H)⁺

### EXAMPLE 192

tert-Butyl 6-(2-[4-{[(4′-acetyl-1,1′-biphenyl-2-yl)carbonyl]amino}(tert-butoxycarbonyl)anilino]ethyl}-2-pyridinylcarbamate

The title compound was obtained from tert-butyl 4-aminophenyl(2-(6-[(tert-butoxycarbonyl)amino]-2-pyridinyl}ethyl)carbamate and 4′-acetyl-1,1′-biphenyl-2-carboxylic acid in the same manner as in Example 76 as a yellow oil.

¹H-NMR (CDCl₃): δ1.42 (18H, s), 2.61 (3H, s), 3.07 (2H, t, J=7.9 Hz), 3.95 (2H, t, J=7.9 Hz), 7.03-7.88 (12H, m), 8.04 (3H, d, J=8.6 Hz), 8.30 (1H, d, J=8.2 Hz).

### EXAMPLE 193

4′-Acetyl-N-(4-{[2-(6-amino-2-pyridinyl)ethyl]amino}-phenyl)-1,1′-biphenyl-2-carboxamide

The title compound was obtained from tert-butyl 6-{2-[4-{[(4′-acetyl-1,1′-biphenyl-2-yl)carbonyl]amino}(tert-butoxycarbonyl)anilino]ethyl}-2-pyridinylcarbamate in the same manner as in Example 77 as a pale yellow foam.

¹H-NMR (CDCl₃): δ2.60 (3H, s), 2.88 (2H, t, J=6.6 Hz), 3.41 (2H, t, J=6.6 Hz), 5.16 (2H, brs), 6.39 (1H, d, J=8.3 Hz), 6.47-6.50 (3H, m), 6.91 (1H, brs), 6.97 (2H, d, J=8.9 Hz), 7.35-7.59 (6H, m), 7.76 (1H, d, 7.3 Hz), 7.99 (2H, d, J=8.3 Hz).

ESI-MS (m/z): 451 (M+H)⁺

### EXAMPLE 194

To a solution of [2-(formylamino)-1,3-thiazol-4-yl]acetic acid (583 mg) and HOBT (528 mg) in N,N-dimethylformamide (12 ml) was added WSC.HCl (661 mg), followed by addition of a solution of N-(4-aminophenyl)-4′-(trifluoromethyl)-1,1′-biphenyl-2-carboxamide (1.12 g) and triethylamine (0.52 ml) in N,N-dimethylformamide at room temperature. The resulting solution was stirred at 50° C. for 3.5 hours. The reaction mixture was quenched with water and extracted with ethyl acetate. The separated organic layer was washed with water and brine, dried over magnesium sulfate and evaporated in vacuo. The residue was purified by column chromatography on silica gel eluting with chloroform:methanol:triethylamine (10:1:0.1) to give N-[4-({[2-(formylamino)-1,3-thiazol-4-yl]acetyl}amino)phenyl]-4′-(trifluoromethyl)-1,1′-biphenyl-2-carboxamide (484 mg) as a yellow solid.

¹H-NMR (DMSO-d₆): δ3.66 (2H, s), 6.99 (1H, s), 7.4-7.8 (12H, m), 8.44 (1H, s), 10.08 (1H, s), 10.27 (1H, s), 12.21 (1H, brs)

FAB-MS (m/z): 525 (M+H)⁺

### EXAMPLE 195

To a suspension of N-[4-({[2-(formylamino)-1,3-thiazol-4-yl]acetyl}amino)phenyl]-4′-(trifluoromethyl)-1,1-biphenyl-2carboxamide (155 mg) in methanol (5 ml) was added 6N-HCl (0.5 ml) at room temperature. The reaction mixture was refluxed under stirring for 15 minutes to give clear orange solution. After cooling down to room temperature, ethyl acetate (10 ml) and 10% aqueous potassium carbonate solution (10 ml) were added and the separated organic layer was dried over magnesium sulfate and evaporated in vacuo. The obtained foamy solid was recrystallized from ethyl acetate and diisopropyl ether to give N-(4-([(2-amino-1,3-thiazol-4-yl)acetyl]amino}phenyl)-4′-(trifluoromethyl)-1,1′-biphenyl-2-carboxamide (130 mg) as orange crystals.

¹H-NMR (DMSO-d₆): δ3.51 (2H, s), 6.40 (1H, s), 7.41-7.64 (10H, m), 7.75 (2H, d, J=8.2 Hz), 10.07 (1H, brs), 10.27 (1H, brs).

EI-MS (m/z): 496 (M⁺)

### EXAMPLE 196

To a solution of N-(4-aminophenyl)-4′-ethyl-1,1′-biphenyl-2-carboxamide (0.240 g), 12-(formylamino)-1,3-thiazol-4-yl)acetic acid (0.141 g) and HOBT (0.123 g) in tetrahydrofuran (15 ml) was added WSC.HCl (0.174 g), followed by addition of triethylamine (0.16 ml) at room temperature. The reaction mixture was stirred for 12 hours, quehched with water and extracted with ethyl acetate. The organic layer was washed with brine, dried over magnesium sulfate, filtered and concentrated in vacuo. The residue was purified by column chromatography on silica gel eluting with chloroform:methanol (9:1) to give 4′-ethyl-N-[4({[2-(formylamino)-1,3-thiazol-4-yl]acetyl}amino)phenyl]-1,1′-biphenyl-2-carboxamide (0.251 g) as a white soild.

¹H-NMR (DMSO-d₆): δ1.17 (3H, t, J=7.6 Hz), 2.59 (2H, q, J=7.6 Hz), 3.67 (2H, s), 7.00-7.54 (13H, m), 8.45 (1H, s), 10.07 (1H, s), 10.13 (1H, s), 12.20 (1H, br s)

### EXAMPLE 197

To a solution of 4′-ethyl-N-[4-({[2-(formylamino)-1,3-thiazol-4-yl]acetyl}amino)phenyl]-1,1′-biphenyl-2-carboxamide (76 mg) in methanol (5 ml) was added 6N HCl (0.3 ml). The reaction mixture was stirred at 70° C. for 15 hours and concentrated in vacuo. The residue was dissolved in ethyl acetate and 10% aqueous potassium carbonate solution, and extracted with ethyl acetate. The organic layer was washed with brine, dried over magnesium sulfate, filtered and concentrated in vacuo. The residue was recrystallized from ethyl-acetate-diisopropyl ether to give N-(4-{[(2-amino-1,3-thiazol-4-yl)acetyl]amino}phenyl)-4′-ethyl-1,1′-biphenyl-2-carboxamide (52 mg) as a white solid.

¹H-NMR (DMSO-d₆): δ1.16 (3H, t, J=7.6 Hz), 2.58 (2H, q, J=7.6 Hz), 3.43 (2H, s), 6.29 (1H, s), 6.88 (2H, s), 7.20 (2H, d, J=8.2 Hz), 7.35 (2H, d, J=8.2 Hz), 7.41-7.55 (5H, m), 10.00(1H, s), 10.13 (1H, s)

### EXAMPLE 198

4′-Acetyl-N-[4-({[2-(formylamino)-1,3-thiazol-4-yl]acetyl}amino)phenyl]-1,1′-biphenyl-2-carboxamide

The title compound was obtained from 4′-acetyl-N-(4-aminophenyl)-1,1′-biphenyl-2-carboxamide and [2-(formylamino)-1,3-thiazol-4-yl]acetic acid in the same manner as in Example 194 as an orange tar.

¹H-NMR (DMSO-d₆): δ2.57 (3H, s), 3.79 (2H, s), 7.00 (1H, s), 7.43-7.72 (10H, m), 7.96 (2H, d, J=8.2 Hz), 8.46 (1H, brs), 10.07 (1H, brs), 10.27 (1H, brs), 12.21 (1H, brs).

### EXAMPLE 199

4′-Acetyl-N-(4-{[(2-amino-1,3-thiazol-4-yl)acetyl]amino}phenyl)-1,1′-biphenyl-2-carboxamide

The title compound was obtained from 4′-acetyl-N-[4-({[2-(formylamino)-1,3-thiazol-4-yl]acetyl}amino)phenyl]-1,1′-biphenyl-2-carboxamide in the same manner as in Example 195 as yellow crystals.

¹H-NMR (DMSO-d₆): δ2.56 (3H, s), 3.43 (2H, s), 6.29 (1H, s), 6.88 (2H, s), 7.42-7.62 (10H, m), 7.95 (2H, d, J=8.6 Hz), 10.0 (1H, brs), 10.26 (1H, brs).

ESI-MS (m/z): 493 (M+Na)⁺

### EXAMPLE 200

N-{4-[(1,3-Thiazol-4-ylacetyl)amino]phenyl}-4′-(trifluoromethyl)-1,1′-biphenyl-2-carboxamide

The title compound was obtained from N-(4-aminophenyl)-4′-(trifluoromethyl)-1,1′-biphenyl-2-carboxamide and (1,3-thiazol-4yl)acetic acid in the same manner as in Example 194 as a yellow solid.

¹H-NMR (CD₃OD): δ3.90 (2H, s), 7.3-7.7 (14H, m), 8.0-8.1 (2H, m), 8.96 (1H, s).

ESI-MS (m/z): 504 (M+Na)⁺, 482 (M+H)⁺

### EXAMPLE 201

4′-Ethyl-N-{4-[(1,3-thiazol-4-ylacetyl)amino]phenyl}-1,1′-biphenyl-2-carboxamide

The title compound was obtained from N-(4-aminophenyl)-4′-ethyl-1,1′-biphenyl-2-carboxamide and (1,3-thiazol-4-yl)acetic acid in the same manner as in Example 194 as a yellow solid.

¹H-NMR (CDCl₃): δ1.25 (3H, t, J=7.6 Hz), 2.68 (2H, q, J=7.6 Hz), 6.9-7.9 (14H, m), 8.85 (1H, s), 8.98 (1H, s).

FAB-MS (m/z): 442 (M+H)⁺

### EXAMPLE 202

To a solution of N-(4-aminophenyl)-4-(trifluoromethyl)-1,1′-biphenyl-2-carboxamide (276 mg), (2-[(tert-butoxycarbonyl)(methyl)amino)-1,3-thiazol-4-yl acetic acid (211 mg) and HOBT (142 mg) in N,N-dimethylformamide (10 ml) was added WSC.HCl (178 mg), followed by addition of triethylamine (104 mg) at room temperature. The mixture was stirred at 40° C. for 12 hours. N,N-Dimethylformamide was removed under reduced pressure, then ethyl acetate (20 ml) and water (20 ml) were added. The separated organic layer was washed with brine, dried over magnesium sulfate and concentrated in vacuo. The residue was purified by column chromatography on silica gel eluting with chloroform:methanol (39:1) to give tert-butyl methyl(4-(2-oxo-2-[4-({[4′-(trifluoromethyl)-1,1′-biphenyl-2-yl]carbonyl}amino)anilino]ethyl}-1,3-thiazol-2-yl)carbamate (305 mg) as a pale brown oil.

¹H-NMR (CDCl₃): δ1.60 (9H, s), 3.61 (3H, s), 3.71 (2H, s), 6.72 (1H, s), 6.93 (1H, brs), 7.09-7.15 (2H, m), 7.38-7.69 (9H, m), 7.80 (1H, d, J=6.0 Hz), 9.21 (1H, brs).

### EXAMPLE 203

To a solution of tert-butyl methyl(4-{2-oxo-2-[4-({[4′-(trifluoromethyl)-1,1′-biphenyl-2-yl]carbonyl}amino)anilino]-ethyl}-1,3-thiazol-2-yl)carbamate (301 mg) in dichloromethane (6 ml) was added trifluoroacetic acid (0.89 g) at room temperature and the reaction mixture was stirred for 13 hours. The mixture was basified with 10% aqueous potassium carbonate solution and the separated organic layer was washed with brine, dried over magnesium sulfate and concentrated in vacuo. The residue was recrystallized from ethyl acetate-diisopropyl ether to give N-[4-({[2-(methylamino)-1,3-thiazol-4-yl]acetyl}amino)phenyl]-4′-(trifluoromethyl)-1,1′-biphenyl-2-carboxamide (58 mg) as pale yellow crystals.

¹H-NMR (DMSO-d₆): δ3.31 (3H, s), 3.48 (2H, s), 6.36 (1H, s), 7.41-7.64 (10H, m), 7.75 (2H, d, J=8.2 Hz), 10.03 (1H, brs), 10.26 (1H, s)

ESI-MS (m/z): 511 (M+H)⁺

### EXAMPLE 204

N-(4-{[(2-Methyl-1,3-thiazol-4-yl)acetyl]amino}phenyl)-4′-(trifluoromethyl)-1,1′-biphenyl-2-carboxamide

The title compound was obtained from N-(4-aminophenyl)-4′-(trifluoromethyl)-1,1′-biphenyl-2-carboxamide and (2-methyl-1,3thiazol-4-yl)acetic acid in the same manner as in Example 194 as faintly brown crystals.

¹H-MMR (DMSO-d₆): δ2.62 (3H, s), 3.72 (2H, s), 7.25 (1H, s), 7.41-7.64 (10H, m), 7.75 (2H, d, J=8.2 Hz), 10.10 (1H, brs), 10.26 (1H, brs).

ESI-MS (m/z): 518 (M+Na)⁺

Preparation 63

To a solution of 1,3-thiazole-2-carbaldehyde (1.043 g) in tetrahydrofuran (30 ml) were added N-(4-aminophenyl)-4′-(trifluoromethyl)-1,1′-biphenyl-2-carboxamide (3.12 g) and magnesium sulfate (2.108 g). The reaction mixture was stirred for 24 hours and filtered. The filtrate was concentrated in vacuo and the residue was recrystallized from ethyl acetate to give N-{4-[(1,3-thiazol-2-ylmethylene)amino]phenyl}-4′-(trifluoromethyl)-1,1′-biphenyl-2-carboxamide (3.162 g) as a yellow solid.

### EXAMPLE 205

To a solution of N-{4-[(1,3-thiazol-2-ylmethylene)amino]phenyl}-4′-(trifluoromethyl)-1,1′-biphenyl-2-carboxamide (0.289 g) in methanol (15 ml) was added NaBH₄ (0.024 g) at 0° C. The reaction mixture was warmed to room temperature and stirred for 15 hours. The reaction mixture was quenched with water and concentrated in vacuo. The residue was dissolved in ethyl acetate and water, and extracted with ethyl acetate. The organic layer was washed with brine, dried over magnesium sulfate, filtered and concentrated in vacuo. The residue was recrystallized from ethyl acetate-diisopropyl ether to give N-{4-[(1,3-thiazol-2-ylmethyl)amino]phenyl}-4′-(trifluoromethyl)-1,1′-biphenyl-2-carboxamide (0.209 g) as a pale yellow solid.

¹H-NMR (DMSO-d₆): δ4.53 (2H, d, J=9.4 Hz), 3.35 (2H, s), 6.51 (2H, d, J=8.9 Hz), 7.19 (2H, d, J=8.9 Hz), 7.17-7.76(11H, m), 9.95 (1H, s)

### EXAMPLE 206

To a suspension of N-(4-aminophenyl)-4′-(trifluoromethyl)-1,1′-biphenyl-2-carboxamide (100 mg) and N-[4-(chloromethyl)-1,3-thiazol-2-yl]acetamide (268 mg) in N,N-dimethylformamide (10 ml) were added potassium iodide (233 mg) and cesium carbonate, and the mixture was stirred at 60° C. for 48 hours. N,N-Dimethylformamide was removed under reduced pressure, and then ethyl acetate (10 ml) and water (10 ml) were added. The separated organic layer was washed with brine, dried over magnesium sulfate and concentrated in vacuo. The residue was purified by column chromatography on silica gel eluting with chloroform:methanol (9:1) to give N-[4-({[2-(acetylamino)-1,3-thiazol-4-yl]methyl}amino)phenyl]-4′-(trifluoromethyl)-1,1′-biphenyl-2-carboxamide (143 mg) as a brown solid.

¹H-NMR (CDCl₃): δ2.24 (3H, s), 4.25 (2H, s), 6.51 (2H, d, J=8.9 Hz), 6.73 (1H, s), 6.81 (1H, brs), 6.92 (2H, d, J=8.9 Hz), 7.40-7.83 (8H, m)

### EXAMPLE 207

To a suspension of N-[4-({[2-(acetylamino) -1,3-thiazol-4-yl]methyl}amino)phenyl)-4′-(trifluoromethyl)-1,1′-biphenyl-2carboxamide (143 mg) in methanol (5 ml) was added 6N-HCl (0.5 ml) at room temperature. The reaction mixture was refluxed under stirring for 14 hours to give clear orange solution. Methanol was removed under reduced pressure, and then ethyl acetate (20 ml) and water (10 ml) were added. The separated organic layer was washed with brine, dried over magnesium sulfate and concentrated in vacuo. The residue was purified by preparative thin-layer chromatography on silica gel by developing with chloroform:methanol (10:1) to give N-(4-{[(2-amino-1,3-thiazol-4-yl)methyl]amino}phenyl)-4′-(trifluoromethyl)-1,1′-biphenyl-2-carboxamide (17 mg) as orange crystals.

¹H-NMR (CDCl₃): δ4.03 (2H, s), 5.21 (2H, brs), 6.49 (2H, d, J=8.9 Hz), 6.65 (1H, s), 6.83 (1H, brs), 6.94 (2H, d, J=8.9 Hz), 7.41-7.81 (8H, m).

ESI-MS(m/z): 469 (M+H)⁺

### EXAMPLE 208

tert-Butyl 2-{2-[(tert-butoxycarbonyl)amino]-1,3-thiazol-4-yl}ethyl(4-{[(4′-ethyl-1,1′-biphenyl-2-yl)carbonyl]amino}phenyl)carbamate

The title compound was obtained from tert-butyl N-4-aminophenyl-N-(2-{2-[(tert-butoxycarbonyl)amino]-1,3-thiazol-4-yl}ethyl)carbamate and 4′-ethyl-1,1′-biphenyl-2-carboxylic acid in the same manner as in Example 74 as a white soild.

¹H-NMR (CDCl₃): δ1.27 (3H, t, J=7.6 Hz), 1.51 (18H, s), 2.69 (2H, q, J=7.6 Hz), 2.93 (2H, t, J=6.6 Hz), 3.38 (2H, t, J=6.6 Hz), 6.47 (2H, d, J=8.9 Hz), 6.69 (1H, s), 6.74 (1H, s), 6.86 (2H, d, 8.9 Hz), 7.25-7.88 (8H, m)

### EXAMPLE 209

N-(4-}[2-(2-Amino-1,3-thiazol-4-yl)ethyl]amino}phenyl)-4′-ethyl-1,1′-biphenyl-2-carboxamide

The title compound was obtained from tert-butyl 2-{2-[(tert-butoxycarbonyl)amino]-1,3-thiazol-4-yl}ethyl(4-{[(4′-ethyl-1,1′-biphenyl-2-yl)carbonyl]amino}phenyl)carbamate in the same manner as in Example 75 as a yellow soild.

¹H-NMR (DMSO-d₆): δ1.18 (3H, t, J=7.6 Hz), 2.62 (4H, m), 3.20 (2H, q, J=7.0 Hz), 5.42 (1H, t, J=5.6 Hz), 6.20 (1H, s), 6.48 (2H, d, J=8.9 Hz), 6.83 (2H, s), 7.19-7.54 (8H, m), 9.76 (1H, s)

### EXAMPLE 210

tert-Butyl 2-{2-[(tert-butoxycarbonyl) amino]-1,3-thiazol-4yl}ethyl(4-{[(4′-methyl-1,1′-biphenyl-2-yl) carbonyl]amino}phenyl)carbamate

The title compound was obtained from tert-butyl N-4aminophenyl-N-(2-{2-[(tert-butoxycarbonyl)amino]-1,3-thiazol-4-yl}ethyl)carbamate and 4′-methyl-1,1′-biphenyl-2-carboxylic acid in the same manner as in Example 74 as a yellow oil.

### EXAMPLE 211

N-(4-{[2-(2-Amino-1,3-thiazol-4-yl)ethyl]amino}phenyl)-4′-methyl-1,1′-biphenyl-2-carboxamide

The title compound was obtained from tert-butyl 2-{2-[(tert-butoxycarbonyl)amino]-1,3-thiazol-4-yl}ethyl(4-{[(4′-methyl-1,1′-biphenyl-2-yl)carbonyl]amino}phenyl)carbamate in the same manner as in Example 75 as a yellow foam.

¹H-NMR (CDCl₃): δ2.38 (3H, s), 2.78 (2H, t, J=6.6 Hz), 3.34 (2H, t, J=6.6 Hz), 5.04 (2H, br s), 6.15 (1H, s), 6.48 (2H, d, J=6.9 Hz), 6.79 (1H, s), 6.89 (2H, d, J=6.9 Hz), 7.21-7.85 (8H, m)

### EXAMPLE 212

tert-Butyl 2-{2-[(tert-butoxycarbonyl)amino]-1,3-thiazol-4-yl}ethyl(4-{[(4′-methoxy-1,1′-biphenyl-2-yl)carbonyl]amino}phenyl)carbamate

The title compound was obtained from tert-butyl N-4-aminophenyl-N-(2-{2-[(tert-butoxycarbonyl)amino]-1,3-thiazol-4-yl}ethyl)carbamate and 4′-methoxy-1,1′-biphenyl-2-carboxylic acid in the same manner as in Example 74 as a yellow foam.

### EXAMPLE 213

N-(4-{[2-(2-Amino-1,3-thiazol-4-yl)ethyl]amino}phenyl)-4′-methoxy-1,1′-biphenyl-2-carboxamide

The title compound was obtained from tert-butyl 2-{2-[(tert-butoxycarbonyl)amino]-1,3-thiazol-4-yl}ethyl(4-{[4′-methoxy-1,1′-biphenyl-2-yl)carbonyl]amino}phenyl)carbamate in the same manner as in Example 75 as a yellow foam.

¹H-NMR (CDCl₃): δ2.78 (2H, d, J=6.6 Hz), 3.34 (2H, d, J=6.6 Hz), 3.82 (3H, s), 6.14 (1H, s), 6.83 (1H, s), 6.93-6.97 (4H, m), 7.36-7.83 (6H, m)

### EXAMPLE 214

2-({4-[(tert-Butoxycarbonyl)(2-{2-[(tert-butoxycarbonyl)amino]-1,3-thiazol-4-yl}ethyl)amino]anilino}carbonyl)-4′-chloro-1,1′-biphenyl

The title compound was obtained from tert-butyl N-4-aminophenyl-N-(2-{2-[(tert-butoxycarbonyl)amino]-1,3-thiazol-4-yl}ethyl)carbamate and 4′-chloro-1,1′-biphenyl-2-carboxylic acid in the same manner as in Example 74 as a brown tar.

¹H-NMR (CDCl₃): δ1.26 (9H, s), 1.49 (9H, s), 2.92 (2H, t, J=7.9 Hz), 3.88 (2H, t, J=7.9 Hz), 6.76 (1H, s), 7.03-7.08 (3H, m), 7.18 (2H, d, J=8.9 Hz), 7.33-7.54 (7H, m), 7.80 (1H, d, J=7.6 Hz).

### EXAMPLE 215

N-(4-{[2-(2-Amino-1,3-thiazol-4-yl)ethyl]amino}phenyl)-4′-chloro-1,1′-biphenyl-2-carboxamide

The title compound was obtained from 2-({4-[(tert-butoxycarbonyl)(2-{2-[(tert-butoxycarbonyl)amino]-1,3-thiazol-4-yl}ethyl)amino]anilino}carbonyl)-4′-chloro-1,1′-biphenyl in the same manner as in Example 75 as a yellow foam.

¹H-NMR (CDCl₃): δ2.80 (2H, d, J=6.6 Hz), 3.36 (2H, d, J=6.6 Hz), 4.95 (2H, brs), 6.17 (1H, s), 6.51 (2H, d, J=8.9 Hz), 6.78 (1H, brs), 6.98 (2H, d, J=8.9 Hz), 7.37-7.55 (7H, m), 7.76-7.79 (1H, m).

ESI-MS(m/z): 449 (M+H)⁺

Preparation 64

A solution of 2-[(4-nitrophenoxy)methyl]-1,3-thiazole (0.382 g) in methanol (20 ml) was hydrogenated over 10% palladium on carbon at room temperature under atmospheric pressure of hydrogen for 2 hours. The reaction mixture was filtered through a pad of celite, and the filtrate was concentrated in vacuo to give 4-(1,3-thiazol-2-ylmethoxy) aniline (0.317 g). The product was used for the next step without further purification.

### EXAMPLE 216

To a solution of 4-(1,3-thiazol-2-ylmethoxy)aniline (0.237 g), 4′-(trifluoromethyl)-1,1′-biphenyl-2-carboxylic acid (0.306 g) and HOBT (0.171 g) in tetrahydrofran (15 ml) was added WSC.HCl, (0.242 g), followed by addition of triethylamine (0.21 ml) at room temperature. The reaction mixture was stirred at 50° C. for 18 hours and concentrated in vacuo. The residue was dissolved in ethyl acetate and water and extracted with ethyl acetate. The organic layer was washed with water and brine, dried over magnesium sulfate, filtered and concentrated in vacuo. The residue was purified by column chromatography on silica gel eluting with chloroform:methanol (39:1) to give N-[4-(1,3-thiazol-2-ylmethoxy)phenyl]-4′-(trifluoromethyl)-1,1′-biphenyl-2carboxamide (0.339 g) as a pale yellow soild.

¹H-NMR (CDCl₃): δ5.32 (2H, s), 6.89 (2H, d, J=8.9 Hz), 7.10 (2H, d, J=8.9 Hz), 7.35-7.79 (10H, m)

Preparation 65

To a solution of p-nitrophenol (0.768 g) in N,N-dimethylformamide (50 ml) was added cesium carbonate (2.569 g) at room temperature, and the mixture was stirred for 1 hour. N-{4-(Chloromethyl)-1,3-thiazol-2-yl}acetamide (1.002 g) was added to the reaction mixture, and the mixture was heated at 50° C. for 8hours. The reaction mixture was cooled to room temperature and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with hexane:ethyl acetate (1:1) to give N-{4-[(4-nitrophenoxy)methyl]-1,3-thiazol-2-yl}acetamide (0.597 g) as a pale yellow oil.

Preparation 66

A solution of N-{4-[(4-nitrophenoxy)methyl]-1,3-thiazol-2-yl}acetamide (0.259 g) in methanol (20 ml) was hydrogenated over 10% palladium on carbon at room temperature under atmospheric pressure of hydrogen for 2 hours. The reaction mixture was filtered through a pad of celite, and the filtlate was concentrated in vacuo to give N-{4-[(4-aminophenoxy)methyl]-1,3-thiazol-2-yl}acetamide (0.219 g). The product was used for the next step without further purification.

### EXAMPLE 217

To a solution of N-{4-[(4-aminophenoxy)methyl]-1,3-thiazol-2-yl}acetamide (0.219 g), 4′-(trifluoromethyl)-1,1′-biphenyl-2-carboxylic acid (0.221 g) and HOBT (0.123 g) in tetrahydrofuran (15 ml) was added WSC.HCl (0.175 g), followed by addition of triethylaminne (0.15 ml) at room temperature. The reaction mixture was stirred for 12 hours and concentrated in vacuo. The residue was dissolved in ethyl acetate and water, and extracted with ethyl acetate. The organic layer was washed with water and brine, dried over magnesium sulfate, filtered and concentrated in vacuo. The residue was purified by column chromatography on silica gel eluting with chloroform:methanol (39:1) to give N-(4-{[2-(acetylamino)-1,3-thiazol-4-yl]methoxy}phenyl)-4′-(trifluoromethyl)-1,1′-biphenyl-2-carboxamide (0.284 g) as a pale pink soild.

### EXAMPLE 218

To a solution of N-(4-{[2-(acetylamino)-1,3-thiazol-4-yl]methoxy}phenyl)-4′-(trifluoromethyl)-1,1′-biphenyl-2-carboxamide (0.263 g) in methanol (20 ml) and tetrahydrofuran (5 ml) was added conc. HCl (1 ml), and the mixture was refluxed for 6 hours. The reaction mixture was cooled to room temperature and evaporated in vacuo. The residue was dissolved in a mixture of ethyl acetate, tetrahydrofuran and saturated aqueous sodium hydrogencarbonate solution. The organic layer was washed with water and brine, dried over magnesium sulfate, filtered and concentrated in vacuo. The residue was purified by column chromatography on silica gel eluting with chloroform:methanol (39:1) to give N-{4-[(2-amino-1,3-thiazol-4-yl)methoxy]phenyl}-4′-(trifluoromethyl)-1,1′-biphenyl-2-carboxamide (0.167 g) as a white soild.

¹H-NMR (DMSO-d₆): δ4.79 (s, 2H), 6.59 (s, 1H), 6.91 (d, 2H, J=9.2 Hz), 7.02 (br s, 1H), 7.41 (d, 2H, J=8.9 Hz), 7.49-7.64 (m, 6H), 7.75 (d, 2H, J=7.9 Hz), 10.19 (s, 1H)

### EXAMPLE 219

To a solution of N-(4-hydroxyphenyl)-4′-(trifluoromethyl)-1,1′-biphenyl-2-carboxamide (120 mg) and 2-{2-[(tert-butoxycarbonyl)(methyl)amino]-1,3-thiazol-4-yl}ethyl 4-methylbenzenesulfonate (140 mg) in N,N-dimethylformamide (10 ml) was added potassium carbonate (100 mg) as a solid by one portion. The reaction mixture was heated to 50° C. and stirred for 12 hours under an argon atmosphere. The solvent was removed under reduced pressure, and then ethyl acetate (20 ml) and water (20 ml) were added. The separated organic layer was washed with brine, dried over magnesium sulfate and concentrated in vacuo. The residue was purified by column chromatography on silica gel eluting with hexane:ethyl acetate (4:1) to give tert-butyl methyl(4-{2-[4-({[4′-(trifluoromethyl)-1,1′-biphenyl-2-yl]carbonyl}amino)phenoxy]ethyl}-1,3-thiazol-2-yl)carbamate (58 mg) as colorless crystals.

¹H-NMR (CDCl₃): δ1.57 (9H, s), 3.09 (2H, t, J=6.6 Hz), 3.52 (3H, s), 4.23 (2H, t, J=6.6 Hz), 6.61 (1H, s), 6.80 (2H, d, J=8.9 Hz), 6.87 (1H, brs), 7.05 (2H, d, J=8.9 Hz), 7.40-7.80 (8H, m).

### EXAMPLE 220

To a solution of tert-butyl methyl(4-{2-[4-({[4′-(trifluoromethyl)-1,1′-biphenyl-2-yl]carbonyl}amino)phenoxy]ethyl}-1,3-thiazol-2-yl)carbamate (58 mg) in dichloromethane (4 ml) was added trifluoroacetic acid (0.30 g) at room temperature and the reaction mixture was stirred for 18 hours. The mixture was basified with 10% aqueous potassium carbonate solution and the separated organic layer was washed with brine, dried over magnesium sulfate and concentrated in vacuo to give N-(4-{2-[2-(methylamino)-1,3-thiazol-4-yl]ethoxy}phenyl)-4′-(trifluoromethyl)-1,1′-biphenyl-2-carboxamide (46 mg) as colorless crystals.

¹H-NM (CDCl₃): δ2.95 (3H, s), 2.99 (2H, t, J=6.9 Hz), 4.19 (2H, t, J=6.9 Hz), 6.22 (1H, s), 6.78-6.82 (3H, m), 7.05 (2H, d, J=8.9 Hz), 7.41-7.81 (8H, m).

ESI-MS (m/z): 498 (M+H)⁺

Preparation 67

2-(1,3-Thiazol-4-yl)ethyl 4-methylbenzenesulfonate

The title compound was obtained from 2-(1,3-thiazol-4-yl)ethanol in the same manner as in Preparation 37 as a yellow oil.

¹H-NMR (DMSO-d₆): δ2.44 (3H, s), 3.18 (2H, t, J=6.6 Hz), 4.37 (2H, t, J=6.6 Hz), 7.06 (1H, dd, J=1.0, 2.0 Hz), 7.29-7.73 (4H, AaBb), 8.67 (1H, d, J=2.0 Hz).

### EXAMPLE 221

N-{4-[2-(1,3-Thiazol-4-yl)ethoxy]phenyl}-4′-(trifluoromethyl)-1,1′-biphenyl-2-carboxamide

The title compound was obtained from N-(4-hydroxyphenyl)-4′-(trifluoromethyl)-1,1′-biphenyl-2-carboxamide and 2-(1,3-thiazol-4-yl)ethyl 4-methylbenzenesulfonate in the same manner as in Example 219 as a white solid.

¹H-NMR (CDCl₃): δ3.27 (2H, t, J=6.4 Hz), 4.27 (2H, t, J=6.6 Hz), 6.8-7.8 (14H, m) 8.03 (1H, s).

ESI-MS(m/z): 491 (M+Na)⁺, 469 (M+H)⁺

### EXAMPLE 222

To a solution of N-{4-[2-(4-aminophenyl)ethyl]-1,3-thiazol-2-yl}acetamide (0.323 g), 4′-(trifluoromethyl)-1,1′-biphenyl-2-carboxylic acid (0.329 g) and HOBT (0.201 g) in N,N-dimethylformamide (10 ml) was added WSC.HCl (0.285 g), followed by addition of triethylamine (0.26 ml) at room temperature. The reaction mixture was stirred at 50° C. for 15 hours, quenched with water and extracted with ethyl acetate. The organic layer was washed with brine, dried over magnesium sulfate, filtered and concentrated in vacuo. The residue was purified by column chromatography on silica gel eluting with hexane:ethyl acetate (2:1) to give N-(4-{2-[2-(acetylamino)-1,3-thiazol-4-yl]ethyl}phenyl)-4′-(trifluoromethyl)-1,1′-biphenyl-2-carboxamide (0.41 g) as a pale brown soild.

¹H-NMR (DMSO-d₆): δ2.23 (3H, s), 2.76-2.84(4H, m), 6.10 (1H, s), 7.45 (2H, d, J=8.9 Hz), 7.53-7.67 (9H, m), 7.78 (2H, d, J=8.2 Hz), 10.23 (1H, s), 10.27 (1H, s)

ESI-MS (m/z): 510 (M⁺+H)⁺

### EXAMPLE 223

To a solution of N-(4-{2-[2-(acetylamino)-1,3-thiazol-4-yl]ethyl}phenyl)-4′-(trifluoromethyl)-1,1′-biphenyl-2-carboxamide (0.410 g) in methanol (20 ml) was added conc. HCl (5 ml), and the mixture was refluxed for 6 hours. The reaction mixture was cooled to room temperature and evaporated in vacuo. The residue was dissolved in a mixture of ethyl acetate, tetrahydrofuran and saturated aqueous sodium hydrogencarbonate solution. The organic layer was washed with water and brine, dried over magnesium sulfate, filtered and concentrated in vacuo. The residue was purified by column chromatography on silica gel eluting with hexane:ethyl acetate (4:1) to give N-{4-[2-(2-amino-1,3-thiazol-4-yl)ethyl]phenyl}-4′-(trifluoromethyl)-1,1′-biphenyl-2-carboxamide (0.314 g) as a pale brown soild.

¹H-NMR (DMSO-d₆): δ2.75-2.82 (4H, m), 6.10 (1H, s), 7.43 (2H, d, J=8.9 Hz), 7.52-7.67 (9H, m), 7.76 (2H, d, J=8.2 Hz), 10.27 (1H, s)

ESI-MS(m/z): 468 (M+H)⁺

### EXAMPLE 224

N-{4-[2-(2-Acetylamino-1,3-thiazol-4-yl)vinyl]phenyl}-4′-(trifluoromethyl)-1,1′-biphenyl-2-carboxamide

The title compound was obtained from N-{4-[2-(4-aminophenyl)vinyl]-1,3-thiazol-2-yl}acetamide and 4′-(trifluoromethyl)-1,1′-biphenyl-2-carboxylic acid in the same manner as in Example 222 as pale brown soild.

¹H-NMR (DMSO-d₆): δ2.25 (3H, s), 6.54 (1H, s), 6.86 (1H, d, J=15.8 Hz), 7.05 (1H, d, J=15.8 Hz), 7.42 (2H, d, J=8.9 Hz), 7.53-7.67 (9H, m), 7.78 (2H, d, J=8.2 Hz), 10.28 (1H, s), 10.41 (1H, s)

ESI-MS(m/z): 508 (M+H)⁺

### EXAMPLE 225

N-{4-[2-(2-Amino-1,3-thiazol-4-yl)vinyl]phenyl}-4′-(trifluoromethyl)-1,1′-biphenyl-2-carboxamide

The title compound was obtained from N-{4-[2-(2-acetylamino-1,3-thiazol-4-yl)vinyl]phenyl}-4′-(trifluoromethyl)-1,1′-biphenyl-2-carboxamide in the same manner as in Example 223 as a pale brown soild.

¹H-NMR (DMSO-d₆): δ6.56 (1H, s), 6.85 (1H, d, J=15.8 Hz), 7.04 (1H, d, J=15.8 Hz), 7.41 (2H, d, J=8.9 Hz), 7.51-7.65 (9H, m), 7.75 (2H, d, J=8.2 Hz), 10.41 (1H, s)

ESI-MS(m/z): 466 (M+H)⁺

Preparation 68

A mixture of 1,2-difluoro-4-nitrobenzene (3.18 g), triethylamine (6.06 g) in N,N-dimethylformamide (30 ml) was stirred at 90-100° C. for 7 hours. The reaction mixture was poured into a mixture of ethyl acetate and water. The organic layer was washed with brine-and dried over magnesium sulfate. The solvent was evaporated in vacuo and the residue was chromatographed on silica gel eluting with ethyl acetate and n-hexane (5:5). The fraction was evaporated in vacuo and the residue was recrystallized from ethyl acetate and diisopropyl ether to give N-(2-fluoro-4-nitrophenyl)-N-methyl-N-[2-(2-pyridinyl)ethyl]amine (5.43 g).

¹H-NMR (DMSO-d₆): δ2.90-3.04 (5H, m), 3.82 (2H, t, J=7.37 Hz), 6.91 (1H, d, J=9.14 Hz), 6.97 (1H, d, J=1.93 Hz), 7.18-7.30 (2H, m), 7.64-7.72 (1H, m), 7.85-7.95 (2H, m), 8.48 (1H, d, J=4.79 Hz)

### EXAMPLE 226

A mixture of N-(2-fluoro-4-nitrophenyl)-N-methyl-N-[2-(2-pyridinyl)ethyl]amine (940 mg) in methanol (30 ml) was hydrogenated over 10% palladium on carbon (400 mg) under an atmospheric pressure of hydrogen at ambient temperature under stirring for 3 hours. After removal of the catalyst, the solvent was evaporated in vacuo and the residue was dissolved in tetrahydrofuran (30 ml) and triethylamine (696 mg). A solution of 4′-(trifluoromethyl)-1,1′-biphenyl-2-carbonyl chloride (971 mg) in tetrahydrofuran (,5 ml) was added to the above solution at ambient temperature under stirring. The resultant mixture was stirred at ambient temperature for 6 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (7:3). The fraction was evaporated in vacuo and the residue was recrystallized from ethyl acetate and diisopropyl ether to give N-(3-fluoro-4-{methyl[2-(2-pyridinyl)ethyl]amino}phenyl)-4′-(trifluoromethyl)-1,1′-biphenyl-2-carboxamide (0.9 g).

¹H-NMR (DMSO-d₆): δ2.77 (3H, s), 2.89 (2H, t, J=6.88 Hz), 3.43 (2H, t, J=6.88 Hz), 6.88-6.97 (1H, m), 7.15-7.25 (3H, m), 7.35-7.70(8H, m), 7.77 (2H, d, J=8.32 Hz), 8.46 (4H, d, J=4.14 Hz), 10.35 (1H, s)

Preparation 69

N-Methyl-N-(2-methyl-4-nitrophenyl)-N-[2-(2-pyridinyl)ethyl]amine was obtained from 2-fluoro-5-nitrotoluene and 2-(2-methylaminoethyl)pyridine in the same manner as in Preparation 68.

¹H-NMR (DMSO-d₆): δ2.35 (3H, s), 2.89 (3H, s), 2.99 (2H, t, J=7.00 Hz), 3.50 (2H, t, J=7.00 Hz), 7.04-7.25 (3H, m), 7.62-7.66 (1H, m), 7.95-7.98 (2H, m), 7.84-8.47 (1H, m)

### EXAMPLE 227

N-(3-Methyl-4-{methyl[2-(2-pyridinyl)ethyl]amino}phenyl)-4′-(trifluoromethyl)-1,1′-biphenyl-2-carboxamide was obtained from N-methyl-N-(2-methyl-4-nitrophenyl)-N-[2-(2-pyridinyl)ethyl]amine in the same manner as in Example 226.

¹H-NMR (DMSO-d₆); δ2.05 (3H, s), 2.62 (3H, s), 2.85 (2H, t, J=7.00 Hz), 3.17 (2H, t, J=7.00 Hz), 7.02 (1H, d, J=8.86 Hz), 7.17-7.32 (4H, m), 7.47-7.65 (7H, m), 7.74 (2H, d, J=8.34 Hz), 8.45 (1H, d, J=4.04 Hz), 10.16 (1H, s)

### EXAMPLE 228

A mixture of N-(4-fluoro-3-nitrophenyl)-4′-(trifluoromethyl)-1,1′-biphenyl-2-carboxamide (4.04 g), 2-(2-methylaminoethyl)pyridine (2.72 g) and triethylamine (3.03 g) in N,N-dimethylformamide (30 ml) was stirred at 60-65° C. for 4.5 hours. The reaction mixture was poured into a mixture of ethyl acetate and water. The organic layer was washed with brine and dried over magnesium sulfate. The solvent was evaporated in vacuo and the residue was chromatographed on silica gel eluting with ethyl acetate and n-hexane (5:5-7:3). The fraction was evaporated in vacuo and the residue was recrystallized from ethyl acetate and diisopropyl ether to give N-(4-{methyl[2-(2-pyridinyl)ethyl]amino}-3-nitrophenyl)-4′-(trifluoromethyl)-1,1′-biphenyl-2-carboxamide (4.43 g).

¹H-NMR (DMSO-d₆): δ2.94 (2H, t, J=6.72 Hz), 3.40 (3H, s), 3.44 (2H, t, J=6.72 Hz), 7.17-7.25 (3H, m), 7.51-7.70 (8H, m), 7.78 (2H, d, J=8.26 Hz), 8.04 (1H, d, J=2.98 Hz), 8.45 (1H, d, J=4.00 Hz), 10.58 (1H, s)

### EXAMPLE 229

A mixture of N-(4-{methyl[2-(2-pyridinyl)ethyl]amino}-3-nitrophenyl)-4′-(trifluoromethyl)-1,1′-biphenyl-2-carboxamide (2.6 g) in methanol (80 ml) was hydrogenated over 10% palladium on carbon (500 mg) under an atmospheric pressure of hydrogen at ambient temperature under stirring for 9 hours. After removal of the catalyst, the solvent was evaporated in vacuo to give N-(3-amino-4-{(methyl [2-(2-pyridinyl)ethyl]amino}phenyl)-4′-(trifluoromethyl)-1,1′-biphenyl-2-carboxamide (2.3 g). The crude N-(3-amino-4-{methyl[2-(2-pyridinyl)ethyl]amino}phenyl)-4′-(trifluoromethyl)-1,1′-biphenyl-2-carboxamide (1.3 g) was chromatographed on silica gel eluting with ethyl acetate and n-hexane (7:3-8:2). The fraction was evaporated in vacuo and the residue was recrystallized from ethyl acetate and dilsopropyl ether to give pure N-(3-amino-4-{methyl[2-(2-pyridinyl)ethyl]amino}phenyl)-4′-(trifluoromethyl)-1,1′-biphenyl-2-carboxamide (0.885 mg)

¹H-NMR (DMSO-d₆): δ2.56 (3H, s), 2.86 (2H, t, J=7.50 Hz), 3.11 (2H, t, J=7.50 Hz), 4.71 (2H, s), 6.68-6.69 (1H, m), 6.87 (1H, d J=8.46 Hz), 7.00 (1H, d, J=2.28 Hz), 7.22-7.26 (2H, m), 7.50-7.78 (7H, m), 7.76 (2H, d, J=8.32 Hz), 8.45 (1H, d, J=4.02 Hz), 10.07 (1H, s)

### EXAMPLE 230

A mixture of N-(3-amino-4-{methyl[2-(2-pyridinyl)ethyl]amino}phenyl)-4′-(trifluoromethyl)-1,1′-biphenyl-2-carboxamide (491 mg), acetyl chloride (157 mg) and triethylamine (303 mg) in tetrahydrofuran (20 ml) was stirred at ambient temperature for 5 hours. The reaction mixture was poured into a mixture of ethyl acetate and water. The organic layer was washed with brine and dried over magnesium sulfate. The solvent was evaporated in vacuo and the residue was chromatographed on silica gel eluting with ethyl acetate and n-hexane (7:3-10:0). The fraction was evaporated in vacuo and the residue was recrystallized from ethyl acetate and diisopropyl ether to give N-(3-(acetylamino)-4-{methyl[2-(2-pyridinyl)ethyl]amino}phenyl)-4′-(trifluoromethyl)-1,1′-biphenyl-2-carboxamide (396 mg).

¹H-NMR (DMSO-d₆): δ1.98 (3H, s), 2.58 (3H, s), 2.86 (2H, t, J=7.06 Hz), 3.18 (2H, t, J=7.06 Hz), 7.15-7.36 (4H, m), 7.40-7.74 (6H, m), 7.76 (2H, d, J=8.16 Hz), 8.21 (1H, s), 8.49 (1H, d, J=4.26 Hz), 8.81 (1H, s), 10.36 (1H, s)

Preparation 70

1-(5-Nitro-2-{[2-(2-pyridinyl)ethyl]amino}phenyl)ethanone was obtained from 2-chloro-5-nitroacetophenone and 2-(2-aminoethyl)pyridine in the same manner as in Preparation 68.

¹H-NMR (DMSO-d₆): δ2.63 (3H, s), 3.10 (2H, t, J=6.78 Hz), 3.72-3.81 (2H, m), 6.99 (1H, d, J=9.50 Hz), 7.23-7.29 (1H, m), 7.35 (1H, d, J=7.77 Hz), 7.70-7.78 (1H, m), 8.19 (1H, dd, J=2.50 Hz, 9.50 Hz), 8.52-8.55 (1H, m), 8.65 (1H, d, J=2.68 Hz), 9.66-9.68 (1H, m)

Preparation 71

A mixture of 1-(5-nitro-2-{[2-(2-pyridinyl)ethyl]amino}phenyl)ethanone (1.71 g) in methanol (50 ml) and tetrahydrofuran (50 ml) was hydrogenated over 10% palladium on carbon (600 mg) under an atmospheric pressure of hydrogen at ambient temperature under stirring for 6 hours. After removal of the catalyst, the solvent was evaporated in vacuo and the residue was washed with diisopropyl ether to give 1-(5-amino-2-{[2-(2-pyridinyl)ethyl]amino}phenyl)ethanone (1.51 g).

¹H-NMR (DMSO-d₆): δ2.42 (3H, s), 3.00 (2H, t, J=6.88 Hz), 3.43-3.53 (2H, m), 4.42 (2H, s), 6.65 (1H, d, J=8.87 Hz), 6.85 (1H, dd, J=2.60 Hz,8.87 Hz), 7.07 (1H, d, J=2.60 Hz), 7.19-7.32 (2H, m), 7.66-7.75 (1H, m), 8.16 (1H, t, J=5.55 Hz), 8.49-8.53 (1H, m)

### EXAMPLE 231

A mixture of 1-(5-amino-2-{[2-(2-pyridinyl)ethyl]amino}phenyl)ethanone (1.51 g), 4-′(trifluoromethyl)-1,1′-biphenyl-2-carboxylic acid (1.57 g), HOBT.H₂O (0.88 g) and WSC.HCl (1.24 g) in N,N-dimethylformamide (20 ml) was stirred at ambient temperature for 15 hours. The reaction mixture was poured into a mixture of ethyl acetate and water. The organic layer was washed with brine and dried over magnesium sulfate. The solvent was evaporated in vacuo and the residue was chromatographed on silica gel eluting with ethyl acetate and n-hexane (5:5-7:3). The fraction was evaporated in vacuo and the residue was recrystallized from ethyl acetate and diisopropyl ether to give N-(3-acetyl-4-{[2-(2-pyridinyl)ethyl]amino}phenyl)-4′-(trifluoromethyl)-1,1′-biphenyl2-carboxamide (1.99 g).

¹H-NMR (DMSO-d₆): δ2.41 (3H, s), 3.04 (2H, d, J=6.78 Hz), 3.53-3.60 (2H, m), 6.80 (1H, d, J=9.20 Hz), 7.20-7.27 (1H, m), 7.32 (1H, d, J=7.78Hz), 7.50-7.80 (10H, m), 7.92 (1H, d, J=2.38 Hz), 8.51-8.53 (10.01 (1H, s)

### EXAMPLE 232

A mixture of 3′-(trifluoromethyl)-1,1′-biphenyl-2-carboxylic acid (1.065 g), tert-butyl 4-aminophenyl[2-(2-pyridinyl)ethyl]carbamate (1.316 g) and HOBT.H₂O (594 mg) and WSC.HCl (840 mg) in N,N-diinethylformamide (30 ml) was stirred at ambient temperature for 15 hours. The reaction mixture was poured into a mixture of ethyl acetate and water. The organic layer was washed with brine and dried over magnesium sulfate and the solvent was evaporated in vacuo. A mixture of the residue and trifluoroacetic acid (10 ml) was stirred at ambient temperature for 2 hours. The reaction mixture was poured into a mixture of ethyl acetate and water and adjusted to pH 8.0 with 20% aqueous potassium carbonate solution. The organic layer was washed with brine and dried over magnesium sulfate. The solvent was evaporated in-vacuo and the residue was chromatographed on silica gel eluting with ethyl acetate and n-hexane (7:3-9:1). The fraction was evaporated in vacuo and the residue was recrystallized from ethyl acetate and diisopropyl ether to give N-(4-{[2-(2-pyridinyl)ethyl]amino}phenyl)-3′-(trifluoromethyl)-1,1′-biphenyl-2-carboxamide (1.14 g).

¹H-NMR (DMSO-d₆): δ2.96 (2H, d, J=7.37 Hz), 3.29-3.39 (2H, m), 5.54 (1H, t, J=5.75 Hz), 6.50 (2H, d, J=8.82 Hz), 7.15-7.32 (4H, m), 7.47-7.76 (9H, m), 8.51 (1H, d, J=3.99 Hz), 9.90 (1H, s)

### EXAMPLE 233

3′-Methoxy-N-(4-{[2-(2-pyridinyl)ethyl]amino}phenyl)-1,1′-biphenyl-2-carboxamide was obtained from 3′-methoxy-1,1′-biphenyl-2-carboxylic acid and tert-butyl 4-aminophenyl(2-(2-pyridinyl)ethyl)carbamate in the same manner as in Example 232.

¹H-NMR (DMSO-d₆): δ2.96 (2H, d, J=7.33 Hz), 3.28-3.35 (2H, m), 3.70 (3H, s), 5.52 (1H, t, J=5.78 Hz), 6.50 (2H, d, J=8.83 Hz), 6.56-6.60 (1H, m), 6.86-6.89 (2H, m), 7.01-7.52 (5H, m), 7.66-7.93 (1H, m), 8.51 (1H, d, J=4.80 Hz), 9.82 (1H, s)

### EXAMPLE 234

3′-Chloro-N-(4-{[2-(2-pyridinyl)ethyl]amino}phenyl)-1,1′-biphenyl-2-carboxamide was obtained from 3′-chloro-1,1′-biphenyl-2-carboxylic acid and tert-butyl 4-aminophenyl(2-(2-pyridinyl)ethyl)carbamate in the same manner as in Example 232.

¹H-NMR (DMSO-d₆): δ2.96 (2H, d, J=7.40 Hz), 3.29-3.38 (2H, m), 5.54 (1H, m), 6.52 (2H, d, J=8.82 Hz), 7.20 (2H, d, J=8.82 Hz), 7.24-6.74(11H, m), 8.49-8.52 (1H, m), 9.56 (1H, s)

Preparation 72

A mixture of tert-butyl 6-(hydroxymethyl)-2-pyridinylcarbamate (0.66 g) and potassium tert-butoxide (396 mg) in tetrahydrofuran (20 ml) was stirred at ambient temperature for an hour. 2-Chloro-5-nitropyridine (467 mg) was added to the above solution and the resultant mixture was stirred at ambient temperature for 20 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (8:2). The fraction was evaporated in vacuo to give tert-butyl 6-{[(5-nitro-2-pyridinyl)oxy]methyl}-2-pyridinylcarbamate (0.37 g).

¹H-NMR (DO-d₆): δ1.56 (9H, s), 5.44 (2H, s), 7.08-7.13 (1H, m), 7.16 (1H, d, J=9.13 Hz), 7.71-7.87 (2H, m), 8.52 (1H, dd, J=2.90 Hz,9.13 Hz), 9.07 (1H, d, J=2.90 Hz), 9.81 (1H, s)

### EXAMPLE 235

A mixture of tert-butyl 6-{[(5-nitro-2-pyridinyl)oxy]methyl}-2-pyridinylcarbamate (370 mg), iron powder (320 mg) and ammonium chloride (36 mg) in ethanol (30 ml) and water (6 ml) was refluxed under stirring for 2.5 hours. After removal of the insoluble materials by filtration, the solvent was evaporated in vacuo and, the residue was dissolved in ethyl acetate and water. The organic layer was washed with brine and dried over magnesium sulfate. The solvent was evaporated in vacuo and the residue was dissolved in tetrahydrofuran (20 ml) and triethylamine (216 mg). To the above solution was added a solution of 4′-(trifluoromethyl)-1,1′-biphenyl-2-carbonyl chloride (304 mg) in tetrahydrofuran (10 ml) at ambient temperature and stirred for 2 hours: The resultant mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (6:4). The fraction was evaporated in vacuo to give 2-({6-({6-[(tert-butoxycarbonyl)amino]-2-pyridinyl}methoxy)-3-pyridinyl]amino}carbonyl)-4′-(trifluoromethyl)-1,1′-biphenyl (0.64 g).

¹H-NMR (DMSO-d₆): δ1.57 (9H, s), 5.26 (2H, s), 6.91 (1H, d, J=8.92 Hz), 7.00-7.08 (1H, m), 7.44-7.99 (11H, m), 8.54 (1H, d, J=2.62 Hz), 9.79 (1H, s), 10.40 (1H, s)

### EXAMPLE 236

A mixture of 2-({6-({6-](tert-butoxycarbonyl)amino]-2-pyridinyl}methoxy) -3-pyridinyl]amino}carbonyl)-4′-(trifluoromethyl)-1,1′-biphenyl (540 mg), anisole (413 mg) and trifluoroacetic acid (1.07 g) in dichlorometane (10 ml) was stirred at ambient temperature for 2 hours. The reaction mixture was evaporated in vacuo and the residue was dissolved in a mixture of ethyl acetate and water and adjusted to pH 9.0 with 5% aqueous potassium carbonate solution. The organic layer was washed with brine and dried over magnesium sulfate. The solvent was evaporated in vacuo and the residue was chromatographed on silica gel eluting with ethyl acetate and n-hexane (6:4-9:1). The fraction was evaporated and the residue was recrystallized from ethyl acetate and diisopropyl ether to give N-{6-[(6-amino-2-pyridinyl)methoxy]-3-pyridinyl}-4′-(trifluoromethyl)-1,1′-biphenyl-2-carboxamide (71 mg).

¹H-NMR (DMSO-d₆): δ5.12 (2H, s), 5.96 (2H, s), 6.35 (1H, d, J=8.14 Hz), 6.50 (1H, d, J=7.12 Hz), 6.87 (1H, d, J=8.86 Hz), 7.30-7.37 (1H, m), 7.51-7.86(9H, m), 8.25 (1H, d, J=2.44 Hz), 10.38 (1H, s)

### EXAMPLE 237

A mixture of N-{6-[(6-amino-2-pyridinyl)methoxy]-3-pyridinyl}-4′-(trifluoromethyl)-1,1′-biphenyl-2-carboxamide (300 mg) and acetic anhydride (1 ml) in ethyl acetate (20 ml) was refluxed under stirring for 5 hours. The reaction mixture was poured into a mixture of ethyl acetate and water and adjusted to pH 9.0 with 5% aqueous potassium carbonate solution and stirred at ambient temperature for 0.5 hour. The organic layer was washed with brine and dried over magnesium sulfate. The solvent was evaporated in vacuo and the residue was chromatographed on silica gel eluting with ethyl acetate and n-hexane (5:5-7:3). The fraction was evaporated and the residue was recrystallized from ethyl acetate and diisopropyl ether to give N-(6-{[6-(acetylamino)-2-pyridinyl]methoxy}-3-pyridinyl)-4′-(trifluoromethyl)-1,1′-biphenyl-2-carboxamide (180 mg).

¹H-NMR (DMSO-d₆): δ2.09 (3H, s), 5.29 (2H, s), 6.90 (1H, d, J=8.84 Hz), 7.12 (1H, d, J=7.38 Hz), 7.51-7.88 (9H, m), 8.01 (2H, d, J=8.20 Hz), 8.25 (1H, d, J=2.36 Hz), 10.40 (1H, s), 10.56 (1H, s)

Preparation 73

A mixture of 2-chloro-5-nitropyridine (3.13 g), 2-(2-aminoethyl)pyridine (2.93 g) and triethylamine (3.03 g) in N,N-dimethylformamide (20 ml) was stirred at ambient temperature for 20 hours. The reaction mixture was poured into water and the precipitate was collected by filtration. The precipitate was dissolved in a mixture of ethyl acetate and tetrahydrofuran and washed with brine and dried over magnesium sulfate. The solvent was concentrated in vacuo and the precipitate was collected by filtration to give 5-nitro-N-[2-(2-pyridinyl)ethyl]-2-pyridinamine (4.42 g).

¹H-NMR (DMSO-d₆): δ3.04 (2H, t, J=7.39 Hz), 3.98-4.09 (2H, m), 6.56 (1H, d, J=9.38 Hz), 7.20-7.27 (2H, m), 7.67-7.75 (1H, m), 8.09 (1H, d, J=7.55 Hz), 8.20-8.25 (1H, m), 8.51-8.53 (1H, m), 8.93 (1H, d, J=2.72 Hz).

### EXAMPLE 238

A mixture of 5-nitro-N-[2-(2-pyridinyl)ethyl]-2-pyridinamine (733 mg) in methanol (30 ml) and tetrahydrofuran (20. ml) was hydrogenated over 10% palladium on carbon (300 mg) under an atmospheric pressure of hydrogen at ambient temperature under stirring for 3 hours. After removal of the catalyst, the solvent was evaporated in vacuo. The residue and 4′-(trifluoromethoxy)-1,1′-biphenyl-2-carboxylic acid (846 mg), HOBT.H₂O (446 mg) and WSC.HCl (630 mg) in N,N-dimethylformamide (20 ml) was stirred at ambient temperature for 15 hours. The reaction mixture was poured into a mixture of ethyl acetate and water. The organic layer was washed with 5% aqueous potassium carbonate solution and brine and dried over magesium sulfate. The solvent was evaporated in vacuo and the residue was chromatographed on silica gel eluting with ethyl acetate and n-hexane (7:3-10:0). The fraction was evaporated in vacuo and the residue was recrystallized from ethyl acetate and diisopropyl ether to give N-{6-[2-(2-pyridinyl)ethyl]amino-3-pyridinyl)-4′-(trifluoromethoxy)-1,1′-biphenyl-2-carboxamide (771 mg).

¹H-NMR (DMSO-d₆): δ2.96 (2H, t, J=7.45 Hz), 3.50-3.60 (2H, m), 6.41 (1H, d, J=9.02 Hz), 6.43-6.49 (1H, m), 7.20-7.24 (1H, m), 7.26 (1H, d, J=8.10 Hz), 7.39-7.69 (10H, m), 8.03 (1H, d, J=2.45 Hz), 8.50 (1H, d, J=5.10 Hz), 9.91 (1H, s)

Preparation 74

A mixture of 5-nitro-N-[2-(2-pyridinyl)ethyl]-2-pyridinamine (710 mg) in methanol (40 ml) and tetrahydrofuran (10. ml) was hydrogenated over 10% palladium on carbon (230 mg) under an atmospheric pressure of hydrogen at ambient temperature under stirring for 3 hours. After removal of the catalyst, the solvent was evaporated in vacuo to give N²-[2-(2-pyridinyl)ethyl]-2,5-pyridinediamine (621 mg).

### EXAMPLE 239

A solution of 4′-(trifluoromethyl)-1,1′-biphenyl-2-carbonyl chloride (826 mg) in tetrahydrofuran (5 ml) was added to a solution of N²-[2-(2-pyridinyl)ethyl]-2,5-pyridinediamine (621 mg) and triethylamine (586 mg) in tetrahydrofuran (20 ml) at ambient temperature and stirred at ambient temperature for 4 hours. The reactin mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (7:3-10:0). The fraction was evaporated in vacuo and the residue was recrystallized from ethyl acetate and diisopropyl ether to give N-{6-[2-(2-pyridinyl)ethyl]amino-3-pyridinyl}-4′-(trifluoromethyl)-1,1′-biphenyl-2-carboxamide (601 mg).

¹H-NMR (DMSO-d₆): δ3.96 (2H, t, J=7.42 Hz), 3.44-3.60 (2H, m), 6.42 (1H, d, J=9.00 Hz), 6.47-6.50 (1H, m), 7.18-7.28 (2H, m), 7.44-7.73 (8H, m), 7.78 (2H, d, J=8.32 Hz), 8.05 (1H, d, J=2.42 Hz), 8.49 (1H, d, J=4.04 Hz), 10.01 (1H, s)

Preparation 75

3-Methyl-5-nitro-N-[2-(2-pyridinyl)ethyl]-2-pyridinamine was obtained from 2-chloro-3-methyl-5-nitropyridine and 2-(2-aminoethyl)pyridine in the same manner as in Preparation 73.

¹H-NMR (DMSO-d₆): δ2.12 (3H, s), 3.06 (2H, t, J=7.76 Hz), 3.79-4.05 (2H, m), 7.19-7.29 (2H, m), 7.55 (1H, t, J=5.59 Hz), 7.67-7.75 (1H, m), 8.00-8.02 (1H, m), 8.50 (1H, m), 8.84 (1H, d, J=2.69 Hz)

### EXAMPLE 240

N-(5-Methyl-6-{[2-(2-pyridinyl)ethyl]amino}-3-pyridinyl)-4′-(trifluoromethyl)-1,1′-biphenyl-2-carboxamide was obtained from 3-methyl-5-nitro-N-[2-(2-pyridinyl)ethyl]-2-pyridinamine in the same manner as in Example 238.

¹H-NMR (DMSO-d₆): δ2.03 (3H, s), 3.38-3.53 (4H, m), 6.82 (1H, d, J=9.10 Hz), 7.50-7.75 (10H, m), 7.72 (2H, d, J=8.26 Hz), 8.22 (1H, d, J=2.52 Hz), 10.19 (1H, s)

Preparation 76

4-Methyl-5-nitro-N-[2-(2-pyridinyl)ethyl]-2-pyridinamine was obtained from 2-chloro-4-methyl-5-nitropyridine and 2-(2-aminoethyl)pyridine in the same manner as in Preparation 73.

¹H-NMR (DMSO-d₆): δ2.50 (3H, s), 3.01 (2H, t, J=7.26 Hz), 3.68-3.77 (2H, m), 6.37 (1H, s), 7.19-7.30 (2H, m), 7.66-7.75 (1H, m), 7.90-7.96 (1H, m), 8.51 (1H, d, J=4.81 Hz), 8.83 (1H, s)

### EXAMPLE 241

A mixture of 4-methyl-5-nitro-N-[2-(2-pyridinyl)ethyl]-2-pyridinamine (517 mg) in methanol (30 ml) and tetrahydrofuran (20 ml) was hydrogenated over 10% palladium on carbon (300 mg) under an atmospheric pressure of hydrogen at ambient temperature under stirring for 3 hours. After removal of the catalyst and the solvent was evaporated in vacuo. The residue -was dissolved in tetrahydrofuran (20 ml) and triethylamine (404 mg) and to an above solution was added to a 4′-(trifluoronethyl)-1,1′-biphenyl-2-carbonylchloride (570 mg) in tetrahydrofuran (5 ml) at ambient temperature under stirring. The resultant mixture was stirred at ambient temperature for 15 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (7:3-10:0). The fraction was evaporated in vacuo and the residue was recrystallized from ethyl acetate and diisopropyl ether to give N-(4-methyl-6-{[2-(2-pyridinyl)ethyl]amino}-3-pyridinyl)-4′-(trifluoromethyl)-1,1′-biphenyl-2-carboxamide (311 mg).

¹H-NMR (DMSO-d₆): δ1.85 (3H, s), 2.96 (2H, t, J=7.30 Hz), 3.50-3.60 (2H, m), 6.28 (1H, s), 6.43 (1H, t, J=5.62 Hz), 7.20-7.28 (2H, m), 7.51-7.72 (8H, m), 7.82 (2H, d, J=8.26 Hz), 8.50 (1H, d, J=4.00 Hz), 9.53 (1H, s)

APCI-MS (m/z): 477 (M+H)⁺

Preparation 77

A mixture of 5-nitro-N-[2-(2-pyridinyl)ethyl]-2-pyridinamine (1.22 g) and N-chlorosuccinimide (835 mg) in dichloromethane (20 ml) was stirred at 50° C. for 7 hours. The reaction mixture was poured into a mixture of ethyl acetate and water. The organic layer was washed with brine and dried over magnesium sulfate. The solvent was evaporated in vacuo and the residue was chromatographed on silica gel eluting with ethyl acetate and n-hexane (6:4-7:3). The fraction was evaporated in vacuo to give 3-chloro-5-nitro-N-[2-(2-pyridinyl)ethyl]-2-pyridinamine (0.71 g).

¹H-NMR (DMSO-d₆): δ3.34 (2H, t, J=6.28 Hz), 3.90-3.99 (2H, m), 7.82-7.84 (2H, m), 8.14 (1H, t, J=5.63 Hz), 8.34-8.41 (2H, m), 8.76-8.81 (2H, m)

### EXAMPLE 242

A mixture of 3-chloro-5-nitro-N-[2-(2-pyridinyl)ethyl]-2-pyridinamine (418 mg), iron powder (450 mg) and ammonium chloride (51 mg) in ethanol (30 ml) and water (6 ml) was refluxed under stirring for 2.5 hours. After removal of the insoluble materials by filtration and the solvent was evaporated in vacuo and the residue was dissolved in ethyl acetate and water. The organic layer was washed with brine and dried over magnesium sulfate. The solvent was evaporated in vacuo and the residue was dissolved in tetrahydrofuran (20 ml) and triethylamine (303 mg). To an above solution was added a solution of 4′-(trifluoromethyl)-1,1′-biphenyl-2-carbonyl chloride (427 mg) in tetrahydrofuran (5 ml) at ambient temperature and stirred for 3 hours. The resultant mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (7:3). The fraction was evaporated in vacuo and the residue was recrystallized from ethyl acetate and diisopropyl ether to give N-(5-chloro-6-{[2-(2-pyridinyl)ethyl]amino}-3-pyridinyl)-4′-(trifluoromethyl)-1,1′-biphenyl-2-carboxamide (425 mg).

¹H-NMR (DMSO-d₆): δ3.01 (2H, t, J=7.38 Hz), 3.60-3.73 (2H, m), 6.50 (1H, t, J=5.60 Hz), 7.220-7.28 (2H, m), 7.54-7.80 (10H, m), 8.08 (1H, d, J=2.24 Hz), 8.50 (1H, d, J=4.00 Hz), 10.23 (1H, s)

APCI-MS (m/z): 497 (M+H)⁺

Preparation 78

N-Methyl-5-nitro-N-[2-(2-pyridinyl)ethyl]-2-pyridinamine was obtained from 2-chloro-5-nitropyridine and 2-(2-methylaminoethyl)pyridine in the same manner as in Preparation 73.

¹H-NMR (DMSO-d₆): δ3.08 (2H, t, J=7.12 Hz), 3.19 (3H, s), 4.02 (2H, t, J=7.12 Hz), 6.72 (1H, d, J=9.58 Hz), 7.19-7.31 (2H, m), 7.65-7.73 (1H, m), 7.82 (1H, dd, J=2.86 Hz, 9.58 Hz), 8.56 (1H, m), 8.94 (1H, d, J=2.73 Hz)

Preparation 79

N²-Methyl-N²-[2-(2-pyridinyl)ethyl]-2,5-pyridinediamine was obtained from N-Methyl-5-nitro-N-[2-(2-pyridinyl)ethyl]-2-pyridinamine in the same manner as in Preparation 74.

### EXAMPLE 243

N-(6-{Methyl[2-(2-pyridinyl)ethyl]amino}-3-pyridinyl)-4′-(trifluoromethyl)-1,1′-biphenyl-2-carboxamide was obtained from N²-methyl-N²-[2-(2-pyridinyl)ethyl]-2,5-pyridinediamine in the same manner as in Example 239.

¹H-NMR (DMSO-d₆): δ2.90 (3H, s), 2.94 (2H, t, J=6.98 Hz), 3.82 (2H, t, J=6.98 Hz), 7.78 (2H, d, J=8.34 Hz), 8.16 (1H, d, J=2.52 Hz), 8.50 (1H, d, J=4.66 Hz), 10.10 (1H, s)

Preparation 80

2-Chloro-5-nitropyridine (4.76 g) was added portionwise to a solution of 2-hydroxyethylpyridine (4.43 g) and potassium tert-butoxide (4.04 g) in tetrahydrofuran (60 ml) was stirred at a temperature between 5 to 20° C. under ice-cooling and the resultant mixture was stirred at ambient temperature for 3 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (5:5). The fraction was concentrated in vacuo and the precipitate was collected by filtration to give 5-nitro-2-[2-(2-pyridinyl)ethoxy]pyridine (2.42 g).

¹H-NMR (DMSO-d₆): δ3.24 (2H, t, J=6.68 Hz), 4.80 (2H, t, J=6.68 Hz), 6.98 (1H, d, J=9.16 Hz), 7.24-7.28 (1H, m), 7.35 (1H, d, J=7.78 Hz), 7.69-7.77(1H, m), 8.42-8.52 (2H, m), 9.09 (1H, d, J=2.86 Hz)

### EXAMPLE 244

A mixture of 5-nitro-2-[2-(2-pyridinyl)ethoxy]pyridine (490 mg), iron powder (600 mg) and ammonium chloride (68 mg) in ethanol (30 ml) and water (6 ml) was refluxed under stirring for 2.5 hours. After removal of the insoluble materials by filtration, the solvent was evaporated in vacuo and the residue was dissolved in ethyl acetate and water. The organic layer was washed with brine and dried over magnesium sulfate. The solvent was evaporated in vacuo. The residue and 4′-(trifluoromethoxy)-1,1′-biphenyl-2-carboxylic acid (565 mg), HOBT.H₂O (297 mg) and WSC.HCl (420 mg) in N,N-dimethylformamide (20 ml) was stirred at ambient temperature for 15 hours. The reaction mixture was poured into a mixture-of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (5:5-7:3). The fraction was evaporated in vacuo and the residue was recrystallized from ethyl acetate and diisopropyl ether to give N-{6-[2-(2-pyridinyl)ethoxy]-3-pyridinyl}-4′-(trifluoromethoxy)-1,1′-biphenyl-2-carboxamide (488 mg).

¹H-NMR (DMSO-d₆): δ3.17 (2H, t, J=6.74 Hz), 4.58 (2H, t, J=6.74 Hz), 6.71 (1H, d, J=8.86 Hz), 7.20-7.78 (12H, m), 8.25 (1H, d, J=2.46 Hz), 8.50 (1H, d, J=4.00 Hz), 10.26 (1H, s)

### EXAMPLE 245

N-{6-[(2-Pyridinylmethyl)amino]-3-pyridinyl)-4′-(trifluoromethyl)-1,1′-biphenyl-2-carboxamide was obtained from N²-(2-pyridinyl)methyl-2,5-pyridinediamine in the same manner as in Example 239.

¹H-NMR (DMSO-d₆): δ4.52 (2H, d, J=6.04 Hz), 6.52 (1H, d, J=8.88 Hz), 7.04-7.21 (1H, m), 7.22-7.30 (2H, m), 7.48-7.79 (10H, m), 8.00 (1H, d, J=2.40 Hz), 8.49 (1H, d, J=4.00 Hz), 10.02 (1H, s)

Preparation 81

A mixture of 5-nitro-2-[2-(2-pyridinyl)ethoxy]pyridine (736 mg), iron powder (900 mg) and ammonium chloride (101 mg) in ethanol (40 ml) and water (8 ml) was refluxed under stirring for 2.5 hours. After removal of the insoluble materials by filtration, the solvent was evaporated in vacuo and the residue was dissolved in ethyl acetate and water. The .organic layer was washed with brine and dried over magnesium sulfate. The solvent was evaporated in vacuo to give 6-[2-(2-pyridinyl)ethoxy]-3-pyridinamine (664 mg).

### EXAMPLE 246

A solution of 4′-(trifluoromethyl)-1,1′-biphenyl-2-carbonyl chloride (854 mg) in tetrahydrofuran (5 ml) was added to a solution of 6-[2-(2-pyridinyl)ethoxy]-3-pyridinamine (665 mg) and triethylamine (606 mg) in tetrahydrofuran (20 ml) at ambient temperature and stirred at ambient temperature for 4 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (5:5-6:4). The fraction was evaporated in vacuo and the residue was recrystallized from ethyl acetate and diisopropyl ether to give N-{6-[2-(2-pyridinyl)ethoxy]-3-pyridinyl}-4′-(trifluoromethyl)-1,1′-biphenyl-2-carboxamide (723 mg).

¹H-NMR (DMSO-d₆): δ3.17 (2H, t, J=6.76 Hz), 4.59 (2H, t, J=6.76 Hz), 7.51-7.82 (10H, m), 8.29 (1H, d, J=2.48 Hz), 8.49-8.52 (1H, m), 10.37 (1H, s)

Preparation 82

5-Nitro-2-[3-(2-pyridinyl)propoxylpyridine was obtained from 2-chloro-5-nitropyridine and 2-pyridinepropanol in the same manner as in Preparation 80.

¹H-NMR (DMSO-d₆): δ2.10-2.21 (2H, m), 2.89 (2H, t, J=7.20 Hz), 4.44 (2H, t, J=6.54 Hz), 7.02 (2H, d, J=8.88 Hz), 7.17-7.24 (1H, m), 7.61-7.74 (1H, m), 8.44-8.50 (2H, m), 9.07 (1H, d, J=2.56 Hz)

Preparation 83

A mixture of 5-nitro-2-[3-(2-pyridinyl)propoxy]pyridine (778 mg), iron powder (900 mg) and ammonium chloride (101 mg) in ethanol (40 ml) and water (8 ml) was refluxed under stirring for 2.5 hours. After removal of the insoluble materials by filtration, the solvent was evaporated in vacuo and the residue was dissolved in ethyl acetate and water. The organic layer was washed with brine and dried over magnesium sulfate. The solvent was evaporated in vacuo to give 6-[3-(2-pyridinyl)propoxy)-3-pyridinamine (688 mg).

¹H-NMR (DMSO-d₆): δ1.92-2.14 (2H, m), 2.85 (2H, t, J=7.27 Hz), 4.20 (2H, t, J=7.27 Hz), 4.74 (2H, s), 6.54 (1H, d, J=8.30 Hz), 7.01 (1H, dd, J=2.91 Hz, 6.66 Hz), 7.18-7.28 (2H, m), 8.10 (1H, d, J=2.96 Hz), 8.46-8.49 (1H, m), 8.52 (1H, d, J=4.80 Hz)

### EXAMPLE 247

N-{6-[3-(2-Pyridinyl)propoxy]-3-pyridinyl}-4′-(trifluoromethyl)-1,1′-biphenyl-2-carboxamide was obtained from 6-[3-(2-pyridinyl)propoxyl-3-pyridinamine in the same manner as in Example 246.

¹H-NMR (DMSO-d₆): δ1.99-2.17 (2H, m), 2.86 (2H, t, J=-6.54 Hz), 4.22 (2H, t, J=6.54 Hz), 6.76 (1H, d, J=8.86 Hz), 7.16-7.28 (2H, m), 7.51-7.82 (10 H, m), 8.23 (1H, d, J=2.58 Hz), 8.46-8.48 (1H, m), 10.35 (1H, s)

### EXAMPLE 248

N-[6-(2-Pyridinylmethoxy)-3-pyridinyl]-4′-(trifluoromethyl)-1,1′-biphenyl-2-carboxamide was obtained from 6-(2-pyridinyl) methoxy-3-pyridinamine in the same manner as in Example 246.

¹H-NMR (DMSO-d₆): δ5.39 (2H, s), 6.92 (1H, d, J=8.86 Hz), 7.29-7.90 (12H, m), 8.27 (1H, d, J=2.42 Hz), 8.55 (1H, d, J=4.10 Hz), 10.41 (1H, s)

Preparation 84

A mixture of 2-ethynylpyridine (2.06 g), 2-chloro-5-nitropyridine (3.17 g), potassium acetate (2.94 g) and tetrakis(triphenylphosphine)palladium(O) (2.31 g) in N,N-dimethylformamide (40 ml) was stirred at 100° C. for 4 hours. The reaction mixture was poured into a mixture of ethyl acetate and water. The separated organic layer was washed with 5% aqueous potassium carbonate solution and brine, and dried over magnesium sulfate. The solvent was evaporated in vacuo and the residue was chromatographed on silica gel eluting with ethyl acetate and n-hexane (5:5). The fraction was evaporated in vacuo. The residue was triturated with diisopropyl ether and the powder was collected by filtration to give 5-nitro-2-(2-pyridinylethynyl)pyridine (0.75 g).

¹H-NMR (DMSO-d₆): δ7.31-7.41 (1H, m), 7.72 (1H, d, J=7.77 Hz), 7.81-7.99 (2H, m), 8.59-8.67 (2H, m), 9.38 (1H, d, J=2.64 Hz)

Preparation 85

A mixture of 5-nitro-2-(2-pyridinylethynyl)pyridine (451 mg) in methanol (40 ml) and tetrahydrofuran (20 ml) was hydrogenated over 10% palladium on carbon (200 mg) under an atmospheric pressure of hydrogen at ambient temperature under stirring for 4 hours. After removal of the catalyst, the solvent was evaporated in vacuo to give 6-[2-(2-pyridinyl)ethyl]-3-pyridinamine (0.4 g).

¹H-NMR (DMSO-d₆): δ2.88-3.16 (4H, m), 5.26 (2H, s), 6.78-6.89 (2H, m), 7.14-7.22 (2H, m), 7.60-7.79 (1H, m), 7.85 (1H, s), 7.48 (1H, d, J=3.10 Hz)

### EXAMPLE 249

A solution of 4′-(trifluoromethyl)-1,1′-biphenyl-2-carbonyl chloride (570 mg) in tetrahydrofuran (5 ml) was added to a mixture of 6-[2-(2-pyridinyl)ethyl]-3-pyridinamine (399 mg) and triethylamine (404 mg) in tetrahydrofuran (20 ml) at ambient temperature. The mixture was stirred at ambient temperature for 2 hours. The reaction mixture was poured into a mixture of ethyl acetate and water. The organic layer was washed with 5% hydrochloric acid and 5% aqueous potassium carbonate solution and brine and dried over magnesium sulfate. The solvent was evaporated in vacuo and the residue was chromatographed on silica gel eluting with ethyl acetate and n-hexane (5:5). The fraction was evaporated and the residue was collected by filtration to give N-{6-[2-(2-pyridinyl)ethyl]-3-pyridinyl}-4′-(trifluoromethyl)-1,1′-biphenyl-2-carboxamide (0.38 g).

¹H-NMR (DMSO-d₆): δ3.11-3.66 (4H, m), 6.70 (1H, d, J=9.10 Hz), 7.42-7.83 (13H, m), 8.19 (1H, d, J=2.42 Hz), 10.18 (1H, s)

Preparation 86

A solution of 4′-(trifluororaethyl)-1,1′-biphenyl-2-carbonyl chloride (2.85 g) in tetrahydrofuran (5 ml) was added to a mixture of ethyl 6-aminonicotinate (1.66 g) and triethylamine (2.02 g) in tetrahydrofuran (40 ml) at ambient temperature. The mixture was stirred at-ambient temperature for 5 hours. The reaction mixture was poured into a mixture of ethyl acetate and water and organic layer was washed successively with 10% hydrochloric acid, 5% aqueous potassium carbonate solution and brine and dried over magnesium sulfate. The solvent was evaporated in vacuo and the residue was recrystallized from ethyl acetate and diisopropyl ether to give ethyl 6-({[4′-(trifluoromethyl)-1,1′-biphenyl-2-yl]carbonyl}amino)nicotinate (1.483 g).

¹H-NMR (DMSO-d₆): δ1.29 (3H, t, J=7.10 Hz), 4.30 (2H, q, J=7.10 Hz), 7.24-7.30 (2H, m), 7.38 (2H, d, J=7.46 Hz), 7.49-7.56 (2H, m), 7.73-7.84 (4H, m), 8.40 (1H, dd, J=2.30 Hz, 8.40 Hz), 8.80 (1H, d, J=1.76 Hz)

### EXAMPLE 250

A mixture of ethyl 6-({[4′-(trifluoromethyl)-1,1′-biphenyl-2-yl]carbonyl}amino)nicotinate (622 mg) and 2(aminomethyl)pyridine (491 mg) in N,N-dimethylformamide (2 ml) was stirred at ambient temperature for 48 hours. The reaction mixture was dissolved in a mixture of ethyl acetate and water and adjusted to pH 1.0 with 10% hydrochloric acid. The aqueous layer was adjusted to pH 8.5 with 5% aqueous potassium carbonate solution and extracted with ethyl acetate. The organic layer was washed with brine and dried over magnesium sulfate. The solvent was evaporated in vacuo and the residue was recrystallized from ethyl acetate and diisopropyl ether to give N-(2-pyridinylmethyl)-6-({[4′-(trifluoromethyl)-1,1′-biphenyl-2-yl]carbonyl}amino)nicotinamide (154 mg).

¹H-NMR (DMSO-d₆): δ4.39 (2H, d, J=6.00 Hz), 6.96 (1H, d, J=7.83 Hz), 7.21-7.42 (1H, m), 7.43-7.91 (10H, m), 7.69 (2H, d, J=8.46 Hz), 8.46 (1H, d, J=4.13 Hz), 8.86-8.92 (1H, m)

### EXAMPLE 251

N-{4-[2-(2-Pyridinyl)ethoxylphenyl}-3′-(trifluoromethyl)-1,1′-biphenyl-2-carboxamide was obtained from 5-nitro-2-[2-(2-pyridinyl)ethoxy]pyridine and 3′-(trifluoromethyl)-1,1′-biphenyl2-carboxylic acid in the same manner as in Example 244.

¹H-NMR (DMSO-d₆): δ3.16 (2H, d, J=6.60 Hz), 4.30 (2H, t, J=6.60 Hz), 6.84 (2H, d, J=9.00 Hz), 7.20-7.40 (4H, m), 7.50−6.75(9H, m), 8.51 (1H, d, J=4.02 Hz), 10.15 (1H, s)

Preparation 87

A solution of 2-hydroxyethylpyridine (8.6 g) and potassium. tert-butoxide (7.85 g) in tetrahydrofuran (80 ml) was stirred at ambient temperature for 2 hours. 1-Fluoro-4-nitrobenzene (7.0 g) was added to the above mixture and the resultant mixture was stirred at ambient temperature for 3 hours. The reaction mixture was poured into a mixture of ethyl acetate and water. The organic layer was washed with brine and dried over magnesium sulfate. The solvent was evaporated in vacuo and the residue was chromatographed on silica gel eluting with ethyl acetate and n-hexane (4:6-6:4). The fraction was evaporated in vacuo to give 2-[2-(4-nitrophenoxy)ethyl]pyridine (4.47 g).

¹H-NMR (DMSO-d₆): δ3.24 (2H, t, J=6.62 Hz), 4.53 (2H, t, J=6.62 Hz), 7.12-7.28 (3H, m), 7.38 (1H, d, J=7.77 Hz), 7.70-7.78 (1H, m), 8.14-8.21 (2H, m), 8.50-8.54 (1H, m)

### EXAMPLE 252

A mixture of 2-[2-(4-nitrophenoxy)ethyl]pyridine (733 mg) in methanol (30 ml) was hydrogenated over 10% palladium on carbon (400 mg) under an atmospheric pressure of hydrogen at ambient temperature under stirring for 3 hours. After removal of the catalyst, the solvent was evaporated in vacuo. The residue and 4′-(trifluoromethoxy)-1,1′-biphenyl-2-carboxylic acid (846 mg), HOBT.H₂O (446 mg) and WSC.HCl (630 mg) in N,N-dimethylformamide (20 ml) was stirred at ambient temperature for 15 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (4:6). The fraction was evaporated in vacuo and the residue was recrystallized from ethyl acetate and diisopropyl ether to give N-{4-[2-(2-pyridinyl)ethoxy]phenyl}-4′-(trifluoromethoxy)-1,1′-biphenyl-2-carboxamide (0.93 g).

¹H-NMR (DMSO-d₆): δ3.16 (2H, t, J=6.70 Hz), 4.31 (2H, t, J=6.54 Hz), 6.85 (2H, d, J=9.02 Hz), 7.23-7.26 (1H, m), 7.33-7.96 (12H, m), 8.51 (1H, d, J=4.04 Hz), 10.11 (1H, s)

APCI-MS (m/z): 479 (M+H)⁺

### EXAMPLE 253

A mixture of 2-[2-(4-nitrophenoxy)ethyl]pyridine (1.22 g) in methanol (40 ml) was hydrogenated over 10% palladium on carbon (400 mg) under an atmospheric pressure of hydrogen at ambient temperature under stirring for 3 hours. After removal of the catalyst, the solvent was evaporated in vacuo. The residue was dissolved in tetrahydrofuran (20 ml) and triethylamine (1.01 g) and to the above solution was,added 4′-(trifluoromethyl)-1,1′-biphenyl-2-carbonyl chloride (1.42 g) in tetrahydrofuran (10 ml) at ambient temperature under stirring. The resultant mixture was stirred at ambient temperature for 15 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (6:4). The fraction was evaporated in vacuo and the residue was recrystallized from ethyl acetate and diisopropyl ether to give N-{4-[2-(2-pyridinyl)ethoxy]phenyl}-4′-(trifluoromethyl)-1,1′-biphenyl-2-carboxamide (1.368 g).

¹H-NMR (DMSO-d₆): δ3.16 (2H, t, J=6.54 Hz), 4.30 (2H, t, J=6.54 Hz), 6.84 (2H, d, J=9.02 Hz), 7.22-7.26 (1H, m), 7.33-7.78 (12H, m), 8.51 (1H, d, J=4.00 Hz), 10.19 (1H, s)

### EXAMPLE 254

A solution of 2-hydroxyethylpyridine (443 mg) and potassium tert-butoxide (404 mg) in tetrahydrofuran (30 ml) was stirred at ambient temperature for an hour. A N-(4-fluoro-3-nitrophenyl)-4′-(trifluoromethyl)-1,1′-biphenyl-2-carboxamide (1.22 g) was added to an above mixture and the resultant mixture was stirred at ambient temperature for 15 hours. The reaction mixture was poured into a mixture of ethyl acetate and Water. The organic layer was washed with brine and dried over magnesium sulfate. The solvent was evaporated in vacuo and the residue was chromatographed on silica gel eluting with ethyl acetate and n-hexane (5:5-7:3). The fraction was evaporated in vacuo to give N-{3-nitro-4-[2-(2-pyridinyl)ethoxy]phenyl}-4′-(trifluoromethyl)-1,1′-biphenyl-2-carboxamide (0.374 g).

¹H-NMR (DMSO-d₆): δ3.18 (2H, t, J=6.56 Hz), 4.49 (2H, t, J=6.56 Hz), 7.23-7.39(3H, m), 7.54-7.78 (10H, m), 8.11 (1H, d, J=2.58 Hz), 8.49-8.51 (1H, m), 10.58 (1H, s)

### EXAMPLE 255

A mixture of N-(3-nitro-4-[2-(2-pyridinyl)ethoxy]phenyl}-4′-(trifluoromethyl)-1,1′-biphenyl-2-carboxamide (370 mg) in methanol (30 ml) was hydrogenated over 10% palladium on carbon (150 mg) under an atmospheric pressure of hydrogen at ambient temperature under stirring for 3 hours. After removal of the catalyst, the solvent was evaporated in vacuo and the residue and triethylamine (221 mg) were dissolved in tetrahydrofuran (20 ml). Acetyl chloride (115 mg) was added to the above solution at ambient temperature under stirring. The resultant mixture was stirred at ambient temperature for 6 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (6:4). The fraction was evaporated in vacuo and the residue was recrystallized from ethyl acetate and diisopropyl ether to give N-{3-(acetylamino)-4-[2-(2-pyridinyl)ethoxy]phenyl}-4′-(trifluoromethyl)-1,1′-biphenyl-2-carboxamide (67 mg).

¹H-NMR (DMSO-d₆): δ2.04 (3H, s), 3.20 (2H, t, J=6.60 Hz), 4.330 (2H, t, J=6.60 Hz), 6.98 (2H, d, J=8.88 Hz), 7.22-7.30 (2H, m), 7.38-7.77 (9H, m), 8.07 (1H, s), 8.51 (1H, d, J=4.02 Hz), 8.84 (1H, s), 10.25 (1H, s)

Preparation 88

2-[2-(2-Fluoro-4-nitrophenoxy)ethyl]pyridine was obtained from 1,2-difluoro-4-nitrobenzene and 2-hydroxyethylpyridine in the same manner as in Preparation 87.

¹H-NMR (DMSO-d₆): δ3.03 (2H, t, J=6.87 Hz), 3.79 (2H, t, J=6.87 Hz), 6.80-6.93 (1H, m), 7.19-7.30 (2H, m), 7.64-7.69 (1H, m), 7.86-7.95 (2H, m), 8.46-8.49 (1H, m)

### EXAMPLE 256

N-{3-Fluoro-4-[2-(2-pyridinyl)ethoxy]phenyl}-4′-(trifluoromethyl)-1,1′-biphenyl-2-carboxamide was obtained from 2-[2-(2-fluoro-4-nitrophenoxy)ethyl]pyridine in the same manner as in Example 253.

¹H-NMR (DMSO-d₆): δ3.18 (2H, t, J=6.68 Hz), 4.38 (2H, t, J=6.68 Hz), 7.13-7.27 (3H, m), 7.34-7.78 (11H, m), 8.51 (1H, d, J=4.70Hz), 10.38 (1H, s)

### EXAMPLE 257

A solution of [4-({[4′-(trifluoromethyl)-1,1′-biphenyl-2-yl]carbonyl}amino)phenyl]acetic acid (600 mg), triethylamine (150 mg) and diphenylphosphorylazide (454 mg) in benzene (20 ml) was refluxed at under stirring-for an hour. 2-Aminopyridine (169 mg) was added to the above solution and the reaction mixture was refluxed under stirring for 2 hours. The resultant mixture was poured into a mixture of ethyl acetate and water. The organic layer was washed with brine and dried over magnesium sulfate. The solvent was evaporated in vacuo and the residue was chromatographed on silica gel eluting with ethyl acetate and n-hexane (5:5-8:2). The fraction was evaporated in vacuo and the residue was recrystallized from ethyl acetate and diisopropyl ether to give N-[4-({[(2-pyridinylamino)carbonyl]amino}methyl)-phenyl]-4′-(trifluoromethyl)-1,1′-biphenyl-2-carboxamide (248 mg).

¹H-NMR (DMSO-d₆): δ4.34 (2H, d, J=5.76 Hz), 6.89-6.96 (1H, m), 7.22 (2H, d, J=8.36 Hz), 7.35 (2H, d, J=8.42 Hz), 7.47-7.65 (9H, m), 7.76 (2H, d, J=8.32 Hz), 8.16 (1H, d, J=3.60 Hz), 8.58 (1H, m), 9.31 (1H, s), 10.36 (1H, s)

### EXAMPLE 258

N-(4-{[(2-Pyridinylamino)carbonyl]amino}phenyl)-4′-(trifluoromethyl)-1,1′-biphenyl-2-carboxamide was obtained from 4-({[4′-(trifluoromethyl)-1,1′-biphenyl-2-yl]carbonyl}amino)benzoic acid and 2-aminopyridine in the same manner as in Example 257.

¹H-NMR (DMSO-d₆): δ7.00-7.03 (1H, m), 7.45-7.79 (14H, m), 8.26 (1H, m), 9.41 (1H, s), 10.28 (1H, s), 10.46 (1H, s)

### EXAMPLE 259

N-(4-{2-Oxo-2-[(2-pyridinylmethyl)amino]ethyl}phenyl)-4′-(trifluoromethyl)-1,1′-biphenyl-2-carboxamide was obtained in the same manner as in Example 1.

¹H-NMR (DMSO-d₆): δ3.45 (2H, s), 4.34 (2H, d, J=5.88 Hz), 7.17-7.27 (4H, m), 7.28-7.78 (11H, m), 8.49 (1H, d, J=4.66 Hz), 8.59 (1H, m), 10.33 (1H, s)

APCI-MS (m/z); 490 (M+H)⁺

### EXAMPLE 260

N-(4-{2-Oxo-2-[(4-pyridinylmethyl)amino]ethyl}phenyl)-4′-(trifluoromethyl)-1,1′-biphenyl-2-carboxamide was obtained in the same manner as in Example 1.

¹H-NMR (DMSO-d₆): δ3.46 (2H, s), 4.29 (2H, d, J=5.94 Hz), 7.17-7.21 (4H, m), 7.46 (2H, d, J=8.46 Hz), 7.48-7.66 (6H, m), 7.76 (2H, d, J=8.30 Hz), 8.47 (2H, d, J=5.94 Hz), 8.59 (1H, t, J=5.94 Hz), 10.34 (1H, s)

APCI-MS (m/z); 490 (M+H)⁺

### EXAMPLE 261

N-(4-{2-[(6-Methyl-2-pyridinyl)amino]-2-oxoethyl}phenyl)-4′-(trifluoromethyl)-1,1′-biphenyl-2-carboxamide was obtained in the same manner as in Example 1.

¹H-NMR (DMSO-d₆): δ2.40 (3H, s), 3.63 (2H, s), 6.95 (1H, d, J=5.36 Hz), 7.24 (2H, d, J=8.40 Hz), 7.47 (2H, d, J=8.40 Hz), 7.47-7.65 (7H, m), 7.76 (2H, d, J=8.22 Hz), 7.85 (1H, d, J=6.40 Hz), 10.35 (1H, s), 10.58 (1H, s)

### EXAMPLE 262

N-{4-[2-Oxo-2-(2-quinolinylamino)ethyl]phenyll-4′-(trifluoromethyl)-1,1′-biphenyl-2-carboxamide was obtained in the same manner as in Example 1.

¹H-NMR (DMSO-d₆): δ3.73 (2H, s), 7.29 (2H, d, J=8.44 Hz), 7.47-7.93 (14H, m), 8.28-8.37 (1H, m), 10.37 (1H, s), 11.01 (1H, s)

### EXAMPLE 263

N-{4-[2-(1-Isoquinolinylamino)-2-oxoethyl]phenyl}-4′-(trifluoromethyl)-1,1′-biphenyl-2-carboxamide was obtained in the same manner as in Example 1.

¹H-NMR (DMSO-d₆): δ3.78 (2H, s), 7.31 (2H, d, J=8.40 Hz), 7.49-7.79 (13H, m), 7.88-7.99 (2H, m), 8.31 (1H, d, J=5.70 Hz), 10.37 (1H, s), 11.64 (1H, s)

### EXAMPLE 264

N-{4-[2-Oxo-2-(1,3-thiazol-2-ylamino)ethyl]phenyl}-4′-(trifluoromethyl)-1,1′-biphenyl-2-carboxamide was obtained in the same manner as in Example 1.

¹H-NMR (DMSO-d₆): δ3.70 (2H, s), 7.19-7.25 (3H, m), 7.46-7.65 (9H, m), 7.76 (2H, d, J=8.26 Hz), 10.37 (1H, s), 12.31 (1H, s)

### EXAMPLE 265

N-(4-{2-[(1-Methyl-1H-benzimidazol-2-yl)amino]-2-oxoethyl}phenyl)-4′-(trifluoromethyl)-1,1′-biphenyl-2-carboxamide was obtained in the same manner as in Example 1.

¹H-NMR (DMSO-d₆): δ3.54 (3H, s), 3.68 (2H, s), 7.18-7.28 (3H, m), 7.46-7.66 (11H, m) 7.76 (2H, d, J=8.24 Hz), 10.34 (1H, s), 10.80 (1H, s)

### EXAMPLE 266

N-{4-[2-(1H-Benzimidazol-2-ylamino)-2-oxoethyl]phenyl}-4′-(trifluoromethyl)-1,1′-biphenyl-2-carboxamide was obtained in the same manner as in Example 1.

¹H-NMR (DMSO-d₆): δ3.71 (2H, s), 7.05-7.09 (2H, m), 7.28 (2H, d, J=8.40 Hz), 7.41-7.65 (10H, m), 7.76 (2H, d, J=8.24 Hz), 10.37 (1H, s), 11.72 (1H, s), 12.00 (1H, s)

### EXAMPLE 267

N-(4-{2-[(1-Ethyl-1H-pyrazol-5-yl)amino]-2-oxoethyl)phenyl)-4′-(trifluoromethyl)-1,1′-biphenyl-2-carboxamide was obtained in the same manner as in Example 1.

¹H-NMR (DMSO-d₆) δ1.22 (3H, t, J=7.14 Hz), 3.62 (2H, s), 3.95 (2H, q, J=7.14 Hz), 6.15 (1H, d, J=1.80 Hz), 7.23 (2H, d, J=8.40 Hz), 7.33 (1H, d, J=1.80 Hz), 7.46-7.66 (8H, m), 7.76 (2H, d, J=8.30 Hz); 10.01 (1H, s), 10.34 (1H, s)

APCI-MS (m/z); 493 (M+H)⁺

Preparation 89

Methyl (2E)-3-[4-({[4′-(trifluoromethyl)-1,1′-biphenyl-2yl)carbonyl}amino)phenyl]-2-propenoate was obtained from 4′-(trifluoromethyl)-1,1′-biphenyl-2-carboxylic acid and methyl 3-(4-aminophenyl)-2-propenoate in the same manner as in Preparation 1.

¹H-NMR (DMSO-d₆): δ3.71 (3H, s), 6.54 (1H, d, J=16.03 Hz), 7.36-7.78 (13H, m), 10.59 (1H, s)

Preparation 90

(2E)-3-(4-({[4′-(Trifluoromethyl)-1,1′-biphenyl-2-yl]carbonyl}amino)phenyl)-2-propenoic acid was obtained from methyl (2E)-3-[4-({[4′-(trifluoromethyl)-1,1′-biphenyl-2-yl]carbonyl}amino)phenyl]-2-propenoate in the same manner as in Preparation 2.

¹H-NMR (DMSO-d₆): δ6.43 (1H, d, J=15.98 Hz), 7.48-7.78 (13H, m), 10.57 (1H, s), 12.28 (1H, br.s)

### EXAMPLE 268

N-{4-[(1E)-3-Oxo-3-(2-pyridinylamino)-1-propenyl]phenyl}-4′-(trifluoromethyl)-1,1′-biphenyl-2-carboxamide was obtained from (2E)-3-(4-({[4′-(trifluoromethyl)-1,1′-biphenyl-2-yl]carbonyl}amino)phenyl)-2-propenoic acid and ²-aminopyridine in the same manner as in Example 1.

¹H-NMR (DMSO-d₆): δ6.96 (1H, d, J=15.68 Hz), 7.08-7.14 (1H, m), 7.52-7.85 (12H, m), 8.25 (1H, d, J=8.30 Hz), 8.34 (1H, d, J=3.73 Hz), 10.59 (1H, s), 10.64 (1H, s)

### EXAMPLE 269

A mixture of N-{4-[(1E)-3-oxo-3-(2-pyridinylamino)-1-propenyl]phenyl}-4′-(trifluoromethyl)-1,1′-biphenyl-2-carboxamide (260 mg) in methanol (20 ml) was hydrogenated over 10% palladium on carbon (100 mg) under an atmospheric pressure of hydrogen at ambient temperature under stirring for 5 hours. After removal of the catalyst, the solvent was evaporated in vacuo and the residue was recrystallized from ethyl acetate and diisopropyl ether to give N-{4-[3-oxo-3-(2-pyridinylamino)propyl]phenyl}-4′-(trifluoromethyl)-1,1′-biphenyl-2-carboxamide (154 mg).

¹H-MMR (DMSO-d₆): δ2.67-2.71 (2H, m), 2.81-2.85 (2H, m), 7.04-7.65 (11H, m), 8.09 (1H, d, J=8.26 Hz), 8.29 (1H, d, J=4.32 Hz), 10.30 (1H, s), 10.47 (1H, s)

Preparation 91

Methyl 4-[4-({[4′-(trifluoromethyl)-1,1′-biphenyl-2-yl]carbonyl}amino)phenyl]butanoate was obtained from 4′-(trifluoromethyl)-1,1′-biphenyl-2-carboxylic acid and methyl 4-(4-aminophenyl)butanoate in the same manner as in Preparation 1.

¹H-NMR (DMSO-d₆): δ1.71-1.86 (2H, m), 2.28 (2H, t, J=7.40 Hz), 2.49-2.56 (2H, m), 3.57 (3H, s), 7.09 (2H, d, J=8.36 Hz), 7.45 (2H, d, J=8.36 Hz), 7.47-7.66 (6H, m), 7.75 (2H, d, J=8.38 Hz), 10.30 (1H, s)

Preparation 92

4-[4-({[4′-(Trifluoromethyl)-1,1′-biphenyl-2-yl]carbonyl}amino)phenyl]butanoic acid was obtained from methyl 4-[4-({[4′-(trifluoromethyl)-1,1′-biphenyl-2-yl]carbonyl}amino)phenyl]butanoate in the same manner as in Preparation 2.

¹H-NMR (DMSO-d₆): δ1.68-1.83 (2H, m), 2.20 (2H, t, J=7.36 Hz), 2.49-2.57 (2H, m), 7.09 (2H, d, J=8.38 Hz), 7.45 (2H, d, J=8.38 Hz), 7.47-7.66 (6H, m), 7.76 (2H, d, J=8.38 Hz), 12.05 (1H, s)

### EXAMPLE 270

N-{4-[4-Oxo-4-(2-pyridinylamino)butyl]phenyl}-4′-(trifluoromethyl)-1,1′-biphenyl-2-carboxamide was obtained from 4-[4-({[4′-(trifluoromethyl)-1,1′-biphenyl-2-yl]carbonyl}amino)phenyl]butanoic acid and 2-aminopyridine in the same manner as in Example 1.

¹H-NMR (DMSO-d₆): δ1.81-1.88 (2H, m), 2.37-2.40 (2H, m), 2.43-2.59 (2H, m), 7.04-7.14 (3H, m), 7.42-7.78 (11H, m), 8.09 (1H, d, J=8.32 Hz), 8.29 (1H, d, J=3.80 Hz), 10.30 (1H, s), 10.43 (1H, s)

Preparation 93

Methyl [3-({[4′-(trifluoromethyl)-1,1′-biphenyl-2-yl]carbonyl}amino)phenyl]acetate was obtained from 4′-(trifluoromethyl)-1,1′-biphenyl-2-carboxylic acid and methyl 3aminophenylacetate in the same manner as in Preparation 1.

¹H-NMR (DMSO-d₆): δ3.60 (3H, s), 3.62 (2H, s), 6.96 (1H, d, J=7.52 Hz), 7.19-7.27 (1H, m), 7.41 (2H, d, J=8.32 Hz), 7.43-7.66 (6H, m), 7.76 (2H, d, J=8.32 Hz), 10.39 (1H, s)

Preparation 94

[3-({[4′-(Trifluoromethyl)-1,1′-biphenyl-2-yl]carbonyl}amino)phenyl]acetic acid was obtained from methyl [3-({[4′-(trifluoromethyl)-1,1′-biphenyl-2-yl]carbonyl}amino)-phenyl]acetate in the same manner as in Preparation 2.

¹H-NMR (DMSO-d₆): δ3.54 (2H, s), 6.96 (1H, d, J=7.52 Hz), 7.18-7.25 (1H, m), 7.40 (1H, d, J=8.36 Hz), 7.49-7.66 (7H, m), 7.76 (2H, d, J=8.32 Hz), 10.39 (1H, s)

### EXAMPLE 271

N-(3-[2-Oxo-2-(2-pyridinylamino)ethyl]phenyl}-4′-(trifluoromethyl)-1,1′-biphenyl-2-carboxamide was obtained from [3-({[4′-(trifluoromethyl)-1,1′-biphenyl-2-yl]carbonyl}amino)phenyl]acetic acid and 2-aminopyridine in the same manner as in Example 1.

¹H-NMR (DMSO-d₆): δ3.68 (2H, s), 7.03-7.26 (3H, m), 7.40 (1H, d, J=8.32 Hz), 7.49-7.95 (10H, m), 8.06 (1H, d, J=8.36 Hz), 8.31 (1H, d, J=3.82 Hz), 10.41 (1H, s), 10.71 (1H, s)

Preparation 95

Methyl N-(tert-butoxycarbonyl)-4-({[4′-(trifluoromethyl)-1,1′-biphenyl-2-yl]carbonyl}amino)phenylalaninate was obtained from 4′-(trifluoromethyl)-1,1′-biphenyl-2-carboxylic acid and 4-amino-N-(tert-butoxycarbonyl)phenylalaninate in the same manner as in Preparation 1.

¹H-NMR (DMSO-d₆): δ1.32 (9H, s), 2.72-2.98 (2H, m), 3.60 (3H, s), 4.07-4.18 (1H, m), 7.13 (2H, d, J=8.38 Hz), 7.25 (1H, d, J=8.06 Hz), 7.43 (2H, d, J=8.38 Hz), 7.48-7.65 (5H, m), 7.75 (2H, d, J=8.36 Hz), 10.31 (1H, s)

Preparation 96

N-(tert-Butoxycarbonyl)-4-({[4′-(trifluoromethyl)-1,1′-biphenyl-2-yl]carbonyl}amino)phenylalanine was obtained from methyl N-(tert-butoxycarbonyl)-4-({[4′-(trifluoromethyl)-1,1′-biphenyl-2-yl]carbonyl}amino)phenylalaninate in the same manner as in Preparation 2.

¹H-NMR (DMSO-d₆): δ1.33 (9H, s), 2.71-3.02 (2H, m), 4.05-4.07 (1H, m), 7.06 (1H, d, J=8.30 Hz), 7.16 (2H, d, J=8.32 Hz), 7.45 (2H, d, J=8.32 Hz), 7.47-7.66 (5H, m), 7.75 (2H, d, J=8.36 Hz), 10.33 (1H, s), 12.54 (1H, br.s)

### EXAMPLE 272

2-[(4-{2-[(tert-Butoxycarbonyl)amino]-3-oxo-3-(2-pyridinylamino)propyl}anilino)carbonyl]-4′-(trifluoromethyl)-1,1′-biphenyl was obtained from N-(tert-butoxycarbonyl)-4-({[4′-(trifluoromethyl)-1,1′-biphenyl-2-yl]carbonyl}amino)phenylalaine and 2-aminopyridine in the same manner as in Example 1.

¹H-NMR (DMSO-d₆): δ1.31 (9H, s), 2.74-2.76 (1H, m), 2.95-3.04 (1H, m), 4.39 (1H, m), 7.08-7.12 (2H, m), 7.27 (2H, d, J=8.34 Hz), 7.43 (2H, d, J=8.34 Hz), 7.50-7.82 (8H, m), 8.08 (1H, d, J=8.28 Hz), 8.33 (1H, d, J=3.96 Hz), 10.31 (1H, s), 10.61 (1H, s)

### EXAMPLE 273

A mixture of 2-[(4-{2-[(tert-butoxycarbbnyl)amino]-3-oxo-3-(2-pyridinylamino)propyl}anilino)carbonyl]-4′-(trifluoromethyl)-1,1′-biphenyl (0.64 g) and 4N hydrogen chloride-dioxane solution (3 ml) in methanol (20 ml) was stirred at ambient temperature for 1.5 hours. The reaction mixture was evaporated in vacuo. The residue was dissolved in a mixture of ethyl acetate and water and adjusted to pH 8.0 with 20% aqueous potassium carbonate solution. The organic layer was washed with brine and dried over magnesium sulfate. The solvent was evaporated in vacuo to give N-(4-(2-amino-3-oxo-3-(2-pyridinylamino)propyl)phenyl)-4′-(trifluoromethyl)-1,1′-biphenyl-2-carboxamide (480 mg).

¹H-NM (DMSO-d₆): δ2.60-2.71 (1H, m), 2.96-3.04 (1H, m), 3.57-3.66 (1H, m), 7.07-7.19 (3H, m), 7.46-7.83 (13H, m), 8.12 (1H, d, J=8.30 Hz), 8.30 (1H, d, J=3.86 Hz), 10.31 (1H, s)

### EXAMPLE 274

A solution of acetyl chloride (56 mg) in tetrahydrofuran (3 ml) was added to a mixture of N-{4-[2-amino-3-oxo-3-(2-pyridinylamino)propyl]phenyl}-4′-(trifluoromethyl)-1,1′-biphenyl-2-carboxamide (300 mg) and triethylamine (120 mg) in tetrahydrofuran (10 ml) at ambient temperature and the resultant mixture was stirred at ambient temperature for 2 hours. The reaction mixture was poured into a mixture of ethyl acetate. The organic layer was washed with 5% aqueous potassium carbonate solution and brine and dried over magnesium sulfate. The solvent was evaporated in vacuo and the residue was recrystallized from a ethyl acetate and diisopropyl ether to give N-{4-[2-(acetylamino)-3-oxo-3-(2-pyridinylamino)propyl]phenyl}-4′-(trifluoromethyl)-1,1′-biphenyl-2-carboxamide (211 mg).

¹H-NMR (DMSO-d₆): δ1.78 (3H, s), 2.69-2.80 (1H, m), 2.99-3.04 (1H, m), 4.74 (1H, m), 7.08-7.14 (1H, m), 7.25 (2H, d, J=8.40 Hz), 7.42 (2H, d, J=8.40 Hz), 7.50-7.82 (6H, m), 7.76 (2H, d, J=8.36 Hz), 8.07 (1H, d, J=8.26 Hz), 8.20 (1H, d, J=8.02 Hz), 8.32 (1H, d, J=3.56 Hz), 10.31 (1H, s), 10.86 (1H, s)

Preparation 97

A mixture of [4-{[4′-(trifluoromethyl)-1,1′-biphenyl-2-yl]carbonyl}amino)phenyl]acetic acid (998 mg), 1,2-phenylenediamine (405 mg), HOBT.H₂O (372 mg) and WSC.HCl (525 mg) in N,N-dimethylformamide (20 ml) was stirred at ambient temperature for 15 hours. The reaction mixture was poured into a mixture of ethyl acetate and water. The organic layer was washed with 3N hydrochloric acid, 5% aqueous potassium carbonate solution and brine and dried over magnesium sulfate. The solvent was evaporated in vacuo and the residue was recrystallized from ethyl acetate and diisopropyl ether to give N-{4-[2-(2aminoanilino)-2-oxoethyl]phenyl}-4′-(trifluoromethyl)-1,1′-biphenyl-2-carboxamide (1.0 g).

¹H-NMR (DMSO-d₆): δ3.58 (2H, s), 4.81 (2H, s), 6.52-6.53 (1H, m), 6.71 (1H, d, J=6.62 Hz), 6.73-6.89 (1H, m), 7.13 (1H, dd, J=1.34 Hz, 7.83 Hz), 7.25 (2H, d, J=8.44 Hz), 7.45-7.65 (8H, m), 7.76 (2H, J=8.32 Hz), 9.32 (1H, s), 10.34 (1H, s)

### EXAMPLE 275

A mixture of N-{4-[2-(2-aminoanilino)-2-oxoethyl]phenyl}-4′-(trifluoromethyl)-1,1′-biphenyl-2-carboxamide (344 mg) and conc. hydrochloric acid (2 ml) in ethanol (20 ml) was refluxed under stirring for 3 hours. The reaction mixture was evaporated in vacuo and the residue was dissolved. in a mixture of ethyl acetate and water and adjusted to pH 8.0 with 20% aqueous potassium carbonate solution. The organic layer was washed with brine and dried over magnesium sulfate. The solvent was evaporated in vacuo and the residue was recrystallized from ethyl acetate and diisopropyl ether to give N-[4-(1H-benzimidazol-2-ylmethyl)phenyl]-4′-(trifluoromethyl)-1,1′-biphenyl-2-carboxamide (300 mg).

¹H-NMR (DMSO-d₆): δ3.35 (2H, s), 7.09-7.13 (2H, m), 7.24 (2H, d, J=8.48 Hz), 7.44-7.60 (10H, m), 7.62 (2H, d, J=6.04 Hz), 10.35 (1H, s), 12.21 (1H, br.s)

Preparation 98

A mixture of methyl (2E)-3-[4-({[4′-(trifluoromethyl)-1,1′-biphenyl-2-yl]carbonyl}amino)phenyl]-2-propenoate (1.9 g) in methanol (60 ml) and tetrahydrofuran (20 ml) was hydrogenated over 10% palladium on carbon (0.6 g) under an atmospheric pressure of hydrogen at ambient temperature under stirring for 5 hours. After removal of the catalyst, the solvent was evaporated in vacuo and the residue was recrystallized from ethyl acetate and diisopropyl ether to give methyl 3-[4-({[4′-(trifluoromethyl)-1,1′-biphenyl-2-yl]carbonyl}amino)phenyl]-propanoate (1.57 g).

¹H-NMR (DMSO-d₆): δ2.59(2H, t, J=7.14 Hz), 2.75 (2H, d, J=7.14 Hz), 3.57 (3H, s), 7.12 (2H, d, J=8.40 Hz), 7.42 (2H, d, J=8.40 Hz), 7.49-7.66 (5H, m), 7.76 (2H, d, J=8.34 Hz), 10.31 (1H, s)

Preparation 99

3-[4-({[4′-(Trifluoromethyl)-1,1′-biphenyl-2-yl]carbonyl}amino)phenyl]propanoic acid was obtained from methyl 3-[4-({[4′-(trifluoromethyl)-1,1′-biphenyl-2-yl]carbonyl}amino)phenyl]propanoate in the same manner as in Preparation 2.

¹H-NMR (DMSO-d₆): δ2.49 (2H, t, J=7.42 Hz), 2.76 (2H, d, J=7.42 Hz), 7.13 (2H, d, J=8.38 Hz), 7.43 (2H, d, J=8.38 Hz), 7.50-7.66 (5H, m), 7.75 (2H, d, J=8.32 Hz), 10.31 (1H, s), 12.11 (1H, br.s)

Preparation 100

N-{4-[3-(2-Aminoanilino)-3-oxopropyl]phenyl}-4′-(trifluoromethyl)-1,1′-biphenyl-2-carboxamide was obtained from 3-[4-({[4′-(trifluoromethyl)-1,1′-biphenyl-2-yl]carbonyl}amino)phenyl]propanoic acid and 1,2-phenylenediamine in the same manner as in Preparation 97.

¹H-NMR-(DMSO-d₆): δ2.55-2.63 (2H, m), 2.82-2.89 (2H, m), 4.78 (2H, s), 6.49-6.56 (1H, m), 6.70 (1H, d, J=6.80 Hz), 6.85-6.92 (1H, m), 7.10-7.14 (1H, m), 7.16 (2H, d, J=8.58 Hz), 7.42-7.65 (8H, m), 7.76 (2H, d, J=8.32 Hz), 9.10 (1H, s), 10.30 (1H, s)

### EXAMPLE 276

N-{4-[2-(1H-Benzimidazol-2-yl)ethyl]phenyl}-4′-(trifluoromethyl)-1,1′-biphenyl-2-carboxamide was obtained from N-{4-[3-(2-aminoanilino)-3-oxopropyl]phenyl}-4′-(trifluoromethyl)-1,1′-biphenyl-2-carboxamide in the same manner as in Example 275.

¹H-NMR (DMSO-d₆): δ3.07 (4H, s), 7.09-7.18 (2H, m), 7.16 (2H, d, J=8.58 Hz), 7.41-7.65 (10H, m), 7.75 (2H, d, J=8.32 Hz), 10.31 (1H, s), 12.20 (1H, s)

Preparation 101

N-{4-[(1E)-3-(2-Aminoanilino)-3-oxo-1-propenyl]phenyl}-4′-(trifluoromethyl)-1,1′-biphenyl-2-carboxamide was obtained from (2E)-3-[4-({[4′-(trifluoromethyl)-1,1′-biphenyl-2-yl]carbonyl}amino)phenyl-2-propenoic acid and 1,2-phenylenediamine in the same manner as in Preparation 97.

¹H-NMR (DMSO-d₆): δ4.92 (2H, s), 6.54-6.62 (1H, m), 6.75(1H, d, J=6.88 Hz), 6.85-6.96 (2H, m), 7.34 (1H, d, J=7.72 Hz), 7.46-7.66 (11H, m), 7.77 (2H, d, J=8.31 Hz), 9.37 (1H, s), 10.57 (1H, s)

### EXAMPLE 277

N-{4-[(E)-2-(1H-Benzimidazol-2-yl)ethenyl]phenyl}-4′-(trifluoromethyl)-1,1′-biphenyl-2-carboxamide was obtained from N-{4-[(1E)-3-(2-aminoanilino)-3-oxo-1-propenyl]phenyl}-4′-(trifluoromethyl)-1,1′-biphenyl-2-carboxamide in the same manner as in Example 275.

¹H-NMR (DMSO-d₆): δ7.08-7.20 (3H, m), 7.45-7.71 (13H, m), 7.72 (2H, d, J=8.36 Hz), 10.53 (1H, s), 12.57 (1H, s)

Preparation 102

N-{4-[4-(2-Aminoanilino)-4-oxobutyl]phenyl}-4′-(trifluoromethyl)-1,1′-biphenyl-2-carboxamide was obtained from 4-[4-({[4′-(trifluoromethyl)-1,1′-biphenyl-2-yl]carbonyl}amino)phenyl]butanoic acid and 1,2-phenylenediamine in the same manner as in Preparation 97.

¹H-NMR (DMSO-d₆): δ1.86-1.89 (2H, m), 2.31 (2H, t, J=7.38 Hz), 2.57 (2H, t, J=7.38 Hz), 4.81 (2H, s), 6.50-6.57 (1H, m), 6.71 (1H, d, J=6.74 Hz), 6.85-6.92 (1H, m), 7.11-7.15 (3H, m), 7.43-7.66 (8H, m), 7.76 (2H, d, J=8.32 Hz), 9.10 (1H, s), 10.30 (1H, s)

### EXAMPLE 278

N-(4-(3-(1H-Benzimidazol-2-yl)propyl)phenyl)-4′-(trifluoromethyl)-1,1′-biphenyl-2-carboxamide was obtained from N-{4-[4-(2-aminoanilino)-4-oxobutyl]phenyl)-4′-(trifluoromethyl)-1,1′-biphenyl-2-carboxamide in the same manner as in Example 275.

¹H-NMR (DMSO-d₆): δ1.97-2.08 (2H, m), 2.61 (2H, t, J=7.40 Hz), 2.80 (2H, t, J=7.40 Hz), 7.06-7.16 (3H, m), 7.44-7.66 (11H, m), 7.76 (2H, d, J=8.32 Hz), 10.31 (1H, s), 12.16 (1H, s)

Preparation 103

A mixture of 4-[(1R)-1-aminoethyl]aniline hydrochloride (406 mg), 2-pyridinecarboxylic acid (271 mg) HOBT.H₂O (327 mg) and WSC.HCl (462 mg) in N,N-dimethylformamide was stirred at ambient temperature for 15 hours. The reaction mixture was poured into a mixture of ethyl acetate and water. The organic layer was washed with 5% aqueous potassium carbonate solution and brine and dried over magnesium sulfate. The solvent was evaporated in vacuo to give N-[(1R)-1-(4-nitrophenyl)ethyl]-2-pyridinecarboxamide (0.55 g).

¹H-NMR (DMSO-d₆): δ1.57 (3H, d J=7.10 Hz), 5.21-5.26 (1H, m), 6.60-6.68 (1H, m), 7.70 (2H, d, J=8.73 Hz), 7.96-8.02 (3H, m), 8.21 (2H, d, J=8.73 Hz), 8.70 (1H, d, J=5.92 Hz), 9.33 (1H, d, J=8.17 Hz)

Preparation 104

A mixture of N-[(1R)-1-(4-nitrophenyl)ethyl]-2-pyridinecarboxamide (0.55 g) in methanol (50 ml) was hydrogenated over 10% palladium on carbon (0.1 g) under an atmospheric pressure of hydrogen at ambient temperature under stirring for 4 hours. After removal of the catalyst, the solvent was evaporated in vacuo to give N-[(1R)-1-(4-aminophenyl)ethyl]-2-pyridinecarboxamide (0.49 g).

¹H-NMR (DMSO-d₆); δ1.46 (3H, d, J=6.95 Hz), 4.98 (2H, s), 4.98-5.09 (1H, m),6.52 (2H, d, J=8.38 Hz), 7.08 (2H, d, J=8.38 Hz), 7.55-7.66 (1H, m), 7.94-8.05 (2H, m), 8.62-8.70 (2H, m)

### EXAMPLE 279

A solution of 4′-(trifluoromethyl)-1,1′-biphenyl-2-carbonyl chloride (570 mg) in tetrahydrofuran (5 ml) was added to a solution of N-[(1R)-1-(4-aminophenyl)ethyl]-2-pyridinecarboxamide (482 mg) and triethylamine (404 mg) in tetrahydrofuran (20 ml) at ambient temperature under stirring. The resultant mixture was stirred at ambient temperature for 2 hours. The reaction mixture was poured into a mixture of ethyl acetate and water and adjusted to pH 1.0 with 6N hydrochloric acid. The organic layer was washed with 5% aqueous potassium carbonate solution and brine and dried over magnesium sulfate. The solvent was evaporated in vacuo and the residue was recrystallized from ethyl acetate and diisopropyl ether to give N-{(1R)-1-[4-({[4′-(trifluoromethyl)-1,1′-biphenyl-2-yl]carbonyl}amino)phenyl]ethyl}-2-pyridinecarboxamide (713 mg).

¹H-NMR (DMSO-d₆): δ1.51 (3H, d, J=6.98 Hz), 5.05-5.20 (1H, m), 7.34 (2H, d, J=8.54 Hz), 7.48 (2H, d, J=8.48 Hz), 7.53-7.65 (7H, m), 7.76 (2H, d, J=8.32 Hz), 7.94-8.04 (2H, m), 8.66 (1H, d, J=4.74 Hz), 8.96 (1H, d, J=8.44 Hz), 10.37 (1H, s)

Preparation 105

N-[(1S)-1-(4-Nitrophenyl)ethyl]-2-pyridinecarboxamide was obtained from 4-[(1S)-1-aminoethyl]aniline hydrochloride and 2-pyridinecarboxylic acid in the same manner as in Preparation 103.

¹H-NMP (DMSO-d₆): δ1.58 (3H, d, J=7.09 Hz), 5.22-5.37 (1H, m), 7.62-7.73 (3H, m), 7.97-8.03 (2H, m), 8.18-8.23 (2H, m), 8.68-8.71 (1H, m), 9.33 (1H, d, J=8.18 Hz)

Preparation 106

N-[(1S)-1-(4-Aminophenyl)ethyl]-2-pyridinecarboxamide was obtained from N-[(1S)-1-(4-nitrophenyl)ethyl]-2-pyridinecarboxamide in the same manner as in Preparation 104.

¹H-NMR (DMSO-d₆): δ1.47 (3H, d, J=6.58 Hz), 3.36 (2H, s), 4.95-5.09 (1H, m), 6.53 (2H, d, J=8.40 Hz), 7.08 (2H, d, J=8.40 Hz), 7.57-7.62 (1H, m), 7.94-8.05 (2H, m), 8.61-8.70 (2H, m)

### EXAMPLE 280

N-{(1S)-1-[4-({[4′-(Trifluoromethyl)-1,1′-biphenyl-2yl]carbonyl}amino)phenyl]ethyl}-2-pyridinecarboxamide was obtained from N-[(1S)-1-(4-aminophenyl)ethyl]-2-pyridinecarboxamide in the same manner as in Example 279.

¹H-NMR (DMSO-d₆): δ1.50 (3H, d, J=6.98 Hz), 5.08-5.13 (1H, m), 7.34 (2H, d, J=8.52 Hz), 7.48 (2H, d, J=8.52 Hz), 7.45-7.65 (8H, m), 7.76 (2H, d, J=8.32 Hz), 7.99-8.02 (1H, m), 8.66 (1H, d, J=4.72 Hz), 8.96 (1H, d, J=8.42 Hz), 10.36 (1H, s)

### EXAMPLE 281

A solution of 4′-methoxy-1,1′-biphenyl-2-carbonyl chloride (300 mg) in tetrahydrofuran (5 ml) was added to a solution of N-{6-[2-(4-aminophenyl)ethyl]-2-pyridinyl}acetamide (319 mg) and triethylamine (246 mg) in tetrahydrofuran (20 ml) at ambient temperature and stirred at ambient temperature for 4 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (5:5-7:3). The fraction was evaporated in vacuo and the residue was recrystallized from ethyl acetate and diisopropyl ether to give N-(4-{2-[6-(acetylamino)-2-pyridinyl]ethyl}phenyl)-4′-methoxy-1,1′-biphenyl-2-carboxamide (538 mg).

¹H-NMR (DMSO-d₆): δ2.08 (3H, s), 2.91 (3H, s), 3.74 (3H, s), 6.91-6.95 (3H, m), 7.11 (2H, d, J=8.46 Hz), 7.33-7.68 (9H, m), 7.90 (1H, d, J=8.14 Hz), 10.13 (1H, s), 10.41 (1H, s)

### EXAMPLE 282

A mixture of N-(4-{2-[6-(acetylamino)-2-pyridinyl)ethyl}phenyl)-4′-methoxy-1,1′-biphenyl-2-carboxamide (420 mg) and 6N hydrochloric acid (10 ml) in methanol (10 ml) was refluxed under stirring for 2 hours. The reaction mixture was evaporated in vacuo. The residue was dissolved in a mixture of ethyl acetate and water and adjusted to pH 8.0 with 20% aqueous potassium carbonate solution. The organic layer was washed with brine and dried over magnesium sulfate. The solvent was evaporated in vacuo and the residue was recrystallized from ethyl acetate and diisopropyl ether to-give N-{4-[2-(6-amino-2-pyridinyl)ethyl]phenyl}-4′-methoxy-1,1-biphenyl-2-carboxamide (300 mg).

¹H-NMR (DMSO-d₆): δ2.67-2.88 (4H, m), 3.74 (3H, s), 5.81 (2H, s), 6.25 (1H, d, J=7.82 Hz), 6.29 (1H, d, J=8.78 Hz), 6.93 (2H, d, J=6.84 Hz), 7.11 (2H, d, J=8.44 Hz), 7.20-7.25 (1H, m), 7.30-7.53 (9H, m), 10.13 (1H, s)

Preparation 107

A mixture of 6-[(tert-butoxycarbonyl)amino]-2-pyridinecarboxylic acid (596 mg) and HOBT.H₂O (372 mg) and WSC.HCl (525 mg) in N,N-dimethylformamide (10 ml) was stirred at ambient temperature for an hour. To a resultant mixture was added to a mixture of 4-nitrobenzylamine hydrochloride (519 mg) and triethylamine (333 mg) in N,N-dimethylformamide (10 ml) and stirred at ambient temperature for 15 hours. The reaction mixture was poured into a mixture of ethyl acetate and water. The organic layer was washed with 5% aqueous potassium carbonate solution and brine and dried over magnesium sulfate. The solvent was evaporated in vacuo to give tert-butyl 6-{[(4-nitrobenzyl)amino]carbonyl}-2-pyridinylcarbamate (1.1 g).

¹H-NMR (DMSO-d₆): δ1.48 (9H, s), 4.66 (2H, d, J=6.29 Hz), 7.60 (2H, d, J=8.76 Hz), 7.90-8.00 (3H, m), 8.23 (2H, d, J=8.76 Hz), 8.94 (1H, m), 9.62 (1H, s)

Preparation 108

A mixture of tert-butyl 6-{[(4-nitrobenzyl)amino]carbonyl}-2-pyridinylcarbamate (931 mg) in methanol (30 ml) was hydrogenated over 10% palladium on carbon (0.4 g) under an atmospheric pressure of hydrogen at ambient temperature under stirring for 5 hours. After removal of the catalyst, the solvent was evaporated in vacuo to give tert-butyl 6-{[(4-aminobenzyl)amino]carbonyl}-2-pyridinylcarbamate (8 60 mg).

¹H-NMR (DMSO-d₆): δ1.55 (9H, s), 4.33 (2H, d, J=5.89 Hz), 5.03 (2H, s), 6.53 (2H, d, J=8.33 Hz), 7.00 (2H, d, J=8.33 Hz), 7.61-7.68 (1H, m), 7.87-7.96 (2H, m), 8.03 (1H, m), 9.78 (1H, s)

### EXAMPLE 283

A solution of 4′-(trifluoromethyl)-1,1′-biphenyl-2-carbonyl chloride (856 mg) in tetrahydrofuran (5 ml) was added to solution of tert-butyl 6-{[(4-aminobenzyl)amino]carbonyl}-2-pyridinylcarbamate (856 mg) and triethylamine (303 mg) in tetrahydrofuran (20 ml) at ambient temperature under stirring. The resultant mixture was stirred at ambient temperature for 2 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (5:5). The fraction was evaporated in vacuo and the residue was recrystallized from ethyl acetate to give 2-[(4-{[({6-[(tert-butoxycarbonyl)amino]-2-pyridinyl}carbonyl)amino]methyl}anilino)carbonyl]-4′-(trifluoromethyl)-1,1′-biphenyl (1.25 g).

¹H-NMR (DMSO-d₆): δ1.47 (9H, s), 4.45 (2H, d, J=4.54 Hz), 7.26 (2H, d, J=8.46 Hz), 7.48-7.70 (9H, m), 7.75 (2H, d, J=8.32 Hz), 7.89-7.98 (2H, m), 8.58 (1H, m), 9.71 (1H, s), 10.38 (1H, s)

### EXAMPLE 284

A mixture of 2-[(4-{{({6-[(tert-butoxycarbonyl) amino]-2-pyridinyl}carbonyl)amino]methyl}anilino)carbonyl]-4′-(trifluoromethyl)-1,1′-biphenyl (1.08 g) and 4N hydrogen chloride-dioxane solution (5.5 ml) in methanol (20 ml) was stirred at ambient temperature for 6 hours. The reaction mixture was evaporated in vacuo. The residue was dissolved in a mixture of ethyl acetate and water and adjusted to pH 8.0 with 20% aqueous potassium carbonate solution. The organic layer was washed with brine and dried over magnesium sulfate. The solvent was evaporated in vacuo and the residue was chromatographed on silica gel eluting with ethyl acetate and n-hexane (5:5-7:3). The fraction was evaporated in vacuo and the residue was recrystallized from ethyl acetate and diisopropyl ether to give 6-amino-N-4-({[4′-(trifluoromethyl)-1,1′-biphenyl-2yl]-carbonyl}amino)benzyl]-2-pyridinecarboxamide (265 mg).

₁H-NMR (DMSO-d₆): δ4.41 (2H, d, J=6.14 Hz), 6.12 (2H, s), 6.63 (1H, d, J=7.78 Hz); 7.18 (1H, d, J=7.42 Hz), 7.23 (2H, d, J=8.60 Hz), 7.46-7.65 (11H, m), 7.75 (2H, d, J=8.30 Hz), 8.57 (1H, t, J=6.14 Hz), 10.36 (1H, s)

### EXAMPLE 285

A mixture of 6-amino-N-[4-({[4′-(trifluoromethyl)-1,1′-biphenyl-2-yl]carbonyl}amino)benzyl]-2-pyridinecarboxamide (298 mg) and acetic anhydride (1 ml) in ethyl acetate (20 ml) was refluxed under stirring for 3 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate and n-hexane (5:5-7:3). The fraction was evaporated in vacuo and the residue was recrystallized from ethyl acetate and diisopropyl ether to give 6-(acetylamino)-N-[4-({[4′-(trifluoromethyl)-1,1′-biphenyl-2-yl]carbonyl}amino)benzyl]-2-pyridinecarboxamide (142 mg).

¹H-NMR (DMSO-d₆): δ2.10 (3H, s), 4.46 (2H, d, J=5.96 Hz), 7.26 (2H, d, J=8.34 Hz), 7.48 (2H, d, J=8.34 Hz), 7.50-7.77 (9H, m), 7.78-7.90 (1H, m), 8.20 (1H, d, J=8.28 Hz), 8.47 (1H, t, J=5.96 Hz), 10.36 (1H, s), 10.50 (1H, s)

### EXAMPLE 286

A solution of 4′-(trifluoromethyl)-1,1′-biphenyl-2-carbonyl chloride (854 mg) in tetrahydrofuran (5 ml) was added to a solution of N-(4-aminobenzyl)-N-(2-pyridinyl)amine (598 mg) and triethylamine (606 mg) in tetrahydrofuran (20 ml) at ambient temperature under stirring. The resultant mixture was stirred at ambient temperature for 6 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel (30g) eluting with ethyl acetate and n-hexane (5:5). The fraction was evaporated in vacuo and the residue was recrystallized from ethyl acetate and diisopropyl ether to give N-{4-[(2-pyridinylamino)methyl]phenyl}-4′-(trifluoromethyl)-1,1-biphenyl-2-carboxamide (220 mg).

¹H-NMR (DMSO-d₆): δ4.39 (2H, d, J=5.98 Hz), 6.40-6.50 (2H, m), 6.94 (1H, t, J=5.98 Hz), 7.23 (2H, d, J=8.44 Hz), 7.45 (2H, d, J=8.44 Hz), 7.31-7.65 (7H, m), 7.75 (2H, d, J=8.30 Hz), 7.94 (1H, d, d=3.98 Hz), 10.31 (1H, s)

Preparation 109

A solution of 4′-(trifluoromethyl)-1,1′-biphenyl-2-carbonyl chloride (2.84 g) in tetrahydrofuran (5 ml) was added to solution of 4-ethynylaniline-(1.17 g) and triethylamine (2.02 g) in tetrahydrofuran (50 ml) at ambient temperature under stirring. The resultant mixture was stirred at ambient temperature for 6 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was recrystallized from ethyl acetate and diisopropyl ether to give N-(4-ethynylphenyl)-4′-(trifluoromethyl)-1,1′-biphenyl-2carboxamide (3.15 g).

¹H-NMR (DMSO-d₆): δ5.53 (0.5H, d, J=2.20 Hz), 5.98 (0.5H, d, J=2.20 Hz), 7.40 (2H, d, J=8.56 Hz), 7.51-7.77 (8H, m), 7.91 (2H, d, J=8.90 Hz), 10.55 (1H, s)

### EXAMPLE 287

A mixture of N-(4-ethynylphenyl)-4′-(trifluoromethyl)-1,1′-biphenyl-2-carboxamide (731 mg), 2-chloropyrimidine (252 mg), potassium acetate (294 mg) and tetrakis(triphenylphosphine)-palladium(0) (2.31 g) in N,N-dimethylformamide (40 ml) was stirred at 100° C. for 6 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel (30 g) eluting with ethyl acetate and n-hexane (5:5). The fraction was evaporated to give N-[4-(2-pyrimidinylethynyl)phenyl]-4′-(trifluoromethyl)-1,1′-biphenyl-2-carboxamide (430 mg).

¹H-NMR (DMSO-d₆): δ7.07-7.79 (12H, m), 7.96 (1H, s), 8.83 (1H, d, J=4.94 Hz), 10.69 (1H, s)

### EXAMPLE 288

A mixture of N-[4-(2-pyrimidinylethynyl)phenyl]-4′-(trifluoromethyl)-1,1′-biphenyl-2-carboxamide (430 mg) in methanol (30 ml) was hydrogenated over 10% palladium on carbon (150 mg) under an atmospheric pressure of hydrogen at ambient temperature under stirring for 4 hours. After removal of the catalyst, the solvent was evaporated in vacuo and the residue was chromatographed on silica gel (25 g) eluting with ethyl acetate and n-hexane (5:5-7:3). The fraction was evaporated in vacuo and the residue was triturated with diisopropyl ether to give N-{4-[2-(2-pyrimidinyl)ethyl]phenyl}-4′-(trifluoromethyl)-1,1′-biphenyl-2-carboxamide (100 mg).

¹H-NMR (DMSO-d₆): δ3.02-3.18 (4H, m), 7.12 (2H, d, J=8.34 Hz), 7.24-7.65 (8H, m), 7.41 (2H, d, J=8.34 Hz), 7.76 (2H, d, J=8.28 Hz), 8.72 (1H, d, J=1.92 Hz), 10.28 (1H, s)

Preparation 110

A solution of 4′-(trifluoromethyl)-1,1′-biphenyl-2-carbonyl chloride (2.84 g) in tetrahydrofuran (5 ml) was added to solution of methyl 2-(4-aminophenyl)propanoate hydrochloride (2.32 g) and triethylamine (3.03 g) in tetrahydrofuran (30 ml) at ambient temperature under stirring. The resultant mixture was stirred at ambient temperature for 6 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was crystallized from diisopropyl ether to give methyl 2-[4-({[4′-(trifluoromethyl)-1,1′-biphenyl-2-yl]carbonyl}amino)phenyl]propanoate (1.83 g).

¹H-NMR (DMSO-d₆): δ1.35 (3H, d, J=7.10 Hz), 3.57 (3H, s), 3.73 (1H, q, J=7.10 Hz), 7.18 (2H, d, J=8.50 Hz), 7.46-7.65 (8H, m), 7.76 (2H, d, J=8.30 Hz), 10.39 (1H, s)

Preparation 111

2-[4-({[4′-(trifluoromethyl)-1,1′-biphenyl-2-yl]carbonyl}amino)phenyl]propanoic acid was obtained from methyl 2-[4-({[4′-(trifluoromethyl)-1,1′-biphenyl-2-yl]carbonyl}amino)phenyl]propanoate in the same manner as in Preparation 2.

¹H-NMR (DMSO-d₆): δ1.33 (3H, d, J=7.14 Hz), 3.61 (1H, q, J=7.14 Hz), 7.20 (2H, d, J=8.50 Hz), 7.48 (2H, d, J=8.46 Hz), 7.50-7.66 (6H, m), 7.76 (2H, d, J=8.32 Hz), 10.36 (1H, s), 12.25 (1H, s)

### EXAMPLE 289

N-{4-[1-Methyl-2-oxo-2-(2-pyridinylamino)ethyl]phenyl}-4′-(trifluoromethyl)-1,1′-biphenyl-2-carboxamide was obtained from 2-[4-({[4′-(trifluoromethyl)-1,1′-biphenyl-2-yl]carbonyl}amino)phenyl]propanoic acid and 2-aminopyridine in the same manner as in Preparation 97.

¹H-NMR (DMSO-d₆) δ1.37 (3H, d, J=6.96 Hz), 3.97 (1H, q, J=6.96 Hz), 7.07-7.10 (1H, m), 7.31 (2H, d, J=8.50 Hz), 7.47 (2H, d, J=8.52 Hz), 7.50-7.64 (7H, m), 7.75 (2H, d, J=8.30 Hz), 8.05 (1H, d, J=8.26 Hz), 8.28 (1H, dd, J=1.02 Hz,4.96 Hz), 10.35 (1H, s), 10.55 (1H, s)

### EXAMPLE 290

A solution of N-{4-[2-oxo-2-(2-pyridinylamino)ethyl]phenyl}-4′-(trifluoromethyl)-1,1′-biphenyl-2-carboxamide (1.165 g) in tetrahydrofuran (12 ml) was dropwise added to a mixture of lithium.aluminum hydride (186 mg) in tetrahydrofuran (50 ml) under an atmospheric pressure of nitrogen at 60-65° C. under stirring. The reaction mixture was refluxed under stirring for 2 hours. The resultant mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel (30 g) eluting with ethyl acetate and n-hexane (5:5). The fraction was evaporated in vacuo and the residue was recrystallized from ethyl acetate and diisopropyl ether to give N-{4-[2-(2-pyridinylamino)ethyl}phenyl}-4′-(trifluoromethyl)-1,1′-biphenyl-2-carboxamide (0.64 g).

¹H-NMR (DMSO-d₆): δ2.76 (2H, t, J=7.64 Hz), 3.34-3.46 (2H, m), 6.42-6.51 (3H, m), 7.11 (2H, d, J=7.16 Hz), 7.30-7.66 (9H, m), 7.76 (2H, d, J=8.32 Hz), 7.98 (1H, d, J=1.92 Hz), 10.31 (1H, s)

Preparation 112

A solution of 4′-(trifluoromethyl)-1,1′-biphenyl-2-carbonyl chloride (5.69 g) in ethyl acetate (5 ml) was added to solution of 3-aminobenzoic acid (3.02 g) and N,O-bis(trimethylsilyl)acetamide (6 ml) in ethyl acetate (100 ml) at ambient temperature under stirring. The resultant mixture was stirred at ambient temperature for 6 hours. The reaction mixture was poured into a mixture of ethyl acetate and water. The organic layer was washed with water and brine and dried over magnesium sulfate. The solvent was concentrated in vacuo and the precipitate was collected by filtration to give 3-({[4′-(trifluoromethyl)-1,1′-biphenyl-2-yl]carbonyl}amino)benzoic acid (7.0 g).

¹H-NMR (DMSO-d₆): δ7.37-8.21 (11H, m), 8.22 (1H, s), 10.55 (1H, s), 12.95 (1H, s)

### EXAMPLE 291

N-(3-{[(2-Pyridinylmethyl)amino]carbonyl}phenyl)-4′-(trifluoromethyl)-1,1′-biphenyl-2-carboxamide was obtained from 3-({[4′-(trifluoromethyl)-1,1′-biphenyl-2-yl]carbonyl}amino)benzoic acid and 2-(aminomethyl)pyridine in the same manner as in Preparation 97.

¹H-NMR (DMSO-d₆): δ4.55 (2H, d, J=5.86 Hz), 7.28-7.43 (3H, m), 7.53-7.78 (l1H, m), 8.10 (1H, s), 8.49-8.52 (1H, m), 9.05 (1H, t, J=5.86 Hz), 10.53 (1H, s)

### EXAMPLE 292

N-[3-({[2-(2-Pyridinyl)ethyl]amino}carbonyl)phenyl]-4′-(trifluoromethyl)-1,1′-biphenyl-2-carboxamide was obtaine from 3-({[4′-(trifluoromethyl)-1,1′-biphenyl-2-yl]carbonyl}amino)benzoic acid and 2-(aminoethyl)pyridine in the same manner as in Preparation 97.

¹H-NMR (DMSO-d₆): δ2.98 (2H, t, J=7.60 Hz), 3.55-3.65 (2H, m), 7.24-7.35 (3H, m), 7.46-7.78 (11H, m), 8.04 (1H, s), 8.49-8.52 (2H, m), 10.51 (1H, s)

### EXAMPLE 293

WSC (0.17 g) was added to the solution of 4-({[4′-(trifluoromethyl)-1,1′-biphenyl-2-yl]carbonyl}amino)benzoic acid (0.39 g), 2-pyridinemethanol (0.11 ml), HOBT.H₂O (0.17 g) and 4-dimethylaminopyridine (6 mg) in dichloromethane (5 ml) under ice-cooling and the mixture was stirred at ambient temperature for 18 hours.

The reaction mixture was poured into ethyl acetate and the mixture was washed with saturated aqueous sodium hydrogencarbonate solution and water. The organic layer was dried over magnesium sulfate and evaporated in vacuo. The residue was crystallized from ether to give 2-pyridinylmethyl 4-({[4′-(trifluoromethyl)-1,1′-biphenyl-2-yl]carbonyl}amino)benzoate (0.23 g).

¹H-NMR (DMSO-d₆): δ5.39 (2H, s), 7.32-7.39 (1H, m), 7.46-7.80 (11H, m), 7.84 (1H, dt, J=1.8 Hz, 7.7 Hz), 7.97 (2H, d, J=8.7 Hz), 8.57 (1H, dd, J=0.7 Hz, 4.8 Hz), 10.75 (1H, s)

(−)APCI-MS: 475 (M+H)⁻

Preparation 113

4′-(Trifluoromethyl)-1,1′-biphenyl-2-carbonyl-chloride (3.5 g) was added to a solution of (4-aminophenyl)methanol (1.5 g) and pyridine (1.0 ml) in dichloromethane (60 ml) under ice-cooling and the mixture was stirred under ice-cooling for 3 hours.

The reaction mixture was poured into ethyl acetate and the mixture was washed with water. The organic layer was dried over magnesium sulfate and evaporated in vacuo. The residue was crystallized from diisopropyl ether to give N-[4-(hydroxymethyl)phenyl]-4′-(trifluoromethyl)-1,1′-biphenyl-2-carboxamide (4.07 g).

¹H-NMR (DMSO-d₆): δ4.43 (2H, d, J=5.6 Hz), 5.09 (1H, t, J==5.6 Hz), 7.21 (2H, d, J=8.4 Hz), 7.44-7.66 (6H, m), 7.47 (2H, d, J=8.4 Hz), 7.75 (2H, d, J=8.3 Hz), 10.29 (1H, s)

### EXAMPLE 294

WSC (0.17 g) was added to a solution of N-[4-(hydroxymethyl)phenyl]-4′-(trifluoromethyl)-1,1′-biphenyl-2-carboxamide (0.37 g), picolinic acid (0.14 g), HOBT.H₂O (0.17 g) and 4-dimethylaminopyridine (6 mg) in dichloromethane (5 ml) under ice-cooling and the mixture was stirred at ambient temperature for 20 hours.

The reaction mixture was poured into a mixture of ethyl acetate and tetrahydrofuran, and the mixture was washed with saturated aqueous sodium hydrogencarbonate solution and water. The organic layer was dried over magnesium sulfate and evaporated in vacuo. The residue was crystallized from ethyl acetate to give 4-{[4′-(trifluoromethyl)-1,1′-biphenyl-2-yl]carbonylamino}benzyl 2-pyridinecarboxylate (0.27 g).

¹H-NMR (DMSO-d₆): δ5.31 (2H, s), 7.40 (2H, d, J=8.5 Hz), 7.54-7.69 (9H, m), 7.76 (2H, d, J=8.3 Hz), 7.94-8.12 (2H, m), 8.69-8.74 (1H, m), 10.45 (1H, s)

(+) ESI-MS: 499 (M+Na)⁺

### EXAMPLE 295

N-{4-[2-(5-Ethyl-2-pyridinyl)ethoxy]phenyl}-4′-(trifluoromethyl)-1,1′-biphenyl-2-carboxamide was obtained in the same manner as in Example 10.

¹H-NMR (DMSO-d₆): δ1.18 (3H, t, J=7.6 Hz), 2.59 (2H, q, J=7.6 Hz), 3.12 (2H, t, J=6.6 Hz), 4.28 (2H, t, J=6.6 Hz), 6.84 (2H, d, J=9.0 Hz), 7.27 (1H, d, J=7.9 Hz), 7.41 (2H, d, J=9.0 Hz), 7.47-7.66 (7H, m), 7.75 (2H, d, J=8.3 Hz), 8.37 (1H, d, J=1.9 Hz), 10.19 (1H, s)

(+)APCI-MS: 491 (M+H)⁺

Preparation 114

A mixture of 4-nitrobenzyl bromide (25.0 g), 2-pyridinemethanol (11.2 ml), and 1N sodium hydroxide (116 ml) in tetrahydrofuran (375 ml) was stirred for 24 hours at ambient temperature. The solvent was removed by concentration and to the residue was added a mixture of ethyl acetate and water. The mixture was adjusted to pH 1 with 6N hydrochloric acid. The separated aqueous layer was adjusted to pH 8 with 20% aqueous potassium carbonate solution and extracted with an ethyl acetate. The extract was washed with water, dried over magnesium sulfate and evaporated in vacuo to give 2-{[(4-nitrobenzyl)oxy]methyl}pyridine (9.55 g) as an oil.

¹H-NMR (DMSO-d₆): δ4.68 (2H, s), 4.78 (2H, s), 7.29-7.36 (1H, m), 7.51 (1H, d, J=7.8 Hz), 7.67 (2H, d, J=8.8 Hz), 7.83 (1H, dt, J=1.7 Hz, 7.8 Hz), 8.24 (2H, d, J=8.8 Hz), 8.52-8.55 (1H, m)

Preparation 115

4-[(2-Pyridinylmethoxy)methyl]aniline was obtained in the same manner as in Preparation 16.

¹H-NMR (DMSO-d₆): δ4.39 (2H, s), 4.52 (2H, s), 5.07 (2H, s), 6.54 (2H, d, J=8.3 Hz), 7.02 (2H, d, J=8.3 Hz), 7.27-7.31 (1H, m), 7.43 (1H, d, J=7.8 Hz), 7.79 (1H, dt, J=1.7 Hz, 7.8 Hz), 8.48-8.53 (1H, m)

### EXAMPLE 296

N-{4-[(2-Pyridinylmethoxy)methyl]phenyl}-4′-(trifluoromethyl)-1,1′-biphenyl-2-carboxamide was obtained in the same manner as in Example 10.

¹H-NMR (DMSO-d₆): δ4.53 (2H, s), 4.58 (2H, s), 7.26-7.32 (3H, m), 7.43-7.67 (9H, m), 7.72-7.84 (3H, m), 8.52 (1H, d, J=4.2 Hz), 10.39 (1H, s)

(−)APCI-MS: 461 (M+H)⁻

Preparation 116

A solution of 4′-methyl-1,1′-biphenyl-2-carbonyl chloride (4.3 g) in acetonitrile (8.7 ml) was dropwise added to the solution of 1,4-phenylenediamine (2.4 g) and triethylamine (3.2 ml) in acetonitrile (72 ml) under ice-cooling and the mixture was stirred under ice-cooling for 4 hours. The solvent was removed by concentration and to the residue was added a mixture of ethyl acetate and water. The mixture was adjusted to pH 7 with 1N hydrochloric acid. The separated organic layer was washed with water and dried over magnesium sulfate. To the organic layer was added methanesulfonic acid (1.5 ml) and the mixture was stirred at ambient temperature for 2 hours. The isolated crystals were collected by filtration and recrystallized from a mixture of methanol,.tetrahydrofuran and ethyl acetate to give N-(4-aminophenyl)-4′-methyl-1,1′-biphenyl-2-carboxamide methanesulfonate (6.37 g).

¹H-NMR (DMSO-d₆): δ2.28 (3H, s), 2.34 (3H, s), 7.17 (2H, d, J=8.0 Hz), 7.25 (2H, d, J=8.8 Hz), 7.33 (2H, d, J=8.0 Hz), 7.42-7.58 (4H, m), 7.63 (2H, d, J=8.8 Hz), 9.68 (2H, s), 10.41 (1H, s)

### EXAMPLE 297

A mixture of N-(4-aminophenyl)-4′-methyl-1,1′-biphenyl-2-carboxamide methanesulfonate (1.99 g), 2-(2-pyridinyl)ethanol (1.68 ml) and raney nickel (0.2 ml, Kawaken Fine Chemicals Co., Ltd NDT-65) in dioxane (20 ml) was stirred at 120° C. for 45 hours. The raney nickel was filtered off and the filtrate was evaporated in vacuo. To the residue was added a mixture of ethyl acetate and water, and the mixture was adjusted to pH 9 with 20% aqueous potassium carbonate solution. The separated organic layer was washed with water, dried over magnesium sulfate and evaporated in vacuo. The residue was purified by column chromatography on silica gel eluting with ethyl acetate and diisopropyl ether (1:1 v/v) to give 4′-methyl-N-(4-{[2-(2-pyridinyl)ethyl]amino}phenyl)-1,1′-biphenyl-2-carboxamide (1.29 g).

¹H-NMR (DMSO-d₆): δ2.30 (3H, s), 2.96 (2H, t, J=7.4 Hz), 3.34 (2H, td, J=7.4 Hz, 5.8 Hz), 5.51 (1H, t, J=5.8 Hz), 6.50 (2H, d, J=8.9 Hz), 7.2-7.6 (15H, m), 7.65-7.8 (1H, m), 8.52 (1H, d, J=4.9 Hz), 9.80 (1H, s)

(+)APCI-MS: 408 (M+H)⁺

Preparation 117

A solution of 4′-methyl-1,1′-biphenyl-2-carbonyl chloride (6.9 g) in acetonitrile (14 ml) was dropwise added to a solution of 1,4-phenylenediamine (3.9 g) and triethylamine (5.0 ml) in acetonitrile (117 ml) under ice-cooling and the mixture was stirred under ice-cooling for 4 hours. The solvent was-removed by concentration and to the residue was added a solution of ethyl acetate and water. The mixture was adjusted to pH 7.5 with 1N hydrochloric acid. The separated organic layer was washed with water and dried over magnesium sulfate. To the organic layer was added a 4N methanolic hydrogen chloride (9 ml) and the mixture was stirred at ambient temperature for 2 hours. The isolated crystals were collected by filtration and recrystallized from a mixture of methanol, tetrahydrofuran and ethyl acetate to give N-(4-aminophenyl)-4′-methyl-1,1′-biphenyl-2-carboxamide hydrochloride (8.92 g).

¹H-NMR (DMSO-d₆): δ2.28 (3H, s), 7.17 (2H, d, J=7.9 Hz), 7.25-7.36 (4H, m), 7.41-7.58 (4H, m), 7.63 (2H, d, J=8.8 Hz), 10.19 (2H, br.s), 10.41 (1H, s)

### EXAMPLE 298

A mixture of N-(4-aminophenyl)-4′-methyl-1,1′-biphenyl-2-carboxamide hydrochloride (5.0 g) and 2-vinylpyridine (1.6 ml) in n-propanol (50 ml) was stirred at 90° C. for 30 hours. The reaction mixture was evaporated in vacuo. To the residue was added a mixture of ethyl acetate, tetrahydrofuran and water, and the mixture was adjusted to pH 9 with 20% aqueous potassium carbonate solution. The separated organic layer was washed with water, dried over magnesium sulfate and evaporated in vacuo. The residue was purified by column chromatography on silica gel eluting with ethyl acetate and diisopropyl ether (1:1 v/v) to give 4′-methyl-N-(4-{[2-(2-pyridinyl)ethyl]amino}phenyl)-1,1′-biphenyl-2-carboxamide (2.14 g).

¹H-NMR (DMSO-d₆): δ2.30 (3H, s), 2.96 (2H, t, J=7.4 Hz), 3.34 (2H, td, J=7.4 and 5.6 Hz), 5.51 (1H, t, J=5.8 Hz), 6.50 (2H, d, J=8.9 Hz), 7.2-7.6 (15H, m), 7.65-7.8 (1H, m), 8.52 (1H, d, J=4.9 Hz), 9.80 (1H, s)

(+)APCI-MS: 408 (M+H)⁺

### EXAMPLE 299

N-[4-(1H-Imidazol-1-yl)phenyl]-4′-(trifluoromethyl)-1,1′-biphenyl-2-carboxamide was obtained in the same manner as in Example 10.

¹H-NMR (DMSO-d₆) δ7.09 (1H, s), 7.52-7.72 (11H, m), 7.77 (2H, d, J=8.4 Hz), 8.18 (1H, s), 10.54 (1H, s)

(+)APCI-MS: 408 (M+H)⁺

### EXAMPLE 300

N-[4-(1H-Imidazol-1-ylmethyl) phenyl]-4′-(trifluoromethyl)-1,1′-biphenyl-2-carboxamide was obtained in the same manner as in Example 10.

¹H-NMR (DMSO-d₆) δ5.11 (2H, s), 6.88 (1H, s), 7.14 (1H, s), 7.19 (2H, d, J=8.4 Hz), 7.46-7.65 (8H, m), 7.70-7.77 (3H, m), 10.41(1H, s)

(+)APCI-MS: 422 (M+H)⁺

### EXAMPLE 301

N-{4-[2-(1H-Imidazol-1-yl)ethyl]phenyl}-4′-(trifluoromethyl)-1,1′-biphenyl-2-carboxamide was obtained in the same manner as in Example 10.

¹H-NMR (DMSO-d₆): δ2.96 (2H, t, J=7.2 Hz), 4.16 (2H, t, J=7.2 Hz), 6.84 (1H, s), 7.04-7.13 (3H, m), 7.39-7.66 (9H, m), 7.75 (2H, d, J=8.3 Hz), 10.52 (1H, s)

(+)APCI-MS: 436 (M+H)⁺

### EXAMPLE 302

N-{6-[(6-Methyl-2-pyridinyl)methoxy]-3-pyridinyl}-4′-(trifluoromethyl)-1,1′-biphenyl-2-carboxamide was obtained in the same manner as in Example 10.

¹H-NMR (DMSO-d₆): δ2.47. (3H, s), 5.33 (2H, s), 6.91 (1H, d, J=8.9 Hz), 7.14-7.22 (2H, m), 7.50-7.80 (9H, m), 7.86 (1H, dd, J=2.5 Hz, 8.9 Hz), 8.25 (1H, d, J=2.5 Hz), 10.40 (1H, s)

(+)APCI-MS: 464 (M+H)⁺

### EXAMPLE 303

N-[4-(2-Pyridinylmethyl)phenyl]-2-[4-(trifluoromethyl)-benzyl]benzamide was obtained in the same manner as in Example 56.

¹H-NMR (DMSO-d₆): δ4.04 (2H, s), 4.21 (2H, s), 7.16-7.74 (15H, m), 8.48 (1H, d, J=4.3 Hz), 10.31 (1H, s)

(+)APCI-MS: 447 (M+H)⁺

### EXAMPLE 304

N-(4-{[2-(2-Pyridinyl)ethyl]amino}phenyl)-2-[4-(trifluoromethyl)benzyl]benzamide was obtained in the same manner as in Example 101.

¹H-NMR (DMSO-d₆): δ2.99 (2H, t, J=7.2 Hz), 3.29-3.43 (2H, m), 4.22 (2H, s), 5.56 (1H, t, J=5.8 Hz), 6.57 (2H, d, J=8.9 Hz), 7.18-7.27 (1H, m), 7.28-7.49 (9H, m), 7.59 (2H, d, J=8.1 Hz), 7.71 (1H, dt, J=1.9 Hz, 7.6 Hz), 8.49-8.54 (1H, m,) 9.96 (1H, s).

(+)APCI-MS: 476 (M+H)⁺

### EXAMPLE 305

N-(4-{[2-(2-Pyridinyl)ethyl]amino}phenyl)-2-[3-(trifluoromethyl)benzyl]benzamide was obtained in the sane manner as in Example 101.

¹H-NMR (DMSO-d₆): δ2.98 (2H, t, J=7.2 Hz), 3.30-3.42 (2H, m), 4.23 (2H, s), 5.56 (1H, t, J=5.7 Hz), 6.57 (2H, d, J=8.8 Hz), 7.19-7.27 (1H, m), 7.28-7.62 (11H, m), 7.65-7.76 (1H, m), 8.49-8.54 (1H, m), 9.98 (1H, s)

(+)APCI-MS: 476 (M+H)⁺

### EXAMPLE 306

2-[3,5-Bis(trifluoromethyl)benzyl]-N-(4-{[2-(2-pyridinyl)ethyl]amino}phenyl)benzamide was obtained in the same manner as in Example 101.

¹H-NMR (DMSO-d₆): δ2.99 (2H, t, J=7.2 Hz), 3.30-3.43 (2H, m), 4.34 (2H, s), 5.56 (1H, t, J=5.6 Hz), 6.57 (2H, d, J=8.8 Hz), 7.22 (1H, dd, J=5.0 Hz, 6.6 Hz), 7.28-7.52 (7H, m), 7.71 (1H, dt, J=1.7 Hz, 7.6 Hz), 7.87-7.94 (3H, m), 8.52 (1H, d, J=4.1 Hz), 10.02 (1H, s)

(+)APCI-MS: 544 (M+H)⁺

Preparation 118

A mixture of 2-(1,3-thiazol-4-yl)ethylamine (1.269 g), 1-fluoro-4-nitrobenzene (1.397 g) and triethylamine (1.00 g) in 1,3-dimethyl-2-imidazolidinone (15 ml) was heated to 50° C. for 11 hours. The reaction mixture was cooled to room temperature, poured into water and extracted with ethyl acetate. The organic layer was washed with brine, dried over magnesium sulfate, filtered, and concentrated in vacuo. The residue was purified by column chromatography on silica gel eluting with hexane:ethyl acetate (2:1) to give 4-[2-(4-nitroanilino)ethyl]-1,3-thiazole (1.374 g) as a yellow oil.

¹H-NMR (CDCl₃): δ3.17 (2H, t, J=6.4 Hz), 3.60 (2H, q, J=6.1 Hz), 6.53-8.09(4H, AaBb), 7.08 (1H, d, J=2.0 Hz), 8.80 (1H, s)

Preparation 119

To a solution of 4-[2-(4-nitroanilino)ethyl]-1,3-thiazole (1.945 g) and 4-(N,N-dimethylamino)pyridine (286 mg) in tetrahydrofuran (20 ml) was added di-tert-butyl dicarbonate (2.214 g) and the mixture was heated to 50° C. for 11 hours. The reaction mixture was cooled to room temperature and concentrated in vacuo. The residue was dissolved in ethyl acetate and water, and extracted with ethyl acetate. The organic layer was washed with water and brine, dried over magnesium sulfate, filtered, and concentrated in vacuo. The residue was purified by column chromatography on silica gel eluting with hexane:ethyl acetate (4:1) to give tert-butyl 4-nitrophenyl[2-(1,3-thiazol-4-yl)ethyl]carbamate (2.501 g) as a dark orange oil.

¹H-NMR (CDCl₃): δ1.46 (9H, s), 3.14 (2H, t, J=6.8 Hz), 4.11 (2H, t, J=7.1 Hz), 7.01 (1H, d, J=2.0 Hz), 7.26-8.16 (4H, AaBb), 8.69 (1H, d, J=2.0 Hz)

Preparataion 120

A solution of tert-butyl 4-nitrophenyl[2-(1,3-thiazol-4-yl)ethyl]carbamate (2.501 g) in methanol (50 ml) was hydrogenated over 10% palladium on carbon at room temperature under atmospheric pressure of hydrogen for 2 hours. The reaction mixture was filtered through a short pad of celite, and the filtrate was concentrated in vacuo to give tert-butyl 4-aminophenyl[2-(1,3-thiazol-4-yl)ethyl]carbamate (75 mg) as an orange oil.

¹H-NMR (CDCl₃): δ1.39 (9H, s), 3.07 (2H, t, J=7.4 Hz), 3.93 (2H, t, J=7.4 Hz), 6.71 (2H, d, J=8.6 Hz), 6.9 (2H, br. s), 7;00 (1H, br. s), 8.7 (1H, d, J=2.0 Hz)

### EXAMPLE 307

To a solution of 4′-chloro-1,1′-biphenyl-2-carboxylic acid (164 mg) in toluene (5 ml) were added thionyl chloride (168 mg) and N,N-dimethylformamide (1 drop) and the mixture was stirred at 80° C. for 2 hours. The mixture was evaporated in vacuo and the residue was dissolved in tetrahydrofuran (2 ml). The obtained acid chloride solution in tetrahydrofuran was added to a solution of tert-butyl 4-aminophenyl(2-{2-[(tert-butoxycarbonyl)amino]-1,3-thiazol-4-yl}ethyl)carbamate (205 mg) and triethylamine (130 mg) in tetrahydrofuran (5 ml) at room temperature, and the mixture was stirred at the same temperature for 30 minutes. The mixture was poured into water and extracted with ethyl acetate. The organic layer was washed with brine, dried over magnesium sulfate, and evaporated in vacuo. The residue was purified by column chromatography on silica gel eluting with hexane:ethyl acetate (1:1) to give tert-butyl 4-{[(4′-chloro-1,1′-biphenyl-2-yl)carbonyl]amino}phenyl[2-(1,3-thiazol-4-yl)ethyl]carbamate (332 mg) as an orange foam.

¹H-NMR (CDCl₃): δ1.40 (9H, s), 3.06 (2H, t, J=7.2 Hz), 3.97 (2H, t, J=7.2 Hz), 6.95-7.02 (4H, m), 7.15 (2H, d, J=8.9 Hz), 7.39-7.57 (7H, m), 7.81 (1H, d, J=7.2 Hz), 8.69 (1H, d, J=2.0 Hz)

### EXAMPLE 308

To a solution of tert-butyl 4-{[(4′-chloro-1,1′-biphenyl-2-yl)carbonyl]amino}phenyl[2-(1,3-thiazol-4-yl)ethyl]carbamate (313 mg) in dichloromethane (10 ml) was added trifluoroacetic acid (0.68 ml). The reaction mixture was stirred for 15 hours, quenched with 10% aqueous potassium carbonate solution, and extracted with dichloromethane. The organic layer was washed with brine, dried over magnesium sulfate, filtered, and concentrated in vacuo. The residue was recrystallized from ethyl acetate-diisopropyl ether to give 4′-chloro-N-(4-{[2-(1,3-thiazol-4-yl)ethyl]amino}phenyl)-1,1′-biphenyl-2-carboxamide (161 mg) as a pale purple solid.

¹H-NMR (CDCl₃): δ3.11 (2H, t, J=6.6 Hz), 3.47 (2H, t, J=6.4 Hz), 4.05 (1H, br.s), 6.53 (2H, d, J=8.9 Hz), 6.74 (1H, br.s), 6.99 (2H, d, J=8.9 Hz), 7.02 (1H, d, J=2.0 Hz), 7.37-7.55 (7H, m), 7.78 (1H, d, J=7.2 Hz), 8.77 (1H, d, J=2.0 Hz)

ESI-MS (m/z): 434 (M+H)⁺

Preparation 121

A mixture of 2-(2-methyl-1,3-thiazol-4-yl)ethylamine (6.823 g), 1-fluoro-4-nitrobenzene (8.123 g) and triethylamine (5.829 g) in 1,3-dimethyl-2-imidazolidinone (50 ml) was heated at 50° C. for 16 hours. The reaction mixture was cooled to room temperature, poured into water and extracted with ethyl acetate. The organic layer was washed with brine, dried over magnesium sulfate, filtered, and concentrated in vacuo. The residue was purified by column chromatography on silica gel eluting with hexane:ethyl acetate (2:1) to give N-[2-(2-methyl-1,3-thiazol-4-yl)ethyl]-4-nitroaniline (7.764 g) as a yellow oil.

¹H-NMR (CDCl₃): δ2.78 (3H, s), 3.05 (2H, t, J=6.3 Hz), 3.54 (2H, t, J=6.3 Hz), 6.54 (2H, d, J=8.9 Hz), 6.83 (1H, s), 8.09 (2H, d, J=9.2 Hz)

Preparation 122

To a solution of N-[2-(2-methyl-1,3-thiazol-4-yl)ethyl]-4-nitroaniline (7.764 g) and 4-(N,N-dimethylamino)pyridine (1.081 mg) in tetrahydrofuran (100 ml) was added di-tert-butyl dicarbonate (8.366 g) and heated at 50° C. for 12 hours. The reaction mixture was cooled to room temperature and concentrated in vacuo. The residue was dissolved in ethyl acetate and water, and extracted with ethyl acetate. The organic layer was washed with water and brine, dried over magnesium sulfate, filtered, and concentrated in vacuo. The residue was purified by column chromatography on silica gel eluting with hexane:ethyl acetate (4:1) to give tert-butyl 2-(2-methyl-1,3-thiazol-4-yl)ethyl(4-nitrophenyl)carbamate (10.63 g) as a dark orange oil.

¹H-NMR (CDCl₃): δ1.47 (9H, s), 2.60 (3H, s), 3.03 (2H, t, J=7.0 Hz), 4.08 (2H, t, J=7.0 Hz), 6.76 (1H, s), 7.31 (2H, d, J=9.2 Hz), 8.14 (2H, d, J=9.2 Hz)

Prepration 123

A solution of tert-butyl 2-(2-methyl-1,3-thiazol-4-yl)ethyl(4-nitrophenyl)carbamate (10.63 g) in methanol (100 ml) was hydrogenated over 10% palladium on carbon at room temperature under atmospheric pressure of hydrogen for 4.5 hours. The reaction mixture was filtered through a pad of celite, and the filtrate was concentrated in vacuo. The residue was purified by column chromatography on silica gel eluting with chloroform:methanol (19:1) to give tert-butyl 4-aminophenyl[2-(2-methyl-1,3-thiazol-4-yl)ethyl]carbamate (9.295 g) as yellow crystals.

¹H-NMR (CDCl₃): δ1.39 (9H, s), 2.64 (3H, s), 2.96 (2H, t, J=7.6 Hz), 3.63 (2H,br.s), 3.90 (2H, t, J=7.6 Hz), 6.67 (2H, d, J=7.9 Hz), 6.78 (1H, s), 6.90 (2H, d, J=7.9 Hz)

### EXAMPLE 309

To a solution of 4′-methoxy-1,1′-biphenyl-2-carboxylic acid (205.4 mg) in toluene (2 ml) were added thionyl chloride (214.2 mg) and N,N-dimethylformamide (1 drop) and the mixture was stirred at 80° C. for 2 hours. The mixture was evaporated in vacuo and the residue was dissolved in tetrahydrofuran (2 ml). The obtained acid chloride solution in tetrahydrofuran was added to a solution of tert-butyl 4-aminophenyl[2-(2-methyl-1,3-thiazol-4-yl)ethyl]carbamate (250 mg) and triethylamine (151.8 mg) in tetrahydrofuran (5 ml) at room temperature, and the mixture was stirred at room temperature for 30 minutes. The mixture was poured into water and extracted with ethyl acetate. The organic layer was washed with brine, dried over magnesium sulfate, and evaporated in vacuo to give tert-butyl 4-{[(4′-methoxy-1,1′-biphenyl-2-yl)carbonyl]amino}phenyl[2-(2-methyl-1,3-thiazol-4-yl)ethyl]carbamate (404 mg) as a yellow foam.

### EXAMPLE 310

To a solution of tert-butyl 4-{[(4′-methoxy-1,1′-biphenyl-2-yl)carbonyl]amino}phenyl[2-(2-methyl-1,3-thiazol-4-yl)ethyl]carbamate (400 mg) in dichloromethane (10 ml) was added trifluoroacetic acid (1.98 ml). The reaction mixture was stirred for 15 hours, quenched with 10% aqueous potassium carbonate solution, and extracted with dichloromethane. The organic layer was washed with brine, dried over magnesium sulfate, filtered, and concentrated in vacuo. The residue was recrystallized from ethyl acetate-hexane to give 4′-methoxy-N-(4-{[2-(2-methyl-1,3-thiazol-4-yl)ethyl]amino}phenyl)-1,1′-biphenyl-2-carboxamide (224.6 mg) as pale yellow crystals.

¹H-NMR (CDCl₃): δ2.70 (3H, s), 2.99 (2H, t, J=6.6 Hz), 3.42 (2H, t, J=6.6 Hz), 3.84 (3H, s), 6.51 (2H, d, J=8.6 Hz), 6.75 (1H, s), 6.77 (1H, s), 6.95 (2H, d, J=8.2 Hz), 7.26-7.52 (3H, m), 7.41 (2H, d, J=8.2 Hz), 7.84 (1H, dd, J=7.3 Hz, 1.6 Hz).

ESI-MS (m/z): 444 (M+H)⁺

### EXAMPLE 311

tert-Butyl 4-{[(4′-chloro-1,1′-biphenyl-2-yl)carbonyl]amino}phenyl[2-(2-methyl-1,3-thiazol-4-yl)ethyl]carbamate was obtained in the same manner as in Example 309 as a pale yellow foam.

### EXAMPLE 312

4′-Chloro-N-(4-{[2-(2-methyl-1,3-thiazol-4-yl)ethyl]amino}phenyl)-1,1′-biphenyl-2-carboxamide was in the same manner as in Example 310 as pale yellow crystals

¹H-NMR (CDCl₃): δ2.70 (3H, s), 3.00 (2H, t, J=6.6 Hz), 3.43 (2H, t, J=6.3 Hz), 6.53 (2H, d, J=8.9 Hz), 6.73 (1H, s), 6.77 (1H, s), 7.37-7.54 (7H, m), 7.78 (1H, dd, J=6.9 Hz, 1.6 Hz)

ESI-MS (m/z): 448 (M+H)⁺

### EXAMPLE 313

To a solution of N-(4-aminophenyl)-4′-(trifluoromethyl)-1,1′-biphenyl-2-carboxamide (0.792 g), 4-pyrimidinylacetic acid (0.307 g) and HOBT (0.360 g) in N,N-dimethylformamide (10 ml) was added WSC.HCl (0.511 g), followed by triethylamine (0.47 ml) at room temperature. The reaction mixture was stirred at 50° C. for 12 hours and concentrated in vacuo. The residue was dissolved in ethyl acetate and water, and extracted with ethyl acetate. The organic layer was washed with water and brine, dried over magnesium sulfate, filtered, and concentrated in vacuo. The residue was recrystallized from ethyl acetate-diisopropyl ether to give N-{4-[(4-pyrimidinylacetyl)amino]phenyl}-4′-(trifluoromethyl)-1,1′-biphenyl-2-carboxamide (0.732 g) as a yellow brown solid.

¹H-NMR (DMSO-d₆): δ3.86 (2H, s), 7.43-7.76 (13H, m), 8.74 (1H, d, J=5.3 Hz), 9.10 (1H, s), 10.25 (1H, s), 10.29 (1H, s)

### EXAMPLE 314

To a solution of 2-(1,3-thiazol-4-yl)ethylamine (94 mg), 4-({[4′-(trifluoromethyl)-1,1′-biphenyl-2-]carbonyl}amino)benzoic acid (0.273 g) and HOBT (0.119 g) in N,N-dimethylformamide (15 ml) was added WSC.HCl (0.169 g), followed by triethylamine (0.15 ml) at room temperature. The reaction mixture was stirred at 50° C. for 12 hours and concentrated in vacuo. The residue was dissolved in ethyl acetate and water, and extracted with ethyl acetate. The organic layer was washed with water and brine, dried over magnesium sulfate, filtered, and concentrated in vacuo. The residue was purified by column chromatography on silica gel eluting with hexane:ethyl acetate (1:1) to give N-[4-({[2-(1,3thiazol-4-yl)ethyl]amino}carbonyl)phenyl]-4′-(trifluoromethyl)-1,1′-biphenyl-2-carboxamide (0.254 g) as a white solid.

¹H-NMR (DMSO-d₆): δ3.01 (2H, t, J=7.3 Hz), 3.57 (2H, q, J=7.1 Hz), 7.40 (1H, s), 7.41-7.78 (12H, m), 8.47 (1H, t, J=5.6 Hz), 9.04 (1H, d, J=2.0 Hz), 10.58 (1H, s)

ESI-MS (m/z): 496 (M+H)⁺

### EXAMPLE 315

To a solution of 4-({[4′-(trifluoromethyl)-1,1′-biphenyl-2-yl]carbonyl}amino)benzoic acid (241 mg), tert-butyl 6-(2-aminoethyl)-2-pyridinylcarbamate (154 mg) and HOBT.H₂O (119 mg) in N,N-dimethylformamide (10 ml) was added WSC.HCl (149 mg), followed by triethylamine (85 mg) at room temperature. The reaction mixture was stirred at 45° C. for 11 hours and concentrated in vacuo. The residue was dissolved in ethyl acetate and water, and extracted with ethyl acetate. The organic layer was washed with brine, dried over magnesium sulfate, filtered and concentrated in vacuo. The residue was purified by column chromatography on silica gel eluting with chloroform:methanol (19:1) to give tert-butyl 6-(2-{[4-({[4′-(trifluoromethyl)-1,1′-biphenyl-2-yl]carbonyl}amino)benzoyl]amino}ethyl)-2-pyridinylcarbamate (373 mg) as a yellow oil.

¹H-NMR (CDCl₃): δ1.52 (9 Hs), 2.94 (2H, t, J=6.8 Hz), 3.76 (2H, q, J=6.1 Hz), 6.82 (1H, d, J=7.3 Hz), 6.90 (1H, br.s), 7.23-7.30 (2H, m), 7.41-7.66 (11H, m), 7.72-7.77 (2H, m)

### EXAMPLE 316

To a solution of tert-butyl 6-(2-{[4-({[4′-(trifluoromethyl)-1,1′-biphenyl-2-yl]carbonyl}amino)benzoyl]-amino}ethyl)-2-pyridinylcarbamate (373 mg) in dichloromethane (10 ml) was added trifluoroacetic acid (1.05 g) by a syringe at 0° C. The reaction mixture was allowed to warm to room temperature and stirred for 16 hours. The reaction was quenched with 10% aqueous potassium carbonate solution. The separated organic layer was washed with brine, dried over magnesium sulfate, filtered and concentrated in vacuo to give N-[4-({[2-(6-amino-2-pyridinyl)ethyl]amino}carbonyl)phenyl]-4′-(trifluoromethyl)-1,1′-biphenyl-2-carboxamide (215 mg) as colorless crystals.

¹H-NMR (CDCl₃): δ2.90 (2H, t, J=6.5 Hz), 3.59 (2H, q, J=5.6 Hz), 6.60 (1H, d, J=7.0 Hz), 6.72 (1H, d, J=8.9 Hz), 7.52-7.77 (13H, m), 8.50 (1H, t, J=5.3 Hz), 10.59 (1H, s)

ESI-MS (m/z): 527 (M+Na)⁺, 505 (M+H)⁺

### EXAMPLE 317

To a solution of tert-butyl 4-(2-aminoethyl)-1,3-thiazol-2-ylcarbamate (0.256 g), 4-({[4′-(trifluoromethyl)-1,1′-biphenyl-2-yl]carbonyl}amino)benzoic acid (0.391 g) and HOBT.H₂O (0.171 g) in N,N-dimethylformamide (20 ml) was added WSC.HCl (0.242 g), followed by triethylamine (0.22 ml) at room temperature. The reaction mixture was stirred at 50° C. for 12 hours and concentrated in vacuo. The residue was dissolved in ethyl acetate and water, and extracted with ethyl acetate. The organic layer was washed with water and brine, dried over magnesium sulfate, filtered, and concentrated in vacuo. The residue was purified by column chromatography on silica gel eluting with hexane:ethyl acetate (3:7) to give tert-butyl 4-(2-{[4-({[4′-(trifluoromethyl)-1,1′-biphenyl-2-yl]carbonyl}amino)ethyl)-1,3-thiazol-2-ylcarbamate (0.630 g) as a pale yellow oil.

¹H-NMR (CDCl₃): δ1.48 (9H, s), 2.80 (2H, t, J=7.2 Hz), 3.50 (2H, dd, J=6.9 Hz, 5.9 Hz), 6.78 (1H, s), 7.52-7.77 (13H, m), 8.41 (1H, t, J=5.4 Hz), 10.57 (1H, s), 11.38 (1H, s)

### EXAMPLE 318

To a solution of tert-butyl 4-(2-{[4-({[4′-(trifluoromethyl)-1,1′-biphenyl-2-yl]carbonyl}amino)benzoyl]amino}ethyl)-1,3-thiazol-2-ylcarbamate (0.618 g) in dichloromethane (30 ml) was added trifluoroacetic acid (1.6 ml). The reaction mixture was stirred for 15 hours, quenched with 10% aqueous potassium carbonate solution, and extracted with dichloromethane. The organic layer was washed with brine, dried over magnesium sulfate, filtered, and concentrated in vacuo. The residue was recrystallized from ethyl acetate-diisopropyl ether to give N-[4-({[2-(2-amino-1,3-thiazol-4yl)ethyl]amino}carbonyl)phenyl]-4′-(trifluoromethyl)-1,1′-biphenyl-2-carboxamide (0.201 g) as a white solid.

¹H-NMR (CDCl₃): δ2.65 (2H, t, J=7.4 Hz), 3.46 (2H, dd, J=6.9 Hz, 5.9 Hz), 6.19 (1H, s), 6.86 (2H, br. s), 7.51-7.79 (12H, m), 8.41 (1H, t, J=5.6 Hz), 10.58 (1H, s)

ESI-MS (m/z): 511 (M+H)⁺

### EXAMPLE 319

To a solution of N-(4-aminophenyl)-4′-(trifluoromethyl)-1,1′-biphenyl-2-carboxamide (0.140 g) and 2-vinylpyrazine (50 mg) in methoxyethanol (4 ml) was added acetic acid (20 μl) at room temperature and the mixture was refluxed for 2 days. The reaction mixture was cooled to room temperature, and extracted with ethyl acetate. The organic layer was washed with brine and dried over anhydrous magnesium sulfate. The solvent was evaporated in vacua and the residue was purified by column chromatography on silica gel eluting with hexane and ethyl acetate (1:2). The fraction containing the objective compound was evaporated to give N-(4-{[2-(2-pyrazinyl)ethyl]amino}phenyl)-4′-(trifluoromethyl)-1,1′-biphenyl-2-carboxamide (39 mg) as a pale brown solid.

¹H-NMR (CDCl₃): δ3.09 (2H, t, J=6.6 Hz), 3.54 (2H, t, J=6.6 Hz), 6.53 (2H, d, J=8.9 Hz), 6.74 (1H, s), 6.95 (2H, d, J=8.9 Hz), 7.40-7.81 (10H, m), 8.44 (1H, d, J=2.6 Hz), 8.46 (1H, d, J=1.6 Hz), 8.52 (1H, dd, J=2.6 Hz, 1.6 Hz)

ESI-MS (m/z: 463 (M+H)⁺

Preparation 124

A solution of tert-butyl 6-[2-(4-nitrophenoxy)ethyl]-2-pyridinylcarbamate (51.0 g) in methanol (1000 ml) was hydrogenated over 10% palladium on carbon (25.0 g) at room temperature under atmospheric pressure of hydrogen gas for 3 hours. The reaction mixture was filtered through a pad of celite, and the filtrate was concentrated in vacuo. The residue was purified by column chromatography on silica gel eluting with hexane and ethyl acetate (2:1 to 1:1). The fraction containing the objective compound was evaporated to give tert-butyl 6-{2-[4-(methylamino)phenoxy]ethyl}-2-pyridinylcarbamate as a yellow solid (2.22 g).

¹H-NMR (CDCl₃): δ1.51 (9H, s), 2.79 (3H, s), 3.08 (2H, t, J=6.7 Hz), 4.23 (2H, t, J=6.7 Hz), 6.55 (2H, d, J=8.6 Hz), 6.79 (2H, d, J=8.6 Hz), 6.90 (1H, d, J=7.2 Hz), 7.22 (1H, br.s), 7.57 (1H, t, J=7.8 Hz), 7.75 (1H, d, J=8.2 Hz)

ESI-MS (m/z): 366 (M+Na)⁺, 344 (M+H)⁺

### EXAMPLE 320

To a solution of 4′-(trifluoromethyl)-1,1′-biphenyl-2-carboxylic acid (500 mg) in toluene (5 ml) were added thionyl chloride (0.273 ml) and N,N-dimethylformamide (1 drop) and the mixture was stirred at 100° C. for 4 hours. The resultant mixture was cooled down to ambient temperature, and then the solvent was evaporated in vacuo. The excess thionyl chloride was removed as the toluene azeotrope twice. The residue was dissolved in tetrahydrofuran (2 ml) and the solution was added to a solution of tert-butyl 6-{2-[4-(methylamino)phenoxy]ethyl}-2-pyridinylcarbamate (645 mg) and triethylamine (285 mg) in tetrahydrofuran (5 ml) at room temperature. The reaction mixture was stirred at room temperature for 4 hours. The mixture was poured into water and extracted with ethyl acetate. The organic layer was washed with brine, dried over anhydrous magnesium sulfate and concentrated in vacuo to give tert-butyl 6-{2-[4-(methyl{[4′-(trifluoromethyl)-1,1′-biphenyl-2-yl]carbonyl}amino)phenoxy]ethyl}-2-pyridinylcarbamate (1.056 g) as a brown oil.

### EXAMPLE 321

To a solution of tert-butyl 6-{2-[4-(methyl{[4′-(trifluoromethyl)-1,1′-biphenyl-2-yl]carbonyl}amino)phenoxy]ethyl}-2-pyridinylcarbamate (1.05 g) in dichloromethane (20 ml) was added trifluoroacetic acid (4.8 ml) at room temperature. The reaction mixture was stirred for 12 hours, quenched with 10% aqueous potassium carbonate solution, and extracted with dichloromethane. The organic layer was washed with brine, dried over anhydrous magnesium sulfate and, concentrated in vacuo. The residue was purified by column chromatography on silica gel eluting with hexane and ethyl acetate (3:7 to 1:4). The fraction containing the objective compound was evaporated to give N-{4-[2-(6-amino-2pyridinyl)ethoxy]phenyl}-N-methyl-4′-(trifluoromethyl)-1,1′-biphenyl-2-carboxamide (0.602 g) as a white solid.

¹H-NMR (CDCl₃): δ3.17 (2H, t, J=6.3 Hz), 3.19 (3H, s), 4.17 (2H, t, J=6.0 Hz), 6.12 (2H, d, J=8.9 Hz), 6.44 (2H, d, J=8.9 Hz), 6.60-6.87 (2H, m), 7.10 (1H, d, J=7.3 Hz), 7.24-7.39 (3H, m), 7.53-7.71 (5H, m)

ESI-MS (m/z): 493 (M+H)⁺

Preparation 125

To a solution of 2-fluorobenzaldehyde (6.21 g) and 3-(trifluoromethyl)phenol (8.11 g) in N,N-dimethylformamide (150 ml) was added powdered potassium carbonate (6.91 g) at room temperature and the mixture was stirred at 150° C. for 40 hours under nitrogen. The mixture was cooled to room temperature and poured into a mixture of ethyl acetate and ice water. The separated organic layer was washed with water and brine, dried over magnesium sulfate and evaporated in vacuo. The residue was purified by column chromatography on silica gel eluting with hexane:ethyl acetate (4:1) to give 2-[3-(trifluoromethyl)-phenoxy]benzaldehyde (12.26 g) as a yellow oil.

¹H-NMR (DMSO-d₆): δ7.05-8.0 (8H, m), 10.35 (1H, s)

ESI-MS (m/z): 289 (M+Na)⁺

Preparation 126

To a solution of 2-[3-(trifluoromethyl)phenoxy]-benzaldehyde (12.1 g) and 2-methyl-2-butene (15.96 g) in tert-butanol (60 ml) and acetone (60 ml) was added a solution of sodium dihydrogenphosohate (16.44 g) in water (80 ml) at room temperature. To this mixture was added portionwise sodium chlorite (6.17 g) at room temperature and the reaction mixture was stirred at room temperature for 20 hours. To the mixture was added 10% aqueous sodium thiosulfate solution (100 ml). The mixture was stirred for 15 minutes and evaporated in vacuo to remove the oraganic solvents. To the aqueous layer was added ethyl acetate and the mixture was adjusted to pH 2 by addition of 6N HCl. The separated organic layer was washed with brine, dried over magnesium sulfate and evaporated in vacuo. The residue was purified by column chromatography on silica gel eluting with hexane:ethyl acetate (1:1) to give 2-[3-(trifluoromethyl)phenoxy]benzoic acid (12.35 g) as a yellow oil.

¹H-NMR (DMSO-d₆): δ7.1-7.25 (3H, m), 7.3-7.45 (2H, m), 7.5-7.7 (2H, m), 7.85-7.95 (1H, m), 12.98 (1H, br)

ESI-MS (m/z): 305 (M+Na)⁺

### EXAMPLE 322

tert-Butyl 2-(2-pyridinyl)ethyl[4-({2-[3-(trifluoromethyl)phenoxy]benzoyl}amino)phenyl]carbamate was obtained in the same manner as in Example 56 as a light brown amorphous powder.

¹H-NMR (DMSO-d₆): δ1.30 (9H, s), 2.87 (2H, t, J=7.7 Hz), 3.89 (2H, t, J=7.7 Hz), 7.1-7.75 (15H, m), 8.45 (1H, d, J=4.0 Hz), 10.42 (1H, s)

ESI-MS (m/z): 600 (M+Na)+, 578 (M+H)⁺

### EXAMPLE 323

N-(4-{[2-(2-Pyridinyl)ethyl]amino}phenyl)-2-[3-(trifluoromethyl)phenoxy]benzamide was obtained in the same manner as in Example 59 as a light yellow amorphous powder.

¹H-NMR (DMSO-d₆): δ2.96 (2H, t, J=7.0 Hz), 3.35 (2H, td, J=7.0 and 5.8 Hz), 5.55 (1H, t, J=5.8 Hz), 6.52 (2H, d, J=8.9 Hz), 7.2-7.75 (15H, m), 8.50 (1H, d, J=4.0 Hz), 9.95 (1H, s)

ESI-MS (m/z): 500 (M+Na)+, 478 (M+H)⁺

### EXAMPLE 324

To a solution of N-{4-[2-(6-amino-2-pyridinyl)ethoxy]phenyl}-4′-(trifluoromethyl)-1,1′-biphenyl-2-carboxamide (478 mg) in acetonitrile (30 ml) and methanol (30 ml) was added dropwise a solution of sodium bicarbonate (177 mg) in water (10 ml), followed by addition of a solution of OXONE® (615 mg) in water (5 ml) at room temperature. The resulting suspension was stirred at room temperature for 20 hours and extracted with ethyl acetate. The separated organic layer was washed with water and brine, dried over magnesium sulfate and evaporated in vacuo. The residue was purified by column chromatography on silica gel eluting with ethyl acetate:methanol (9:1) to give N-{4-[2-(6-amino-1-oxido-2-pyridinyl)ethoxy]phenyl}-4′-trifluoromethyl)-1,1′-biphenyl-2-carboxamide (189 mg) as a white amorphous powder.

¹H-NMR (DMSO-d6):d 3.17(2H, d, J=6.7 Hz), 4.28 (2H, d, J=6.7 Hz), 6.6-6.75 (2H, m), 6.81 (2H, d, J=9.0 Hz), 7.0-7.15 (1H, m), 7.41 (2H, d, J=9.0 Hz), 7.5-7.75 (6H, m), 7.76 (2H, d, J=8.3 Hz), 10.19 (1H, s)

APCI-MS (m/z): 494 (M−H)⁺

### EXAMPLE 325

To a solution of N-{4-[2-(6-amino-2-pyridinyl)ethoxy]phenyl}-4′-(trifluoromethyl)-1,1′-biphenyl-2-carboxamide (1.432 g) in methanol (20 ml) was added 10% HCl in methanol (3 ml) at 5° C. and the mixture was stirred at the same temperature for 30 minutes. To this solution was added dropwise diisopropyl ether (40 ml) and the mixture was warmed to room temperature and stirred at room temperature for 2 hours. The precipitate was collected by filtration, washed with methanol:diisopropyl ether (1:3), and dried in vacuo to give N-{4-[2-(6-amino-2-pyridinyl)ethoxy]phenyl}-4′-(trifluoromethyl)-1,1′-biphenyl-2-carboxamide hydrochloride (1.10 g) as white crystals.

¹H-NMR (DMSO-d₆): δ3.15 (2H, d, J=6.2 Hz), 4.28 (2H, d, J=6.2 Hz), 6.75-6.9 (4H, ), 7.35-7.9 (11H, m), 10.21 (1H, s), 14.10 (3H, m)

APCI-MS (m/z): 478 (M+H)⁺ (free)

### EXAMPLE 326

To a solution of tert-butyl 4-[(2-iodobenzoyl)amino]phenyl[2-(2-pyridinyl)ethyl]carbamate (2.72 g) and 4-(dihydroxyboryl)-1,1′-biphenyl (1.19 g) in N,N-dimethylformamide (40 ml) were added triethylamine (2.53 g) and tetrakis(triphenylphosphine) palladium (289 mg) at room temperature and the mixture was stirred at 150° C. for 16 hours under nitrogen. The mixture was cooled to room temperature and poured into a mixture of ethyl acetate and water. The separated organic layer was washed with water and brine, dried over magnesium sulfate and evaporated in vacuo. The residue was purified by column chromatography on silica gel eluting with hexane:ethyl acetate (3;1) to give crude tert-butyl 2-(2-pyridinyl)ethyl{4-[(1,1′:4′,1″-terphenyl-2-ylcarbonyl)amino]phenyl}carbamate (1.24 g) as a light brown amorphous powder.

¹H-NMR (DMSO-d₆): δ1.30 (9H, s), 2.95 (2H, t, J=7.0 Hz), 3.29 (2H, t, J=7.0 Hz), 6.51 (2H, d, J=8.8 Hz), 7.2-7.7 (20H, m), 8.45-8.55 (1H, m), 10.23 (1H, s)

ESI-MS (m/z): 592 (M+Na)⁺, 570 (M+H)⁺

### EXAMPLE 327

N-(4-{[2-(2-Pyridinyl)ethyl]amino}phenyl)-1,1′:4′,1″-terphenyl-2-carboxamide was obtained in the same manner as in Example 59 as white crystals.

¹H-NMR (DMSO-d₆): δ2.95 (2H, t, J=7.0 Hz), 3.29 (2H, td, J=7.0 and 5.8 Hz), 5.51 (1H, t, J=5.8 Hz), 6.51 (2H, d, J=8.8 Hz), 7.2-7.7 (20H, m), 8.45-8.55 (1H, m), 9.88 (1H, s)

ESI-MS (m/z): 492 (M+Na)⁺, 470 (M+H)⁺

Preparation 127

To a solution of 4′-hydroxy-1,1′-biphenyl-2-carboxylic acid (1.21 g) and triethylamine (2.02 g) in dichloromethane (40 ml) was added dropwise a solution of methanesulfonyl chloride (1.43 g) in dichloromethane (20 ml) at 5° C. and the mixture was stirred at the same temperature for 4 hours under nitrogen. To the mixture was added water and the separated organic layer was washed with water and brine, dried over magnesium sulfate and evaporated in vacuo to give crude 4′-methanesulfonyloxy-1,1′-biphenyl-2-carboxylic acid methanesulfonate (1.81 g) as a yellow oil. The crude product was used for the next step without purification.

### EXAMPLE 328

To a solution of crude 4′-methanesulfonyloxy-1,1′-biphenyl-2-carboxylic acid methanesulfonate (1.80 g) and 4-aminophenyl[2-(2-pyridinyl)ethyl]formamide (938 mg) in N,N-dimethylformamide (30 ml) was added triethylamine (983 mg) at room temperature and the mixture stirred at 80° C. for 6 hours under nitrogen. The mixture was cooled to room temperature and poured into a mixture of ethyl acetate and water. The separated organic layer was washed with water and brine, dried over magnesium sulfate and evaporated in vacuo. The residue was purified by column chromatography on silica gel eluting with ethyl acetate to give 2′-{[(4-{formyl[2-(2-pyridinyl)ethyl]amino}phenyl)amino]-carbonyl}-1,1′-biphenyl-4-yl methanesulfonate (1.29 g) as a light brown amorphous powder.

¹H-NMR (DMSO-d₆): δ2.89 (2H, d, J=8.1 Hz), 3.32 (3H, s), 4.07 (2H, t, J=8.1 Hz), 7.2-7.75 (15H, m), 8.30 (1H, s), 8.46 (1H, d, J=4.9 Hz), 10.34 (1H, s)

ESI-MS (m/z): 538 (M+Na)⁺, 516 (M+H)⁺

### EXAMPLE 329

To a solution of 2′-{[(4-{formyl[2-(2-pyridinyl)ethyl]aminophenyl)amino]carbonyl}-1,1′-biphenyl-4-yl methanesulfonate (1.26 g) in methanol (13 ml) was added conc. HCl (1.0 ml) at room temperature and the mixture was stirred at room temperature for 18 hours. The mixture was poured into a mixture of ethyl acetate and ice water and adjusted to pH 8 by addition of 50% aqueous potassium carbonate solution. The separated organic layer was washed with water and brine, dried over magnesium sulfate and evaporated in vacuo. The residue was purified by column chromatography on silica gel eluting with ethyl acetate and recystalized from ethyl acetate to give 2′-{[(4-{[2-(2-pyridinyl)ethyl]amino}phenyl)amino]carbonyl}-1,1′-biphenyl-4-yl methanesulfonate (763 mg) as white crystals.

¹H-NMR (DMSO-d₆): δ2.96 (2H, d, J=7.0 Hz), 3.36 (3H, s), 3.38 (2H, dd, J=7.0 and 5.7 Hz), 4.07 (2H, t, J=5.7 Hz), 6.50 (2H, d, J=8.8 Hz), 7.16 (2H, d, J=8.8 Hz), 7.2-7.6 (10H, m), 7.65-7.75 (1H, m), 8.50 (1H, d, J=4.7 Hz), 9.78 (1H, s)

ESI-MS (m/z): 510 (M+Na)+, 488 (M+H)+

Preparation 128

2-Fluoro-4-nitro-N-[2-(2-pyridinyl)ethyl]aniline was obtained in the same manner as in Preparation 21 as a yellow solid.

¹H-NMR (DMSO-d₆): δ3.05 (2H, t, J=6.9 Hz), 3.63 (2H, td, J=6.9 and 6.7 Hz), 6.8-6.95 (1H, m), 7.15-7.3 (2H, m), 7.32 (1H, d, J=7.8 Hz), 7.65-7.8(1H, m), 7.9-8.05 (2H, m), 8.5-8.6 (1H, m)

ESI-MS (m/z): 284 (M+Na)⁺

Preparation 129

2-Fluoro-N¹-[2-(2-pyridinyl)ethyl]-1,4-benzenediamine was obtained in the same manner as in Preparation 16 as a brown amorphous powder.

¹H-NMR (DMSO-d₆): δ2.96 (2H, t, J=7.1 Hz), 3.28 (2H, td, J=7.1 and 6.2 Hz), 4.52 (1H, t, J=6.2 Hz), 4.55 (2H, brs), 6.25-6.4 (2H, m), 6.55 (1H, dd, J=8.5 and 8.5 Hz), 7.15-7.3 (2H, m), 7.65-7.75 (1H, m), 8.49 (1H, d, J=4.8 Hz)

APCI-MS (m/z): 232 (M+H)⁺

### EXAMPLE 330

N-(3-Fluoro-4-{[2-(2-pyridinyl)ethyl]amino}phenyl)-4′-(trifluoromethyl)-1,1′-biphenyl-2-carboxamide was obtained in the same manner as in Preparation 19 as white crystals.

¹H-NMR (DMSO-d₆): δ2.99 (2H, t, J=7.0 Hz), 3.40 (2H, td, J=7.0 and 4.7 Hz), 5.36 (1H, t, J=4.7 Hz), 6.69 (1H, dd, J=9.5 and 9.5 Hz), 7.14 (1H, d, J=8.8 Hz), 7.2-7.4 (3H, m), 7.5-7.8 (9H, m), 8,51 (1H, d, J=3.9 Hz), 10.13 (1H, s)

APCI-MS (m/z): 480 (M+H)⁺

### EXAMPLE 331

N-(3-Fluoro-4-{[2-(2-pyridinyl)ethyl]amino}phenyl)-4′-methoxy-1,1′-biphenyl-2-carboxamide was obtained in the same manner as in Example 59 as white crystals.

¹H-NMR (DMSO-d₆): δ3.00 (2H, t, J=7.0 Hz), 3.38 (2H, td, J=7.0 and 5.4 Hz), 3.75 (3H, s), 5.34 (1H, t, J=5.4 Hz), 6.69 (1H, dd, J=9.1 and 9.1 Hz), 6.94 (1H, d, J=8.7 Hz), 7.08 (1H, d, J=8.7 Hz), 7.2-7.6 (10H, m), 7.71 (1H, ddd, J=7.7 and 7.6 and 1.8 Hz), 8.51 (1H, d, J=4.4 Hz), 10.01 (1H, s)

APCI-MS (m/z): 442 (M+H)⁺

### EXAMPLE 332

N-(3-Fluoro-4-{[2-(2-pyridinyl) ethyl]amino}phenyl)-2-[3-(trifluoromethyl)anilino]benzamide was obtained in the same manner as in Example 59 as white crystals.

¹H-NMR (DMSO-d₆): δ3.01 (2H, t, J=7.3 Hz), 3.42 (2H, td, J=7.3 and 5.6 Hz), 5.41 (1H, t, J=5.6 Hz), 6.75 (1H, dd, J=9.4 and 9.4Hz), 7.0-7.8 (13H, m), 8.51 (1H, d, J=4.8 Hz), 10.19 (1H, s)

ESI-MS (m/z): 517 (M+Na)⁺, 495 (M+H) +

Preparation 130

To a mixture of 1-bromo-2-methoxy-4-nitrobenzene (11.60 g) and 2-(2-pyridinyl)ethanamine (12.2 g) was added N,N-diisopropylethylamine (19.4 g) and the mixture was stirred at 160° C. for 20 hours. The mixture was cooled to room temperature and extracted with ethyl acetate:tetrahydrofuran (2:1). After the insoluble materials were removed by filtration, the separated oraganic layer was washed with water and brine, dried over magnesium sulfate and evaporated in vacuo. The residue was purified by column chromatography on silica gel eluting with hexane:ethyl acetate (1:2) to give N-(2-methoxy-4-nitrophenyl)-N-[2-(2-pyridinyl)ethyl]amine (2.76 g) as a brown solid.

¹H-NMR (DMSO-d₆): δ3.04 (2H, t, J=7.7 Hz), 3.60 (2H, td, J=7.7 and 5.6 Hz), 3.89 (3H, s), 6.18 (1H, t, J=5.6 Hz), 7.26 (1H, dd, J=7.8 and 4.8 Hz), 7.32 (1H, d, J=7.8 Hz), 7.56 (1H, d, J=2.5 Hz), 7.7-7.8 (1H, m), 7.83 (1H, dd, J=9.0 and 2.4 Hz), 8.52 (1H, d, J=4.8 Hz)

APCI-MS (m/z): 274 (M+H)⁺

Preparation 131

2-Methoxy-N¹-[2-(2-pyridinyl)ethyl]-1,4-benzenediamine was obtained in the same manner as in Preparation 16 as a brown amorphous powder.

¹H-NMR (DMSO-d₆): δ2.96 (2H, t, J=7.2 Hz), 3.30 (2H, td, J=7.2 and 6.1 Hz), 3.66 (3H, s), 4.14 (1H, t, J=6.1 Hz), 4.32 (2H, brs), 6.07 (1H, dd, J=8.2 and 2.3 Hz), 6.21 (1H, d, J=2.3 Hz), 6.37 (1H, d, J=8.2 Hz), 7.15-7.3 (2H, m), 7.65-7.8 (1H, m), 8.50 (1H, d, J=4.8 Hz).

APCI-MS (m/z): 244 (M+H)⁺

### EXAMPLE 333

N-(3-Methoxy-4-{[2-(2-pyridinyl)ethyl]amino}phenyl)-4′-(trifluoromethyl)-1,1′-biphenyl-2-carboxamide was obtained in the same manner as in Preparation 19 as white crystals.

¹H-NMR (DMSO-d₆): δ2.99 (2H, t, J=7.2 Hz), 3.38 (2H, td, J=7.2 and 5.5 Hz), 3.68 (3H, s), 4.84 (1H, t, J=5.5 Hz), 6.50 (1H, d, J=8.5 Hz), 6.93 (1H, dd, J=8.5 and 2.1 Hz), 7.02 (1H, d, J=2.1 Hz), 7.2-7.35 (2H, m), 1.45-7.8 (9H, m), 8.51 (1H, d, J=4.0 Hz), 9.96 (1H, s)

ESI-MS (m/z): 514 (M+Na)⁺, 492 (M+H)⁺

### EXAMPLE 334

To a solution of N-(4-{[2-(2-pyridinyl)ethyl]amino}phenyl)-4′-(trifluoromethyl)-1,1′-biphenyl-2-carboxamide (923 mg) in dichloromethane (30 ml) were added N-bromosuccinimide (534 mg) and V-70 (31 mg) at room temperature and the mixture was refluxed for 6 hours under nitrogen. The mixture was cooled to room temperature and poured into water. The separated oraganic layer was washed with water and brine, dried over magnesium sulfate and evaporated in vacuo. The residue was purified by column chromatography on silica gel fluting with ethyl acetate and recrystallized from ethyl acetate to give N-(3-bromo-4-{[2-(2-pyridinyl)ethyl]amino}phenyl)-4′-(trifluoromethyl)-1,1′-biphenyl-2-carboxamide (471 mg) as white crystals.

¹H-NMR (DMSO-d₆): δ3.02 (2H, t, J=6.7 Hz), 3.44 (2H, td, J=6.7 and 5.4 Hz), 5.25 (1H, t, J=5.4 Hz), 6.69 (1H, d, J=8.8 Hz), 7.2-7.35 (3H, m), 7.5-7.8 (7H, m), 8.52 (1H, d, J=4.0 Hz), 10.11 (1H, s)

APCI-MS (m/z): 542, 540 (M+H)⁺

Preparation 132

N-(2-Methyl-4-nitrophenyl)-4′-(trifluoromethyl)-1,1′-biphenyl-2-carboxamide was obtained in the same manner as in Preparation 19 as a yellow solid.

¹H-NMR (DMSO-d₆): δ1.74 (3H, s), 6.64 (1H, d, J=8.6 Hz), 7.08 (1H, d, J=8.6 Hz), 7.4-7.7 (7H, m), 7.8-7.95 (3H, m) negative ESI-MS (m/z): 399 (M−H)⁻

Preparation 133

N-(4-Amino-2-methylphenyl)-4′-(trifluoromethyl)-1,1′-biphenyl-2-carboxamide was obtained in the same manner as in Preparation 16 as a brown amorphous powder.

¹H-NMR (DMSO-d₆): δ1.83 (3H, s), 4.90 (2H, brs), 6.3-6.4 (2H, m), 6.77 (1H, d, J=8.4 Hz), 7.45-7.7 (6H, m), 7.79 (2H, m), 9.32 (1H, s)

ESI-MS (m/z): 393 (M+Na)⁺, 371 (M+H)⁺

### EXAMPLE 335

To a solution of N-(4-amino-2-methylphenyl)-4′-(trifluoromethyl)-1,1′-biphenyl-2-carboxamide (3.70 g) and 2-vinylpyridine (1.16 g) in 2-methoxyethanol (15 ml) was added methanesulfonic acid (1.06 g) at room temperature and the mixture was stirred at 160° C. for 17 hours. The mixture was purified by column chromatography on silica gel eluting with ethyl acetate and recrystallized from ethyl acetate to give N-(2-methyl-4-{[2-(2pyridinyl)ethyl]amino}phenyl)-4′-(trifluoromethyl)-1,1′-biphenyl-2-carboxamide (1.96 g) as white crystals.

¹H-NMR (DMSO-d₆): δ1.86 (3H, s), 2.95 (2H, t, J=7.4 Hz), 3.30 (2H, td, J=7.4 and 5.7 Hz), 5.53 (1H, t, J=5.7 Hz), 6.35-6.45 (1H, m), 6.85-6.9 (1H, m), 7.2-7.35 (2H, m), 7.5-7.9 (10H, m), 8.5-8.55 (1H, m), 9.36 (1H, s)

ESI-MS (m/z): 498 (M+Na)⁺, 476 (M+H)⁺

### EXAMPLE 336

To a solution of N-(4-aminophenyl)-4′-(trifluoromethyl)-1,1′-biphenyl-2-carboxamide (1.78 g) was added 2-vinylpyridine (630 mg) in 2-methoxyethanol (20 ml) at room temperature and the mixture was stirred at 160° C. for 16 hours. The mixture was purified by column chromatography on silica gel eluting with ethyl acetate to give N-(4-{[2-(2-pyridinyl)ethyl]amino}phenyl)-4′-(trifluoromethyl)-1,1′-biphenyl-2-carboxamide (1.73 g) as white crystals, and eluting with ethyl acetate:methanol (5:1) to give N-(4-{bis[2-(2-pyridinyl)ethyl]amino}phenyl)-4′-(trifluoromethyl)-1,1′-biphenyl-2-carboxamide (265 mg).

N-(4-{[2-(2-Pyridinyl)ethyl]amino}phenyl)-4′-(trifluoromethyl)-1,1′-biphenyl-2-carboxamide

¹H-NMR (DMSO-d₆): δ2.96 (2H, t, J=7.4 Hz), 3.34 (2H, td, J=7.4 and 5.8 Hz), 5.54 (1H, t, J=5.8 Hz), 6.50 (2H, d, J=8.8 Hz), 6.85-6.9 (1H, m), 7.20 (2H, d, J=8.8 Hz), 7.29 (1H, d, J=7.4 Hz), 7.45-7.8 (10H, m), 8.50 (1H, d, J=4.0 Hz), 9.92 (1H, s)

APCI-MS (m/z): 462 (M+H)⁺

N-(4-{Bis[2-(2-pyridinyl)ethyl]amino}phenyl)-4′-(trifluoromethyl)-1,1′-biphenyl-2-carboxamide

¹H-NMR (DMSO-d₆): δ2.90 (4H, t, J=7.8 Hz), 3.58 (4H, t, J=7.8 Hz), 6.71 (2H, d, J=8.9 Hz), 7.2-7.2 (6H, m), 7.5-7.85 (10H, m), 8.52 (2H, d, J=4.1 Hz), 10.03 (1H, s)

APCI-MS (m/z): 567 (M+H)⁺

### EXAMPLE 337

N-(4-{[2-(6-Methyl-2-pyridinyl)ethyl]amino}phenyl)-4′-(trifluoromethyl)-1,1′-biphenyl-2-carboxamide was obtained in the same manner as in Example 336 as white crystals.

¹H-NMR (DMSO-d₆): δ2.45 (3H, s), 2.91 (2H, t, J=6.9 Hz), 3.31 (2H, td, J=6.9 and 5.7 Hz), 5.54 (1H, t, J=5.7 Hz), 6.50 (2H, d, J=8.8 Hz), 7.08 (1H, dd, J=7.4 and 2.7 Hz), 7.20 (2H, d, J=8.8 Hz), 7.45-7.6 (6H, m), 7.64 (2H, d, J=8.3 Hz), 7.76 (2H, d, J=8.3 Hz), 9.92 (1H, s)

APCI-MS (m/z): 476 (M+H)⁺

Preparation 134

To a suspension of 4-nitroaniline (6.91 g) and 2-vinylpyridine (6.31 g) in 2-propanol (21 ml) was added dropwise conc. HCl (4.2 ml) at room temperature and the mixture was refluxed for 24 hours. After cooling, to the resulting solution was added dropwise ethyl acetate (62 ml) and the mixture was stirred at room temperature for 40 minutes. The precipitate (N-(4-nitrophenyl)-N-[2-(2-pyridinyl)ethyl]amine hydrochloride) was collected by filtration, washed with ethyl acetate, and dried in vacuo. The crude hydrochloride was dissolved in water (54 ml) and 5N aqueous sodium hydroxide solution (15 ml) was added to the solution. The mixture was stirred at room temperature for 3 hours and the precipitate was collected by filtration, washed with water and diisopropyl ether and dried over phosphorus pentoxide in vacuo to give N-(4-nitrophenyl)-N-[2-(2-pyridinyl)ethyl]amine (10.79 g) as a yellow solid.

¹H-NMR (DMSO-d₆): δ3.02 (2H, t, J=7.4 Hz), 3.54 (2H, td, J=7.4 and 5.3 Hz), 6.6-6.7 (2H, m), 7.2-7.45 (3H, m), 7.65-7.8 (1H, m), 7.95-8.05 (2H, m), 8.5-8.55 (1H, m)

ESI-MS (m/z): 266 (M+Na)⁺, 244 (M+H)⁺

Preparation 135

Formic acid (8.11 g) was added dropwise to acetic anhydride (8.99 g) at room temperature and the mixture was stirred at 50° C. for 30 minutes. The mixture was cooled to room temperature and tetrahydrofuran (21 ml) was added. To this solution was added portionwise N-(4-nitrophenyl)-N-[2-(2-pyridinyl)ethyl]amine (10.71 g) and the mixture was stirred at 60° C. for 18 hours. The mixture was evaporated in vacuo and triturated with diisopropyl ether to give 4-nitrophenyl[2-(2-pyridinyl)ethyl]formamide (11.27 g) as a yellow solid.

¹H-NMR (DMSO-d₆): δ2.96 (2H, t, J=7.1 Hz), 4.26 (2H, t, J=7.1 Hz), 7.15-7.25 (2H, m), 7.5-7.8 (3H, m), 8.24 (2H, d, J=9.0 Hz), 8.45 (1H, d, J=4.7 Hz), 8.71 (1H, s)

ESI-MS (m/z): 294 (M+Na)⁺, 272 (M+H)⁺

Prepration 136

A suspension of iron powder (6.86 g) and ammonium chloride (2.19 g) in methanol (83 ml) and water (28 ml) was heated to 90° C. To this suspension was added 4-nitrophenyl[2-(2-pyridinyl)ethyl]formamide (11.15 g) by portions and the mixture was stirred at 90° C. for 20 hours. The mixture was cooled to room temperature and the insoluble materials were filtered off by celite and washed with methanol. The filtrate was evaporated in vacuo to remove methanol and to the residue was added ethyl acetate (90 ml). The mixture was adjusted to pH 8 by addition of 5N aqueous sodium hydroxide solution. The separated organic layer was washed with brine, dried over magnesium sulfate, and evaporated in vacuo. The residue was recrystallized from ethyl acetate:diisopropyl ether (1:1), washed with the same solvent and dried in vacuo to give 4-aminophenyl[2-(2-pyridinyl)ethyl]formamide (7.07 g) as pale brown crystals.

¹H-NMR (DMSO-d₆): δ2.87 (2H, t, J=7.3 Hz), 3.95 (2H, t, J=7.3 Hz), 5.19 (2H, brs), 6.5-6.6 (3H, m), 6.85-6.95 (2H, m), 7.2-7.3 (2H, m), 7.6-7.75 (1H, m), 8.11 (1H, s), 8.45-8.55 (1H, m)

ESI-MS (m/z): 264 (M+Na)⁺, 242 (M+H)⁺

### EXAMPLE 338

To a solution of 4-aminophenyl[2-(2-pyridinyl)ethyl]formamide (7.0 g), triethylamine (3.23 g) and 4,4,-dimethylaminopyridine (71 mg) in tetrahydrofuran (55 ml) was added dropwise a solution of 4′-methyl-1,1′-biphenyl-2-carbonyl chloride (6.69 g) in tetrahydrofuran (14 ml) at 5° C. The mixture was stirred at room temperature for 19 hours and poured into a mixture of ethyl acetate and water. The separated organic layer was washed with water and brine, dried over magnesium sulfate, and evaporated in vacuo. The residue was crystallized from ethyl acetate:diisopropyl ether (1:1) to give N-(4-{formyl[2-(2-pyridinyl)ethyl]amino}phenyl)-4′-methyl-1,1′-biphenyl-2-carboxamide (11.9.g) as white crystals.

¹H-NMR (DMSO-d₆): δ2.29 (3H, s), 3.44 (2H, t, J=7.1 Hz), 3.67 (2H, t, J=7.1 Hz), 7.1-7.6 (12H, .m), 7.85-8.0 (2H, m), 8.35-8.5 (1H, m), 8.78 (1H, d, J=5.5 Hz), 10.24 (1H, s)

ESI-MS (m/z): 458 (M+Na)⁺, 436 (M+H)⁺

### EXAMPLE 339

To a suspension of N-(4-{formyl[2-(2-pyridinyl)ethyl]amino}phenyl)-4′-methyl-1,1′-biphenyl-2-carboxamide (11.8 g) in methanol (59 ml) was added conc. HCl (11.3 ml) at room temperature and the resulting solution was stirred at room temperature for 40 hours. The precipitate was formed and the suspension was diluted with ethyl acetate (59 ml). The precipitate was collected by filtration, washed with methanol:ethyl acetate (1:1), and dried in vacuo. The crude product was recrystallized from methanol:diisopropyl ether (1:1) to give 4′-methyl-N-(4-{(2-(2-pyridinyl)ethyl]amino}phenyl)-1,1′-biphenyl-2-carboxamide dihydrochloride (10.69 g) as white crystals.

¹H-NMR (DMSO-d₆): δ2.29 (3H, s), 3.44 (2H, t, J=7.1 Hz), 3.67 (2H, t, J=7.1 Hz), 7.1-7.6 (12H, .m), 7.85-8.0 (2H, m), 8.35-8.5 (1H, m), 8.78 (1H, d, J=5.5 Hz), 10.24 (1H, s)

ESI-MS (m/z): 430 (M+Na)⁺, 408 (M+H) ⁺

### EXAMPLE 340

To a suspension of 4′-methyl-N-(4-{[2-(2-pyridinyl)ethyl]amino}phenyl)-1,1′-biphenyl-2-carboxamide dihydrochloride (10.68 g) in a mixture of water (86 ml), ethyl acetate (48 ml) and tetrahydrofuran (24 ml) was added dropwise 5N aqueous sodium hydroxide solution (9.5 ml) at room temperature and the mixture was stirred at room temperature for 3 hours. The precipitate was collected by filtration, washed with water and diisopropyl ether, and dried in vacuo. The crude product was recrystallized from methanol:tetrahydrofuran:diisopropyl ether (1:1:2) to give 4′-methyl-N-(4-{[2-(2-pyridinyl)ethyl]amino}-phenyl)-1,1′-biphenyl-2-carboxamide (7.70 g) as light yellow crystals.

¹H-NMR (DMSO-d₆): δ2.29 (3H, s), 2.96 (2H, t, J=7.0 Hz), 3.34 (2H, td, J=7.0 and 5.8 Hz), 5.58 (1H, t, J=5.8 Hz), 6.50 (2H, d, J=8.8 Hz), 7.15-7.5 (12H, m), 7.65-7.75 (1H, m), 8.45-8.55 (1H, m), 9.79 (1H, s)

ESI-MS (m/z): 430 (M+Na)⁺, 408 (M+H)⁺

Preparation 137

To a suspension of 4-nitroaniline hydrochloride (19.22 g) in 2-propanol (60 ml) was added 2-vinylpyridine (13.9 g) at room temperature and the mixture was refluxed for 24 hours. After cooling, to the resulting solution was added dropwise ethyl acetate (240 ml) and the mixture was stirred at room temperature for 40 minutes. The precipitate (N-(4-nitrophenyl)-N-[2-(2-pyridinyl)ethyl]amine hydrochloride) was collected by filtration, washed with ethyl acetate, and dried in vacuo. The crude hydrochloride was dissolved in water (110 ml) and 5N aqueous sodium hydroxide solution (32 ml) was added to the solution. The mixture was stirred at room temperature for 3 hours, and the precipitate was collected by filtration, washed with water and diisopropyl ether and dried in vacuo over phosphorus pentoxide to give N-(4-nitrophenyl)-N-[2-(2-pyridinyl)ethyl]amine (20.90 g) as a yellow solid.

¹H-NMR (DMSO-d₆): δ3.02 (2H, t, J=7.4 Hz), 3.54 (2H, td, J=7.4 and 5.3 Hz), 6.6-6.7 (2H, m), 7.2-7.45 (3H, m), 7.65-7.8 (1H, m), 7.95-8.05 (2H, m), 8.5-8.55 (1H, m)

ESI-MS (m/z): 266 (M+Na)⁺, 244 (M+H)⁺

Preparation 138

To a solution of N-(4-nitrophenyl)-N-[2-(2-pyridinyl)ethyl]amine (4.87 g) in acetic acid (50 ml) were added acetic anhydride (4.08 g) and 4-dimethylaminopyridine (244 mg) at room temperature and the mixture was refluxed for 8 hours. The mixture was evaporated in vacuo and the residue was extracted with ethyl acetate. The organic layer was washed with brine, dried over magnesium sulfate and evaporated in vacuo. The residue was triturated with diisopropyl ether to give N-(4-nitrophenyl)-N-[2-(2-pyridinyl)ethyl]acetamide (4.46 g) as a yellow solid.

¹H-NMR (DMSO-d₆): δ1.68 (3H, s), 2.97 (2H, t, J=7.1 Hz), 4.25 (2H, td, J=7.1 Hz), 7.1-7.8 (6H, m), 8.2-8.3 (3H, m), 8.4-8.5 (1H, m)

ESI-MS (m/z): 308 (M+Na)⁺, 286 (M+H)⁺

Preparation 139

To a solution of N-(4-nitrophenyl)-N-[2-(2-pyridinyl)ethyl]acetamide (4.44 g) in tetrahydrofuran (30 ml) and methanol (30 ml) was added 10% palladium on carbon (50% wet) (1 g) and the mixture was hydrogenated under atomospheric pressure at room temperature for 6 hours. The catalysts were removed by filtration, and the filtrate was evaporated in vacuo. The residue was purified by column chromatography on silica gel eluting with hexane:ethyl acetate (1:2) to give N-(4-aminophenyl)-N-[2-(2-pyridinyl)ethyl]acetamide (3.23 g) as a pale brown solid.

¹H-NMR (DMSO-d₆): δ1.68 (3H, s), 2.88 (2H, t, J=7.6 Hz), 3.84 (2H, td, J=7.6 Hz), 5.24 (2H, br.s), 6.5-6.6 (2H, m), 6.8-6.9 (2H, m), 7.15-7.3 (2H, m), 7.6-7.75 (1H, m), 8.4-8.5 (1H, m)

ESI-MS (m/z): 278 (M+Na)⁺, 256 (M+H)⁺

### EXAMPLE 341

N-(4-{Acetyl[2-(2-pyridinyl)ethyl]amino}phenyl)-4′-methyl-1,1′-biphenyl-2-carboxamide was obtained in the same manner as in Example 338 as white crystals.

¹H-NMR (DMSO-d₆): δ1.72 (3H, s), 2.29 (3H, s), 2.85-2.95 (2H, m), 3.9-4.0 (2H, m), 7.2-7.7 (15H, m), 8.44 (1H, d, J=4.2 Hz), 10.42 (1H, s)

ESI-MS (m/z): 472 (M+Na)⁺, 450 (M+H)⁺

### EXAMPLE 342

To a suspension of-N-(4-{acetyl[2-(2-pyridinyl)ethyl]amino}phenyl)-4′-methyl-1,1′-biphenyl-2-carboxamide (5.10 g) was added conc. HCl (4.7 ml) at room temperature and the resulting solution was refluxed for 8 hours. The mixture was poured into a mixture of ethyl acetate and ice water and adjusted to pH 8 by addition of 50% aqueous potassium carbonate solution. The separated organic layer was washed with water and brine, dried over magnesium sulfate and evaporated in vacuo. The residue was purified by column chromatography on silica gel eluting with ethyl acetate and recrystallized from ethanol:diisopropylether (1:2) to give 4′-methyl-N-(4-((2-(2-pyridinyl)ethyl)amino)phenyl)-1,1′-biphenyl-2-carboxamide (1.46 g) as pale yellow crystals.

¹H-NMR (DMSO-d₆): δ2.29 (3H, s), 2.96 (2H, t, J=7.0 Hz), 3.34 (2H, td, J=7.0 and 5.8 Hz), 5.58 (1H, t, J=5.8 Hz), 6.50 (2H, d, J=8.8 Hz), 7.15-7.5 (12H, m), 7.65-7.75 (1H, m), 8.45-8.55 (1H, m), 9.79 (1H, s)

ESI-MS (m/z): 430 (M+Na)⁺, 408 (M+H)⁺

Preparation 140

N-(4-Nitrophenyl)-N-[2-(3-pyridinyl)ethyl]amine was obtained in the same manner as in Preparation 21 as a yellow solid.

¹H-NMR (DMSO-d₆): δ2.88 (2H, t, J=7.1 Hz), 3.46 (2H, td, J=7.1 and 6.9 Hz), 6.68 (2H, d, J=9.3 Hz), 7.25-7.4 (2H, m), 7.71 (1H, d, J=7.9 Hz), 7.98 (2H, d, J=9.3 Hz), 8.45-8.5 (1H, m), 8.50 (1H, d, J=2.1 Hz)

APCI-MS (m/z): 244 (M+H)⁺

Preparation 141

N¹-[2-(3-pyridinyl)ethyl]-1,4-benzenediamine was obtained in the same manner as in Preparation 16 as a brown oil.

¹H-NMR (DMSO-d₆): δ2.80 (2H, t, J=7.1 Hz), 3.15 (2H, td, J=7.1 and 6.1 Hz), 4.23 (2H, brs), 4.75 (1H, t, J=6.1 Hz), 6.4-6.5 (4H, m), 7.31 (1H, dd, J=7.8 and 4.7 Hz), 7.65-7.75 (1H, m), 8.40 (1H, dd, J=4.7 and 1.8 Hz), 8.46 (1H, d, J=1.8 Hz)

APCI-MS (m/z): 214 (M+H)⁺

### EXAMPLE 343

N-(4-([2-(3-Pyridinyl)ethyl]amino}phenyl)-4′-(trifluoromethyl)-1,1′-biphenyl-2-carboxamide was obtained in the same manner as in Example 56 as white crystals.

¹H-NMR (DMSO-d₆): δ2.82 (2H, t, J=7.1 Hz), 3.23 (2H, td, J=7.1 and 5.8 Hz), 5.55 (1H, t, J=5.8 Hz), 6.51 (2H, d, J=8.8 Hz), 7.20 (2H, d, J=8.8 Hz), 7.31 (1H, dd, J=5.1 and 4.8 Hz), 7.45-7.8 (7H, m), 8.41 (1H, dd, J=4.8 and 1.7 Hz), 8.48 (1H, d, J=1.7 Hz), 9.91 (1H, s)

APCI-MS (m/z): 462 (M+H)⁺

### EXAMPLE 344

N-(4-{[2-(4-Pyridinyl)ethyl]amino}phenyl)-4′-(trifluoromethyl)-1,1′-biphenyl-2-carboxamide was obtained in the same manner as in Example 336 as pale brown crystals.

¹H-NMR (DMSO-d₆): δ2.82 (2H, t, J=7.2 Hz), 3.25 (2H, td, J=7.2 and 5.6 Hz), 5.57 (1H, t, J=5.7 Hz), 6.51 (2H, d, J=8.7 Hz), 7.20 (2H, d, J=8.7 Hz), 7.29 (2H, dJ=5.8 Hz), 7.45-7.8 (8H, m), 8.45 (2H, d, J=5.8 Hz), 9.92 (1H, s)

APCI-MS (m/z): 462 (M+H)⁺

Preparation 142

A mixture of 4-nitroaniline (13.81 g) and methyl 2-pyridinylacetate (22. 70 g) was stirred at 150° C. for 20 hours. The mixture was cooled to room temperature and the residue was purified by column chromatography on silica gel eluting with hexane:ethyl acetate (1:1) to give N-(4-nitrophenyl)-2-(2-pyridinyl)acetamide (12.12 g) as a yellow solid.

¹H-NMR (DMSO-d₆): δ3.93 (2H, s), 7.25-7.35 (1H, m), 7.41 (1H, d, J=7.8 Hz), 7.7-7.9 (3H, m), 8.2-8.3 (2H, m), 8.5-8.55 (1H, m), 10.87 (1H, s)

APCI-MS (m/z): 258 (M+H)⁺

Preparation 143

To a suspension of sodium hydride (60% oil dispersion) (400 mg) in N,N-dimethylformamide (20 ml) was added dropwise a solution of N-(4-nitrophenyl)-2-(2-pyridinyl)acetamide (2.57 g) in N,N-dimethylformamide (20 ml) at room temperature and the mixture was stirred at room temperature for an hour. To this mixture was added methyl iodide (1.70 g) and the mixture was stirred at room temperature for 4 hours. The mixture was poured into a mixture of ethyl acetate and ice water and the separated organic layer was washed with water and brine, dried over magnesium sulfate and evaporated in vacuo. The residue was purified by column chromatography on silica gel eluting with ethyl acetate and recrystallized from ethanol:diisopropyl ether (2:1) to give N-(4-nitrophenyl)-2-(2-pyridinyl)propanamide (1.87 g) as a yellow solid.

¹H-NMR (DMSO-d₆): δ1.44 (3H, d, J=7.1 Hz), 3.93 (2H, d, J=7.1 Hz), 7.25-7.35 (1H, m), 7.41 (1H, d, J=7.8 Hz), 7.7-7.9 (3H, m), 8.2-8.3 (2H, m), 8.5-8.55 (1H, m), 10.87 (1H, s)

APCI-MS (m/z): 272 (M+H)⁺

Preparation 144

N-(4-Aminophenyl)-2-(2-pyridinyl)propanamide was obtained in the same manner Preparation 139 as pale brown crystals.

¹H-NMR (DMSO-d₄): δ1.44 (3H, d, J=7.1 Hz), 3.93 (2H, d, J=7.1 Hz), 4.83 (2H, brs), 6.47 (2H, d, J=8.7 Hz), 7.24 (2H, d, J=8.7 Hz), 7.42 (1H, d, J=7.9 Hz, 7.7-7.85 (1H, m), 8.45-8.55 (1H, m), 9.71 (1H, s)

APCI-MS (m/z): 242 (M+H)⁺

### EXAMPLE 345

N-(4-{[2-(2-Pyridinyl)propanoyl]amino}phenyl)-4′-(trifluoromethyl)-1,1′-biphenyl-2-carboxamide was obtained in the same manner as in Preparation 19 as white crystals.

¹H-NMR (DMSO-d₆): δ1.44 (3H, d, J=7.1 Hz), 3.93 (2H, d, J=7.1 Hz), 7.25-7.8(15H, m), 8.48 (1H, d, J=4.8 Hz), 10.19 (1H, s), 10.28 (1H, s)

APCI-MS (m/z): 490 (M+H)⁺

Preparation 145

N-(2-Methyl-4-nitrophenyl)-2-(2-pyridinyl)acetamide was obtained in the same manner as in Preparation 142 as a yellow solid.

¹H-NMR (DMSO-d₆): δ2.39 (3H, s), 4.01 (2H, s), 7.32 (1H, dd, J=7.4 and 4.9 Hz), 7.43 (4H, d, J=7.8 Hz), 7.80 (1H, ddd, J=7.8 and 7.7 and 1.8 Hz), 8.05 (1H, d, J=1.1 Hz), 8.15 (1H, s), 8.55 (1H, d, J=4.9 Hz), 10.14 (1H, s)

APCI-MS (m/z): 272 (M+H)⁺

Preparation 146

N-(4-Amino-2-methylphenyl)-2-(2-pyridinyl) acetamide was obtained in the same manner as in Preparation 139 as pale brown crystals.

¹H-NMR (DMSO-d₆): δ2.01 (3H, s), 3.77 (2H, s), 4.87 (2H, brs), 6.3-6.45 (2H, m), 6.91 (1H, d, J=8.2 Hz), 7.2-7.3 (1H, m), 7.39 (1H, d, J=7.8 Hz), 7.7-7.85 (1H, m), 8.50 (1H, d, J=4.8 Hz), 9.28 (1H, s)

APCI-MS (m/z): 242 (M+H)⁺

### EXAMPLE 346

N-{3-Methyl-4-[(2-pyridinylacetyl)amino]phenyl}-4′-(trifluoromethyl)-1,1′-biphenyl-2-carboxamide was obtained in the same manner as in Preparation 19 as white crystals.

¹H-NMR (DMSO-d₆): δ2.13 (3H, s), 3.85 (2H, s), 7.3-7.8 (14H, m), 8.52 (1H, d, J=4.0 Hz), 9.61 (1H, s), 10.26 (1H, s)

APCI-MS (m/z): 490 (M+H)⁺

### EXAMPLE 347

The mixture of N-(4-aminophenyl)-4′-(trifluoromethyl)-1,1′-biphenyl-2-carboxamide (712 mg) and 2-bromopyridine (1.58 g) was stirred at 150° C. for 8 hours. The mixture was cooled to room temperature and the residue was purified by column chromatography on silica gel eluting with hexane:ethyl acetate (1:1) to give N-(4-(2-pyridinylamino)phenyl)-4′-(trifluoromethyl)-1,1′-biphenyl-2-carboxamide (418 mg) as white crystals.

¹H-NMR (DMSO-d₆): δ6.69 (1H, dd, J=8.4 and 5.3 Hz), 6.77 (1H, d, J=8.4 Hz), 7.39 (1H, d, J=8.3 Hz), 7.45-7.7 (9H, m), 7.77 (2H, d, J=8.3 Hz), 8.10 (1H, d, J=3.6 Hz), 8.93 (1H, s), 10.18 (1H, s)

APCI-MS (m/z): 434 (M+H)⁺

Preparation 147

To a suspension of potassium tert-butoxide (4.49 g) in 1,3-dimethylimidazolidinone (80 ml) was added dropwise a solution of 2-[methyl(2-pyridinyl)amino]ethanol (6.09 g) in 1,3-dimethylimidazolidinone (40 ml) at room temperature and the mixture was stirred at room temperature for an hour under nitrogen. To the mixture was added 4-fluoro-1-nitrobenzne (5.64 g) and the mixture was refluxed for 6 hours. The mixture was poured into a mixture of ethyl acetate and ice water. The separated organic layer was washed with water and brine, dried over magnesium sulfate and evaporated in vacuo. The residue was purified by column chromatography on silica gel eluting with hexane:ethyl acetate (4:1) to give N-methyl-N-[2-(4-nitrophenoxy)ethyl]-2-pyridinamine (5.60 g) as yellow crystals.

¹H-NMR (DMSO-d₆): δ3.07 (3H, s), 3.95 (2H, t, J=5.8 Hz), 4.30 (2H, t,=5.8 Hz), 6.57 (1H, dd, J=8.6 and 5.0 Hz), 6.65(1H, d, J=8.6 Hz), 7.1-7.2 (2H, m), 7.45-7,55 (1H, m), 8.05-8.10 (1H, m), 8.15-8.25 (2H, m)

ESI-MS (m/z): 296 (M+Na)⁺, 274 (M+H)⁺

Preparation 148

N-[2-(4-Aminophenoxy)ethyl]-N-methyl-N-(2-pyridinyl)amine was obtained in the same manner as in Preparation 139 as a pale brown oil.

¹H-NMR (DMSO-d₆): δ3.05 (3H, s), 3.83 (2H, t, J=5.8 Hz), 3.98 (2H, t,=5.8 Hz), 4.58 (2H, brs), 6.45-6.7 (6H, m), 7.45-7.6 (1H, m), 8.05-8.15 (1H, m)

ESI-MS (m/z): 266 (M+Na)⁺, 244 (M+H)⁺

### EXAMPLE 348

N-(4-{2-[Methyl(2-pyridinyl)amino]ethoxy}phenyl)-4′-(trifluoromethyl)-1,1′-biphenyl-2-carboxamide was obtained in the same manner as in Preparation 19 as white crystals.

¹H-NMR (DMSO-d₆): δ3.06 (3H, s), 3.88 (2H, t, J=5.5 Hz), 4.08 (2H, t, J=5.5 Hz), 6.57 (1H, dd, J=8.6 and 4.9 Hz), 6.85 (2H, d, J=9.0 Hz), 7.39 (2H, d, J=9.0 Hz), 7.45-7.7 (7H, m), 7.74 (2H, d, J=8.3 Hz), 8.05-8.15 (1H, m), 10.17 (1H, s)

APCI-MS (m/z): 492 (M+H)⁺

### EXAMPLE 349

4′-Methyl-N-(4-{2-[methyl(2-pyridinyl)amino]ethoxy}phenyl)-1,1′-biphenyl-2-carboxamide was obtained in the same manner as in Preparation 19 as white crystals.

¹H-NMR (DMSO-d₆): δ2.28 (3H, s), 3.06 (3H, s), 3.88 (2H, t, J=6.2 Hz), 4.08 (2H, t, J=6.2 Hz), 6.56 (1H, dd, J=8.6 and 5.1 Hz), 6.85(2H, d, J=9.0 Hz), 7.16 (2H, d, J=8.0 Hz), 7.33 (2H, d, J=8.0 Hz), 7.4-7.6597H, m), 8.0-8.1 (1H, m), 10.05 (1H, s)

APCI-MS (m/z): 438 (M+H)⁺

Preparation 149

To a solution of 2-(4-aminophenyl)ethanol (6.86 g) and imidazole (3.40 g) in N,N-dimethylformamide (30 ml) was added dropwise a solution of tert-butyldimethylchlorosilane (7.54 g) in N,N-dimethylformamide (40 ml) at room temperature and the mixture was stirred at room temperature for 24 hours. The reaction mixture was poured into a mixture of ethyl acetate and ice water and the separated organic layer was washed with water and brine, dried over magnesium sulfate and evaporated in vacuo. The residue was purified by column chromatography on silica gel eluting with hexane:ethyl acetate (3:1) to give 4-(2-{[tert-butyl(dimethyl)silyl]oxy}ethyl)aniline (11.34 g) as an oil.

¹H-NMR (DMSO-d₆): δ−0.04 (6H, s), 0.82 (9H, s), 2.55 (2H, t, J=7.1 Hz), 3.64 (2H, t, J=7.1 Hz), 4.82 (2H, brs), 6.46 (2H, d, J=8.2 Hz), 6.84 (2H, d, J=8,.2 Hz)

APCI-MS (m/z): 252 (M+H)⁺

Preparation 150

To a solution of 4-(2-{[tert-butyl(dimethyl)silyl]oxy}-ethyl)aniline (5.02 g) and triethylamine (2.43 g) in dichloromethane (60 ml) was added dropwise a solution of 4′-(trifluoromethyl)-1,1′-biphenyl-2-carbonyl chloride (6.26g) in dichloromethane (40 ml) at 5° C. and the mixture was stirred at room temperature for 20 hours. Water (40 ml) was added and the separated organic layer was washed with water and brine, dried over magnesium sulfate and evaporated in vacuo. The residue was purified by column chromatography on silica gel eluting with hexane:ethyl acetate (3:1) to give N-[4-(2-{(tert-butyl(dimethyl)silyl]oxy}ethyl)phenyl]-4′-(trifluoromethyl)-1,1′-biphenyl-2-carboxamide (7.12 g) as a yellow amorphous powder.

¹H-NMR (DMSO-d₆): δ−0.06 (6H, s), 0.80 (9H, s), 2.66 (2H, t, J=6.7 Hz), 3.72 (2H, t, J=6.7 Hz), 7.10 (2H, d, J=8.4 Hz), 7.43 (2H, d, J=8.4 Hz), 7.5-7.8(8H, ), 10.23 (1H, S)

APCI-MS (m/z): 500 (M+H)⁺

Preparation 151

To a solution of crude N-[4-(2-([tert-butyl(dimethyl)silyl]oxy}ethyl)phenyl]-4′-(trifluoromethyl)-1,1′-biphenyl-2-carboxamide (7.5 g) in tetrahydrofuran (50 ml) and methanol (50 ml) was added 6N HCl (25 ml) at room temperature and the mixture was stirred at room temperature for 22 hours. The mixture was evaporated in vacuo and the residue was extracted with ethyl acetate. The separated organic layer was washed with water and brine, dried over magnesium sulfate and evaporated in vacuo. The residue was purified by column chromatography on silica gel eluting with hexane:ethyl acetate (1:2) to give N-[4-(2-hydroxyethyl)phenyl]-4′-(trifluoromethyl)-1,1′-carboxamide (3.96 g) as white crystals.

¹H-NMR (DMSO-d₆): δ2.65 (2H, t, J=7.1 Hz), 3.55 (2H, t, J=7.1 Hz), 4.59 (1H, br), 6.80 (2H, d, J=8.0 Hz), 7.10 (2H, d, J=8.0 Hz), 7.5-7.8 (8H, m), 10.27 (1H, s)

APCI-MS (m/z): 386 (M+H)⁺

### EXAMPLE 350

To a solution of N-[4-(2-hydroxyethyl)phenyl]-4′-(trifluoromethyl)-1,1′-biphenyl-2-carboxamide (771 mg), 2-hydroxypyridine (190 mg) and triphenylphosphine (525 mg) in tetrahydrofuran.(40 ml) was added diethyl azodicarboxylate (348 mg) at room temperature and the mixture was stirred for 24 hours. The mixture was evaporated in vacuo and the residue was purified by column chromatography on silica gel eluting with hexane:ethyl acetate (1:2) to give N-{4-[2-(2-pyridinyloxy)ethyl]phenyl}-4′-(trifluoromethyl)-1,1′-biphenyl-2-carboxamide (625 mg) as white crystals.

¹H-NMR (DMSO-d₆): δ2.86 (2H, t, J=7.7 Hz), 4.05 (2H, t, J=7.7 Hz), 6.11 (1H, dd, J=6.7 and 6.7 Hz), 6.37 (2H, d, J=9.2 Hz), 7.10 (2H, d, J=8.4 Hz), 7.3-7.8 (9H, m), 10.30 (1H, S)

APCI-MS (m/z): 463 (M+H)⁺

### EXAMPLE 351

To a mixture of N-(4-([2-(2-pyridinyl)ethyl]sulfanyl}phenyl)-4′-(trifluoromethyl)-1,1′-biphenyl-2-carboxamide (1.90 g) and tetrabutylammonium hydrogensulfate (270 mg) in ethyl acetate (60 ml) and water (20 ml) was added dropwise a solution of OXONE® (2.44 g) in water (15 ml) at room temperature and the mixture was stirred at room temperature for 16 hours. After addition of 10% aqueous sodium thiosulfate solution (20 ml), the separated oraganic layer was washed with water and brine, dried over magnesium sulfate and evaporated in vacuo. The residue was purified by column chromatography on silica gel eluting with ethyl acetate to give the sulfone compound, N-(4-{[2-(2-pyridinyl)ethyl]sulfonyl}phenyl)-4′-(trifluoromethyl)-1,1′-biphenyl-2-carboxamide (425 mg) as a brown amorphous solid, and eluting with ethyl acetate:methanol (10:1) to give the sulfoxide compound, N-(4-{[2-(2-pyridinyl)ethyl]sulfinyl}phenyl)-4′-(trifluoromethyl)-1,1′-biphenyl-2-carboxamide (1.42 g) as a pale brown amorphous solid.

N-(4-{[2-(2-Pyridinyl)ethyl]sulfinyl}phenyl)-4′-(trifluoromethyl)-1,1′-biphenyl-2-carboxamide

hu 1H-NMR (DMSO-d₆): δ2.95-3.1 (2H, m), 3.6-3.75 (2H, m), 7.15-7.3 (2H, m), 7.45-7.85 (13H, m), 8.41 (1H, d, J=4.7 Hz), 10.85 (1H, s)

APCI-MS (m/z): 511 (M+H)⁺

N-(4-{[2-(2-pyridinyl)ethyl]sulfonyl}phenyl)-4′-(trifluoromethyl)-1,1′-biphenyl-2-carboxamide

hu 1H-NMR (DMSO-d₆): δ2.8-3.0 (2H, m), 3.1-3.3 (2H, m), 7.2-7.35 (2H, m), 7.5-7.8 (13H, m), 8.46 (1H, d, J=4.0 Hz), 10.67 (1H, s)

APCI-MS (m/z): 495 (M+H)⁺

Preparation 152

To a solution of diisopropylamine (11.1 g) in tetrahydrofuran (80 ml) was added dropwise n-butyllithium (1.59 M hexane solution) (69.1 ml) at −60° C. under nitrogen and the mixture was stirred at the same temperature for 30 minutes. To the mixture was added dropwise a solution of 2-(2,5-dimethyl-1H-pyrrol-1-yl)-6-methylpyridine (18.63 g) in tetrahydrofuran (200 ml) at −60° C. over 50 minutes. A solution of 5 mol/L ethylene oxide in toluene (40 ml) was added carefully thereto and the mixture was gradually warmed to room temperature. The mixture was quenched by addition of saturated aqueous ammonium chloride solution and poured into a mixture of ethyl acetate and water. The mixture was adjusted to pH 2 by addition of 6N HCl. The separated organic layer was washed with water and brine, dried over magnesium sulfate, and evaporated in vacuo. The residue was purified by column chromatography on silica gel to give 3-[6-(2,5-dimethyl-1H-pyrrol-1-yl)-2-pyridinyl]-1-propanol (17.34 g) as an orange oil.

¹H-NMR (DMSO-d₆): δ1.75-1.95 (2H, m), 2.80 (2H, t, J=7.9 Hz), 3.42 (2H, td, J=7.9 and 5.2 Hz), 4.49 (1H, t, J=5.2 Hz), 5.79 (2H, s), 7.18 (1H, d, J=7.8 Hz), 7.29 (1H, d, J=7.2 Hz), 7.87 (1H, dd, J=7.8 and 7.2 Hz)

APCI-MS (m/z): 231 (M+H)⁺

### EXAMPLE 352

N-(4-{3-[6-(2,5-Dimethyl-1H-pyrrol-1-yl)-2-pyridinyl]propoxy}phenyl)-4′-(trifluoromethyl)-1,1′-biphenyl-2-carboxamide was obtained in the same manner as in Example 350 as a pale brown amorphous powder.

APCI-MS (m/z): 570 (M+H)⁺

### EXAMPLE 353

To a solution of N-(4-{3-[6-(2,5-dimethyl-1H-pyrrol-1-yl)-2-pyridinyl]propoxy)phenyl)-4′-(trifluoromethyl-1,1′-biphenyl-2-carboxamide (960 mg) in a mixture of ethanol (20 ml) and water (5 ml) were added hydroxylamine hydrochloride (1.17 g) and triethylamine (341 mg) at room temperature. The mixture was refluxed for 20 hours and evaporated to dryness. The residue was extracted with ethyl acetate and the organic layer was washed with brine, dried over magnesium sulfate, and evaporated in vacuo. The residue was purified by column chromatography on silica gel and preparative HPLC to give N-{4-[3-(6-amino-2-pyridinyl)propoxy]phenyl}-4′-(trifluoromethyl)-1,1′-biphenyl-2-carboxamide (248 mg) as a pale brown amorphous powder.

¹H-NMR (DMSO-d₆): δ1.9-2.15 (2H, m), 2.55 (2H, t, J=7.0 Hz), 3.93 (2H, t, J=7.0 Hz), 5.78 (2H, brs), 6.24 (1H, d, J=7.9 Hz), 6.34 (1H, d, J=7.1 Hz), 6.84 (2H, d, J=9.0 Hz), 7.26 (1H, d, J=7.9 and 7.1 Hz), 7.40 (2H, d, J=9.0 Hz), 7.5-7.7 (8H, m), 7.75 (2H, d, J=8.3 Hz), 10.18 (1H, s)

APCI-MS (m/z): 492 (M+H)⁺

### EXAMPLE 354

To a solution of 2-bromopyridine (2.40 g) in tetrahydrofuran (100 ml) was added dropwise n-butyllithium (1.63 mol/l hexane solution) (9.2 ml) at −30° C. and the mixture was stirred at the same temperature for an hour. To the resulting suspension was added dropwise a solution of N-(4-cyanophenyl)-4′-(trifluoromethyl)-1,1′-biphenyl-2-carboxamide (3.66 g) in tetrahydrofuran (40 ml). The mixture was gradually warmed to 0° C. and stirred at the same temperature for 3 hours. The mixture was poured into a mixture of ethyl acetate and ice water and the separated organic layer was washed with water and brine, dried over magnesium sulfate and evaporated in vacuo. The residue was purified by column chromatography on silica gel eluting with hexane:ethyl acetate (1:2) to give N-[4-(2-pyridinylcarbonyl)phenyl]-4′-(trifluoromethyl)-1,1′-biphenyl-2-carboxamide (2.33 g) as pale brown crystals.

¹H-NMR (DMSO-d₆): δ7.5-7.8 (10H, m), 7.85-8.1 (5H, m), 8.71 (1H, d, J=4.7 Hz), 10.77 (1H, s)

APCI-MS (m/z): 447 (M+H)⁺

### EXAMPLE 355

To a solution of N-[4-(2-pyridinylcarbonyl)phenyl]-4′-(trifluoromethyl)-1,1′-biphenyl-2-carboxamide (893 mg) in ethanol (20 ml) was added sodium borohydride (38 mg) at room temperature and the mixture was stirred at room temperature for 2 hours. The mixture was poured into a mixture of ethyl acetate and ice water and the separated organic layer was washed with water and brine, dried over magnesium sulfate and evaporated in vacuo. The residue was purified by column chromatography on silica gel eluting with ethyl acetate and crystallized from ethyl acetate to give N-{4-[hydroxy(2-pyridinyl)methyl]phenyl}-4═-(trifluoromethyl)-1,1′-biphenyl-2-carboxamide (773 mg) as pale brown crystals.

¹H-NMR (DMSO-d₆): δ5.64 (1H, d, J=4.2 Hz), 6.00 (1H, d, J=4.2 Hz), 7.2-7.9 (15H, m), 8.43 (1H, d, J=4.8 Hz), 10.32 (1H, s)

APCI-MS (m/z): 449 (M+H)⁺

### EXAMPLE 356

To a solution of 4-[2-(1-trityl-1H-imidazol-4-yl)ethyl]aniline (0.46 g), 4′-(trifluoromethyl)-1,1′-biphenyl-2-carboxylic acid (0.29 g) and HOBT (0.16 g) in tetrahydrofuran (25 ml) was added WSC.HCl (0.23 g), followed by triethylamine (0.2 ml) at room temperature. The reaction mixture was stirred at 50° C. for 18 hours and concentrated in vacuo. The residue was dissolved in ethyl acetate and water, and extracted with ethyl acetate. The organic layer was washed with water and brine, dried over magnesium sulfate, filtered, and concentrated in vacuo. The residue was purified by column chromatography on silica gel eluting with chloroform:methanol (19:1) to give 4′-(trifluoromethyl)-N-{4-[2-(1-trityl-1H-imidazol-4-yl)ethyl]phenyl}-1,1′-biphenyl-2-carboxamide (0.49 g) as a pale yellow soild.

### EXAMPLE 357

A solution of 4′-(trifluoromethyl)-N-{4-[2-(1-trityl-1H-imidazol-4-yl)ethyl]phenyl}-1,1′-biphenyl-2-carboxamide (0.402 g) and anisole (1.5 ml) in trifluoroacetic acid (3 ml) was refluxed for 5 hours. The reaction mixture was basified with 10% aqueous potassium carbonate solution and extracted with ethyl acetate. The organic layer was washed with water and brine, dried over magnesium sulfate, and concentrated in vacuo. The residue was purified by column chromatography on silica gel eluting with hexane:ethyl acetate (1:1) to give N-{4-[2-(1H-imidazol-4-yl)ethyl]phenyl}-4′-(trifluoromethyl)-1,1′-biphenyl-2-carboxamide (0.18 g) as a pale yellow soild.

¹H-NMR (DMSO-d₆): δ2.76-2.79 (4H, m), 6.70 (1H, s), 7.10 (2H, d, J=8.2 Hz), 7.42 (2H, d, J=8.9 Hz), 7.50-7.76 (12H, m), 10.28 (1H, s)

### EXAMPLE 358

4′-(Trifluoromethyl)-N-{4-[2-(1-trityl-1H-imidazol-2-yl)ethyl]phenyl}-1,1′-biphenyl-2-carboxamide was obtained in the same manner as in Example 356 as a yellow foam.

### EXAMPLE 359

N-{4-[2-(1H-Imidazol-2-yl)ethyl]phenyl}-4′-(trifluoromethyl)-1,1′-biphenyl-2-carboxamide was obtained in the same manner as in Example 357 as a pale yellow soild.

¹H-NMR (DMSO-d₆): δ2.85-2.87 (4H, m), 6.86 (2H, s), 7.09 (2H, d, J=8.6 Hz), 1.41 (2H, d, J=8.2 Hz), 10.29 (1H, s)

### EXAMPLE 360

To a solution of N-(4-aminophenyl)-4′-(trifluoromethyl)-1,1′-biphenyl-2-carboxamide (0.792 g), 4-pyrimidinylacetic acid (0.307 g) and HOBT (0.360 g) in N,N-dimethylformamide (10 ml) was added WSC.HCl (0.511 g), followed by triethylamine (0.47 ml) at room temperature. The reaction mixture was stirred at 50° C. for 12 hours and concentrated in vacuo. The residue was dissolved in ethyl acetate and water, and extracted with ethyl acetate. The organic layer was washed with water and brine, dried over magnesium sulfate, filtered, and concentrated in vacuo. The residue was recrystallized from ethyl acetate-diisopropyl ether to give N-{4-[(4-pyrimidinylacetyl)amino]phenyl}-4′-(trifluoromethyl)-1,1′-biphenyl-2-carboxamide (0.732 g) as a yellow brown solid.

¹H-NMR (DMSO-d₆): δ3.86 (2H, s), 7.43-7.76 (13H, m), 8.74 (1H, d, J=5.3 Hz), 9.10 (1H, s), 10.25 (1H, s), 10.29 (1H, s)

### EXAMPLE 361

N-{4-[(1H-Imidazol-4-ylacetyl)amino]phenyl}-4′-(trifluoromethyl)-1,1′-biphenyl-2-carboxamide was obtained in the same manner as in Example 194 as a yellow solid.

1H-NMR (CD₃OD): δ3.66 (2H, s), 6.99 (1H, s), 7.2-7.7 (16H, m)

FAB-MS (m/z): 465 (M+H)⁺

### EXAMPLE 362

tert-Butyl 2-(1,3-thiazol-4-yl)ethyl(4-{[(4′-ethyl-1,1′-biphenyl-2-yl)carbonyl]amino}phenyl)carbamate (158 mg) was obtained in the same manner as in Example 74 as a clear oil.

¹H-NMR (CDCl₃): δ1.26 (3H, t, J=7 Hz), 1.38 (9H, s), 2.96 (2H, q, J=7.6 Hz), 3.04 (2H, t, J=7.3 Hz), 3.94 (2H, t, J=7.3 Hz), 6.94-7.53 (13H, m), 8.70 (1H, d, J=2.0 Hz)

### EXAMPLE 363

N-(4-{[2-(1,3-Thiazol-4-yl) ethyl]amino}phenyl)-4′-ethyl-1,1′-biphenyl-2-carboxamide was obtained in the same manner as in Example 75 as a brown solid.

¹H-NMR (CDCl₃): δ1.27 (3H, t, J=7.6 Hz), 2.70 (2H, q, J=7.6 Hz), 3.10 (2H, t, J=6.6 Hz), 3.46 (2H, t, J=6.6 Hz), 6.47-6.89 (4H, AaBb), 6.71 (1H, brs), 7.01 (1H, s), 7.26-7.88 (8H, m), 8.77 (1H, d, J=2.0 Hz)

ESI-MS (m/z): 450 (M+Na)⁺, 428 (M+H)⁺

### EXAMPLE 364

tert-Butyl 2-(1,3-thiazol-4-yl)ethyl(4-{[(4′-methyl-1,1′-biphenyl-2-yl)carbonyl]amino}phenyl)carbamate was obtained in the same manner as in Example 74 as an orange oil.

¹H-NMR (CDCl₃): δ1.39 (9H, s), 2.40 (3H, s), 3.05 (2H, t, J=7.3 Hz), 3.95 (2H, t, J=7.3 Hz), 6.95-7.9 (13H, m), 8.70 (1H, d, J=2.0 Hz)

### EXAMPLE 365

N-(4-{[2-(1,3-Thiazol-4-yl)ethyl]amino}phenyl)-4′-methyl-1,1′-biphenyl-2-carboxamide was obtained in the same manner as in Example 75 as a yellow solid.

¹H-NMR (CDCl₃): δ2.39 (3H, s), 3.09 (2H, t, J=6.6 Hz), 3.45 (2H, t, J=6.6 Hz), 6.49-6.94 (4H, AaBb), 6.79 (1H, brs), 7.01 (1H, brs), 7.22-7.53 (8H, m), 7.84 (1H, d, J=7.6 Hz), 8.76 (1H, d, J=2.3 Hz)

ESI-MS (m/z): 494 (M+H)⁺

### EXAMPLE 366

tert-Butyl 2-(1,3-thiazol-4-yl)ethyl(4-{[(4′-methoxy-1,1′-biphenyl-2-yl)carbonyl]amino}phenyl)carbamate was obtained in the same manner as in Example 74 as a yellow oil.

¹H-NMR (CDCl₃): δ1.38 (9H, s) 3.04 (2H, t, J=7.3 Hz), 3.82 (3H, s), 3.95 (2H, t, J=7.3 Hz), 6.94-7.85 (13H, m), 8.69 (1H, d, J=2.0 Hz)

### EXAMPLE 367

N-(4-{[2-(1,3-Thiazol-4-yl)ethyl]amino}phenyl)-4′-methoxy-1,1′-biphenyl-2-carboxamide was obtained in the same manner as in Example 75 as a,yellow solid.

¹H-NMR (CDCl₃): δ3.10 (2H, t, J=6.6 Hz), 3.45 (2H, t, J=6.6 Hz), 6.52-7.88 (13H, m), 8.77 (1H, d, J=2.0 Hz)

ESI-MS (m/z): 430 (M+H)⁺

### EXAMPLE 368

N-{4-[2-(2-Methyl-1,3-thiazol-4-yl)ethoxy]phenyl}-4′-(trifluoromethyl)-1,1′-biphenyl-2-carboxamide was obtained in the same manner as in Example 219 as a colorless solid.

¹H-NMR (CDCl₃): δ2.80 (3H, s), 3.25 (2H, t, J=6.3 Hz), 4.24 (2H, t, J=6.3 Hz), 7,02 (2H, d, J=8.9 Hz), 6.83 (1H, brs), 7.00 (1H, s), 7.06 (2H, d, J=9.2 Hz), 7.42-7.82 (8H, m)

ESI-MS (m/z): 505 (M+Na)⁺, 483 (M+H)⁺

Preparation 153

To a solution of ethyl (2-methyl-1,3-thiazol-4-yl)acetate (5.08 g) in tetrahydrofuran (35 ml) was added lithium tetrahydroborate (1.60 g) as a solid in one portion at 0° C. After the evolution of the gas ceased, the reaction mixture was allowed to warm up to room temperature and stirred for two hours. The reaction mixture was quenched with water and extracted with ethyl acetate (twice). The combined organic layers were washed with brine, dried over magnesium sulfate, filtered and concentrated in vacuo to give 2-(2-methyl-1,3-thiazol-4-yl)ethanol (3.402 g) as a yellow oil.

¹H-NMR (CDCl₃): δ2.69 (3H, s), 2.95 (2H, t, J=5.8 Hz), 3.93 (2H, t, J=5.8 Hz), 6.81 (1H, s)

Preparation 154

To a solution of 2-(2-methyl-1,3-thiazol-4-yl)ethanol (3.402 g), triethylamine (3.20 g) and 4-(N,N-dimethylamino)pyridine (300 mg) in 1,2-dichloroethane (50 ml) was -added dropwise a solution of p-toluenesulfonyl chloride (6.00 g) in 1,2-dichloroethane (20 ml) at 0° C. The reaction mixture was stirred at room temperature for 10 hours, and then washed with saturated aqueous sodium hydrogencarbonate solution, 1N HCl and brine. The separated organic layer was dried over magnesium sulfate, filtered, and concentrated in vacuo. The residue was purified by column chromatography on silica gel eluting with hexane:ethyl acetate (2:1) to give 2-(2-methyl-1,3-thiazol-4-yl)ethyl 4-methylbenzenesulfonate (3.716 g) as a pale yellow oil.

¹H-NMR (CDCl₃): δ2.44 (3H, s), 2.61 (3H, s), 3.06 (2H, t, J=6.6 Hz), 4.33 (2H, t, J=6.6 Hz), 6.81 (1H, s), 7.30 (2H, d, J=7.9 Hz), 7.71 (2H, d, J=8.6 Hz).

Preparation 155

To a solution of 2-(2-methyl-1,3-thiazol-4-yl)ethyl 4-methylbenzenesulfonate (3.35 g) in N,N-dimethylformamide (20 ml) was added sodium azide (1.464 g) as a solid at room temperature. The reaction mixture was stirred at room temperature for 13 hours. After removal of the solvent under the reduced pressure, ethyl acetate and water were added to the residue. The separated organic layer was washed with brine, dried over magnesium sulfate, filtered and concentrated in vacuo to give-4-(2-azidoethyl)-2-methyl-1,3-thiazole (1.609 g) as a brown oil.

¹H-NMR (CDCl₃): δ2.69 (3H, s), 3.00 (2H, t, J=6.9 Hz), 3.62 (2H, t, J=6.9 Hz), 6.86 (1H, s)

Preparation 156

To a solution of 4-(2-azidoethyl)-2-methyl-1,3-thiazole (1.607 g) in methanol (30 ml) was added palladium on charcoal (10% supported, 50% wet; 871 mg), and then a balloon filled with hydrogen gas was equipped. The reaction mixture was stirred at room temperature for 4 hours, filtered through a short pad of celite, dried over magnesium sulfate, filtered again and concentrated in vacuo to give 2-(2-methyl-1,3-thiazol-4-yl)ethylamine (1.359 g) as an orange oil.

¹H-NMR (CDCl₃): δ2.69 (3H, s), 2.86 (2H, t, J=6.5 Hz), 3.04 (2H, t, J=6.5 Hz), 6.78 (1H, s)

Preparation 157

N-[2-(2-methyl-1,3-thiazol-4-yl)ethyl]-4-nitroaniline was obtained in the same manner as in Preparation 33 as a yellow oil.

¹H-NMR (CDCl₃): δ2.71 (3H, s), 3.05 (2H, t, J=6.3 Hz), 3.55 (2H, t, J=6.3 Hz), 6.54 (2H, d, J=8.9 Hz), 6.83 (1H, s), 8.07 (2H, d, J=9.2 Hz)

Preparation 158

To a solution of N-[2-(2-methyl-1,3-thiazol-4-yl)ethyl]-4-nitroaniline (1.367 g) in methanol (30 ml) was added palladium on carbon (10% supported, 50% wet; 1.04 g),and then a balloon filled with hydrogen gas was equipped. The reaction mixture was stirred at room temperature for 14 hours, filtered through a short pad of celite, dried over magnesium sulfate, filtered again and concentrated in vacuo to give N-[2-(2-methyl-1,3-thiazol-4-yl)ethyl]-1,4-benzenediamine (877 mg) as a black oil.

¹H-NMR (CDCl₃): δ2.70 (3H, s), 3.00 (2H, t, J=6.3 Hz), 3.41 (2H, t, J=6.3 Hz), 6.54 (2H, d, J=8.6 Hz), 6.61 (2H, d, J=8.3 Hz), 6.78 (1H, s)

### EXAMPLE 369

To a solution of N-[2-(2-methyl-1,3-thiazol-4-yl)ethyl]-1,4-benzenediamine (233 mg), 4′-(trifluoromethyl)-1,1′-biphenyl-2-carboxylic acid (150 mg) and HOBT (104 mg) in N,N-dimethylformamide (10 ml) was added WSC.HCl (130 mg), followed by triethylamine (74 mg) at room temperature. The reaction mixture was stirred at 40° C. for 13 hours and concentrated in vacuo. The residue was dissolved in ethyl acetate and water, and extracted with ethyl acetate. The organic layer was washed with brine, dried over magnesium sulfate, filtered and concentrated in vacuo. The residue was purified by column chromatography on silica gel eluting with chloroform:methanol (39:1) to give N-(4-{[2-(2-methyl-1,3-thiazol-4-yl)ethyl]amino}phenyl)-4′-(trifluoromethyl)-1,1′-biphenyl-2-carboxamide (165 mg) as pale brown crystals.

¹H-NMR (CDCl₃): δ2.62 (3H, s), 2.88 (2H, t, J=6.9 Hz), 3.27 (2H, t, J=6.9 Hz), 5.50 (1H, brs), 6.49 (2H, d, J=8.9 Hz), 7.15 (1H, s), 7.19 (2H, d, J=8.9 Hz), 7.47-7.65 (6H, m), 7.75 (2H, d, J=8.2 Hz), 9.90 (1H, s)

ESI-MS (m/z): 503 (M+Na)⁺

### EXAMPLE 370

4′-Ethyl-N-(4-([2-(2-methyl-1,3-thiazol-4-yl)ethyl]amino}phenyl)-1,1′-biphenyl-2-carboxamide was obtained in the same manner as in Example 369 as pale brown crystals.

¹H-NMR (DMSO-d₆): δ1.18 (3H, t, J=7.6 Hz), 2.60 (2H, q, J=7.6 Hz), 2.63 (3H, s), 2.88 (2H, t, J=7.3 Hz), 3.27 (2H, t, J=7.3 Hz), 5.49 (1H, t, J=5.6 Hz), 6.50 (2H, d, J=8.9 Hz), 7.15 (1H, s), 7.19-7.22 (4H, m), 7.35-7.55 (6H, m), 9.78 (1H, s)

ESI-MS (m/z): 464 (M+Na)⁺, 442 (M+H)⁺

### EXAMPLE 371

4′-Methyl-N-(4-{[2-(2-methyl-1,3-thiazol-4-yl)ethyl]amino}phenyl)-1,1′-biphenyl-2-carboxamide was obtained in the same manner as in Example 369 as faintly greenish yellow crystals.

¹H-NMR (CDCl₃): δ2.39 (3H, s), 2.69 (3H, s), 2.99 (2H, t, J=6.6 Hz), 3.42 (2H, t, J=6.6 Hz), 6.50 (2H, d, J=8.9 Hz), 6.73 (1H, brs), 6.77 (1H, s), 6.92 (2H, d, J=8.6 Hz), 7.22-7.26 (2H, m), 7.35-7.53 (5H, m), 7.85 (1H, d, J=7.3 Hz)

ESI-MS (m/z).:450 (M+Na)⁺, 428 (M+H)⁺

Preparation 159

Tert-butyl 4-(2-hydroxyethyl)-1,3-thiazol-2-yl(methyl)carbamate was obtained in the same manner as in Preparation 153 as a colorless oil.

¹H-NMR (CDCl₃): δ1.58 (9H, s), 2.87 (2H, t, J=5.9 Hz), 3.52 (3H, s), 3.90 (2H, br.t, J=5.9 Hz), 6.56 (1H, s)

Preparation 160

2-(2-[(tert-Butoxycarbonyl)(methyl)amino]-1,3-thiazol-4-yl}ethyl 4-methylbenzenesulfonate was obtained in the same manner as in Preparation 154 as colorless crystals.

¹H-NMR (CDCl₃): δ1.58 (9H, s), 2.42 (3H, s), 2.95 (2H, d, J=6.5 Hz), 3.37 (3H, s), 4.34 (2H, t, J=6.5 Hz), 6.53 (1H, s), 7.26 (2H, d, J=8.3 Hz), 7.67 (2H, d, J=6.3 Hz)

Preparation 161

tert-Butyl 4-(2-azidoethyl)-1,3-thiazol-2-yl(methyl)carbamate was obtained in the same manner as in Preparation 155 as a pale yellow oil.

¹H-NMR (CDCl₃): δ1.58 (9H, s), 2.92 (2H, t, J=6.9 Hz), 3.53 (3H, s), 3.60 (2H, t, J=6.9 Hz), 6.61 (1H, s)

Preparation 162

Tert-Butyl 4-(2-aminoethyl)-1,3-thiazol-2-yl(methyl)carbamate was obtained in the same manner as in Preparation 156 as an orange oil.

¹H-NMR (CDCl₃): δ1.57 (9H, s), 2.78 (2H, t, J=6.5 Hz), 3.03 (2H, t, J=6.5 Hz), 3.53 (3H, s), 6.54 (1H, s)

Preparation 163

tert-Butyl methyl (4-{2-[(4-nitrophenyl)amino]ethyl}-1,3-thiazol-2-yl)carbamate was obtained in the same manner as in Example 33 as a yellow oil.

¹H-NMR (CDCl₃): δ1.58 (9H, s), 2.97 (2H, t, J=6.2 Hz), 3.51 (2H, brs), 3.57 (3H, s), 5.44 (1H, brs), 6.51 (2H, d, J=9.2 Hz), 6.60 (1H, s), 8.07 (2H, d, J=9.3 Hz)

Preparation 164

Tert-butyl 2-{2-[(tert-butoxycarbonyl)(methyl)amino]-1,3-thiazol-4-yl}ethyl(4-nitrophenyl)carbamate was obtained in the same manner as in Preparation 34 as light yellow crystals.

¹H-NMR (CDCl₃): δ1.47 (9H, s), 1.57 (9H, s), 2.95 (2H, t, J=6.9 Hz), 3.39 (3H, s), 4.06 (2H, t, J=6.9 Hz), 6.52 (1H, s), 7.31 (2H, d, J=9.2 Hz), 8.13 (2H, d, J=9.3 Hz)

Preparation 165

tert-Butyl 4-aminophenyl(2-{2-f[tert-butoxycarbonyl)(methyl)amino]-1,3-thiazol-4-yl}ethyl)carbamate was obtained in the same manner as in Preparation 35 as a faintly yellow oil.

¹H-NMR (CDCl₃): δ1.39 (9H, brs), 1.57 (9H, s), 2.87 (2H, t, J=6.6 Hz), 3.47 (3H, s), 3.64 (2H, brs), 3.87 (2H, t, J=6.6 Hz), 6.52 (1H, s), 6.61 (2H, d, J=8.6 Hz), 6.91 (2H, brs)

### EXAMPLE 372

tert-Butyl 2-{2-[(tert-butoxycarbonyl)(methyl)amino]-1,3-thiazol-4-yl}ethyl[4-({[4′-(trifluoromethyl)-1,1′-biphenyl-2-yl]carbonyl}amino)phenyl]carbamate was obtained in the same manner as in Example 74 as a brown oil

### EXAMPLE 373

N-[4-({2-[2-(Methylamino)-1,3-thiazol-4-yl]ethyl}amino)phenyl]-4′-(trifluoromethyl)-1,1′-biphenyl-2-carboxamide was obtained in the same manner as in Example 75 as pale brown crystals.

¹H-NMR (CDCl₃): δ2.81 (2H, t, J=6.6 Hz),2.96 (3H, s), 3.35 (2H, t, J=6.6 Hz), 5.22 (1H, brs), 6.15 (1H, s), 6.51 (2H, d, J=8.9 Hz), 6.74 (1H, s), 6.94 (2H, d, J=8.6 Hz), 7.41-7.70 (7H, m), 7.79 (1H, d, J=6.9 Hz)

ESI-MS (m/z): 519 (M+Na)⁺, 497 (M+H)⁺

### EXAMPLE 374

tert-Butyl 2-{2-[(tert-butoxycarbonyl)(methyl)amino]-1,3-thiazol-4-yl}ethyl{4-{[(4′-methyl-1,1′-biphenyl-2-yl)carbonyl]amino}phenyl)carbamate was obtained in the same manner as in Example 74 as a yellow oil.

ESI-MS (m/z): 665 (M+Na)⁺

### EXAMPLE 375

4′-Methyl-N-[4-({2-(2-(methylamino)-1,3-thiazol-4-yl)ethyl}amino)phenyl]-1,1′-biphenyl-2-carboxamide was obtained in the same manner as in Example 75 as faintly orange crystals.

¹H-NMR (CDCl₃): δ2.39 (3H, s), 2.81 (2H, t, J=6.3 Hz), 2.96 (3H, s), 3.35 (2H, t, J=6.3 Hz), 5.14 (1H, brs), 6.15 (1H, s), 6.50 (2H, d, J=8.6 Hz), 6.73 (1H, brs), 6.92 (2H, d, J=8.9 Hz), 7.22-7.26 (2H, m), 7.36-7.53 (5H, m), 7.85 (1H, d, J=7.3 Hz)

ESI-MS (m/z): 443 (M+H)⁺

### EXAMPLE 376

To a solution of tert-butyl 4-aminophenyl[2-(2-pyridinyl)ethyl]carbamate (5.166 g), 4′-acetyl-1,1′-biphenyl-2-carboxylic acid (3.964 g) and HOBT (2.814 g) in N,N-dimethylformamide (150 ml) was added WSC.HCl (3.548 g), followed by triethylamine (2.02 g) at room temperature. The mixture was stirred at 40° C. for 24 hours. N,N-Dimethylformamide was removed under reduced pressure, and then ethyl acetate (100 ml) and water (50 ml) were added. The separated organic layer was washed with brine, dried over magnesium sulfate and concentrated in vacuo. The residue was purified by column chromatography on silica gel eluting with hexane:ethyl acetate (1:2) to give 4-acetyl-2′-[(4-}(tert-butoxycarbonyl) [2-(2-pyridinyl) ethyl]amino}anilino)-carbonyl]-1,1′-biphenyl (5.01 g) as a yellow solid.

¹H-NMR (CDCl₃): δ1.38 (9H, s), 2.61 (3H, s), 3.00 (2H, t, J=7.6 Hz), 3.96 (2H, t, J=7.6 Hz), 7.00-7.20 (7H, m), 7.46-7.60 (6H, m), 7.80 (1H, d, J=6.3 Hz), 8.02 (2H, d, J=8.2 Hz), 8.47 (1H, d, J=4.3 Hz)

### EXAMPLE 377

To a solution of 4-acetyl-2′-[(4-{(tert-butoxycarbonyl) [2-(2-pyridinyl)ethyl]amino}anilino)carbonyl]-1,1′-biphenyl (435 mg) in dichloromethane (10 ml) was added trifluoroacetic acid (1.48 g) at room temperature and the reaction mixture was stirred for 18 hours. 10% Aqueous potassium carbonate solution was added until the mixture was basified, and the separated organic layer was washed with brine, dried over magnesium sulfate and concentrated in vacuo. The oily residue was recrystallized from ethyl acetate-diisopropyl ether to give 4′-acetyl-N-(4-{[2-(2-pyridinyl)ethyl]amino}phenyl)-1,1′-biphenyl-2-carboxamide (337 mg) as faintly greenish yellow crystals.

¹H-NMR (CDCl₃): δ2.61 (3H, s), 3.05 (2H, t, J=6.6 Hz), 3.48 (2H, t, J=6.6 Hz), 6.51 (2H, d, J=8.9 Hz), 6.80 (1H, brs), 6.98 (2H, d, J=8.9 Hz), 7.15 (2H, d, J=Hz), 7.44-7.60 (6H, m), 7.79 (1H, d, J=Hz), 8.01 (2H, d, J=Hz), 8.54 (1H, d, J=4.0 Hz)

ESI-MS (m/z): 436 (M+H)⁺

### EXAMPLE 378

To a solution of 4′-acetyl-N-(4-{[2-(2-pyridinyl)ethyl]amino}phenyl)-1,1′-biphenyl-2-carboxamide (337 mg) in methanol (10 ml) was added sodium borohydride (44 mg) at room temperature and the mixture was stirred for 30 minutes. Methanol was removed under the reduced pressure, and then ethyl acetate (20 ml) and water (20 ml) were added. The separated organic layer was washed with brine, dried over magnesium sulfate and concentrated in vacuo. The residue was recrystallized from ethyl acetate-diisopropyl ether to give 4′-(1-hydroxyethyl)-N-(4-{[2-(2-pyridinyl)ethyl]amino}phenyl)-1,1′-biphenyl-2-carboxamide (292 mg) as white crystals.

¹H-NMR (CDCl₃): δ1.52 (3H, d, J=6.3 Hz), 3.04 (2H, t, J=6.6 Hz), 3.47 (2H, t, J=6.6 Hz), 4.95 (2H, q, J=6.3 Hz), 6.49 (2H, d, J=8.9 Hz), 6.70 (1H, brs), 6.89 (2H, d, J=8.9 Hz), 7.15 (2H, d, J=7.6 Hz), 7.39-7.60 (8H, m), 7.82-7.85 (1H, m), 8.54 (1H, d, J=4.0 Hz).

FAB-MS (m/z): 438 (M+H)⁺

### EXAMPLE 379

To a solution of tert-butyl 4-{[(4′-acetyl-1,1′-biphenyl-2-yl)carbonyl]amino}phenyl[2-(2-pyridinyl)ethyl]carbamate (1.011 g) in anhydrous tetrahydrofuran (50 ml) was added dropwise methylmagnesium bromide in dibutyl ether (1M solution, 5.0 ml) at room temperature and the reaction mixture was stirred for 36 hours. The reaction mixture was quenched with 1N HCl, and then basified with 10% aqueous potassium carbonate solution. Tetrahydrofuran was evaporated and the residue was extracted with ethyl acetate, washed with brine, dried over magnesium sulfate and concentrated in vacuo. The residue was purified by column chromatography on silica gel eluting with hexane:ethyl acetate (1:1 to 1:3) to give tert-butyl 4-(([4′-(1-hydroxy-1-methylethyl)-1,1′-biphenyl-2-yl]carbonyl}amino)phenyl[2-(2-pyridinyl)ethyl]carbamate (462 mg) as a yellow tar.

¹H-NMR (CDCl₃): δ1.37 (9H, s), 1.60 (6H, s), 2.99 (2H, t, J=7.6 Hz), 3.95 (2H, t, J=7.6 Hz), 6.87 (1H, brs), 6.98-7.18 (6H, m), 7.42-7.61 (8H, m), 7.88 (1H, d, J=6.2 Hz), 8.48 (1H, d, J=4.0 Hz)

ESI-MS (m/z): 574 (M+Na)⁺

### EXAMPLE 380

To a suspension of tert-butyl 4-({[4′-(1-hydroxy-1-methylethyl)-1,1′-biphenyl-2-yl]carbonyl}amino)phenyl[2-(2-pyridinyl)ethyl]carbamate (345 mg) and sodium borohydride (118 mg) in anhydrous tetrahydrofuran (15 ml) was added dropwise trifluoroacetic acid (710 mg) at 0° C. The mixture was stirred at the same temperature for 1 hour. The reaction mixture was quenched with saturated aqueous sodium hydrogencarbonate solution. Ethyl acetate (30 ml) and water (20 ml) were added and the separated organic layer was washed with brine, dried over magnesium sulfate and concentrated in vacuo. The residue was purified by column chromatography on silica gel eluting with chloroform:methanol (19:1) to give a yellow oil. To a solution of the obtained oil in dichloromethane (15 ml) was added trifluoroacetic acid at room temperature and the mixture was stirred for 13 hours. Then, 10% aqueous potassium carbonate solution was added until the mixture was basified and the separated organic layer was washed with brine, dried over magnesium sulfate and concentrated in vacuo. The residue was purified by column chromatography on silica gel eluting with hexane:ethyl acetate (1:1) to give a brown tar. The obtained tar was recrystallized from ethyl acetate-diisopropyl ether to give 4′-isopropyl-N-(4-{[2-(2-pyridinyl)ethyl]amino}phenyl)-1,1′-biphenyl-2-carboxamide (61 mg) as faintly brown crystals.

¹H-NMR (CDCl₃): δ1.30 (6H, d, J=6.9 Hz), 2.91-3.02 (1H, m), 3.06 (2H, t, J=6.6 Hz), 3.49 (2H, t, J=6.6 Hz), 6.47 (2H, d, J=8.9 Hz), 6.64 (1H, brs), 6.81 (2H, d, J=8.9 Hz), 7.13-7.16 (2H, m), 7.29-7.62 (8H, m), 7.88-7.91 (1H, m), 8.54 (1H, d, J=4.0 Hz)

Preparation 166

To a solution of 4′-methyl-1,1′-biphenyl-2-carboxylic acid (0.400 g), 4-aminophenol (0.206 g) and HOBT (0.346 g) in N,N-dimethylformamide (20 ml) was added WSC.HCl (0.434 g), followed by triethylamine (0.248 g) at room temperature. The mixture was stirred at 40° C. for 4 hours. N,N-Dimethylformamide was removed under reduced pressure, and then ethyl acetate (20 ml) and water (20 ml) were added. The separated organic layer was washed with brine, dried over magnesium sulfate and concentrated in vacuo. The residue was purified by column chromatography on silica gel eluting with chloroform:methanol (9:1) to give N-(4-hydroxyphenyl)-4′-methyl-1,1′-biphenyl-2-carboxamide (0.562 g) as a pale purple oil.

¹H-NMR (CDCl₃): δ2.38 (3H, s), 6.68 (2H, d, J=8.9 Hz), 6.88 (1H, brs), 6.91 (2H, d, J=8.9 Hz), 7.21-7.54 (7H, m), 7.82 (1H, d, J=7.3 Hz)

### EXAMPLE 381

Into a suspension of NaH (60% in oil, 70 mg) in N,N-dimethylformamide (5 ml) was added N-(4-hydroxyphenyl)-4′-methyl-1,1 ′-biphenyl-2-carboxamide (100 mg) as a solid at 0° C. After stirring for 10 minutes, a solution of 2-{2-[(tert-butoxycarbonyl)amino]-2-pyridinyl}ethyl 4-methylbenzenesulfonate (197 mg) in N,N-dimethylformamide (5 ml) was added dropwise at 0° C. The reaction mixture was stirred at room temperature for 10 hours. The reaction mixture was quenched with water and extracted with ethyl acetate. The organic layer was washed with brine, dried over magnesium sulfate, filtered and concentrated in vacuo. The residue was purified by column chromatography on silica gel eluting with hexane:ethyl acetate (2:1) to give tert-butyl 6-[2(4-{[(4′-methyl-1,1′-biphenyl-2-yl)carbonyl]amino}phenoxy)ethyl]-2-pyridinylcarbamate (125 mg) as a pale brown oil.

### EXAMPLE 382

N-{4-[2-(6-Amino-2-pyridinyl)ethoxy]phenyl}-4′-methyl-1,1′-biphenyl-2-carboxamide was obtained in the same manner as in Example 81 as pale brown crystals.

¹H-NMR (CDCl₃): δ2.17 (3H, s), 3.04 (2H, t, J=6.9 Hz), 4.25 (2H, t, J=6.9 Hz), 4.41 (2H, brs), 6.36 (1H, d, J=8.3 Hz), 6.59(1H, d, J=7.6 Hz), 6.78 (2H, d, J=8.9 Hz), 6.80 (1H, brs), 6.98-7.00 (2H, m), 7.01-7.55 (6H, m), 7.86 (1H, dd, J=1.5 and 7.4 Hz)

ESI-MS (m/z): 446 (M+Na)⁺, 424 (M+H)⁺

### EXAMPLE 383

To a solution of N-{4-[2-(6-amino-2-pyridinyl)ethoxy]phenyl}-4′-methyl-1,1′-biphenyl-2-carboxamide (984 mg) in ethyl acetate (30 ml) was slowly added 4N HCl in ethyl acetate (10 ml) at room temperature. The reaction was stirred at ambient temperature for 30 minutes and concentrated in vacuo. The residue was recrystallized from methanol-diisopropyl ether to give N-{4-[2-(6-amino-2-pyridinyl)ethoxy]phenyl}-4′-methyl-1,1′-biphenyl-2-carboxamide hydrochloride (915 mg) as a white powder.

¹H-NMR (DMSO-d₆): δ2.29 (3H, s), 3.15 (2H, t, J=6.3 Hz), 4.29 (2H, t, J=6.3 Hz), 6.80-6.88 (4H, m), 7.17 (2H, d, J=7.9 Hz), 7.33 (2H, d, J=7.9 Hz), 7.41-7.57 (6H, m), 7.82-7.88 (1H, m), 10.08 (1H, s)

ESI-MS (m/z): 424 (M+H)⁺ as a HCl-free form

### EXAMPLE 384

N-(4-{[3-(2-Pyridinyl)propanoyl]amino}phenyl)-4′-(trifluoromethyl)-1,1′-biphenyl-2-carboxamide was obtained in the same manner as in Preparation 15.

¹H-NMR (DMSO-d₆): δ2.75 (2H, t, J=7.16 Hz), 3.06 (2H, t, J=7.16 Hz), 6.57-7.78 (15H, m), 8.49 (1H, d, J=4.06 Hz), 9.93 (1H, s), 10.27 (1H, s)

### EXAMPLE 385

N-{4-[(1,3-Thiazol-2-yl)ethynyl]phenyl}-4′-(trifluoromethyl)-1,1′-biphenyl-2-carboxamide was obtained in the same manner as in Example 287 from N-(4-ethynylphenyl)-4′-(trifluoromethyl)-1,1 ′-biphenyl-2-carboxamide and 2-bromothiazole.

¹H-NMR (DMSO-d₆): δ7.50-7.91 (14H, m), 10.64 (1H, s)

Preparation 167

A 28% sodium methylate-methanol solution (12 ml) was added to a solution of N-[(6-acetyl-2-pyridinyl)methyl]acetamide (3.85 g) and 4-nitrobenzaldehyde (3.02 g) in methanol (60 ml) and tetrahydrofuran (40 ml) in ambient temperature under stirring and the resultant mixture was stirred for 4 hours for ambient temperature. Water (100 ml) was added to the reaction mixture and adjusted to pH 3.0 with 6N hydrochloric acid. The precipitate was collected by filtration, washed with ethyl acetate and diisopropylether and dried to give N-({6-[(2E)-3-(4-nitrophenyl)-2-propenoyl]-2-pyridinyl}methyl)acetamide (2.3 g).

¹H-NMR (DMSO-d₆): δ1.97 (3H, s), 4.52 (2H, d, J=5.90 Hz), 7.61 (1H, d, J=6.63 Hz), 7.94 (1H, d, J=16.20 Hz), 8.00-8.12 (4H, m), 8.31 (2H, d, J=8.59 Hz), 8.42 (1H, d, J=16.20 Hz), 8.58-8.60 (1H, m)

Preparation 168

A mixture of N-({6-[(2E)-3-(4-nitrophenyl)-2-propenoyl]-2-pyridinyl}methyl)acetamide (2.2 g) in methanol (100 ml) and tetrahydrofuran (50 ml) was hydrogenated over 10% palladium on carbon (1.1 g) under an atmospheric pressure of hydrogen at ambient temperature under stirring for 10 hours. After removal of the catalyst, the solvent was evaporated in vacuo to give N-({6-[3-(4-aminophenyl)-1-hydroxypropyl]-2-pyridinyl}methyl)acetamide (1.8 g).

¹H-NMR (DMSO-d₆): δ1.95 (3H, s), 1.58-1.77 (2H, m), 2.45-2.55 (2H, m), 4.31 (2H, d, J=5.97 Hz), 4.50-4.57 (1H, m), 4.81 (2H, s), 5.37 (1H, d, J=5.06 Hz), 6.48 (2H, d, J=8.23 Hz),6.85 (2H, d, J=8.23 Hz), 7.11 (1H, d, J=7.59 Hz), 7.66 (1H, d, J=7.65 Hz), 7.73-7.76 (1H, m), 8.43 (1H, t, J=5.97 Hz)

### EXAMPLE 386

A solution of 4′-(trifluoromethyl)-1,1′-biphenyl-2-carbonyl chloride (1.71 g) in ethyl acetate (5 ml) was added to a solution of N-({6-[3-(4-aminophenyl)-1-hydroxypropyl)-2-pyridinyl}methyl)acetamide (1.8 g) and N,O-bis(trimethylsilyl)acetamide (4.4 ml) in ethyl acetate (50 ml) at ambient temperature under stirring. The resultant mixture was stirred at ambient temperature for 6 hours. The reaction mixture was poured into a mixture of ethyl acetate and water and the organic layer was washed with water and brine and dried over magnesium sulfate. The solvent was concentrated in vacuo and the residue was chromatographed on silica gel (50 g) eluting with ethyl acetate and n-hexane (5:5-7:3). The fraction was evaporated in vacuo and the residue was triturated with diisopropyl ether to give N-[4-(3-{6-[(acetylamino)methyl]-2-pyridinyl}-3-hydroxypropyl)phenyl]-4′-(trifluoromethyl)-1,1′-biphenyl-2-carboxamide (1.95 g).

¹H-NMR (DMSO-d₆): δ1.95 (3H, s), 1.76-1.98 (2H, m), 2.45-2.57 (2H, m), 4.27 (2H, d, J=6.00 Hz), 4.64-4.70 (1H, m), 7.04-7.12 (3H, m), 7.29 (1H, d, J=7.62 Hz), 7.39 (1H, d, J=8.42 Hz), 7.45-7.75 (10H, m), 8.38 (1H, t, J=6.00 Hz), 10.24 (1H, s)

### EXAMPLE 387

N-{4-[3-Hydroxy-3-(2-pyridinyl)propyl]phenyl}-4′-(trifluoromethyl)-1,1′-biphenyl-2-carboxamide was obtained in the same manner as in Example 25.

¹H-NMR (DMSO-d₆): δ1.89 (3H, s), 1.89-1.99 (2H, m), 2.56-2.66 (2H, m), 2.69 (2H, t, J=7.38 Hz), 4.29 (2H, d, J=5.98 Hz), 7.05-7.14 (3H, m), 7.44 (2H, d, J=8.40 Hz), 7.49-7.69 (7H, m), 7.75 (2H, d, J=8.32 Hz), 8.40 (1H, t, J=5.98 Hz), 10.29 (1H, s)

### EXAMPLE 388

N-(4-{2-[Methyl(2-pyridinyl)amino]-2-oxoethyl}phenyl)-4′-(trifluoromethyl)-1,1′-biphenyl-2-carboxamide was obtained in the same manner as in Example 1 as white crystals.

¹H-NMR (DMSO-d₆): δ3.27 (3H, s), 3.62 (2H, s), 7.01 (2H, d, J=8.4 Hz), 7.25-7.35 (1H, m), 7.40 (2H, d, J=8.4 Hz), 7.5-7.7 (7H, m), 7.76 (2H, d, J=8.3 Hz), 7.85-7.95 (1H, m), 8.45-8.55 (1H, m), 10.30 (1H, s)

ESI-MS (m/z): 512 (M+Na)⁺, 490 (M+H)⁺

Preparation 169

To a suspension of lithium aluminum hydride (190 mg) in tetrahydrofuran (100 ml) was added dropwise a solution of 2-[(4-{[(tert-butoxycarbonyl)amino]methyl}anilino)carbonyl]-4′-(trifluoromethyl)-1,1′-biphenyl (2.353 g) in tetrahydrofuran (40 ml) at room temperature under nitrogen and the mixture was refluxed for 2 hours. The mixture was cooled to room temperature and sodium fluoride (840 mg) was added followed by addition of water (270 mg). The mixture was vigorously stirred for 30 minutes and the insoluble materials were filtered off and washed with tetrahydrofuran. The filtrate was evaporated in vacuo and the residue was purified by column chromatography on silica gel eluting with ethyl acetate:methanol (10:1) to give N-{4-[(methylamino)methyl]phenyl}-4′-(trifluoromethyl)-1,1′-biphenyl-2-carboxamide (1.06 g) as a yellow powder.

¹H-NMR (DMSO-d₆): δ2.24 (3H, s), 3.58 (2H, s), 7.17 (2H, d, J=7.5 Hz), 7.45 (2H, d, J=7.5 Hz), 7.5-7.8 (8H, m), 10.30 (1H, s)

ESI-MS (m/z): 385 (M+H)⁺

### EXAMPLE 389

N-Methyl-N-[4-({[4′-(trifluoromethyl)-1,1′-biphenyl-2-yl]carbonyl}amino)benzyl]-2-pyridinecarboxamide was obtained in the same manner as in Example 53 as a white powder.

¹H-NMR (DMSO-d₆): δ2.74, 2.83 (total 3H, s), 4.48, 4.63 (total 2H, s), 7.18, 7.22 (total 2H, d, J=8.5 Hz), 7.45-8.05 (11H, m), 8.55-8.65 (1H, m), 10.38, 10.41 (total 1H, s)

ESI-MS (m/z): 512 (M+Na)⁺

Preparation 170

N-[4-(Cyanomethyl)phenyl]-4′-(trifluoromethyl)-1,1′-biphenyl-2-carboxamide was in the same manner as in Preparation 19 as white crystals.

hu 1H-NMR (DMSO-d₆): δ3.96 (2H, s), 7.25 (2H, d, J=8.4 Hz), 7.5-7.8 (8H, m), 7.76 (2H, d, J=8.4 Hz), 10.43 (1H, s)

APCI-MS (m/z): 381 (M+H)⁺

Preparation 171

N-[4-(2-Aminoethyl)phenyl]-4′-(trifluoromethyl)-1,1′-biphenyl-2-carboxamide was obtained in the same manner as in Preparation 20 as a pale brown oil.

¹H-NMR (DMSO-d₆): δ2.75-2.9 (2H, m), 2.9-3.1 (2H, m), 7.16 (2H, d, J=8.3 Hz), 7.48 (2H, d, J=8.3 Hz), 7.5-7.8 (1H, m), 10.38 (1H, s)

APCI-MS (m/z): 385 (M+H)⁺

### EXAMPLE 390

N-{2-[4-({[4′-(trifluoromethyl)-1,1′-biphenyl-2-yl}carbonyl}amino)phenyl]ethyl}-2-pyridinecarboxamide was obtained in the same manner as in Example 53 as a white powder.

¹H-NMR (DMSO-d₆): δ2.80 (2H, d, J=7.7 Hz), 3.51 (2H, d, J=7.7 Hz), 7.15 (2H d, J=8.4 Hz), 7.44 (2H, d, J=8.4 Hz), 7.5-7.8 (9H, m), 7.9-8.1 (2H, m), 8.55-8.65 (1H, m), 8.79 (1H, t, J=6.7 Hz), 10.30 (1H, s)

APCI-MS (m/z): 490 (M+H)⁺

### EXAMPLES 391-455

Loading of 4-nitro-N-(2-(2-pyridinyl)ethyl)aniline to Wang Resin

Wang Resin (Nova01-64-0105, 2% DVB; 0.63 mmol/g; 10.0 g, 6.3 mmol) was treated with 1,1-carbonyldiimidazole (10 eq, 10.2 g, 63.0 mmol) and pyridine (5.1 ml, 63.0 mol) in N-methyl-2-pyrrolidone (NMP) (100 ml). After stirring at 50° C. for 1 hour, the resin was filtered and washed with NMP three times. The resultant resin was diluted in NMP (100 ml) and treated with 4-nitro-N-(2-(2-pyridinyl)ethyl)aniline (15.3 g, 63.0 mmol) and 4,4-dimethylaminopyridine (15.3 g, 63.0 mmol) in NMP (100 ml) at 50° C. for an hour. The resin was filtered, washed with NMP, methanol (MeOH), and dichloromethane (DCM), successively, and dried in vacuo.

Loading Check: The dried resin (100 mg) was swollen with 1,2-dichloroethane (DCE) (200 ml) and treated with trifluoroacetic acid (TFA)/water (95/5, 500 ml) at 50° C. for 1 hour to give 15.82 mg (0.0335 mmol) of crude starting material. Purification by preparative HPLC gave 10.21 mg (0.0216 mmol) of the pure starting amine. M.W.: 243.26 (free); 471.31 (2TEA) Loading Level=0.22−0.33 mmol/g

Reduction of Nitro Group and Reaction of the Subsequent Aniline with 2-iodobenzoic Acid

The resin (2500 mg, 0.63 mmol) was treated with a solution of SnCl₂ (10 eq, 6.3 mmol, 1.42 g) in NMP/ethanol (EtOH) (12.5 ml/5 ml) and the mixture was shaked at 50° C. for 3 hours. The resin was filtered and washed with NMP, MeOH, and DCM, successively. The resin was suspended in NMP (10 ml) and the suspension was treated with a solution of 2-iodobenzoic acid (5 eq, 781 mg, 3.15 mmol) and O-(7-azabenzotriazol-1-yl)-N,N,N′,N′-tetramethyluronium hexafluorophosphate (5 eq, 1.20 g) in NMP (10 ml). The resin was filtered, washed with NMP, MeOH, and DCM, succsesively, and dried in vacuo.

Typical Procedure for Suzuki Coupling and Acid Treatment

To a mixture of the resin (50 mg, 0.0315 mmol), 1,1′-bis(diphenylphosphino)ferrocenedichloropalladium(II) (5.0 mg, 0.0063 mmol), and ArB(OH)₂ (0.1575 mmol) were added triethylamine (44 ml, 0.315 mmol) and N,N-dimethylformamide (500 ml). After shaking at 70° C. for 24 hours, the resin was filtered, washed with NMP, MeOH, and DCM, succsesively.

The washed resin was treated with 150 ml of DCE and 300 ml of TFA/H₂O (95/5) at 50° C. for 1 hour. Purification by preparative HPLC gave 5.4 mg of the desired product.

As ArB(OH)₂, a compound of the following formula:

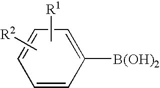

was used.

The following compounds were obtained according to the above-mentioned method.

| | IUPAC name | M.W. |
|---|---|---|
| Ex. 391 | 4′-bromo-N-(4-{[2-(2-pyridinyl)ethyl]amino}ph nyl)-1,1′-biphenyl-2- | 472 |
| | carboxamide | |
| Ex. 392 | 4′-methoxy-N-(4-((2-(2-pyridinyl)ethy)amino)phenyl)-1,1′-biphenyl-2- | 424 |
| | carboxamide | |
| Ex. 393 | 4′-(ethylthio)-N-(4-{[2-(2-pyridinyl)ethyl]amino}phenyl)-1,1′-biphenyl-2- | 454 |
| | carboxamide | |
| Ex. 394 | 3′-methyl-N-(4-{[2-(2-pyridinyl)ethyl}amino)phenyl)-1,1′-biphenyl-2- | 408 |
| | carboxamide | |
| Ex. 395 | N-(4-{[2-(2-pyridinyl)ethyl]amino}phenyl)-3′-(trifluoromethyl)-1,1′- | 461 |
| | biphenyl-2-carboxamide | |
| Ex. 396 | 3′-isopropyl-N-(4-{[2-(2-pyridinyl)ethyl]amino}phenyl)-1,1′-biphenyl-2- | 436 |
| | carboxamide | |
| Ex. 397 | 3′-formyl-N-(4-{[2-(2-pyridinyl)ethyl]amino)phenyl)-1,1-biphenyl-2- | 421 |
| | carboxamide | |
| Ex. 398 | 3′-acetyl-N-(4-{[2-(2-pyridinyl)ethyl]amino)phenyl)-1,1′-biphenyl-2- | 436 |
| | carboxamide | |
| Ex. 399 | N-(4-{[2-(2-pyridinyl)ethyl]amino}phenyl)-1,1′:3′,1″-terphenyl-2- | 470 |
| | carboxamide | |
| Ex. 400 | 3′-chloro-N-(4-{[2-(2-pyridinyl)ethyl]amino}phenyl)-1,1′-biphenyl-2- | 428 |
| | carboxamide | |
| Ex. 401 | 3′-bromo-N-(4-{[2-(2-pyridinyl)ethyl)amino}phenyl)-1,1′-biphenyl-2- | 472 |
| | carboxamide | |
| Ex. 402 | 3′-methoxy-N-(4-{[2-(2-pyridinyl)ethyl]amino}phenyl)-1,1′-biphenyl,2- | 424 |
| | carboxamide | |
| Ex. 403 | 3′-(acetylamino)-N-(4-{[2-(2-pyridinyl)ethyl]amino}phenyl)-1,1′-biphenyl- | 451 |
| | 2-carboxamide | |
| Ex. 404 | 3′-nitro-N-(4-{[2-(2-pyridinyl)ethyl]amino)phenyl)-1,1′-biphenyl-2- | 438 |
| | carboxamide | |
| Ex. 405 | 2′-methyl-N-(4-((2-(2-pyridinyl)ethyl)amino)phenyl)-1,1′-biphenyl-2- | 408 |
| | carboxamide | |
| Ex. 406 | 2′-chloro-N-(4-{[2-(2-pyridinyl)ethyl]amino}phenyl)-1,1′-biphenyl-2- | 428 |
| | carboxamide | |
| Ex. 407 | N-(4-{[2-(2-pyridinyl)ethyl]amino)phenyl)-2′-(trifluoromethyl)-1,1′- | 461 |
| | biphenyl-2-carboxamide | |
| Ex. 408 | 2′-methoxy-N-(4-{[2-(2-pyridinyl)ethyl]amino}phenyl)-1,1′-biphenyl-2- | 424 |
| | carboxamide | |
| Ex. 409 | 2′-(methylthio)-N-(4-{[2-(2-pyridinyl)ethyl]amino)phenyl)-1,1′-biphenyl- | 440 |
| | 2-carboxamide | |
| Ex. 410 | 4′-ethyl-N-(4-{[2-(2-pyridinyl)ethyl]amino}phenyl)-1,1′-biphenyl-2- | 422 |
| | carboxamide | |
| Ex. 411 | 4′-isopropyl-N-(4-{[2-(2-pyridinyl)ethyl]amino)phenyl)-1,1′-biphenyl-2- | 436 |
| | carboxamide | |
| Ex. 412 | 4′-tert-butyl-N-(4-{[2-(2-pyridinyl)ethyl]amino)phenyl)-1,1′-biphenyl-2- | 450 |
| | carboxamide | |
| Ex. 413 | N-(4-{[2-(2-pyridinyl)ethyl]amino}phenyl)-1,1′:4′,1″-terphenyl-2- | 470 |
| | carboxamide | |
| Ex.414 | 4′-formyl-N-(4-{[2-(2-pyridinyl)ethyl]amino}phenyl)-1,1′-biphenyl-2- | 421 |
| | carboxamide | |
| Ex. 415 | 3-(2′-{[(4-{[2-(2-pyridinyl)ethyl]amino}phenyl)amino]carbonyl}-1,1′- | 466 |
| | biphenyl-4-yl)propanoic acid | |
| Ex. 416 | 2′-{[(4-{[2-(2-pyridinyl)ethyl]amino)phenyl)amino]carbonyl}-1,1′- | 437 |
| | biphenyl-4-carboxylic acid | |
| Ex. 417 | 4′-acetyl-N-(4-{[2-(2-pyridinyl)ethyl]amino}phenyl)-1,1′-biphenyl-2- | 436 |
| | carboxamide | |
| Ex. 418 | 2′-formyl-N-(4-{[2-(2-pyridinyl)ethyl]amino}phenyl)-1,1′-biphenyl-2- | 421 |
| | carboxamide | |
| Ex. 419 | 2′,4′-dichloro-N-(4-{[2-(2-pyridinyl)ethyl]amino}phenyl)-1,1′-biphenyl-2- | 462 |
| | carboxamide | |
| Ex. 420 | 2′-chloro-N-(4-{[2-(2-pyridinyl)ethyl]amino}phenyl)-5′-(trifluoromethyl)- | 496 |
| | 1,1′-biphenyl-2-carboxamide | |
| Ex. 421 | 5′-chloro-2′-methyl-N-(4-{[2-(2-pyridinyl)ethyl]amino}phenyl)1,1′- | 442 |
| | biphenyl-2-carboxamide | |
| Ex. 422 | 2′,5′-difluoro-N-(4-{[2-(2-pyridinyl)ethyl]amino}phenyl)-1,1′-biphenyl-2- | 429 |
| | carboxamide | |
| Ex. 423 | 2′-chloro-5′methyl-N-(4-{[2-(-pyridinyl)ethyl]amino}phenyl)-1-1′- | 442 |
| | biphenyl-2-carboxamide | |
| Ex. 424 | 5′-isopropyl-2′-methoxy-N-(4-{[2-(2-pyridinyl)ethyl]amino}phenyl)-1,1′- | 466 |
| | biphenyl-2-carboxamide | |
| Ex. 425 | 5′-chloro-2′-methoxy-N-(4-{[2-(2-pyridinyl)ethyl]amino}phenyl)-1,1′- | 458 |
| | biphenyl-2-carboxamide | |
| Ex. 426 | 4′-methyl-3′-nitro-N-(4-{[2-(2-pyridinyl)ethyl]amino}phenyl)-1,1′- | 453 |
| | biphenyl-2-carboxamide | |
| Ex. 427 | 3′,4′-dimethoxy-N-(4-{[2-(2-pyridinyl)ethyl]amino}phenyl)1,1′-biphenyl- | 454 |
| | 2-carboxamide | |
| Ex. 428 | 3′-fluoro-N-(4-{[2-(2-pyridinyl)ethyl]amino}phenyl)1,1′:4′,1″-terphenyl | 488 |
| | 2-carboxamide | |
| Ex. 429 | 3′,4′-dimethyl-N-(4-{[2-(2-pyridinyl)ethyl]amino}phenyl)-1,1′-biphenyl-2- | 422 |
| | carboxamide | |
| Ex. 430 | 2-(1,3-benzodioxol-5-yl)-N-(4-{[2-(2- | 437 |
| | pyridinyl)ethyl]amino}phenyl)benzamide | |
| Ex. 431 | 2′,3′-dichloro-N-(4-{[2-(2-pyridinyl)ethyl]amino}phenyl)-1,1′-biphenyl-2- | 462 |
| | carboxamide | |
| Ex. 432 | 4′-bromo-2′-fluoro-N-(4-{[2-(2-pyridinyl)ethyl]amino}phenyl)-1,1′- | 490 |
| | biphenyl-2-carboxamide | |
| Ex. 433 | 2′-ethoxy-N-(4-{[2-(2-pyridinyl)ethyl]amino}phenyl)-1,1′-biphenyl-2- | 438 |
| | carboxamide | |
| Ex. 434 | 2′-acetyl-N-(4-{[2-(2-pyridinyl)ethyl]amino}phenyl)-1,1′-biphenyl-2- | 436 |
| | carboxamide | |
| Ex. 435 | 2′-bromo-N-(4-{[2-(2-pyridinyl)ethyl]amino}phenyl)-1,1′-biphenyl-2- | 472 |
| | carboxamide | |
| Ex. 436 | 3′-(hydroxymethyl)-N-(4-{[2-(2-pyridinyl)ethyl[amino}phenyl)-1,1′- | 424 |
| | biphenyl-2-carboxamide | |
| Ex. 437 | 2′-{[(4-{[2-(2-pyridinyl)ethyl]amino}phenyl)amino]carbonyl}-1,1′- | 437 |
| | biphenyl-3-carboxylic acid | |
| Ex. 438 | 3′-fluoro-N-(4-{[2-(2-pyridinyl)ethyl]amino}phenyl)-1,1′-biphenyl-2- | 411 |
| | carboxamide | |
| Ex. 439 | N-(4-{[2-(2-pyridinyl)ethyl]amino}phenyl)-3′-(trifluoromethoxy)-1,1′- | 477 |
| | biphenyl-2-carboxamide | |
| Ex. 440 | 3′-cyano-N-(4-{[2-(2-pyridinyl)ethyl]amino}phenyl)-1-1′-biphenyl-2- | 418 |
| | carboxamide | |
| Ex. 441 | 4′-ethoxy-N-(4-{[2-(2-pyridinyl)ethyl]amino}phenyl)-1,1′-biphenyl-2- | 438 |
| | carboxamide | |
| Ex. 442 | 4′-phenoxy-N-(4-{[2-(2-pyridinyl)ethyl]amino}phenyl)-1,1′-biphenyl-2- | 486 |
| | carboxamide | |
| Ex. 443 | 4′-(hydroxymethyl)-N-(4-{[2-(2-pyridinyl)ethyl]amino}phenyl)-1,1′- | 424 |
| | biphenyl-2-carboxamide | |
| Ex. 444 | 2′,3′-dimethyl-N-(4-{[2-(2-pyridinyl)ethyl]amino}phenyl)-1,1′-biphenyl-2- | 422 |
| | carboxamide | |
| Ex. 445 | 5′-fluoro-2′-methoxy-N-(4-{[2-(2-pyridinyl)ethyl]amino}phenyl)-1,1′- | 442 |
| | biphenyl-2-carboxamide | |
| Ex. 446 | 2′,5′-dimethyl-N-(4-{[2-(2-pyridinyl)ethyl]amino}phenyl)-1,1′-biphenyl-2- | 422 |
| | carboxamide | |
| Ex. 447 | 2′,5′-dimethoxy-N-(4-{[2-(2-pyridinyl)ethyl]amino}phenyl)1-1′-biphenyl- | 454 |
| | 2-carboxamide | |
| Ex. 448 | 2′,6′-difluoro-N-(4-{[2-(2-pyridinyl)ethyl]amino}phenyl)-1,1′-biphenyl-2- | 429 |
| | carboxamide | |
| Ex. 449 | 3′,4′-dichloro-N-(4-{[2-(2-pyridinyl)ethyl]amino}phenyl)-1,1′-biphenyl-2- | 462 |
| | carboxamide | |
| Ex. 450 | 4′-fluoro-3′-methyl-N-(4-{[2-(2-pyridinyl)ethyl]amino}phenyl)-1-1′- | 426 |
| | biphenyl-2-carboxamide | |
| Ex. 451 | 3′,4′-difluoro-N-(4-{[2-(2-pyridinyl)ethyl]amino}phenyl)-1-1′-biphenyl-2- | 429 |
| | carboxamide | |
| Ex. 452 | 3′-formyl-4′-methoxy-N-(4-{[2-(2-pyridinyl)ethyl]amino}phenyl)-1,1′- | 452 |
| | biphenyl-2-carboxamide | |
| Ex. 453 | 3′,5′-dibromo-N-(4-{[2-(2-pyridinyl)ethyl]amino}phenyl)-1-1′-biphenyl-2- | 551 |
| | carboxamide | |
| Ex. 454 | 3′,5′-dimethyl-N-(4-{[2-(2-pyridinyl)ethyl]amino}phenyl)-1,1′-biphenyl-2- | 422 |
| | carboxamide | |
| Ex. 455 | N-(4-{[2-(2-pyridinyl)ethyl]amino}phenyl)-4′-vinyl-1,1′-biphenyl-2- | 420 |
| | carboxamide | |

This application is based on application No. PR 0583 filed in Australia on Oct. 5, 2000, and application No. PR 6666 filed in Australia on Jul. 27, 2001, the content of which is incorporated hereinto by reference.

1. A compound of the formula (I) 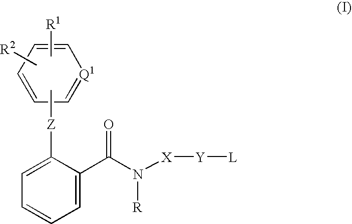
wherein
Q¹ is N or CH;
R¹ and R² are each independently lower alkyl, lower alkenyl, acyl, amino, lower alkoxy, lower cycloalkyloxy, aryl, aryloxy, sulfooxy, mercapto or sulfo, each of which is optionally substituted by suitable substituent(s), hydrogen, halogen, nitro, cyano or hydroxy, or
R¹ and R² together may form a ring structure,
L is unsaturated 3 to 10-membered heterocyclic group, which is optionally substituted by suitable substituent(s);
X is monocyclic arylene or monocyclic heteroarylene, each of which is optionally substituted by suitable substituent(s);
Y is -(A¹)ₘ-(A²)ₙ-(A⁴)ₖ- in which
A¹ is lower alkylene or lower alkenylene, each of which is optionally substituted by suitable substituent(s),
A² is —N(R³)—, —CO—N(R³)—, —NH—CO—NH—, —CO—O—, —O—, —O—(CH₂)₂—N (R³)—, —S—, —SO— or —SO₂—, wherein R³ is hydrogen or suitable substituent(s),
A⁴ is lower alkylene, lower alkenylene or lower alkynylene, and
k, m and n are each independently 0 or 1;
Z is direct bond, —CH₂—, —NH— or —O—; and
R is hydrogen or lower alkyl,
or a salt thereof.

2. The compound of claim 1 wherein
R¹ and R² are each independently hydrogen, lower alkyl, lower alkenyl, hydroxy(lower)alkyl, lower alkanoyl, carboxy(lower)alkyl, optionally protected carboxy, lower alkylthio, lower alkylsulfonyl, halogen, trihalo(lower)alkyl, cyano, nitro, aryl, —N(R¹²)(R¹³) (wherein R¹² and R¹³ are each independently hydrogen, lower alkyl or amino protective group), hydroxy, aryloxy, lower alkylsulfonyloxy, arylsulfonyloxy, lower cycloalkyloxy, or lower alkoxy which is optionally substituted by suitable substituent(s), or
R¹ and R² together may form 1,3-dioxole,
L is pyridinyl, N-oxidopyridinyl, pyrimidinyl, pyrazinyl, thiazolyl, quinolinyl, isoquinolinyl, pyrazolyl, imidazolyl or benzimidazolyl, each of which is optionally substituted by suitable substituent(s) selected from the group consisting of lower alkyl, aryl(lower)alkyl and —(CH₂)ₛ—N(R¹⁴) (R¹⁵) (wherein R¹⁴ and R¹⁵ are each independently hydrogen, lower alkyl or amino protective group and s is 0 or 1);
X is 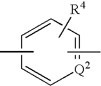
in which
Q² is N or CH, and
R⁴ is hydrogen, lower alkyl, lower alkoxy, lower alkanoyl, nitro, optionally protected amino or halogen; and
Y is -(A¹)ₘ-(A²)ₙ-(A⁴)ₖ- in which
A¹ is lower alkylene or lower alkenylene, each of which is optionally substituted by oxo, hydroxy, hydroxy(lower)alkyl, optionally protected carboxy or optionally protected amino,
A² is —N (R³)—, —CO—N(R³)—, —NH—CO—NH—, —CO—O—, —O—, —O—(CH₂)₂—N(R³)—, —S—, —SO— or SO₂—, wherein R³ is hydrogen, lower alkyl, pyridinyl(lower)alkyl or amino protective group,
A⁴ is lower alkylene, lower alkenylene or lower alkynylene, and
k, m and n are each independently 0 or 1,
or a salt thereof.

3. The compound of claim 2 wherein
R¹ and R² are each independently hydrogen, lower alkyl, lower alkenyl, hydroxy(lower)alkyl, lower alkanoyl, carboxy(lower)alkyl, carboxy, lower alkoxycarbonyl, lower alkylthio, lower alkylsulfonyl, halogen, trihalo(lower)alkyl, cyano, nitro, phenyl, amino, di(lower)alkylamino, lower alkanoylamino, lower alkylsulfonylamino, aryl(lower)alkylsulfonylamino, (lower) alkoxycarbonylamino, bis[(lower) alkylsulfonyl]amino, bis[aryl(lower)alkylsulfonyl]amino, hydroxy, phenyloxy, lower alkylsulfonyloxy, tolylsulfonyloxy, lower cycloalkyloxy or lower alkoxy which is optionally substituted by suitable substituent(s) selected from the group consisting of lower alkoxy, lower alkoxycarbonyl, carboxy, halogen, hydroxy, phenyl, di(lower)alkylamino and optionally substituted carbamoyl, or
R¹ and R² together may form 1,3-dioxole,
or a salt thereof.

4. The compound of claim 3 wherein
R¹ and R² are each independently hydrogen, methyl, ethyl, isopropyl, tert-butyl, vinyl, hydroxymethyl, hydroxyethyl, hydroxypropyl, formyl, acetyl, carboxymethyl, carboxyethyl, carboxy, methoxycarbonyl, methylthio, ethylthio, isopropylthio, methylsulfonyl, isopropylsulfonyl, fluoro, chloro, iodo, bromo, trifluioromethyl, cyano, nitro, phenyl, amino, dimethylamino, acetylamino, methylsulfonylamino, benzylsulfonylamino, methoxycarbonylamino, bis(methylsulfonyl)amino, bis(benzylsulfonyl)amino, hydroxy, methylsulfonyloxy, tolylsulfonyloxy, cyclohexyloxy, methoxy, ethoxy, isopropoxy, methoxyethoxy, ethoxycarbonylmethoxy, carboxymethoxy, trigluoromethoxy, trifluoroethoxy, tetrafluoropropoxy, hydroxyethoxy, phenyloxy, benzyloxy, dimethylaminoethoxy, dimethylaminopropoxy, carbamoylmethoxy, methylcarbamoylmethoxy, phenylcarbamoylmethoxy, methylsulfonylcarbamoylmethoxy or phenylsulfonylcarbamoylmethoxy, or
R¹ and R² together may form 1,3-dioxole;
L is pyridinyl, N-oxidopyridinyl, pyrimidinyl, pyrazinyl, thiazolyl, quinolinyl, isoquinolinyl, pyrazolyl, imidazolyl or benzimidazolyl, each of which is optionally substituted by methyl, ethyl, amino, methylamino, formylamino, acetylamino, tert-butoxycarbonylamino, N-(tert-butoxycarbonyl)-N-methylamino, trityl, dimethylpyrrolyl or acetylaminomethyl;
X is 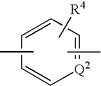
in which
Q² is N or CH, and
R⁴ is hydrogen, methyl, methoxy, nitro, amino, acetyl, acetylamino, fluoro, chloro or bromo; and
Y is direct bond or bivalent residue selected from the group consisting of 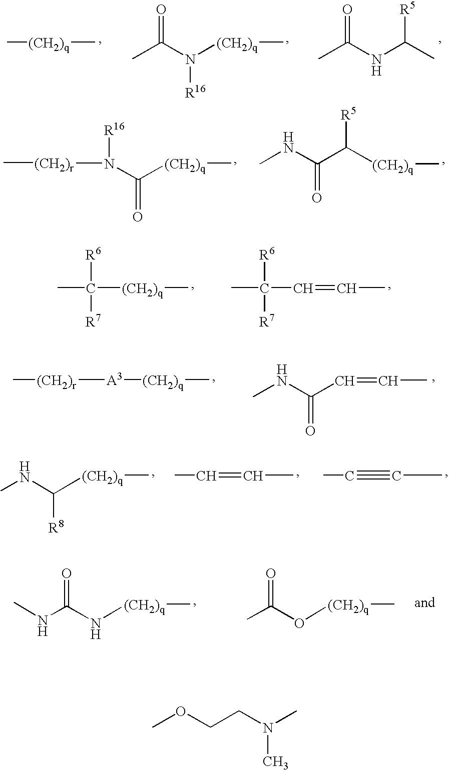
in which
A³ is —NH—, —N (CH₃)—, N(CHO)—, —N(CH₃CO)—, —N(Boc)-, 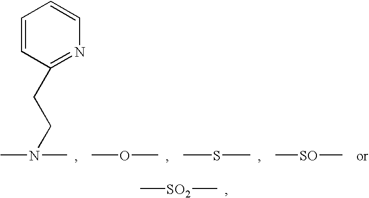
tert-butoxycarbonyl,
R⁵ is methyl, amino, acetylamino or tert-butoxycarbonylamino,
R⁶ is hydroxy,
R⁷ is hydrogen, or
R⁶ and R⁷, together with the carbon atom to which they are bonded, form carbonyl,
R⁸ is hydroxymethyl or ethoxycarbonyl,
R¹⁶ is hydrogen or methyl, and
q and r are independently an integer of 0 to 3,
or a salt thereof.

5. A compound of the formula (II) 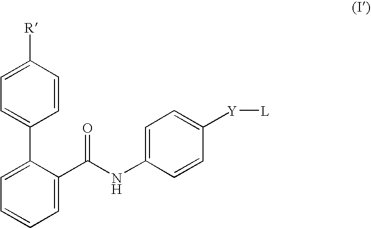
wherein
R′ is methyl or trifluoromethyl;
Y is —CH₂—, —(CH₂)₂—, —(CH₂)₃—, —NH—(CH₂)₂—, —O—(CH₂)₂—, —NH—CO—CH₂—, —CO—NH—CH₂— or —CO—NH—(CH₂)₂—; and
L is pyridinyl or thiazolyl, each of which is optionally substituted by methyl or amino,
or a salt thereof.

6. The compound of claim 5, wherein
Y is —(CH₂)₃—, —NH—(CH₂)₂—, —O—(CH₂)₂—, —NH—CO—CH₂— or —CO—NH—CH₂—; and
L is pyridinyl aminopyridinyl, thiazolyl or aminothiazolyl,
or a salt thereof.

7. The compound of claim 6, which is selected from the group consisting of
N-{4-[3-(2-pyridinyl)propyl]phenyl}-4′-(trifluoromethyl)-1,1′-biphenyl-2-carboxamide,
N-{4-[3-(6-amino-2-pyridinyl)propyl]phenyl}-4′-(trifluoromethyl))-1,1′-biphenyl-2-carboxamide,
N-[4-({[4′-(trifluoromethyl)-1,1′-biphenyl-2-yl]carbonyl}amino)benzyl]-2-pyridinecarboxamide,
N-(4-{[(4′-methyl-1,1′-biphenyl-2-yl)carbonyl]amino}benzyl)-2-pyridinecarboxamide,
N-(4-{[2-(2-pyridinyl)ethyl]amino}phenyl)-4′-(trifluoromethyl)-1,1′-biphenyl-2-carboxamide,
N-{4-[(2-pyridinylacetyl)amino]phenyl]-4′-(trifluoromethyl)-1,1′-biphenyl-2-carboxamide,
4′-methyl-N-(4-([2-(2-pyridinyl)ethyl]amino}phenyl)-1,1′-biphenyl-2-carboxamide,
N-{4-[2-(2-pyridinyl)ethoxy]phenyl}-4′-(trifluoromethyl)-1,1′-biphenyl-2-carboxamide,
N-(4-([2-(2-amino-1,3-thiazol-4-yl)ethyl]amino}phenyl)-4′-(trifluoromethyl)-1,1′-biphenyl-2-carboxamide,
N-(4-{[2-(6-amino-2-pyridinyl)-ethyl]amino}phenyl)-4′-(trifluoromethyl)-1,1′-biphenyl-2-carboxamide,
N-{4-[2-(2-amino-1,3-thiazol-4-yl)ethoxy]phenyl}-4′-(trifluoromethyl)-1,1′-biphenyl-2-carboxamide,
N-{4-[2-(6-amino-2-pyridinyl)ethoxy]phenyl}-4′-(trifluoromethyl)-1,1′-biphenyl-2-carboxamide,
N-(4-{[2-(1,3-thiazol-4-yl)ethyl]amino}phenyl)-4′-(trifluoromethyl)-1,1′-biphenyl-2-carboxamide,
N-(4-{[(6-amino-2-pyridinyl)acetyl]amino}phenyl)-4′-(trifluoromethyl)-1,1′-biphenyl-2-carboxamide,
N-(4-{[2-(6-amino-2-pyridinyl)ethyl]amino}phenyl)-4′-methyl-1,1′-biphenyl-2-carboxamide,
N-(4-{[(2-amino-1,3-thiazol-4-yl)acetyl]amino}phenyl)-4′-(trifluoromethyl)-1,1′-biphenyl-2-carboxamide,
N-{4-[(1,3-thiazol-4-ylacetyl)amino]phenyl}-4′-(trifluoromethyl)-1,1′-biphenyl-2-carboxamide,
N-(4-{[2-(2-amino-1,3-thiazol-4-yl)ethyl]amino}phenyl)-4′-methyl-1,1′-biphenyl-2-carboxamide, and
N-{4-[2-(1,3-thiazol-4-yl)thoxy]phenyl}-4′-(trifluoromethyl)-1,1′-biphenyl-2-carboxamide, or a salt thereof.

8. The compound of claim 1 or a pharmaceutically acceptable salt thereof for use as a medicament.

9. A pharmaceutical composition comprising a compound of claim 1 or a pharmaceutically acceptable salt thereof in admixture with a pharmaceutically acceptable carrier.

10. Use of a compound of claim 1 or a pharmaceutically acceptable salt thereof for preparing a medicament as an apolipoprotein B (Apo B) secretion inhibitor.

11. Use of a compound of claim 1 or a pharmaceutically acceptable salt thereof for preparing a medicament for the prophylaxis or treatment of a disease or condition resulting from elevated circulating levels of Apo B.

12. Use of a compound of claim 1 or a pharmaceutically acceptable salt-thereof for preparing a medicament for the prophylaxis or treatment of hyperlipemia, hyperlipidemia, hyperlipoproteinemia, hypoalphalipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis or Syndrome X.

13. A method for inhibiting or decreasing Apo B secretion in a mammal, which comprises administering an Apo B secretion inhibiting or decreasing amount of a compound of claim 1 or a pharmaceutically acceptable salt thereof to the mammal.

14. A method for preventing or treating a disease or condition resulting from elevated circulating levels of Apo B in a mammal, which comprises administering an effective amount of a compound of claim 1 or a pharmaceutically acceptable salt thereof to the mammal.

15. The method of claim 14 wherein the disease or condition resulting from the elevated circulating levels of Apo B is selected from the group consisting of hyperlipemia, hyperlipidemia, hyperlipoproteinemia, hypoalphalipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis and Syndrome X.
